(19) United States
(12) Patent Application Publication
(10) Pub. No.: US 2024/0051936 A1
(43) Pub. Date: Feb. 15, 2024

(54) ISOINDOLINONE COMPOUNDS

(71) Applicant: Monte Rosa Therapeutics, Inc., Boston, MA (US)

(72) Inventors: Bernhard Fasching, Basel (CH); Thomas Ryckmans, Basel (CH); Alexander Flohr, Basel (CH)

(21) Appl. No.: 18/485,766

(22) Filed: Oct. 12, 2023

Related U.S. Application Data
(63) Continuation of application No. PCT/IB2022/000215, filed on Apr. 14, 2022.

(30) Foreign Application Priority Data
Apr. 14, 2021 (CH) 00385/21

(51) Int. Cl. C07D 401/04; C07D 401/14; C07D 405/14; A61P 35/00

(52) U.S. Cl. CPC C07D 401/04; C07D 401/14; C07D 405/14; A61P 35/00

(57) ABSTRACT

Disclosed herein are compound or pharmaceutically acceptable salts or stereoisomers thereof of formula I:

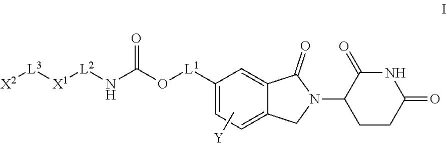

wherein X¹ is selected from the group consisting of linear or branched C₁₋₆ alkyl, C₃₋₆ cycloalkyl, C₆₋₁₀ aryl, 5-10 membered heteroaryl, and 4-8 membered heterocycloalkyl, wherein X¹ is unsubstituted or substituted with one or more substituents independently selected from the group consisting of halogen, linear or branched C₁₋₆ alkyl, linear or branched C₁₋₆ heteroalkyl, CF₃, CHF₂, CMeF₂, —O—CHF₂, —O—(CH₂)₂—OMe, OCF₃, C₁₋₆ alkylamino, —CN, NH₂, C₁₋₄ alkoxy and C₁₋₄ alkylhydroxy; X² is selected from the group consisting of H, C₃₋₆ cycloalkyl, C₆₋₁₀ aryl, 5-10 membered heteroaryl, and 4-8 membered heterocycloalkyl, wherein X² is unsubstituted or substituted with one or more substituents independently selected from the group consisting of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, and C₁₋₄ alkylhydroxy; Y is selected from the group consisting of linear or branched C₁₋₆ alkyl, —C₁₋₄ alkoxy, —CN, halogen, CF₃, CHF₂, CMeF₂, OCF₃, and OCHF₂; L¹ is linear or branched C₁₋₆ alkyl; L² is selected from a covalent bond, and linear or branched C₁₋₆ alkyl; and L³ is selected from the group consisting of a covalent bond, linear or branched C₁₋₆ alkyl, —O—, and —C₁₋₄ alkoxy. Disclosed herein is also their use as modulators of cereblon, methods of preparation of these compounds, compositions comprising these compounds, and methods of using them in the treatment of abnormal cell growth in mammals, especially humans.

### CROSS-REFERENCE

This application is a continuation of International Patent Application No. PCT/IB2022/000215, filed Apr. 14, 2022, which claims priority to Swiss Patent Application Number 00385/21, filed Apr. 14, 2021, the contents of each of which are incorporated herein by reference in its entirety.

### FIELD OF DISCLOSURE

The present disclosure relates to new compounds as modulators of cereblon. The disclosure also relates to methods of preparation of these compounds, compositions comprising these compounds, and methods of using them in the treatment of abnormal cell growth in mammals, especially humans.

### BACKGROUND

The ubiquitin proteasome system can be manipulated with different small molecules to trigger targeted degradation of specific proteins of interest. Promoting the targeted degradation of pathogenic proteins using small molecule degraders is emerging as a new modality in the treatment of diseases. One such modality relies on redirecting the activity of E3 ligases such as cereblon (a phenomenon known as E3 reprogramming) using low molecular weight compounds, which have been termed molecular glues (Tan et al. Nature 2007, 446, 640-645 and Sheard et al. Nature 2010, 468, 400-405) to promote the poly-ubiquitination and ultimately proteasomal degradation of new protein substrates involved in the development of diseases. The molecular glues bind to both the E3 ligase and the target protein, thereby mediating an alteration of the ligase surface and enabling an interaction with the target protein. Particularly relevant compounds for the E3 ligase cereblon are the IMiD (immunomodulatory imide drugs) class including Thalidomide, Lenalidomide and Pomalidomide. These IMiDs have been approved by the FDA for use in hematological cancers. However, compounds for efficiently targeting other diseases, in particular other types of cancers, are still required.

### SUMMARY OF DISCLOSURE

It is therefore an object of the present disclosure to advance the state of the art of cereblon modulators and provide modulators for novel use in different diseases, in particular in different cancers.

In some embodiments, compounds are provided for use in therapy of solid tumors, such as for use in the therapy of lung cancer for example, non-small cell lung cancer (e.g., squamous cell lung cancer) and small cell lung cancer, breast cancer, and neuroendocrine cancer, e.g., neuroendocrine prostate cancer such as castration-resistant neuroendocrine prostate cancer (NEPC) and lung neuroendocrine tumors (Lu-NETs). In some embodiments, compounds are provided for use in therapy of blood-borne (or haematological) cancers such as for use in the therapy of leukemias (e.g. acute myelogenous leukemia (AML)) and myelomas (e.g. multiple myeloma (MM)).

The present disclosure is in a first aspect directed towards a compound or pharmaceutically acceptable salts or stereoisomers thereof of formula I:

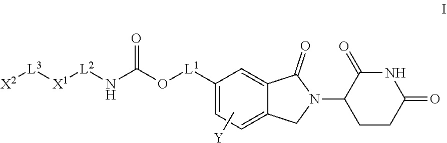

wherein X¹ is linear or branched C₁₋₆ alkyl, C₃₋₆ cycloalkyl, C₆₋₁₀ aryl, 5-10 membered heteroaryl, 4-8 membered heterocycloalkyl, wherein X¹ is unsubstituted or substituted with one or more of halogen, linear or branched C₁₋₆ alkyl, linear or branched C₁₋₆ heteroalkyl, CF₃, CHF₂, CMeF₂, —O—CHF₂, —O—(CH₂)₂—OMe, OCF₃, C₁₋₆ alkylamino, —CN, NH₂, C₁₋₄ alkoxy and C₁₋₄ alkylhydroxy;
X² is H, C₃₋₆ cycloalkyl, C₆₋₁₀ aryl, 5-10 membered heteroaryl, 4-8 membered heterocycloalkyl, wherein X² is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, and C₁₋₄ alkylhydroxy;
Y is linear or branched C₁₋₆ alkyl, —C₁₋₄ alkoxy, —CN, halogen, CF₃, CHF₂, CMeF₂, OCF₃, OCHF₂;
L¹ is linear or branched C₁₋₆ alkyl; L² is a covalent bond, linear or branched C₁₋₆ alkyl; and L³ is a covalent bond, linear or branched C₁₋₆ alkyl, —O—, —C₁₋₄ alkoxy.

In some embodiments of the compounds of the disclosure or pharmaceutically acceptable salts or stereoisomers thereof, Y is in the 4-position or in the 5-position or in the 7-position of the ring.

In some embodiments of the compounds of the disclosure or pharmaceutically acceptable salts or stereoisomers thereof, L¹ is —CH₂—, L² is a covalent bond and L³ is a covalent bond or L¹ is —CH₂—, L² is a covalent bond and L³ is —O—.

In some embodiments of the compounds of the disclosure or pharmaceutically acceptable salts or stereoisomers thereof, X¹ is —C₆₋₁₀ aryl, 5-10 membered heteroaryl, wherein X¹ is unsubstituted or substituted with one or more of linear or branched —C₁₋₄ alkyl, halogen, —CF₃, —CHF₂, —CMeF₂, —O—(CH₂)₂—OMe, —OCF₃, —OCHF₂, C₁₋₆ alkylamino, —CN, —NH₂, —C₁₋₄ alkylhydroxy, and —C₁₋₄ alkoxy.

In some embodiments of the compounds of the disclosure or pharmaceutically acceptable salts or stereoisomers thereof, X² is H, C₃₋₆ cycloalkyl, C₆ aryl, 6-membered heteroaryl, 5-6 membered heterocycloalkyl, wherein X² is unsubstituted or substituted with one or more of linear or branched C₁₋₄ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy.

In some embodiments, the present disclosure is directed towards a compound or pharmaceutically acceptable salts or stereoisomers thereof of the formula III, such as formula IIIa, IIIb or IIIc:

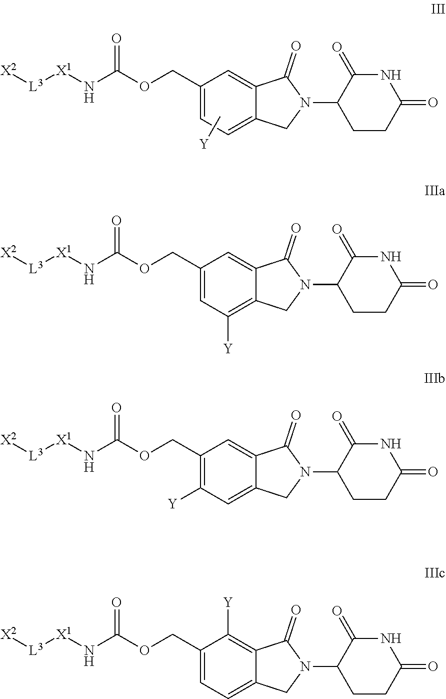

wherein X¹ is linear or branched C₁₋₆ alkyl, C₃₋₆ cycloalkyl, C₆₋₁₀ aryl, 5-10 membered heteroaryl, 4-8 membered heterocycloalkyl, wherein X¹ is unsubstituted or substituted with one or more of halogen, linear or branched C₁₋₆ alkyl, linear or branched C₁₋₆ heteroalkyl, CF₃, CHF₂, CMeF₂, —O—CHF₂, —O—(CH₂)₂—OMe, OCF₃, C₁₋₆ alkylamino, —CN, NH₂, C₁₋₄ alkoxy and C₁₋₄ alkylhydroxy;
X² is H, C₃₋₆ cycloalkyl, C₆₋₁₀ aryl, 5-10 membered heteroaryl, 4-8 membered heterocycloalkyl, wherein X² is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, and C₁₋₄ alkylhydroxy;
Y is linear or branched C₁₋₆ alkyl, —C₁₋₄ alkoxy, —CN, halogen, CF₃, CHF₂, CMeF₂, OCF₃, OCHF₂; and L³ is a covalent bond, linear or branched C₁₋₆ alkyl, —O—, —C₁₋₄ alkoxy.

In some embodiments, the present disclosure is directed towards a compound or pharmaceutically acceptable salts or stereoisomers thereof of the formula IV, such as IVa, IVb or IVc:

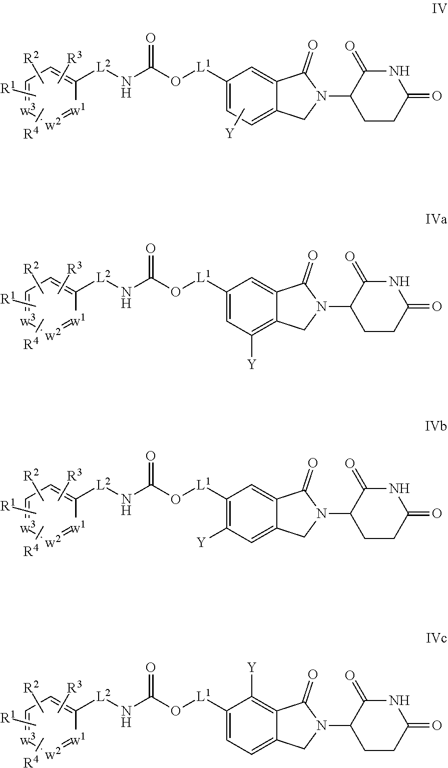

wherein Y is linear or branched C₁₋₄ alkyl, —C₁₋₄ alkoxy, —CN, halogen, CF₃, CHF₂, CMeF₂, OCF₃, OCHF₂;
w¹, w², w³ are independently of each other selected from C, N, with the proviso that two or three of w¹, w², w³ are C;
R¹, R², R³, R⁴ are independently of each other selected from H, linear or branched —C₁₋₆ alkyl, linear or branched C₁₋₆ heteroalkyl, —C₁₋₄ alkoxy, CF₃, CHF₂, CMeF₂, —O—(CH₂)₂—OMe, OCF₃, OCHF₂, —C₁₋₆ alkylamino, —CN, —NH₂, —C₁₋₄ alkylhydroxy, and halogen, such as F, Cl or Br, e.g. F or Cl, or a group of formula -L³-X², wherein L³ is a covalent bond, linear or branched C₁₋₆ alkyl, —O—, and X² is C₃₋₄ cycloalkyl, C₆₋₁₀ aryl, 5-10 membered heteroaryl, 4-8 membered heterocycloalkyl, wherein X² is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, and C₁₋₄ alkylhydroxy;
L¹ is linear or branched C₁₋₆ alkyl; and L² is a covalent bond, linear or branched C₁₋₆ alkyl.

In some embodiments of the compounds of the disclosure or pharmaceutically acceptable salts or stereoisomers thereof, L¹ is —CH₂—. In some embodiments of the compounds of the disclosure or pharmaceutically acceptable salts or stereoisomers thereof, L² is a covalent bond.

In some embodiments of the compounds of the disclosure or pharmaceutically acceptable salts or stereoisomers thereof, w¹, w², w³ are C. In some embodiments of the compounds of the disclosure or pharmaceutically acceptable salts or stereoisomers thereof, w¹ is N and w², w³ are C. In some embodiments of the compounds of the disclosure or pharmaceutically acceptable salts or stereoisomers thereof, w² is N and w¹, w³ are C, or wherein w³ is N and w¹, w² are C.

In some embodiments, the present disclosure is directed towards a compound or pharmaceutically acceptable salts or stereoisomers thereof of the formula XI, such as XIa, XIb or XIc:

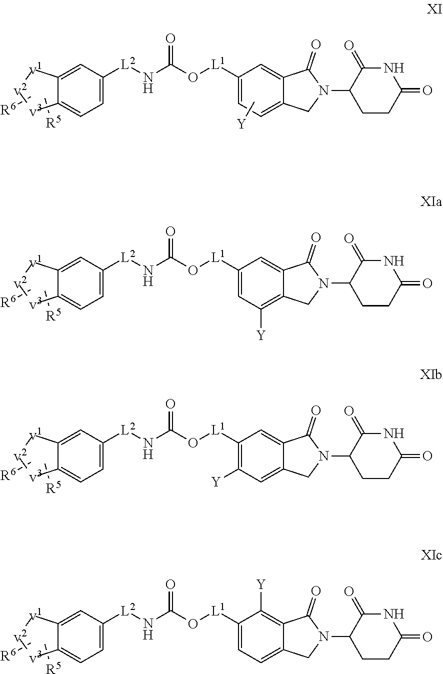

wherein Y is linear or branched C₁₋₆ alkyl, —C₁₋₄ alkoxy, —CN, halogen, CF₃, CHF₂, CMeF₂, OCF₃, OCHF₂;
v¹, v², v³ are independently of each other selected from C, O, with the proviso that at least one of v¹, v², v³ is C;
R⁵, R⁶ are independently of each other selected from H, linear or branched —C₁₋₄ alkyl and halogen, such as F, Cl, e.g. F;
L¹ is linear or branched C₁₋₆ alkyl; and L² is a covalent bond, linear or branched C₁₋₆ alkyl.

In some embodiments, the present disclosure is directed towards a compound or pharmaceutically acceptable salts or stereoisomers thereof of formula XIII, such as XIIIa, XIIIb or XIIIc:

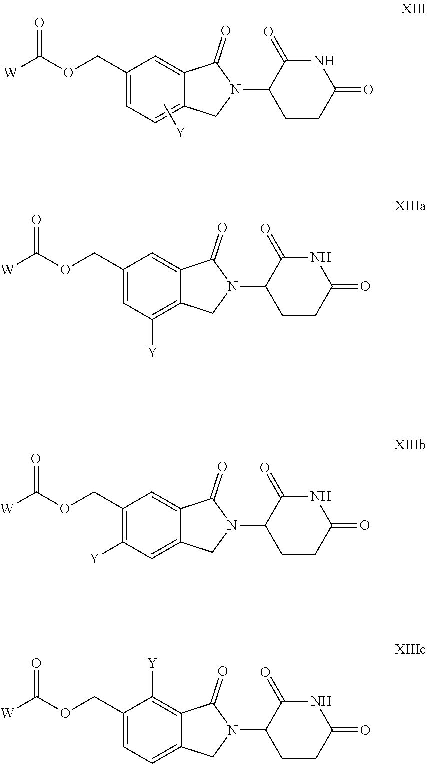

wherein Y is linear or branched C₁₋₆ alkyl, —C₁₋₄ alkoxy, —CN, halogen, CF₃, CHF₂, CMeF₂, OCF₃, OCHF₂, in particular C₁₋₄ alkyl, such as methyl, —C₁₋₄ alkoxy, such as —OMe, —CN, halogen, such as F, Cl, Br; and W is selected from

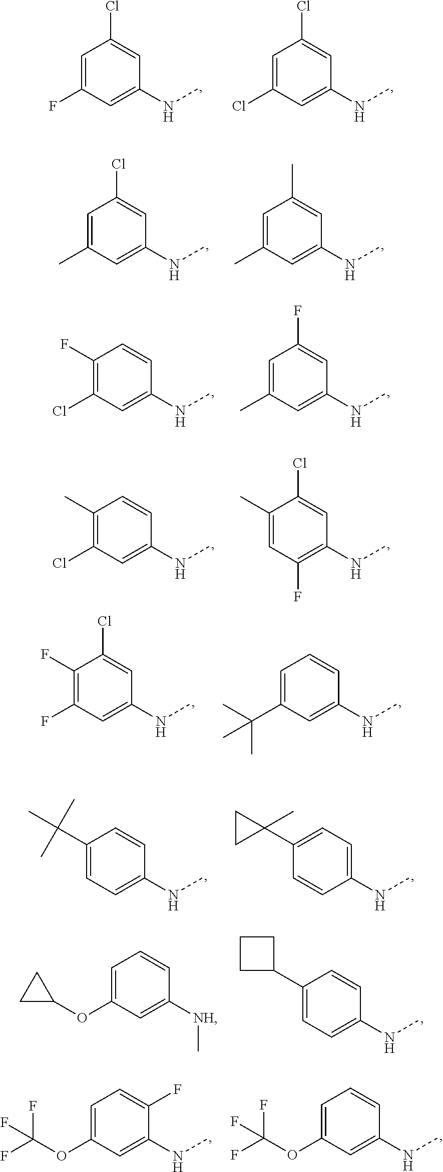

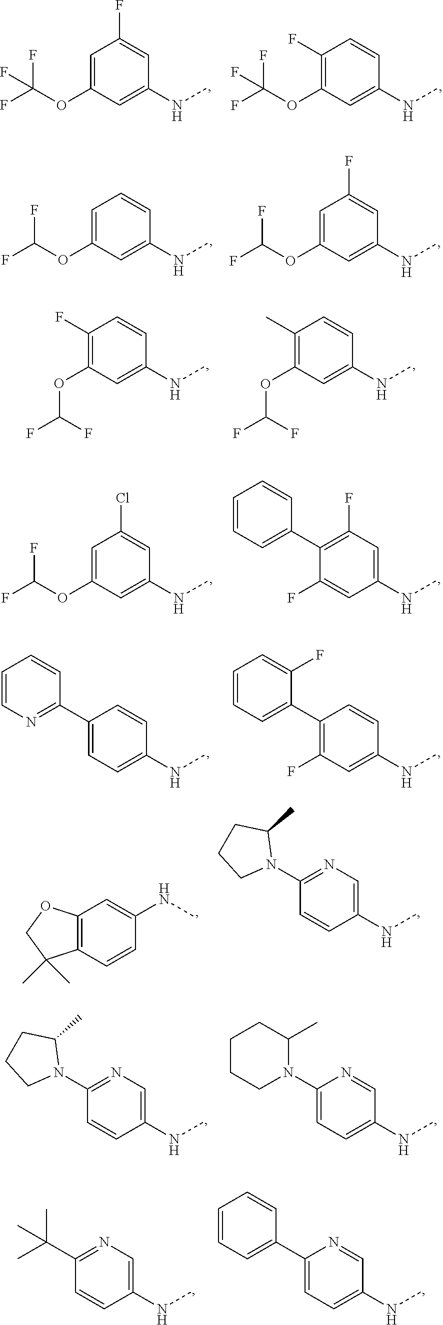

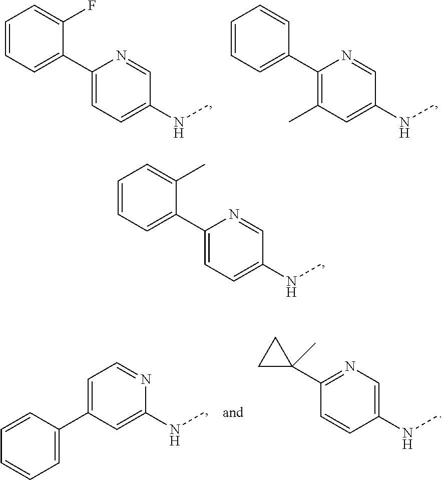

In some embodiments, provided herein is a compound of formula I:

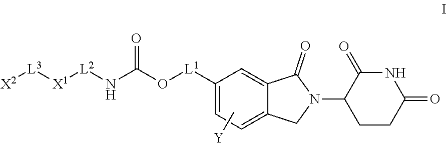

or a pharmaceutically acceptable salt or stereoisomer thereof, wherein
X¹ is selected from the group consisting of linear or branched C₁₋₆ alkyl, C₃₋₄ cycloalkyl, C₆₋₁₀ aryl, 5-10 membered heteroaryl, and 4-8 membered heterocycloalkyl, wherein X¹ is unsubstituted or substituted with one or more substituents independently selected from the group consisting of halogen, linear or branched C₁₋₆ alkyl, linear or branched C₁₋₆ heteroalkyl, CF₃, CHF₂, CMeF₂, —O—CHF₂, —O—(CH₂)₂—OMe, OCF₃, C₁₋₆ alkylamino, —CN, NH₂, C₁₋₄ alkoxy and C₁₋₄ alkylhydroxy;
X² is selected from the group consisting of H, C₃₋₄ cycloalkyl, C₆₋₁₀ aryl, 5-10 membered heteroaryl, and 4-8 membered heterocycloalkyl, wherein X² is unsubstituted or substituted with one or more substituents independently selected from the group consisting of linear or branched C₁₋₄ alkyl, —C₁₋₄ alkoxy, NH₂, NMe₂, halogen, CF₃, CHF₂, CMeF₂, —O—(CH₂)₂—OMe, OCF₃, OCHF₂, and C₁₋₄ alkylhydroxy;
Y is selected from the group consisting of linear or branched C₁₋₄ alkyl, —C₁₋₄ alkoxy, —CN, halogen, CF₃, CHF₂, CMeF₂, OCF₃, and OCHF₂;
L¹ is linear or branched C₁₋₆ alkyl;
L² is selected from a covalent bond, and linear or branched C₁₋₆ alkyl; and
L³ is selected from a covalent bond, linear or branched C₁₋₆ alkyl, —O—, and —C₁₋₄ alkoxy.

In some embodiments, Y is in the 4-position or in the 5-position or in the 7-position of the ring.

In some embodiments, L¹ is —CH₂—, L² is a covalent bond and L³ is a covalent bond or wherein L¹ is —CH₂—, L² is a covalent bond and L³ is —O—.

In some embodiments, X¹ is selected from —C₆₋₁₀ aryl, and 5-10 membered heteroaryl, wherein X¹ is unsubstituted or substituted with one or more substituents independently selected from the group consisting of linear or branched —C₁₋₄ alkyl, halogen, —CF₃, —CHF₂, —CMeF₂, —O—(CH₂)₂—OMe, —OCF₃, —OCHF₂, C₁₋₆ alkylamino, —CN, —NH₂, —C₁₋₄ alkylhydroxy, and —C₁₋₄ alkoxy.

In some embodiments, X² is selected from the group consisting of H, C₃₋₆ cycloalkyl, C₆ aryl, 6-membered heteroaryl, and 5-6 membered heterocycloalkyl, wherein X² is unsubstituted or substituted with one or more substituents independently selected from the group consisting of linear or branched C₁₋₄ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy.

In some embodiments, the compound is of formula II, such as formula IIIa, IIIb or IIIc:

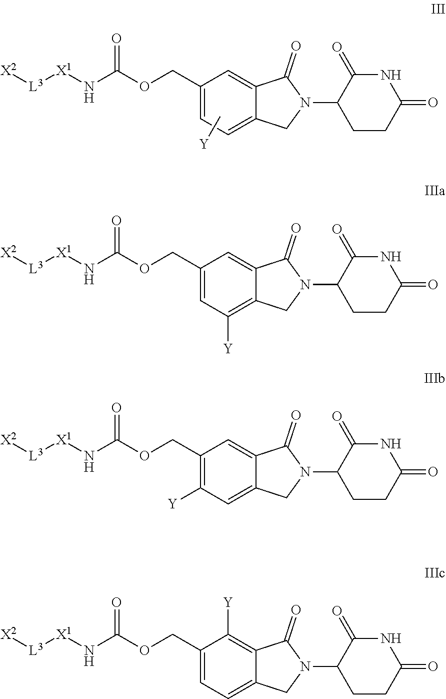

or a pharmaceutically acceptable salt or stereoisomer thereof,
wherein
X¹ is selected from the group consisting of linear or branched C₁₋₆ alkyl, C₃₋₆ cycloalkyl, C₆₋₁₀ aryl, 5-10 membered heteroaryl, and 4-8 membered heterocycloalkyl, wherein X¹ is unsubstituted or substituted with one or more substituents independently selected from the group consisting of halogen, linear or branched C₁₋₆ alkyl, linear or branched C₁₋₆ heteroalkyl, CF₃, CHF₂, CMeF₂, —O—CHF₂, —O—(CH₂)₂—OMe, OCF₃, C₁₋₆ alkylamino, —CN, NH₂, C₁₋₄ alkoxy, and C₁₋₄ alkylhydroxy;
X² is selected from the group consisting of H, C₃₋₆ cycloalkyl, C₆₋₁₀ aryl, 5-10 membered heteroaryl, and 4-8 membered heterocycloalkyl, wherein X² is unsubstituted or substituted with one or more substituents independently selected from the group consisting of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, and C₁₋₄ alkylhydroxy;
Y is selected from the group consisting of linear or branched C₁₋₆ alkyl, —C₁₋₄ alkoxy, —CN, halogen, CF₃, CHF₂, CMeF₂, OCF₃, and OCHF₂; and
L³ is selected from the group consisting of a covalent bond, linear or branched C₁₋₆ alkyl, —O—, and —C₁₋₄ alkoxy.

In some embodiments, X¹ is selected from —C₆₋₁₀ aryl, and 5-10 membered heteroaryl, wherein X¹ is unsubstituted or substituted with one or more substituents independently selected from the group consisting of linear or branched —C₁₋₄ alkyl, halogen, —CF₃, —CHF₂, —CMeF₂, —O—(CH₂)₂—OMe, —OCF₃, —OCHF₂, C₁₋₆ alkylamino, —CN, —NH₂, —C₁₋₄ alkylhydroxy, and —C₁₋₄ alkoxy.

In some embodiments, X² is selected from the group consisting of H, C₃₋₆ cycloalkyl, C₆ aryl, 6-membered heteroaryl, and 5-6 membered heterocycloalkyl, wherein X² is unsubstituted or substituted with one or more substituents independently selected from the group consisting of linear or branched C₁₋₄ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy.

In some embodiments, the compound is of formula IV, such as IVa, IVb or IVc:

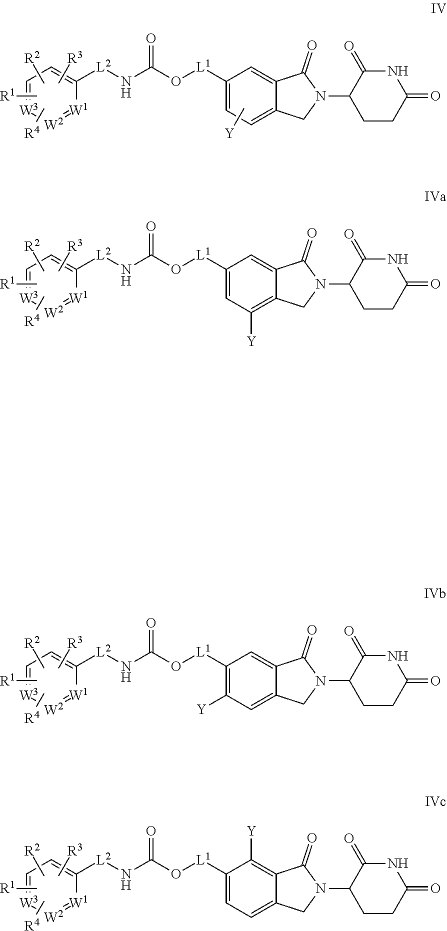

or a pharmaceutically acceptable salt or stereoisomer thereof,
wherein
Y is selected from the group consisting of linear or branched C₁₋₆ alkyl, —C₁₋₄ alkoxy, —CN, halogen, CF₃, CHF₂, CMeF₂, OCF₃, and OCHF₂;
each of w¹, w², and w³ is independently selected from C and N, with the proviso that two or three of w¹, w², w³ are C;
each of R¹, R², R³, and R⁴ is independently selected from the group consisting of H, linear or branched —C₁₋₆ alkyl, linear or branched C₁₋₆ heteroalkyl, —C₁₋₄ alkoxy, CF₃, CHF₂, CMeF₂, —O—(CH₂)₂—OMe, OCF₃, OCHF₂, —C₁₋₆ alkylamino, —CN, —NH₂, —C₁₋₄ alkylhydroxy, and halogen, such as F, Cl or Br, e.g. F or Cl, or a group of formula -L³-X², wherein L³ is selected from the group consisting of a covalent bond, linear or branched C₁₋₆ alkyl, and —O—, and X² is selected from the group consisting of C₃₋₄ cycloalkyl, C₆₋₁₀ aryl, 5-10 membered heteroaryl, and 4-8 membered heterocycloalkyl, wherein X² is unsubstituted or substituted with one or more substituents independently selected from the group consisting of linear or branched C₁₋₄ alkyl, —C₁₋₄ alkoxy, NH₂, NMe₂, halogen, CF₃, CHF₂, CMeF₂, —O—(CH₂)₂—OMe, OCF₃, OCHF₂, and C₁₋₄ alkylhydroxy;
L¹ is linear or branched C₁₋₆ alkyl; and
L² is selected from a covalent bond, and linear or branched C₁₋₆ alkyl.

In some embodiments, L¹ is —CH₂— and L² is a covalent bond.

In some embodiments, w¹, w², and w³ are C, or w¹ is N and w², and w³ are C, or w² is N and w¹, and w³ are C, or w³ is N and w¹, and w² are C.

In some embodiments, the compound is of formula XI, such as XIa, XIb or XIc:

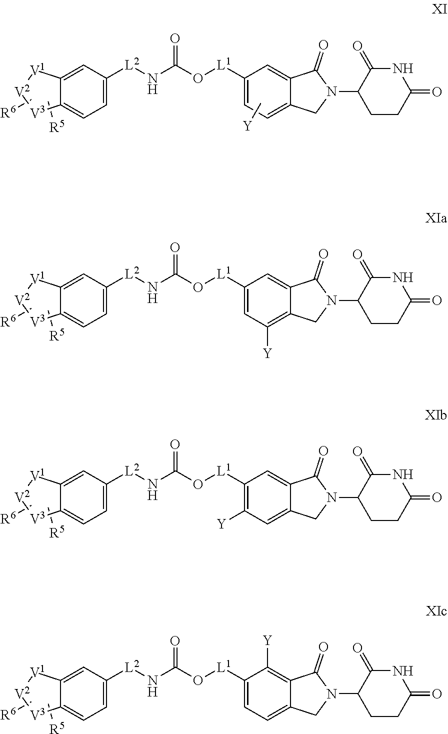

or a pharmaceutically acceptable salt or stereoisomer thereof,
wherein
Y is selected from the group consisting of linear or branched C₁₋₆ alkyl, —C₁₋₄ alkoxy, —CN, halogen, CF₃, CHF₂, CMeF₂, OCF₃, and OCHF₂
each of v¹, v², and v³ is independently selected from C, and O, with the proviso that at least one of v¹, v², and v³ is C;
each of R⁵ and R⁶ is independently selected from the group consisting of H, linear or branched —C₁₋₄ alkyl and halogen, such as F, Cl, e.g. F;
L¹ is linear or branched C₁₋₆ alkyl;
L² is selected from a covalent bond, and linear or branched C₁₋₆ alkyl.

In some embodiments, the compound is of formula XIII, such as XIIIa, XIIIb or XIIIc:

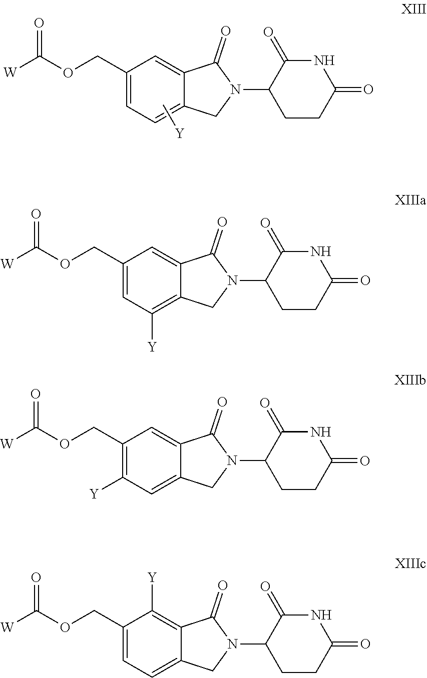

or pharmaceutically acceptable salts or stereoisomers thereof,
wherein
Y is selected from the group consisting of linear or branched C₁₋₆ alkyl, —C₁₋₄ alkoxy, —CN, halogen, CF₃, CHF₂, CMeF₂, OCF₃, and OCHF₂, such as C₁₋₄ alkyl, such as methyl, —C₁₋₄ alkoxy, such as —OMe, —CN, halogen, such as F, Cl, Br; and
W is selected from the group consisting of:

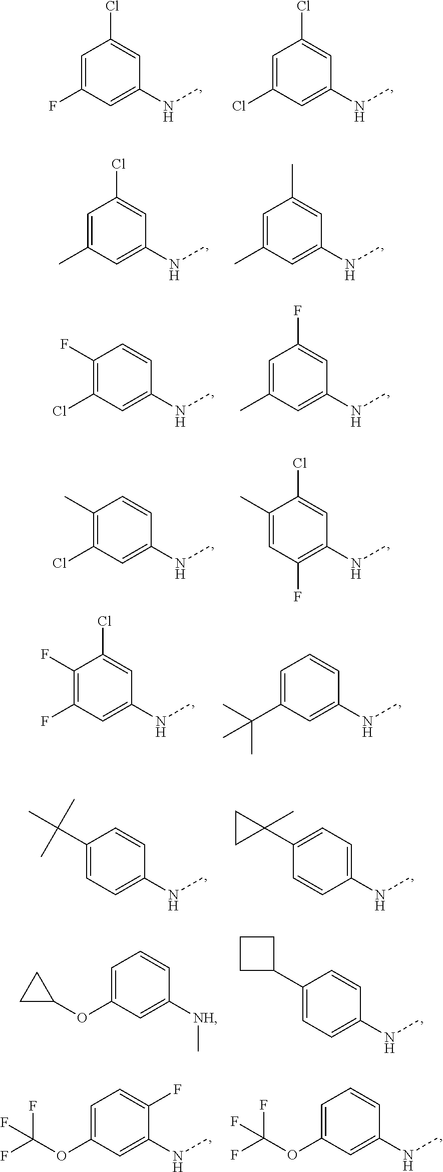

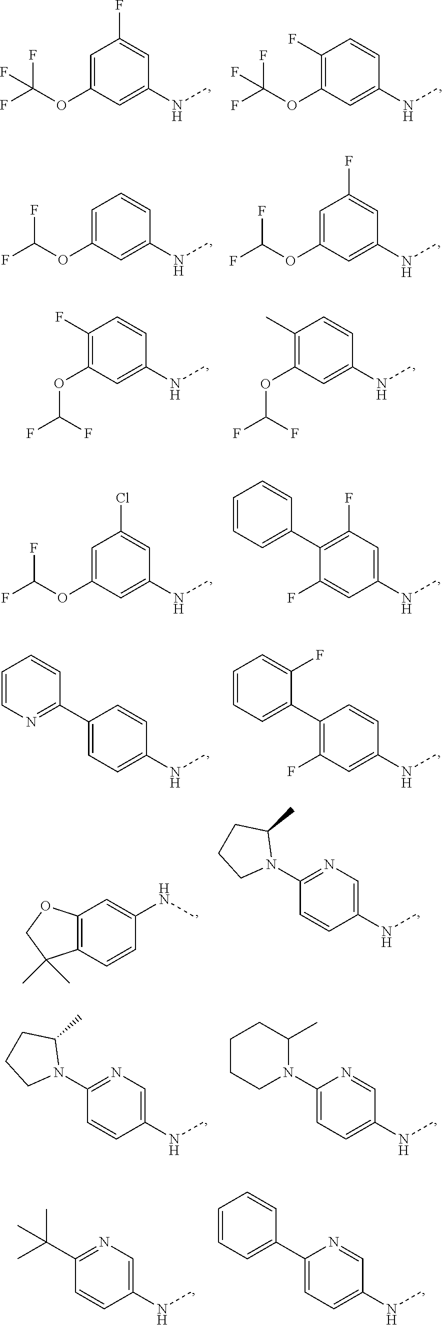

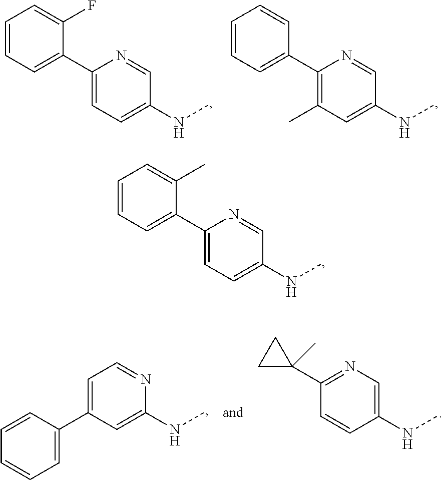

In a second aspect, the disclosure is directed to a composition comprising a compound according to any one of the embodiments or pharmaceutically acceptable salts or stereoisomers thereof described herein.

In some embodiments, the composition further comprises a pharmaceutically acceptable carrier.

In some embodiments, the composition further comprises a second therapeutically active agent.

In a third aspect, the disclosure is directed to a composition according to any of the embodiments described herein, for use in therapy.

In a fourth aspect, some embodiments comprise a compound or pharmaceutically acceptable salts or stereoisomers thereof of formula I-XIII, or a composition described herein for use in the treatment of diseases associated or caused by GSPT1, in particular the treatment of cancer associated with GSPT1, such as solid cancers including but not limited to cancers of the bladder, bone, brain, breast, cervix, chest, colon, endrometrium, esophagus, eye, head, kidney, liver, lymph nodes, lung, upper aerodigestive tract (including nasal cavity and paranasal sinuses, nasopharynx or cavum, oral cavity, oropharynx, larynx, hypopharynx and salivary glands), neck, ovaries, pancreas, prostate, rectum, skin, stomach, testis, throat, uterus, amyloidosis, neuroblastoma, meningioma, hemangiopericytoma, multiple brain metastase, glioblastoma multiforms, glioblastoma, brain stem glioma, poor prognosis malignant brain tumor, malignant glioma, recurrent malignant glioma, anaplastic astrocytoma, anaplastic oligodendroglioma, neuroendocrine tumor, e.g., neuroendocrine prostate cancer such as castration-resistant neuroendocrine prostate cancer (NEPC) and lung neuroendocrine tumors (Lu-NETs), rectal adenocarcinoma, colorectal cancer, including stage 3 and stage 4 colorectal cancer, unresectable colorectal carcinoma, metastatic hepatocellular carcinoma, Kaposi's sarcoma, malignant melanoma, malignant mesothelioma, malignant pleural effusion mesothelioma syndrome, peritoneal carcinoma, papillary serous carcinoma, gynecologic sarcoma, soft tissue sarcoma, scleroderma, cutaneous vasculitis, Langerhans cell histiocytosis, leiomyosarcoma, fibrodysplasia ossificans progressive, hormone refractory prostate cancer, resected high-risk soft tissue sarcoma, unrescectable hepatocellular carcinoma, fallopian tube cancer, androgen independent prostate cancer, androgen dependent stage IV non-metastatic prostate cancer, hormone-insensitive prostate cancer, chemotherapy-insensitive prostate cancer, papillary thyroid carcinoma, follicular thyroid carcinoma, medullary thyroid carcinoma, and leiomyoma; and blood bourne (liquid) or hematological cancers, including but not limited to leukemias, lymphomas, and myelomas, such as diffuse large B-cell lymphoma (DLBCL), B-cell immunoblastic lymphoma, small non-cleaved cell lymphoma, human lymphotropic virus-type 1 (HTLV-1) leukemia/lymphoma, adult T-cell lymphoma, peripheral T-cell lymphoma (PTCL), cutaneous T-cell lymphoma (CTCL), mantle cell lymphoma (MCL), Hodgkin's lymphoma (HL), non-Hodgkin's lymphoma (NHL), AIDS-related lymphoma, follicular lymphoma, small lymphocytic lymphoma, T-cell/histiocyte rich large B-cell lymphoma, transformed lymphoma, primary mediastinal (thymic) large B-cell lymphoma, splenic marginal zone lymphoma, Richter's transformation, nodal marginal zone lymphoma, ALK-positive large B-cell lymphoma, indolent lymphoma (for example, DLBCL, follicular lymphoma, or marginal zone lymphoma), acute myelogenous leukemia (AML), acute lymphocytic leukemia (ALL), adult T-cell leukemia, chronic lymphocytic leukemia (CLL), small lymphocytic lymphoma (SLL), hairy cell leukemia, myelodysplasia, myeloproliferative disorders, chronic myelogenous leukemia (CML), acute monocytic leukemia (AMoL), myelodysplastic syndrome (MDS), human lymphotropic virus-type 1 (HTLV-1) leukemia, mastocytosis, B-cell acute lymphoblastic leukemia, Non-Hodgkin's Lymphoma, Hodgkin's Lymphoma, and multiple myeloma (MM).

Some embodiments comprise the compound or the composition according to any of the embodiments described herein for use in the treatment of breast cancer.

Some embodiments comprise the compound or the composition according to any of the embodiments described herein for use in the treatment of lung cancer, for example, non-small cell lung cancer (e.g., squamous cell lung cancer) and small cell lung cancer.

Some embodiments comprise the use of a compound or a composition according to any of the embodiments described herein for treating neuroendocrine prostate cancer, for example, castration-resistant neuroendocrine prostate cancer (NEPC).

Some embodiments comprise the use of a compound or a composition according to any of the embodiments described herein for treating lung neuroendocrine tumors (Lu-NETs).

Some embodiments comprise the compound or the composition according to any of the embodiments described herein for use in the treatment of acute myelogenous leukemia (AML) and multiple myeloma (MM).

In a fifth aspect, the disclosure is directed to a use of a compound or the composition according to any of the embodiments described herein for binding to cereblon comprising administering to a subject a therapeutically-effective amount of the composition.

Some embodiments comprise the use of a composition according to any of the embodiments described herein for treating cancer associated with GSPT1, such as solid cancers and blood bourne (liquid) or hematological cancers as defined herein.

Some embodiments comprise the use of a compound or a composition according to any of the embodiments described herein for treating breast cancer.

Some embodiments comprise the use of a compound or a composition according to any of the embodiments described herein for treating lung cancer, for example, non-small cell lung cancer (e.g., squamous cell lung cancer) and small cell lung cancer.

Some embodiments comprise the use of a compound or a composition according to any of the embodiments described herein for treating neuroendocrine prostate cancer, for example, castration-resistant neuroendocrine prostate cancer (NEPC).

Some embodiments comprise the use of a compound or a composition according to any of the embodiments described herein for treating lung neuroendocrine tumors (Lu-NETs).

Some embodiments comprise the use of a compound or a composition according to any of the embodiments described herein for treating acute myelogenous leukemia (AML) and multiple myeloma (MM).

In a sixth aspect, the disclosure is directed to a method of treating cancer in a subject, comprising administering to a subject a therapeutically effective amount of the compound or the composition of any of the embodiments as described herein.

In some embodiments, the method comprises a compound according to any of the embodiments as described herein or pharmaceutically acceptable salts or stereoisomers thereof that binds to cereblon.

In a seventh aspect, the disclosure is directed to a method of treating a Myc-driven cancer in a subject in need thereof, comprising administering the subject a therapeutically effective amount of the compound or a composition as described herein. In an eight aspect, the disclosure is directed to a method of degrading GSPT1 in a subject suffering from cancer, comprising administering the subject a therapeutically effective amount of a compound or a composition as described herein.

In a ninth aspect, the disclosure is directed to a method of reducing the level of GSPT1 in a subject suffering from cancer, comprising administering the subject a therapeutically effective amount of a compound or a composition as described herein.

### DETAILED DESCRIPTION OF THE DISCLOSURE

Unless specified otherwise the following general definitions apply to all compounds of the disclosure according to the description.

The term “compound of the disclosure” as used herein, refers to compounds represented by formulae I to XIII (including pharmaceutically acceptable salts and stereoisomers thereof) and any of the specific examples disclosed herein. References to any compound of any formula herein includes also pharmaceutically acceptable salts or stereoisomers thereof,

It is understood that “independently of each other” means that when a group is occurring more than one time in any compound, its definition on each occurrence is independent from any other occurrence.

It is further understood that a dashed line (or a wave being transverse to a bond) or a solid line without attachment, such as —C₁₋₄ alkyl, depicts the site of attachment of a residue (i.e. a partial formula).

It is further understood that the abbreviations “C” and “N” are representative for all possible degrees of saturation, which typically do not result in radicals, nitrenes or carbenes, i.e. N includes —NH— and —N═, C includes —CH₂— and ═CH—. In addition, “C” as an atom in an aromatic or heteroaromatic ring which has a substituent RX at any suitable position, includes ═CH— as well as ═CR^{x}—.

The term “saturated” in reference to ring systems refers to a ring having no double or triple bonds. The term “partially unsaturated” in reference to ring systems refers to a ring that includes at least one double or triple bond, but does not include aromatic systems.

The term “aromatic” refers to monocyclic or multicyclic (e.g. bicyclic) ring systems, which show some or complete conjugation or delocalization of their electrons. Aromatic monocyclic rings, such as aryl or heteroaryl rings as defined herein, include phenyl, pyridinyl, furyl and the like. Aromatic multicyclic rings, such as aryl or heteroaryl rings as defined herein, refer to ring systems, wherein at least one ring is an aromatic ring, and thus include (i) aromatic ring systems, wherein an aromatic ring is fused to one or more aromatic rings, such as in e.g. naphthyl, indolyl, benzimidazolyl, and the like (also referred to as fully aromatic ring systems), and (ii) aromatic ring systems, wherein an aromatic ring is fused to one or more non-aromatic rings, such as in e.g. indanyl, indenyl, phthalimidyl, naphthimidyl, phenanthridinyl, tetrahydronaphthyl, 1,4-dihydronapthyl, and the like (also referred to as partially aromatic ring systems).

The term “non-aromatic” refers to (i) fully saturated rings such as monocyclic rings, e.g. cyclohexyl, and bicyclic rings, e.g. tetrahydronaphthyl, and (ii) partially unsaturated rings such as monocyclic rings, e.g. cyclohexenyl, and bicyclic rings, e.g. 1,4-dihydronapthyl.

The term “C₆₋₁₀ aryl” includes both fully aromatic C₆₋₁₀ aryl and partially aromatic C₆₋₁₀ aryl having 6, 7, 8, 9, or 10 ring atoms and includes monocycles and fused bicycles. Examples of fully aromatic C₆₋₁₀ aryl include e.g. phenyl (fully aromatic C₆ aryl), naphthyl (fully aromatic C₁₀ aryl). Examples of partially aromatic C₆₋₁₀ aryl include e.g. indenyl (partially aromatic C₉ aryl), 2,3-dihydroindenyl (partially aromatic C₉ aryl), 1, 2, 3, 4-tetrahydronaphthyl (partially aromatic C₁₀ aryl). In some embodiments for group X¹, C₆₋₁₀ aryl is phenyl. In some embodiments for group X², C₆₋₁₀ aryl is phenyl. The term “—C₁₋₆ alkyl-C₆₋₁₀ aryl” refers to -L²-X¹— or L³-X²— with L², L³ being a C₁₋₆ alkyl group and X¹, X² being a C₆₋₁₀ aryl, and thus refers to a C₆₋₁₀ aryl, which is linked through a C₁₋₆ alkyl group as defined herein to its neighbouring group. The term “—C₁₋₆ alkoxy-C₆₋₁₀ aryl” refers to -L²-X¹— or L³-X²— with L², L³ being a C₁₋₆ alkoxy group and X¹, X² being a C₆₋₁₀ aryl, and thus refers to a C₆₋₁₀ aryl, which is linked through a C₁₋₆ alkoxy group as defined herein to its neighbouring group. The term “—O—C₆₋₁₀ aryl” or “C₆₋₁₀ aryloxy” refers to -L²-X¹— or L³-X²— with L², L³ being —O— and X¹, X² being a C₆₋₁₀ aryl, and thus refers to a C₆₋₁₀ aryl, which is linked through a —O— group to its neighbouring group. The C₆₋₁₀ aryl group may be unsubstituted or substituted with C₁₋₆ alkyl, such as methyl, ethyl, t-butyl, fluorinated C₁₋₄ alkyl, such as —CF₃, —C(CH₃)F₂, C₁₋₄ alkoxy, such as methoxy, ethoxy, fluorinated C₁₋₄ alkoxy, such as —OCF₃, —OCHF₂, CN, —N(Me)₂, halogen, such as F, Cl, or Br, such as F or Cl.

In some embodiments for X¹, a C₆₋₁₀ aryl group refers to a fully aromatic ring system, e.g. phenyl, which is unsubstituted or substituted with C₁₋₄ alkyl, such as methyl, ethyl, t-butyl, fluorinated C₁₋₆ alkyl, such as —CMeF₂, C₁₋₄ alkoxy, such as methoxy, ethoxy, fluorinated C₁₋₄ alkoxy, such as —OCF₃, —OCHF₂, CN, halogen, such as F or Cl.

In some embodiments for X², a C₆₋₁₀ aryl group refers to a fully aromatic ring system, e.g. phenyl, which is unsubstituted or substituted with C₁₋₄ alkyl, such as methyl, ethyl, C₁₋₄ alkoxy, such as methoxy, ethoxy, halogen, such as F, Cl, or Br, such as F or Cl, e.g. F.

The term “5-10 membered heteroaryl” refers to a fully or partially aromatic ring system in form of monocycles or fused bicycles having 5, 6, 7, 8, 9, 10 ring atoms selected from C, N, O, and S, such as C, N, and O, or C, N, and S, with the number of N atoms being e.g. 0, 1, 2 or 3 and the number of O and S atoms each being 0, 1 or 2. In some embodiments a 5-10 membered heteroaryl refers to a fully aromatic ring system having 5, 6, 7, 8, 9, 10, such as 5 or 6, e.g. 6 ring atoms selected from C and N, with the number of N atoms being 1, 2 or 3, such as 1 or 2. In some embodiments a 5-10 membered heteroaryl refers to a fully aromatic ring system having 5, 6, 7, 8, 9, 10, such as 5 or 6, e.g. 5 ring atoms selected from C, N, O, S with the number of N, S and O atoms each being independently 0, 1 or 2. In some embodiments the total number of N, S and O atoms is 2. In some embodiments a 5-10 membered heteroaryl refers to a fully aromatic ring system having 5 ring atoms selected from C, N, S with the number of N and S atoms each being independently 0 or 1. In some embodiments the total number of N and S atoms is 2. In some embodiments a 5-10 membered heteroaryl refers to a fully aromatic ring system having 6 ring atoms selected from C and N, with the number of N atoms being 1 or 2. In other embodiments a 5-10 membered heteroaryl refers to a partially aromatic ring system having 9 or 10 ring atoms selected from C, N and O, with the number of O atoms being 1, 2 or 3, such as 1 or 2, and the number of N atoms being 1 or 2, such as 1. In some embodiments, examples of “5-10 membered heteroaryl” include furyl, imidazolyl, isoxazolyl, oxazolyl, pyrazinyl, pyrazolyl (pyrazyl), pyridazinyl, pyridinyl, pyrimidinyl, pyrrolyl, thiophenyl, thiazolyl, thienyl, indolyl, quinazolinyl, oxazolinyl, isoxazolinyl, indazolinyl, isothiazolyl, 1,3-benzodioxolyl, 2,2-difluoro-1,3-benzodioxolyl, 2,3-dihydrobenzofuryl, 2-methyl-2,3-dihydrobenzofuryl, 3-methyl-2,3-dihydrobenzofuryl, 3,3-dimethyl-2,3-dihydrobenzofuryl, 2,3-dimethyl-2,3-dihydrobenzofuryl, benzodihydropyrane, 1,2,3,4-tetrahydronaphthyl, 2,3-dihydroindenyl and the like. In some embodiments, examples of “5-10 membered heteroaryl” include 5-membered heteroaryl, such as isothiazole, 6-membered heteroaryl, such as pyridinyl, pyrimidinyl, pyridazinyl, pyrazinyl, 9-membered heteroaryl, such as 2,2-difluoro-1,3-benzodioxolyl, 2,3-dihydrobenzofuryl, 2-methyl-2,3-dihydrobenzofuryl, 3-methyl-2,3-dihydrobenzofuryl, 3,3-dimethyl-2,3-dihydrobenzofuryl, 2,3-dimethyl-2,3-dihydrobenzofuryl, cyclopentenopyridine, and 10-membered heteroaryl, such as benzodihydropyrane (chromane), dihydropyrano-pyridine. The term “—C₁₋₆ alkyl 5-10 membered heteroaryl” refers to -L²-X¹— or L³-X²— with L², L³ being a C₁₋₆ alkyl group and X¹, X² being a 5-10 membered heteroaryl, and thus refers to a 5-10 membered heteroaryl, which is linked through a C₁₋₆ alkyl group as defined herein to its neighbouring group. The term “—C₁₋₆ alkoxy 5-10 membered heteroaryl” refers to -L²-X¹— or L³-X²— with L², L³ being a C₁₋₆ alkoxy group and X¹, X² being a 5-10 membered heteroaryl, and thus refers to a 5-10 membered heteroaryl, which is linked through a C₁₋₆ alkoxy group as defined herein to its neighbouring group. The term “—O-5-10 membered heteroaryl” refers to -L²-X¹— or L³-X²— with L², L³ being —O— and X¹, X² being a 5-10 membered heteroaryl, and thus refers to a 5-10 membered heteroaryl, which is linked through a —O— group to its neighbouring group.

The 5-10 membered heteroaryl group may be unsubstituted or substituted with C₁₋₄ alkyl, such as methyl, ethyl, t-butyl, fluorinated C₁₋₄ alkyl, such as —CF₃, —C(CH₃)F₂, C₁₋₄ alkoxy, such as methoxy, ethoxy, fluorinated C₁₋₄ alkoxy, such as —OCF₃, —OCHF₂, CN, —N(Me)₂, halogen, such as F, Cl, or Br, such as F or Cl. In some embodiments, the 5-10 membered heteroaryl group may be unsubstituted or substituted with C₁₋₄ alkyl, such as methyl, ethyl, t-butyl, fluorinated C₁₋₄ alkyl, such as —CF₃, C₁₋₄ alkoxy, such as methoxy, ethoxy, halogen, such as F or Cl.

In some embodiments for X¹, a 5-10 membered heteroaryl refers to a fully aromatic ring system having 5 ring atoms selected from C, N and S with the number of N and S atoms being independently of each other 0 or 1, e.g. 1 or a fully aromatic ring system having 6 ring atoms selected from C and N, with the number of N atoms being 1 or 2 or a partially aromatic ring system having 9 or 10 ring atoms selected from C, N and O, with the number of 0 atoms being 1 or 2 and the number of N atoms being 0 or 1. In some embodiments for X¹, a 5-10 membered heteroaryl refers to isothiazole, phenyl, pyridinyl, pyrazinyl, pyridazinyl, pyrimidinyl, 2,2-difluoro-1,3-benzodioxolyl, 2,3-dihydrobenzofuryl, 2-methyl-2,3-dihydrobenzofuryl, 3-methyl-2,3-dihydrobenzofuryl, 3,3-dimethyl-2,3-dihydrobenzofuryl, 2,3-dimethyl-2,3-dihydrobenzofuryl, cyclopentenopyridine, benzodihydropyrane, dihydropyrano-pyridine.

In some embodiments for X² a 5-10 membered heteroaryl refers to a fully aromatic ring system having 6 ring atoms selected from C and N, with the number of N atoms being 1 or 2, such as 1. In some embodiments for X² a 5-10 membered heteroaryl refers to pyridinyl.

The term “C₃₋₆ cycloalkyl” refers to a non-aromatic, i.e. saturated or partially unsaturated alkyl ring system, such as monocycles, fused bicycles, bridged bicycles or spirobicycles, containing 3, 4, 5 or 6 carbon atoms. Examples of “C₃₋₈ cycloalkyl” include monocycles, such as cyclopropyl, cyclobutyl, cyclopentyl, cyclohexyl, bridged bicycles, such as bicyclo[1.1.1]pentyl, bicyclo[2.1.1]hexyl, fused bicycles, such as bicyclo[3.1.0]hexyl. The C₃₋₆ cycloalkyl group may be unsubstituted or substituted with C₁₋₄ alkyl, such as methyl, ethyl, t-butyl, fluorinated C₁₋₄ alkyl, such as —CF₃, —CMeF₂, C₁₋₄ alkoxy, such as methoxy, ethoxy, fluorinated C₁₋₄ alkoxy, such as —OCF₃, —OCHF₂, CN, —N(Me)₂, halogen, such as F, Cl, or Br, such as F or Cl. In some embodiments the C₃₋₆ cycloalkyl group may be unsubstituted or substituted by e.g. one or more of C₁₋₄ alkyl, such as methyl and halogen, such as F. In some embodiments for X¹, a C₃₋₆ cycloalkyl refers to cyclopropyl, cyclobutyl.

The term “4-8 membered heterocycloalkyl” refers to a non-aromatic, i.e. saturated or partially unsaturated ring system having 4, 5, 6, 7 or 8 ring atoms (of which at least one is a heteroatom), which ring atoms are selected from C, N, O, and S, such as C, N, and O, the number of N atoms being 0, 1, or 2 and the number of O and S atoms each being 0, 1, or 2.

In some embodiments the term “4-8 membered heterocycloalkyl” comprises saturated or partially unsaturated monocycles, fused bicycles, bridged bicycles or spirobicycles. In some embodiments the term “4-8 membered heterocycloalkyl” comprises fully saturated or partially unsaturated monocycles and bridged bicycles. Examples of 4-8 membered heterocycloalkyl groups include azetidinyl, oxetanyl, pyrrolidinyl, tetrahydrofuranyl, tetrahydrothiopyranyl, dihydropyranyl, tetrahydropyranyl, 1,3-dioxolanyl, 1,4-dioxanyl, 1,4-oxathianyl 1,4-dithianyl, 1,3-dioxanyl, 1,3-dithianyl, piperazinyl, thiomorpholinyl, piperidinyl, morpholinyl, azabicyclo[2.2.1]heptan-5-yl, 8-oxa-3-azabicyclo[3.2.1]octan-3-yl and the like. The 4-8 membered heterocycloalkyl group may be unsubstituted or substituted with C₁₋₄ alkyl, such as methyl, ethyl, C₁₋₄ alkoxy, such as methoxy, ethoxy, halogen, such as F, Cl or Br, e.g. F or Cl.

In some embodiments, the 4-8 membered heterocycloalkyl representing group X² is a non-aromatic ring system having 5 or 6 ring atoms of which at least one is a heteroatom selected from N, the number of N atoms being 1 or 2, such as a non-aromatic 5- or 6-membered ring system having 1 or 2 N-atom. Examples include pyrrolidinyl, piperdinyl, morpholinyl, piperazinyl, N-methyl piperazinyl. In some embodiments, the 4-8 membered heterocycloalkyl representing group X² is a non-aromatic ring system having 5 or 6 ring atoms of which one is a N-heteroatom, such as a non-aromatic 5- or 6-membered ring system having 1 N-atom, such as pyrrolidine, piperidine.

The term “halogen” or “hal” as used herein may be fluoro, chloro, bromo or iodo such as fluoro, chloro or bromo, e.g. fluoro or chloro.

The term “C₁₋₄ alkyl” and “C₁₋₆ alkyl” refer to a fully saturated branched or unbranched hydrocarbon moiety having 1, 2, 3 or 4 and 1, 2, 3, 4, 5 or 6 carbon atoms, respectively.

Representative examples of alkyl include, but are not limited to, methyl, ethyl, n-propyl, iso-propyl, n-butyl, sec-butyl, iso-butyl, tert-butyl, n-pentyl, iso-pentyl, neopentyl, n-hexyl, iso-hexyl or neohexyl.

The term “C₁₋₄ heteroalkyl” refers to an alkyl as defined with 1, 2, 3, 4, 5 or 6 carbon atoms in which at least one carbon atom is replaced with a heteroatom, such as N, O. It is understood that the heteroatom may further be substituted with one or two C₁₋₆ alkyl. Examples include —(CH₂)₂—O-Me, —(CH₂)₃—O-Me, —(CH₂)₂—O—CH₂Me, —(CH₂)₂—NMe₂, —(CH₂)—NMe₂, —(CH₂)₂—NEt₂, —(CH₂)—NEt₂ and the like.

The term “C₁₋₄alkylamino” refers to a fully saturated branched or unbranched C₁₋₄ alkyl, which is substituted with at least one, such as only one, amino group, alkylamino group or dialkylaminogroup, such as NH₂, HN(C₁₋₄alkyl) or N(C₁₋₄alkyl)₂. Thus, a C₁₋₄alkylamino refers to C₁₋₄alkylamino, C₁₋₄alkyl-(C₁₋₄alkyl)amino, C₁₋₄alkyl-(C₁₋₄dialkyl)amino.

Examples include but are not limited to methylaminomethyl, dimethylamonimethyl, aminomethyl, dimethylaminoethyl, aminoethyl, methylaminoethyl, n-propylamino, iso-propylamino, n-butylamino, sec-butylamino, iso-butylamino, tert-butylamino.

The term “C₁₋₄ alkoxy” refers to an unsubstituted or substituted alkyl chain linked to the remainder of the molecule through an oxygen atom, and in particular to methoxy, ethoxy, n-propoxy, iso-propoxy, n-butoxy, iso-butoxy, and t-butoxy.

Based on the definitions given throughout the application the skilled person knows which combinations are synthetically feasible and realistic, e.g. typically combinations of groups leading to some heteroatoms directly linked to each other, e.g. —O—O—, are not contemplated, however synthetically feasible combinations, such as —S—N═ in aisothiazole are contemplated.

In a first aspect the disclosure provides a compound or pharmaceutically acceptable salts or stereoisomers thereof of formula I:

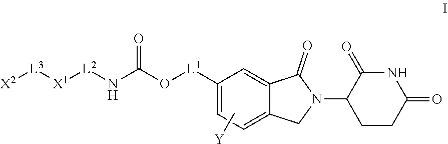

wherein
X¹ is linear or branched C₁₋₆ alkyl, C₃₋₆ cycloalkyl, C₆₋₁₀ aryl, 5-10 membered heteroaryl, 4-8 membered heterocycloalkyl, wherein X¹ is unsubstituted or substituted with one or more of halogen, linear or branched C₁₋₆ alkyl, linear or branched C₁₋₆ heteroalkyl, CF₃, CHF₂, CMeF₂, —O—CHF₂, —O—(CH₂)₂—OMe, OCF₃, C₁₋₆ alkylamino, —CN, NH₂, C₁₋₄ alkoxy and C₁₋₄ alkylhydroxy;
X² is H, C₃₋₆ cycloalkyl, C₆₋₁₀ aryl, 5-10 membered heteroaryl, 4-8 membered heterocycloalkyl, wherein X² is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, and C₁₋₄ alkylhydroxy;
Y is linear or branched C₁₋₆ alkyl, —C₁₋₄ alkoxy, —CN, halogen, CF₃, CHF₂, CMeF₂, OCF₃, OCHF₂;
L¹ is linear or branched C₁₋₆ alkyl;
L² is a covalent bond, linear or branched C₁₋₆ alkyl;
L³ is a covalent bond, linear or branched C₁₋₆ alkyl, —O—, —C₁₋₄ alkoxy.

In some embodiments of a compound of formula I, Y is in the 4-position. In some embodiments of a compound of formula I, Y is in the 5-position. In some embodiments of a compound of formula I, Y is in the 7-position.

In some embodiments, the compound of formula I is a compound or pharmaceutically acceptable salts or stereoisomers thereof of formula Ia, Ib or Ic:

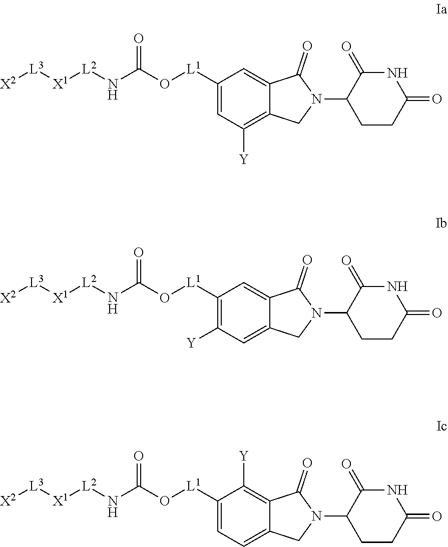

In some embodiments of formula I and formulas Ia, Ib, Ic, Y is C₁₋₄ alkyl, such as methyl, —C₁₋₄ alkoxy, such as —OMe, —CN, halogen, such as F, Cl, Br.

In some embodiments of a compound of formula I and formulas Ia, Ib, Ic, L¹ is linear or branched C₁₋₄ alkyl. In some embodiments of a compound of formula I and formulas Ia, Ib, Ic, L¹ is —CH₂—.

In some embodiments of a compound of formula I and formulas Ia, Ib, Ic, L² is a covalent bond. In some embodiments of a compound of formula I and formulas Ia, Ib, Ic, L² is —CH₂—.

In some embodiments of a compound of formula I and formulas Ia, Ib, Ic, L³ is a covalent bond. In some embodiments of a compound of formula I and formulas Ia, Ib, Ic, L³ is —O—.

In some embodiments of a compound of formula I and formulas Ia, Ib, Ic, L¹ is —CH₂— and L² is a covalent bond.

In some embodiments of a compound of formula I and formulas Ia, Ib, Ic, L¹ is —CH₂—, L² is a covalent bond and L³ is a covalent bond.

In some embodiments of a compound of formula I and formulas Ia, Ib, Ic, L¹ is —CH₂—, L² is a covalent bond and L³ is —O—.

In some embodiments of a compound of formula I and formulas Ia, Ib, Ic, X¹ is —C₆₋₁₀ aryl, 5-10 membered heteroaryl, wherein X¹ is unsubstituted or substituted with one or more of linear or branched —C₁₋₄ alkyl, halogen, —CF₃, —CHF₂, —CMeF₂, —O—(CH₂)₂—OMe, —OCF₃, —OCHF₂, C₁₋₆ alkylamino, —CN, —NH₂, —C₁₋₄ alkylhydroxy, and —C₁₋₄ alkoxy.

In some embodiments of a compound of formula I and formulas Ia, Ib, Ic, X¹ is —C₆ aryl, 6-9 membered heteroaryl, wherein X¹ is unsubstituted or substituted with one or more of linear or branched —C₁₋₄ alkyl, halogen, CF₃, —CHF₂, —CMeF₂, —OCF₃, —OCHF₂, and —C₁₋₄ alkoxy.

In some embodiments of a compound of formula I and formulas Ia, Ib, Ic, X¹ is —C₆ aryl, pyridyl, dihydrobenzofuryl, wherein X¹ is unsubstituted or substituted with one or more of linear or branched —C₁₋₄ alkyl, halogen, —OCF₃, —OCHF₂.

In some embodiments of a compound of formula I and formulas Ia, Ib, Ic, Y is C₁₋₄ alkyl, such as methyl, —C₁₋₄ alkoxy, such as —OMe, —CN, halogen, such as F, Cl, Br and X¹ is —C₆₋₁₀ aryl, 5-10 membered heteroaryl, wherein X¹ is unsubstituted or substituted with one or more of linear or branched —C₁₋₄ alkyl, halogen, —CF₃, —CHF₂, —CMeF₂, —O—(CH₂)₂—OMe, —OCF₃, —OCHF₂, C₁₋₆ alkylamino, —CN, —NH₂, —C₁₋₄ alkylhydroxy, and —C₁₋₄ alkoxy.

In some embodiments of a compound of formula I and formulas Ia, Ib, Ic, Y is C₁₋₄ alkyl, such as methyl, —C₁₋₄ alkoxy, such as —OMe, —CN, halogen, such as F, Cl, Br and X¹ is —C₆ aryl, 6-9 membered heteroaryl, wherein X¹ is unsubstituted or substituted with one or more of linear or branched —C₁₋₄ alkyl, halogen, CF₃, —CHF₂, —CMeF₂, —OCF₃, —OCHF₂, and —C₁₋₄ alkoxy.

In some embodiments of a compound of formula I and formulas Ia, Ib, Ic, Y is C₁₋₄ alkyl, such as methyl, —C₁₋₄ alkoxy, such as —OMe, —CN, halogen, such as F, Cl, Br and X¹ is —C₆ aryl, pyridyl, dihydrobenzofuryl, wherein X¹ is unsubstituted or substituted with one or more of linear or branched —C₁₋₄ alkyl, halogen, —OCF₃, —OCHF₂.

In some embodiments of a compound of formula I and formulas Ia, Ib, Ic, X² is H, C₃₋₆ cycloalkyl, C₆ aryl, 6-membered heteroaryl, 5-6 membered heterocycloalkyl, wherein X² is unsubstituted or substituted with one or more of linear or branched C₁₋₄ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy.

In some embodiments of a compound of formula I and formulas Ia, Ib, Ic, X² is H, C₃₋₆ cycloalkyl, C₆ aryl, 6 membered heteroaryl, 4-8 membered heterocycloalkyl, wherein X² is unsubstituted or substituted with one or more of linear or branched C₁₋₄ alkyl, and halogen, e.g. F.

In some embodiments of a compound of formula I and formulas Ia, Ib, Ic, X² is H, cyclopropyl, cyclobutyl, C₆ aryl, pyridinyl, pyrrolidinyl, piperdinyl, wherein X² is unsubstituted or substituted with one or more of linear or branched C₁₋₄ alkyl, such as methyl, and halogen, such as F.

In some embodiments of a compound of formula I and formulas Ia, Ib, Ic, L¹ is a linear or branched C₁₋₄ alkyl and X¹ is —C₆₋₁₀ aryl, 5-10 membered heteroaryl, wherein X¹ is unsubstituted or substituted with one or more of linear or branched —C₁₋₄ alkyl, halogen, —CF₃, —CHF₂, —CMeF₂, —O—(CH₂)₂—OMe, —OCF₃, —OCHF₂, C₁₋₆ alkylamino, —CN, —NH₂, —C₁₋₄ alkylhydroxy, and —C₁₋₄ alkoxy.

In some embodiments of a compound of formula I and formulas Ia, Ib, Ic, L¹ is a linear or branched C₁₋₄ alkyl and X¹ is —C₆ aryl, 6-9 membered heteroaryl, wherein X¹ is unsubstituted or substituted with one or more of linear or branched —C₁₋₄ alkyl, halogen, CF₃, —CHF₂, —CMeF₂, —OCF₃, —OCHF₂, and —C₁₋₄ alkoxy.

In some embodiments of a compound of formula I and formulas Ia, Ib, Ic, L¹ is a linear or branched C₁₋₄ alkyl and X¹ is —C₆ aryl, pyridyl, dihydrobenzofuryl, wherein X¹ is unsubstituted or substituted with one or more of linear or branched —C₁₋₄ alkyl, halogen, —OCF₃, —OCHF₂.

In some embodiments of a compound of formula I and formulas Ia, Ib, Ic, L¹ is —CH₂— and X¹ is —C₆₋₁₀ aryl, 5-10 membered heteroaryl, wherein X¹ is unsubstituted or substituted with one or more of linear or branched —C₁₋₄ alkyl, halogen, —CF₃, —CHF₂, —CMeF₂, —O—(CH₂)₂—OMe, —OCF₃, —OCHF₂, C₁₋₆ alkylamino, —CN, —NH₂, —C₁₋₄ alkylhydroxy, and —C₁₋₄ alkoxy.

In some embodiments of a compound of formula I and formulas Ia, Ib, Ic, L¹ is —CH₂— and X¹ is —C₆ aryl, 6-9 membered heteroaryl, wherein X¹ is unsubstituted or substituted with one or more of linear or branched —C₁₋₄ alkyl, halogen, CF₃, —CHF₂, —CMeF₂, —OCF₃, —OCHF₂, and —C₁₋₄ alkoxy.

In some embodiments of a compound of formula I and formulas Ia, Ib, Ic, L¹ is —CH₂— and X¹ is —C₆ aryl, pyridyl, dihydrobenzofuryl, wherein X¹ is unsubstituted or substituted with one or more of linear or branched —C₁₋₄ alkyl, halogen, —OCF₃, —OCHF₂.

In some embodiments of a compound of formula I and formulas Ia, Ib, Ic, L² is a covalent bond and X¹ is —C₆₋₁₀ aryl, 5-10 membered heteroaryl, wherein X¹ is unsubstituted or substituted with one or more of linear or branched —C₁₋₄ alkyl, halogen, —CF₃, —CHF₂, —CMeF₂, —O—(CH₂)₂—OMe, —OCF₃, —OCHF₂, C₁₋₆ alkylamino, —CN, —NH₂, —C₁₋₄ alkylhydroxy, and —C₁₋₄ alkoxy.

In some embodiments of a compound of formula I and formulas Ia, Ib, Ic, L² is a covalent bond and X¹ is —C₆ aryl, 6-9 membered heteroaryl, wherein X¹ is unsubstituted or substituted with one or more of linear or branched —C₁₋₄ alkyl, halogen, CF₃, —CHF₂, —CMeF₂, —OCF₃, —OCHF₂, and —C₁₋₄ alkoxy.

In some embodiments of a compound of formula I and formulas Ia, Ib, Ic, L² is a covalent bond and X¹ is —C₆ aryl, pyridyl, dihydrobenzofuryl, wherein X¹ is unsubstituted or substituted with one or more of linear or branched —C₁₋₄ alkyl, halogen, —OCF₃, —OCHF₂.

In some embodiments of a compound of formula I and formulas Ia, Ib, Ic, L² is —CH₂— and X¹ is —C₆₋₁₀ aryl, 5-10 membered heteroaryl, wherein X¹ is unsubstituted or substituted with one or more of linear or branched —C₁₋₄ alkyl, halogen, —CF₃, —CHF₂, —CMeF₂, —O—(CH₂)₂—OMe, —OCF₃, —OCHF₂, C₁₋₆ alkylamino, —CN, —NH₂, —C₁₋₄ alkylhydroxy, and —C₁₋₄ alkoxy.

In some embodiments of a compound of formula I and formulas Ia, Ib, Ic, L² is —CH₂— and X¹ is —C₆ aryl, 6-9 membered heteroaryl, wherein X¹ is unsubstituted or substituted with one or more of linear or branched —C₁₋₄ alkyl, halogen, CF₃, —CHF₂, —CMeF₂, —OCF₃, —OCHF₂, and —C₁₋₄ alkoxy.

In some embodiments of a compound of formula I and formulas Ia, Ib, Ic, L² is —CH₂— and X¹ is —C₆ aryl, pyridyl, dihydrobenzofuryl, wherein X¹ is unsubstituted or substituted with one or more of linear or branched —C₁₋₄ alkyl, halogen, —OCF₃, —OCHF₂.

In some embodiments of a compound of formula I and formulas Ia, Ib, Ic, L¹ is C₁₋₄ alkyl, L² is a covalent bond and X¹ is —C₆₋₁₀ aryl, 5-10 membered heteroaryl, wherein X¹ is unsubstituted or substituted with one or more of linear or branched —C₁₋₄ alkyl, halogen, —CF₃, —CHF₂, —CMeF₂, —O—(CH₂)₂—OMe, —OCF₃, —OCHF₂, C₁₋₆ alkylamino, —CN, —NH₂, —C₁₋₄ alkylhydroxy, and —C₁₋₄ alkoxy.

In some embodiments of a compound of formula I and formulas Ia, Ib, Ic, L¹ is C₁₋₄ alkyl, L² is a covalent bond and X¹ is —C₆ aryl, 6-9 membered heteroaryl, wherein X¹ is unsubstituted or substituted with one or more of linear or branched —C₁₋₄ alkyl, halogen, CF₃, —CHF₂, —CMeF₂, —OCF₃, —OCHF₂, and —C₁₋₄ alkoxy.

In some embodiments of a compound of formula I and formulas Ia, Ib, Ic, L¹ is C₁₋₄ alkyl, L² is a covalent bond and X¹ is —C₆ aryl, pyridyl, dihydrobenzofuryl, wherein X¹ is unsubstituted or substituted with one or more of linear or branched —C₁₋₄ alkyl, halogen, —OCF₃, —OCHF₂.

In some embodiments of a compound of formula I and formulas Ia, Ib, Ic, L¹ is —CH₂—, L² is a covalent bond and X¹ is —C₆₋₁₀ aryl, 5-10 membered heteroaryl, wherein X¹ is unsubstituted or substituted with one or more of linear or branched —C₁₋₄ alkyl, halogen, —CF₃, —CHF₂, —CMeF₂, —O—(CH₂)₂—OMe, —OCF₃, —OCHF₂, C₁₋₆ alkylamino, —CN, —NH₂, —C₁₋₄ alkylhydroxy, and —C₁₋₄ alkoxy.

In some embodiments of a compound of formula I and formulas Ia, Ib, Ic, LI is —CH₂—, L² is a covalent bond and X¹ is —C₆ aryl, 6-9 membered heteroaryl, wherein X¹ is unsubstituted or substituted with one or more of linear or branched —C₁₋₄ alkyl, halogen, CF₃, —CHF₂, —CMeF₂, —OCF₃, —OCHF₂, and —C₁₋₄ alkoxy.

In some embodiments of a compound of formula I and formulas Ia, Ib, Ic, LI is —CH₂—, L² is a covalent bond and X¹ is —C₆ aryl, pyridyl, dihydrobenzofuryl, wherein X¹ is unsubstituted or substituted with one or more of linear or branched —C₁₋₄ alkyl, halogen, —OCF₃, —OCHF₂.

In some embodiments of a compound of formula I and formulas Ia, Ib, Ic, L¹ is C₁₋₄ alkyl, L² is —CH₂— and X¹ is —C₆₋₁₀ aryl, 5-10 membered heteroaryl, wherein X¹ is unsubstituted or substituted with one or more of linear or branched —C₁₋₄ alkyl, halogen, —CF₃, —CHF₂, —CMeF₂, —O—(CH₂)₂—OMe, —OCF₃, —OCHF₂, C₁₋₆ alkylamino, —CN, —NH₂, —C₁₋₄ alkylhydroxy, and —C₁₋₄ alkoxy.

In some embodiments of a compound of formula I and formulas Ia, Ib, Ic, L¹ is C₁₋₄ alkyl, L² is —CH₂— and X¹ is —C₆ aryl, 6-9 membered heteroaryl, wherein X¹ is unsubstituted or substituted with one or more of linear or branched —C₁₋₄ alkyl, halogen, CF₃, —CHF₂, —CMeF₂, —OCF₃, —OCHF₂, and —C₁₋₄ alkoxy.

In some embodiments of a compound of formula I and formulas Ia, Ib, Ic, L¹ is C₁₋₄ alkyl, L² is —CH₂— and X¹ is —C₆ aryl, pyridyl, dihydrobenzofuryl, wherein X¹ is unsubstituted or substituted with one or more of linear or branched —C₁₋₄ alkyl, halogen, —OCF₃, —OCHF₂.

In some embodiments of a compound of formula I and formulas Ia, Ib, Ic, L¹ is —CH₂—, L² is —CH₂— and X¹ is —C₆₋₁₀ aryl, 5-10 membered heteroaryl, wherein X¹ is unsubstituted or substituted with one or more of linear or branched —C₁₋₄ alkyl, halogen, —CF₃, —CHF₂, —CMeF₂, —O—(CH₂)₂—OMe, —OCF₃, —OCHF₂, C₁₋₆ alkylamino, —CN, —NH₂, —C₁₋₄ alkylhydroxy, and —C₁₋₄ alkoxy.

In some embodiments of a compound of formula I and formulas Ia, Ib, Ic, L¹ is —CH₂—, L² is —CH₂— and X¹ is —C₆ aryl, 6-9 membered heteroaryl, wherein X¹ is unsubstituted or substituted with one or more of linear or branched —C₁₋₄ alkyl, halogen, CF₃, —CHF₂, —CMeF₂, —OCF₃, —OCHF₂, and —C₁₋₄ alkoxy.

In some embodiments of a compound of formula I and formulas Ia, Ib, Ic, L¹ is —CH₂—, L² is —CH₂— and X¹ is —C₆ aryl, pyridyl, dihydrobenzofuryl, wherein X¹ is unsubstituted or substituted with one or more of linear or branched —C₁₋₄ alkyl, halogen, —OCF₃, —OCHF₂.

In some embodiments of a compound of formula I and formulas Ia, Ib, Ic, L² is a covalent bond and X² is H, C₃₋₆ cycloalkyl, C₆ aryl, 6-membered heteroaryl, 5-6 membered heterocycloalkyl, wherein X² is unsubstituted or substituted with one or more of linear or branched C₁₋₄ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy.

In some embodiments of a compound of formula I and formulas Ia, Ib, Ic, L² is a covalent bond and X² is H, C₃₋₆ cycloalkyl, C₆ aryl, 6 membered heteroaryl, 4-8 membered heterocycloalkyl, wherein X² is unsubstituted or substituted with one or more of linear or branched C₁₋₄ alkyl, and halogen, e.g. F.

In some embodiments of a compound of formula I and formulas Ia, Ib, Ic, L² is a covalent bond and X² is H, cyclopropyl, cyclobutyl, C₆ aryl, pyridinyl, pyrrolidinyl, piperdinyl, wherein X² is unsubstituted or substituted with one or more of linear or branched C₁₋₄ alkyl, such as methyl, and halogen, such as F.

In some embodiments of a compound of formula I and formulas Ia, Ib, Ic, L² is —CH₂— and X² is H, C₃₋₆ cycloalkyl, C₆ aryl, 6-membered heteroaryl, 5-6 membered heterocycloalkyl, wherein X² is unsubstituted or substituted with one or more of linear or branched C₁₋₄ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy.

In some embodiments of a compound of formula I and formulas Ia, Ib, Ic, L² is —CH₂— and X² is H, C₃₋₆ cycloalkyl, C₆ aryl, 6 membered heteroaryl, 4-8 membered heterocycloalkyl, wherein X² is unsubstituted or substituted with one or more of linear or branched C₁₋₄ alkyl, and halogen, e.g. F.

In some embodiments of a compound of formula I and formulas Ia, Ib, Ic, L² is —CH₂— and X² is H, cyclopropyl, cyclobutyl, C₆ aryl, pyridinyl, pyrrolidinyl, piperdinyl, wherein X² is unsubstituted or substituted with one or more of linear or branched C₁₋₄ alkyl, such as methyl, and halogen, such as F.

In some embodiments of a compound of formula I and formulas Ia, Ib, Ic, L³ is a covalent bond and X² is H, C₃₋₆ cycloalkyl, C₆ aryl, 6-membered heteroaryl, 5-6 membered heterocycloalkyl, wherein X² is unsubstituted or substituted with one or more of linear or branched C₁₋₄ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy.

In some embodiments of a compound of formula I and formulas Ia, Ib, Ic, L³ is a covalent bond and X² is H, C₃₋₆ cycloalkyl, C₆ aryl, 6 membered heteroaryl, 4-8 membered heterocycloalkyl, wherein X² is unsubstituted or substituted with one or more of linear or branched C₁₋₄ alkyl, and halogen, e.g. F.

In some embodiments of a compound of formula I and formulas Ia, Ib, Ic, L³ is a covalent bond and X² is H, cyclopropyl, cyclobutyl, C₆ aryl, pyridinyl, pyrrolidinyl, piperdinyl, wherein X² is unsubstituted or substituted with one or more of linear or branched C₁₋₄ alkyl, such as methyl, and halogen, such as F.

In some embodiments of a compound of formula I and formulas Ia, Ib, Ic, L³ is —O— and X² is H, C₃₋₆ cycloalkyl, C₆ aryl, 6-membered heteroaryl, 5-6 membered heterocycloalkyl, wherein X² is unsubstituted or substituted with one or more of linear or branched C₁₋₄ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy.

In some embodiments of a compound of formula I and formulas Ia, Ib, Ic, L³ is —O— and X² is H, C₃₋₆ cycloalkyl, C₆ aryl, 6 membered heteroaryl, 4-8 membered heterocycloalkyl, wherein X² is unsubstituted or substituted with one or more of linear or branched C₁₋₄ alkyl, and halogen, e.g. F.

In some embodiments of a compound of formula I and formulas Ia, Ib, Ic, L³ is —O— and X² is H, cyclopropyl, cyclobutyl, C₆ aryl, pyridinyl, pyrrolidinyl, piperdinyl, wherein X² is unsubstituted or substituted with one or more of linear or branched C₁₋₄ alkyl, such as methyl, and halogen, such as F.

In some embodiments of a compound of formula I and formulas Ia, Ib, Ic, L³ is —O— and X² is cyclopropyl.

In some embodiments of a compound of formula I and formulas Ia, Ib, Ic, L² is a covalent bond, L³ is a covalent bond and X² is H, C₃₋₆ cycloalkyl, C₆ aryl, 6-membered heteroaryl, 5-6 membered heterocycloalkyl, wherein X² is unsubstituted or substituted with one or more of linear or branched C₁₋₄ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy.

In some embodiments of a compound of formula I and formulas Ia, Ib, Ic, L² is a covalent bond, L³ is a covalent bond and X² is H, C₃₋₆ cycloalkyl, C₆ aryl, 6 membered heteroaryl, 4-8 membered heterocycloalkyl, wherein X² is unsubstituted or substituted with one or more of linear or branched C₁₋₄ alkyl, and halogen, e.g. F.

In some embodiments of a compound of formula I and formulas Ia, Ib, Ic, L² is a covalent bond, L³ is a covalent bond and X² is H, cyclopropyl, cyclobutyl, C₆ aryl, pyridinyl, pyrrolidinyl, piperdinyl, wherein X² is unsubstituted or substituted with one or more of linear or branched C₁₋₄ alkyl, such as methyl, and halogen, such as F.

In some embodiments of a compound of formula I and formulas Ia, Ib, Ic, L² is a covalent bond, L³ is —O— and X² is H, C₃₋₆ cycloalkyl, C₆ aryl, 6-membered heteroaryl, 5-6 membered heterocycloalkyl, wherein X² is unsubstituted or substituted with one or more of linear or branched C₁₋₄ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy.

In some embodiments of a compound of formula I and formulas Ia, Ib, Ic, L² is a covalent bond, L³ is —O— and X² is H, C₃₋₆ cycloalkyl, C₆ aryl, 6 membered heteroaryl, 4-8 membered heterocycloalkyl, wherein X² is unsubstituted or substituted with one or more of linear or branched C₁₋₄ alkyl, and halogen, e.g. F.

In some embodiments of a compound of formula I and formulas Ia, Ib, Ic, L² is a covalent bond, L³ is —O— and X² is H, cyclopropyl, cyclobutyl, C₆ aryl, pyridinyl, pyrrolidinyl, piperdinyl, wherein X² is unsubstituted or substituted with one or more of linear or branched C₁₋₄ alkyl, such as methyl, and halogen, such as F.

In some embodiments of a compound of formula I and formulas Ia, Ib, Ic, L² is a covalent bond, L³ is —O— and X² is cyclopropyl.

In some embodiments of a compound of formula I and formulas Ia, Ib, Ic, L² is —CH₂, L³ is covalent bond and X² is H, C₃₋₆ cycloalkyl, C₆ aryl, 6-membered heteroaryl, 5-6 membered heterocycloalkyl, wherein X² is unsubstituted or substituted with one or more of linear or branched C₁₋₄ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy.

In some embodiments of a compound of formula I and formulas Ia, Ib, Ic, L² is —CH₂, L³ is covalent bond and X² is H, C₃₋₆ cycloalkyl, C₆ aryl, 6 membered heteroaryl, 4-8 membered heterocycloalkyl, wherein X² is unsubstituted or substituted with one or more of linear or branched C₁₋₄ alkyl, and halogen, e.g. F.

In some embodiments of a compound of formula I and formulas Ia, Ib, Ic, L² is —CH₂, L³ is covalent bond and X² is H, cyclopropyl, cyclobutyl, C₆ aryl, pyridinyl, pyrrolidinyl, piperdinyl, wherein X² is unsubstituted or substituted with one or more of linear or branched C₁₋₄ alkyl, such as methyl, and halogen, such as F.

In some embodiments of a compound of formula I and formulas Ia, Ib, Ic, L² is —CH₂—, L³ is —O— and X² is H, C₃₋₆ cycloalkyl, C₆ aryl, 6-membered heteroaryl, 5-6 membered heterocycloalkyl, wherein X² is unsubstituted or substituted with one or more of linear or branched C₁₋₄ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy.

In some embodiments of a compound of formula I and formulas Ia, Ib, Ic, L² is —CH₂—, L³ is —O— and X² is H, C₃₋₆ cycloalkyl, C₆ aryl, 6 membered heteroaryl, 4-8 membered heterocycloalkyl, wherein X² is unsubstituted or substituted with one or more of linear or branched C₁₋₄ alkyl, and halogen, e.g. F.

In some embodiments of a compound of formula I and formulas Ia, Ib, Ic, L² is —CH₂—, L³ is —O— and X² is H, cyclopropyl, cyclobutyl, C₆ aryl, pyridinyl, pyrrolidinyl, piperdinyl, wherein X² is unsubstituted or substituted with one or more of linear or branched C₁₋₄ alkyl, such as methyl, and halogen, such as F.

In some embodiments, the compound of formula I is a compound or pharmaceutically acceptable salts or stereoisomers thereof of formula II, such as formula Ha, Ib or IIc:

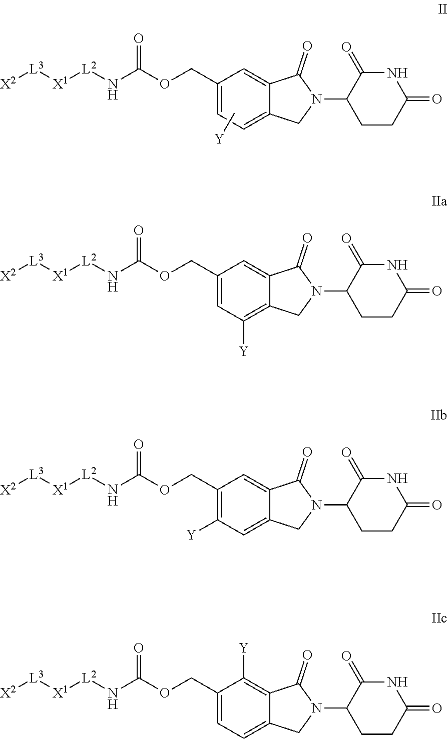

wherein
X¹ is linear or branched C₁₋₆ alkyl, C₃₋₄ cycloalkyl, C₆₋₁₀ aryl, 5-10 membered heteroaryl, 4-8 membered heterocycloalkyl, wherein X¹ is unsubstituted or substituted with one or more of halogen, linear or branched C₁₋₆ alkyl, linear or branched C₁₋₆ heteroalkyl, CF₃, CHF₂, CMeF₂, —O—CHF₂, —O—(CH₂)₂—OMe, OCF₃, C₁₋₆ alkylamino, —CN, NH₂, C₁₋₄ alkoxy and C₁₋₄ alkylhydroxy;
X² is H, C₃₋₄ cycloalkyl, C₆₋₁₀ aryl, 5-10 membered heteroaryl, 4-8 membered heterocycloalkyl, wherein X² is unsubstituted or substituted with one or more of linear or branched C₁₋₄ alkyl, —C₁₋₄ alkoxy, NH₂, NMe₂, halogen, CF₃, CHF₂, CMeF₂, —O—(CH₂)₂—OMe, OCF₃, OCHF₂, and C₁₋₄ alkylhydroxy;
Y is linear or branched C₁₋₆ alkyl, —C₁₋₄ alkoxy, —CN, halogen, CF₃, CHF₂, CMeF₂, OCF₃, OCHF₂;
L² is a covalent bond, linear or branched C₁₋₆ alkyl;
L³ is a covalent bond, linear or branched C₁₋₆ alkyl, —O—, —C₁₋₄ alkoxy.

In some embodiments of formula II and formula Ha, IIb or IIc, Y is C₁₋₄ alkyl, such as methyl, —C₁₋₄ alkoxy, such as —OMe, —CN, halogen, such as F, Cl, Br.

In some embodiments of a compound of formula II and formula IIa, IIb or IIc, L² is a covalent bond. In some embodiments of a compound of formula II and formula IIa, IIb or IIc, L² is —CH₂—.

In some embodiments of a compound of formula II and formula Ha, IIb or IIc, L³ is a covalent bond. In some embodiments of a compound of formula II and formula IIa, IIb or IIc, L³ is —O—.

In some embodiments of a compound of formula II and formula Ha, IIb or IIc, L² is a covalent bond and L³ is a covalent bond. In some embodiments of a compound of formula II and formula Ha, IIb or IIc, L² is a covalent bond and L³ is —O—.

In some embodiments of a compound of formula II and formula Ha, IIb or IIc, X¹ is —C₆₋₁₀ aryl, 5-10 membered heteroaryl, wherein X¹ is unsubstituted or substituted with one or more of linear or branched —C₁₋₄ alkyl, halogen, —CF₃, —CHF₂, —CMeF₂, —O—(CH₂)₂—OMe, —OCF₃, —OCHF₂, C₁₋₆ alkylamino, —CN, —NH₂, —C₁₋₄ alkylhydroxy, and —C₁₋₄ alkoxy.

In some embodiments of a compound of formula II and formula Ha, IIb or IIc, X¹ is —C₆ aryl, 6-9 membered heteroaryl, wherein X¹ is unsubstituted or substituted with one or more of linear or branched —C₁₋₄ alkyl, halogen, CF₃, —CHF₂, —CMeF₂, —OCF₃, —OCHF₂, and —C₁₋₄ alkoxy.

In some embodiments of a compound of formula II and formula Ha, IIb or IIc, X¹ is —C₆ aryl, pyridyl, dihydrobenzofuryl, wherein X¹ is unsubstituted or substituted with one or more of linear or branched —C₁₋₄ alkyl, halogen, —OCF₃, —OCHF₂.

In some embodiments of a compound of formula II and formula IIa, IIb or IIc, Y is C₁₋₄ alkyl, such as methyl, —C₁₋₄ alkoxy, such as —OMe, —CN, halogen, such as F, Cl, Br and X¹ is —C₆₋₁₀ aryl, 5-10 membered heteroaryl, wherein X¹ is unsubstituted or substituted with one or more of linear or branched —C₁₋₄ alkyl, halogen, —CF₃, —CHF₂, —CMeF₂, —O—(CH₂)₂—OMe, —OCF₃, —OCHF₂, C₁₋₆ alkylamino, —CN, —NH₂, —C₁₋₄ alkylhydroxy, and —C₁₋₄ alkoxy.

In some embodiments of a compound of formula II and formula Ha, IIb or IIc, Y is C₁₋₄ alkyl, such as methyl, —C₁₋₄ alkoxy, such as —OMe, —CN, halogen, such as F, Cl, Br and X¹ is —C₆ aryl, 6-9 membered heteroaryl, wherein X¹ is unsubstituted or substituted with one or more of linear or branched —C₁₋₄ alkyl, halogen, CF₃, —CHF₂, —CMeF₂, —OCF₃, —OCHF₂, and —C₁₋₄ alkoxy.

In some embodiments of a compound of formula II and formula Ha, IIb or IIc, Y is C₁₋₄ alkyl, such as methyl, —C₁₋₄ alkoxy, such as —OMe, —CN, halogen, such as F, Cl, Br and X¹ is —C₆ aryl, pyridyl, dihydrobenzofuryl, wherein X¹ is unsubstituted or substituted with one or more of linear or branched —C₁₋₄ alkyl, halogen, —OCF₃, —OCHF₂.

In some embodiments of a compound of formula II and formula Ha, IIb or IIc, X² is H, C₃₋₆ cycloalkyl, C₆ aryl, 6-membered heteroaryl, 5-6 membered heterocycloalkyl, wherein X² is unsubstituted or substituted with one or more of linear or branched C₁₋₄ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy.

In some embodiments of a compound of formula II and formula Ha, IIb or IIc, X² is H, C₃₋₆ cycloalkyl, C₆ aryl, 6 membered heteroaryl, 4-8 membered heterocycloalkyl, wherein X² is unsubstituted or substituted with one or more of linear or branched C₁₋₄ alkyl, and halogen, e.g. F.

In some embodiments of a compound of formula II and formula Ha, IIb or IIc, X² is H, cyclopropyl, cyclobutyl, C₆ aryl, pyridinyl, pyrrolidinyl, piperdinyl, wherein X² is unsubstituted or substituted with one or more of linear or branched C₁₋₄ alkyl, such as methyl, and halogen, such as F.

In some embodiments of a compound of formula II and formula IIa, IIb or IIc, L² is a covalent bond and X¹ is —C₆₋₁₀ aryl, 5-10 membered heteroaryl, wherein X¹ is unsubstituted or substituted with one or more of linear or branched —C₁₋₄ alkyl, halogen, —CF₃, —CHF₂, —CMeF₂, —O—(CH₂)₂—OMe, —OCF₃, —OCHF₂, C₁₋₆ alkylamino, —CN, —NH₂, —C₁₋₄ alkylhydroxy, and —C₁₋₄ alkoxy.

In some embodiments of a compound of formula II and formula Ha, IIb or IIc, L² is a covalent bond and X¹ is —C₆ aryl, 6-9 membered heteroaryl, wherein X¹ is unsubstituted or substituted with one or more of linear or branched —C₁₋₄ alkyl, halogen, CF₃, —CHF₂, —CMeF₂, —OCF₃, —OCHF₂, and —C₁₋₄ alkoxy.

In some embodiments of a compound of formula II and formula IIa, IIb or IIc, L² is a covalent bond and X¹ is —C₆ aryl, pyridyl, dihydrobenzofuryl, wherein X¹ is unsubstituted or substituted with one or more of linear or branched —C₁₋₄ alkyl, halogen, —OCF₃, —OCHF₂.

In some embodiments of a compound of formula II and formula Ha, IIb or IIc, L² is —CH₂— and X¹ is —C₆₋₁₀ aryl, 5-10 membered heteroaryl, wherein X¹ is unsubstituted or substituted with one or more of linear or branched —C₁₋₄ alkyl, halogen, —CF₃, —CHF₂, —CMeF₂, —O—(CH₂)₂—OMe, —OCF₃, —OCHF₂, C₁₋₆ alkylamino, —CN, —NH₂, —C₁₋₄ alkylhydroxy, and —C₁₋₄ alkoxy.

In some embodiments of a compound of formula II and formula IIa, IIb or IIc, L² is —CH₂— and X¹ is —C₆ aryl, 6-9 membered heteroaryl, wherein X¹ is unsubstituted or substituted with one or more of linear or branched —C₁₋₄ alkyl, halogen, CF₃, —CHF₂, —CMeF₂, —OCF₃, —OCHF₂, and —C₁₋₄ alkoxy.

In some embodiments of a compound of formula II and formula Ha, IIb or IIc, L² is —CH₂— and X¹ is —C₆ aryl, pyridyl, dihydrobenzofuryl, wherein X¹ is unsubstituted or substituted with one or more of linear or branched —C₁₋₄ alkyl, halogen, —OCF₃, —OCHF₂.

In some embodiments of a compound of formula II and formula Ha, IIb or IIc, L² is a covalent bond and X² is H, C₃₋₆ cycloalkyl, C₆ aryl, 6-membered heteroaryl, 5-6 membered heterocycloalkyl, wherein X² is unsubstituted or substituted with one or more of linear or branched C₁₋₄ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy.

In some embodiments of a compound of formula II and formula Ha, IIb or IIc, L² is a covalent bond and X² is H, C₃₋₆ cycloalkyl, C₆ aryl, 6 membered heteroaryl, 4-8 membered heterocycloalkyl, wherein X² is unsubstituted or substituted with one or more of linear or branched C₁₋₄ alkyl, and halogen, e.g. F.

In some embodiments of a compound of formula II and formula Ha, IIb or IIc, L² is a covalent bond and X² is H, cyclopropyl, cyclobutyl, C₆ aryl, pyridinyl, pyrrolidinyl, piperdinyl, wherein X² is unsubstituted or substituted with one or more of linear or branched C₁₋₄ alkyl, such as methyl, and halogen, such as F.

In some embodiments of a compound of formula II and formula Ha, Ib or IIc, L² is —CH₂— and X² is H, C₃₋₆ cycloalkyl, C₆ aryl, 6-membered heteroaryl, 5-6 membered heterocycloalkyl, wherein X² is unsubstituted or substituted with one or more of linear or branched C₁₋₄ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy.

In some embodiments of a compound of formula II and formula Ha, Ib or IIc, L² is —CH₂— and X² is H, C₃₋₆ cycloalkyl, C₆ aryl, 6 membered heteroaryl, 4-8 membered heterocycloalkyl, wherein X² is unsubstituted or substituted with one or more of linear or branched C₁₋₄ alkyl, and halogen, e.g. F.

In some embodiments of a compound of formula II and formula IIa, IIb or IIc, L² is —CH₂— and X² is H, cyclopropyl, cyclobutyl, C₆ aryl, pyridinyl, pyrrolidinyl, piperdinyl, wherein X² is unsubstituted or substituted with one or more of linear or branched C₁₋₄ alkyl, such as methyl, and halogen, such as F.

In some embodiments of a compound of formula II and formula Ha, IIb or IIc, L³ is a covalent bond and X² is H, C₃₋₆ cycloalkyl, C₆ aryl, 6-membered heteroaryl, 5-6 membered heterocycloalkyl, wherein X² is unsubstituted or substituted with one or more of linear or branched C₁₋₄ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy.

In some embodiments of a compound of formula II and formula Ha, IIb or IIc, L³ is a covalent bond and X² is H, C₃₋₆ cycloalkyl, C₆ aryl, 6 membered heteroaryl, 4-8 membered heterocycloalkyl, wherein X² is unsubstituted or substituted with one or more of linear or branched C₁₋₄ alkyl, and halogen, e.g. F.

In some embodiments of a compound of formula II and formula Ha, IIb or IIc, L³ is a covalent bond and X² is H, cyclopropyl, cyclobutyl, C₆ aryl, pyridinyl, pyrrolidinyl, piperdinyl, wherein X² is unsubstituted or substituted with one or more of linear or branched C₁₋₄ alkyl, such as methyl, and halogen, such as F.

In some embodiments of a compound of formula II and formula Ha, IIb or IIc, L³ is —O— and X² is H, C₃₋₆ cycloalkyl, C₆ aryl, 6-membered heteroaryl, 5-6 membered heterocycloalkyl, wherein X² is unsubstituted or substituted with one or more of linear or branched C₁₋₄ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy.

In some embodiments of a compound of formula II and formula Ha, IIb or IIc, L³ is —O— and X² is H, C₃₋₆ cycloalkyl, C₆ aryl, 6 membered heteroaryl, 4-8 membered heterocycloalkyl, wherein X² is unsubstituted or substituted with one or more of linear or branched C₁₋₄ alkyl, and halogen, e.g. F.

In some embodiments of a compound of formula II and formula Ha, IIb or IIc, L³ is —O— and X² is H, cyclopropyl, cyclobutyl, C₆ aryl, pyridinyl, pyrrolidinyl, piperdinyl, wherein X² is unsubstituted or substituted with one or more of linear or branched C₁₋₄ alkyl, such as methyl, and halogen, such as F.

In some embodiments of a compound of formula II and formula Ha, IIb or IIc, L³ is —O— and X² is cyclopropyl.

In some embodiments of a compound of formula II and formula Ha, IIb or IIc, L² is a covalent bond, L³ is a covalent bond and X² is H, C₃₋₆ cycloalkyl, C₆ aryl, 6-membered heteroaryl, 5-6 membered heterocycloalkyl, wherein X² is unsubstituted or substituted with one or more of linear or branched C₁₋₄ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy.

In some embodiments of a compound of formula II and formula Ha, IIb or IIc, L² is a covalent bond, L³ is a covalent bond and X² is H, C₃₋₆ cycloalkyl, C₆ aryl, 6 membered heteroaryl, 4-8 membered heterocycloalkyl, wherein X² is unsubstituted or substituted with one or more of linear or branched C₁₋₄ alkyl, and halogen, e.g. F.

In some embodiments of a compound of formula II and formula Ha, IIb or IIc, L² is a covalent bond, L³ is a covalent bond and X² is H, cyclopropyl, cyclobutyl, C₆ aryl, pyridinyl, pyrrolidinyl, piperdinyl, wherein X² is unsubstituted or substituted with one or more of linear or branched C₁₋₄ alkyl, such as methyl, and halogen, such as F.

In some embodiments of a compound of formula II and formula Ha, IIb or IIc, L² is a covalent bond, L³ is —O— and X² is H, C₃₋₆ cycloalkyl, C₆ aryl, 6-membered heteroaryl, 5-6 membered heterocycloalkyl, wherein X² is unsubstituted or substituted with one or more of linear or branched C₁₋₄ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy.

In some embodiments of a compound of formula II and formula Ha, IIb or IIc, L² is a covalent bond, L³ is —O— and X² is H, C₃₋₆ cycloalkyl, C₆ aryl, 6 membered heteroaryl, 4-8 membered heterocycloalkyl, wherein X² is unsubstituted or substituted with one or more of linear or branched C₁₋₄ alkyl, and halogen, e.g. F.

In some embodiments of a compound of formula II and formula Ha, IIb or IIc, L² is a covalent bond, L³ is —O— and X² is H, cyclopropyl, cyclobutyl, C₆ aryl, pyridinyl, pyrrolidinyl, piperdinyl, wherein X² is unsubstituted or substituted with one or more of linear or branched C₁₋₄ alkyl, such as methyl, and halogen, such as F.

In some embodiments of a compound of formula II and formula Ha, IIb or IIc, L² is a covalent bond, L³ is —O— and X² is cyclopropyl.

In some embodiments of a compound of formula II and formula Ha, IIb or IIc, L² is —CH₂, L³ is covalent bond and X² is H, C₃₋₆ cycloalkyl, C₆ aryl, 6-membered heteroaryl, 5-6 membered heterocycloalkyl, wherein X² is unsubstituted or substituted with one or more of linear or branched C₁₋₄ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy.

In some embodiments of a compound of formula II and formula Ha, IIb or IIc, L² is —CH₂, L³ is covalent bond and X² is H, C₃₋₆ cycloalkyl, C₆ aryl, 6 membered heteroaryl, 4-8 membered heterocycloalkyl, wherein X² is unsubstituted or substituted with one or more of linear or branched C₁₋₄ alkyl, and halogen, e.g. F.

In some embodiments of a compound of formula II and formula Ha, IIb or IIc, L² is —CH₂, L³ is covalent bond and X² is H, cyclopropyl, cyclobutyl, C₆ aryl, pyridinyl, pyrrolidinyl, piperdinyl, wherein X² is unsubstituted or substituted with one or more of linear or branched C₁₋₄ alkyl, such as methyl, and halogen, such as F.

In some embodiments of a compound of formula II and formula Ha, IIb or IIc, L² is —CH₂—, L³ is —O— and X² is H, C₃₋₆ cycloalkyl, C₆ aryl, 6-membered heteroaryl, 5-6 membered heterocycloalkyl, wherein X² is unsubstituted or substituted with one or more of linear or branched C₁₋₄ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy.

In some embodiments of a compound of formula II and formula Ha, IIb or IIc, L² is —CH₂—, L³ is —O— and X² is H, C₃₋₆ cycloalkyl, C₆ aryl, 6 membered heteroaryl, 4-8 membered heterocycloalkyl, wherein X² is unsubstituted or substituted with one or more of linear or branched C₁₋₄ alkyl, and halogen, e.g. F.

In some embodiments of a compound of formula II and formula Ha, IIb or IIc, L² is —CH₂—, L³ is —O— and X² is H, cyclopropyl, cyclobutyl, C₆ aryl, pyridinyl, pyrrolidinyl, piperdinyl, wherein X² is unsubstituted or substituted with one or more of linear or branched C₁₋₄ alkyl, such as methyl, and halogen, such as F.

In some embodiments of a compound of formula II and formula Ha, IIb or IIc, L² is —CH₂—, L³ is —O— and X² is cyclopropyl.

In specific embodiments the compound of formula I is a compound or pharmaceutically acceptable salts or stereoisomers thereof of formula III, such as formula IIIa, IIIb or IIIc:

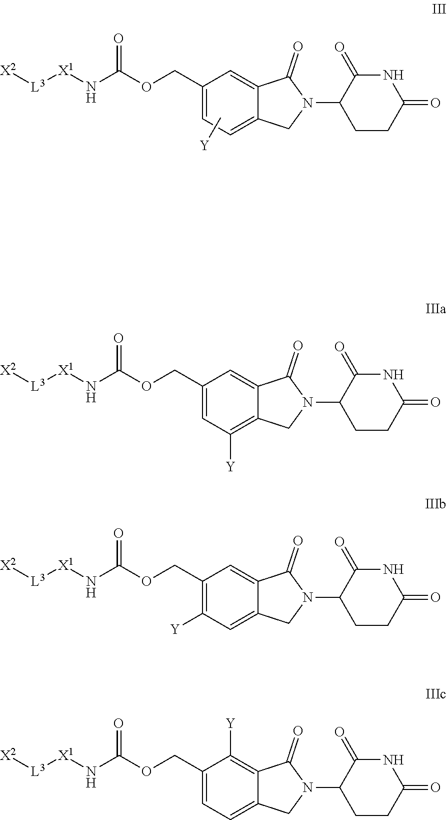

wherein
X¹ is linear or branched C₁₋₆ alkyl, C₃₋₆ cycloalkyl, C₆₋₁₀ aryl, 5-10 membered heteroaryl, 4-8 membered heterocycloalkyl, wherein X¹ is unsubstituted or substituted with one or more of halogen, linear or branched C₁₋₆ alkyl, linear or branched C₁₋₆ heteroalkyl, CF₃, CHF₂, CMeF₂, —O—CHF₂, —O—(CH₂)₂—OMe, OCF₃, C₁₋₆ alkylamino, —CN, NH₂, C₁₋₄ alkoxy and C₁₋₄ alkylhydroxy;
X² is H, C₃₋₆ cycloalkyl, C₆₋₁₀ aryl, 5-10 membered heteroaryl, 4-8 membered heterocycloalkyl, wherein X² is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, and C₁₋₄ alkylhydroxy;
Y is linear or branched C₁₋₆ alkyl, —C₁₋₄ alkoxy, —CN, halogen, CF₃, CHF₂, CMeF₂, OCF₃, OCHF₂;
L³ is a covalent bond, linear or branched C₁₋₆ alkyl, —O—, —C₁₋₄ alkoxy.

In some embodiments of formula III and formulas IIIa, IIIb, IIIc, Y is C₁₋₄ alkyl, such as methyl, —C₁₋₄ alkoxy, such as —OMe, —CN, halogen, such as F, Cl, Br.

In some embodiments of a compound of formula III and formulas IIIa, IIIb, IIIc, L³ is a covalent bond. In some embodiments of a compound of formula III and formulas IIIa, IIIb, IIIc, L³ is —O—.

In some embodiments of a compound of formula III and formulas IIIa, IIIb, IIIc, X¹ is —C₆₋₁₀ aryl, 5-10 membered heteroaryl, wherein X¹ is unsubstituted or substituted with one or more of linear or branched —C₁₋₄ alkyl, halogen, —CF₃, —CHF₂, —CMeF₂, —O—(CH₂)₂—OMe, —OCF₃, —OCHF₂, C₁₋₆ alkylamino, —CN, —NH₂, —C₁₋₄ alkylhydroxy, and —C₁₋₄ alkoxy.

In some embodiments of a compound of formula III and formulas IIIa, IIIb, IIIc, X¹ is —C₆ aryl, 6-9 membered heteroaryl, wherein X¹ is unsubstituted or substituted with one or more of linear or branched —C₁₋₄ alkyl, halogen, CF₃, —CHF₂, —CMeF₂, —OCF₃, —OCHF₂, and —C₁₋₄ alkoxy.

In some embodiments of a compound of formula III and formulas IIIa, IIIb, IIIc, X¹ is —C₆ aryl, pyridyl, dihydrobenzofuryl, wherein X¹ is unsubstituted or substituted with one or more of linear or branched —C₁₋₄ alkyl, halogen, —OCF₃, —OCHF₂.

In some embodiments of a compound of formula III and formulas IIIa, IIIb, IIIc, Y is C₁₋₄ alkyl, such as methyl, —C₁₋₄ alkoxy, such as —OMe, —CN, halogen, such as F, Cl, Br and X¹ is —C₆₋₁₀ aryl, 5-10 membered heteroaryl, wherein X¹ is unsubstituted or substituted with one or more of linear or branched —C₁₋₄ alkyl, halogen, —CF₃, —CHF₂, —CMeF₂, —O—(CH₂)₂—OMe, —OCF₃, —OCHF₂, C₁₋₆ alkylamino, —CN, —NH₂, —C₁₋₄ alkylhydroxy, and —C₁₋₄ alkoxy.

In some embodiments of a compound of formula III and formulas IIIa, IIIb, IIIc, Y is C₁₋₄ alkyl, such as methyl, —C₁₋₄ alkoxy, such as —OMe, —CN, halogen, such as F, Cl, Br and X¹ is —C₆ aryl, 6-9 membered heteroaryl, wherein X¹ is unsubstituted or substituted with one or more of linear or branched —C₁₋₄ alkyl, halogen, CF₃, —CHF₂, —CMeF₂, —OCF₃, —OCHF₂, and —C₁₋₄ alkoxy.

In some embodiments of a compound of formula III and formulas IIIa, IIIb, IIIc, Y is C₁₋₄ alkyl, such as methyl, —C₁₋₄ alkoxy, such as —OMe, —CN, halogen, such as F, Cl, Br and X¹ is —C₆ aryl, pyridyl, dihydrobenzofuryl, wherein X¹ is unsubstituted or substituted with one or more of linear or branched —C₁₋₄ alkyl, halogen, —OCF₃, —OCHF₂.

In some embodiments of a compound of formula III and formulas IIIa, IIIb, IIIc, X² is H, C₃₋₆ cycloalkyl, C₆ aryl, 6-membered heteroaryl, 5-6 membered heterocycloalkyl, wherein X² is unsubstituted or substituted with one or more of linear or branched C₁₋₄ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy.

In some embodiments of a compound of formula III and formulas IIIa, IIIb, IIIc, X² is H, C₃₋₆ cycloalkyl, C₆ aryl, 6 membered heteroaryl, 4-8 membered heterocycloalkyl, wherein X² is unsubstituted or substituted with one or more of linear or branched C₁₋₄ alkyl, and halogen, e.g. F.

In some embodiments of a compound of formula III and formulas IIIa, IIIb, IIIc, X² is H, cyclopropyl, cyclobutyl, C₆ aryl, pyridinyl, pyrrolidinyl, piperdinyl, wherein X² is unsubstituted or substituted with one or more of linear or branched C₁₋₄ alkyl, such as methyl, and halogen, such as F.

In some embodiments of a compound of formula III and formulas IIIa, IIIb, IIIc, L³ is a covalent bond and X² is H, C₃₋₆ cycloalkyl, C₆ aryl, 6-membered heteroaryl, 5-6 membered heterocycloalkyl, wherein X² is unsubstituted or substituted with one or more of linear or branched C₁₋₄ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy.

In some embodiments of a compound of formula III and formulas IIIa, IIIb, IIIc, L³ is a covalent bond and X² is H, C₃₋₆ cycloalkyl, C₆ aryl, 6 membered heteroaryl, 4-8 membered heterocycloalkyl, wherein X² is unsubstituted or substituted with one or more of linear or branched C₁₋₄ alkyl, and halogen, e.g. F.

In some embodiments of a compound of formula III and formulas IIIa, IIIb, IIIc, L³ is a covalent bond and X² is H, cyclopropyl, cyclobutyl, C₆ aryl, pyridinyl, pyrrolidinyl, piperdinyl, wherein X² is unsubstituted or substituted with one or more of linear or branched C₁₋₄ alkyl, such as methyl, and halogen, such as F.

In some embodiments of a compound of formula III and formulas IIIa, IIIb, IIIc, L³ is —O— and X² is H, C₃₋₆ cycloalkyl, C₆ aryl, 6-membered heteroaryl, 5-6 membered heterocycloalkyl, wherein X² is unsubstituted or substituted with one or more of linear or branched C₁₋₄ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy.

In some embodiments of a compound of formula III and formulas IIIa, IIIb, IIIc, L³ is —O— and X² is H, C₃₋₆ cycloalkyl, C₆ aryl, 6 membered heteroaryl, 4-8 membered heterocycloalkyl, wherein X² is unsubstituted or substituted with one or more of linear or branched C₁₋₄ alkyl, and halogen, e.g. F.

In some embodiments of a compound of formula III and formulas IIIa, IIIb, IIIc, L³ is —O— and X² is H, cyclopropyl, cyclobutyl, C₆ aryl, pyridinyl, pyrrolidinyl, piperdinyl, wherein X² is unsubstituted or substituted with one or more of linear or branched C₁₋₄ alkyl, such as methyl, and halogen, such as F.

In some embodiments of a compound of formula III and formulas IIIa, IIIb, IIIc, L³ is —O— and X² is cyclopropyl.

In some embodiments of a compound of formula I, X¹ is a C₆ aryl or 6-membered heteroaryl, such as a pyridine. Thus, in some embodiments the compound of formula I is a compound or pharmaceutically acceptable salts or stereoisomers thereof of formula IV, such as IVa, IVb or IVc:

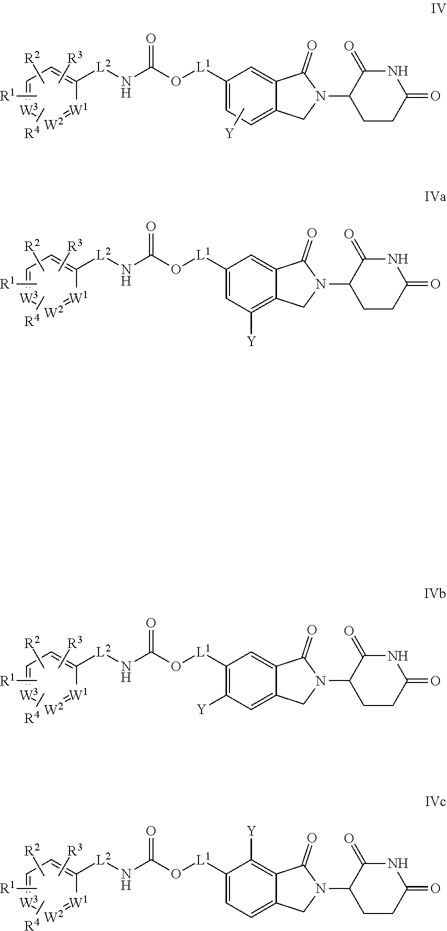

wherein
Y is linear or branched C₁₋₄ alkyl, —C₁₋₄ alkoxy, —CN, halogen, CF₃, CHF₂, CMeF₂, OCF₃, OCHF₂;
w¹, w², w³ are independently of each other selected from C, N, with the proviso that two or three of w¹, w², w³ are C;
R¹, R², R³, R⁴ are independently of each other selected from H, linear or branched —C₁₋₆ alkyl, linear or branched C₁₋₆ heteroalkyl, —C₁₋₄ alkoxy, CF₃, CHF₂, CMeF₂, —O—(CH₂)₂—OMe, OCF₃, OCHF₂, —C₁— alkylamino, —CN, —NH₂, —C₁₋₄ alkylhydroxy, and halogen, such as F, Cl or Br, e.g. F or Cl, or a group of formula -L³-X², wherein L³ is a covalent bond, linear or branched C₁₋₆ alkyl, —O—, and X² is C₃₋₆ cycloalkyl, C₆₋₁₀ aryl, 5-10 membered heteroaryl, 4-8 membered heterocycloalkyl, wherein X² is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, and C₁₋₄ alkylhydroxy;
L¹ is linear or branched C₁₋₆ alkyl; L² is a covalent bond, linear or branched C₁₋₆ alkyl.

In some embodiments of formula IV and formulas IVa, IVb or IVc, Y is C₁₋₄ alkyl, such as methyl, —C₁₋₄ alkoxy, such as —OMe, —CN, halogen, such as F, Cl, Br.

In some embodiments of a compound of formula IV and formulas IVa, IVb or IVc, L¹ is linear or branched C₁₋₄ alkyl. In some embodiments of a compound of formula IV and formulas IVa, IVb or IVc, L¹ is —CH₂—.

In some embodiments of a compound of formula IV and formulas IVa, IVb or IVc, L² is a covalent bond. In some embodiments of a compound of formula IV and formulas IVa, IVb or IVc, L² is —CH₂—.

In some embodiments of a compound of formula IV and formulas IVa, IVb or IVc, L³ is a covalent bond. In some embodiments of a compound of formula IV and formulas IVa, IVb or IVc, L³ is —O—.

In some embodiments of a compound of formula IV and formulas IVa, IVb or IVc, L¹ is —CH₂— and L² is a covalent bond.

In some embodiments of a compound of formula IV and formulas IVa, IVb or IVc, L¹ is —CH₂—, L² is a covalent bond and L³ is a covalent bond. In some embodiments of a compound of formula I, L¹ is —CH₂—, L² is a covalent bond and L³ is —O—.

In some embodiments of a compound of formula IV and formulas IVa, IVb or IVc, w¹, w², w³ are C. In some embodiments of a compound of formula IV and formulas IVa, IVb or IVc, either w¹ is N and w², w³ are C. In some embodiments of a compound of formula IV and formulas IVa, IVb or IVc, w² is N and w¹, w³ are C. In some embodiments of a compound of formula IV and formulas IVa, IVb or IVc, w³ is N and w¹, w² are C.

In some embodiments of a compound of formula IV and formulas IVa, IVb or IVc, R¹, R², R³, and R⁴ are independently of each other selected from H, linear or branched —C₁₋₆ alkyl, linear or branched C₁₋₆ heteroalkyl, —C₁₋₄ alkoxy, CF₃, CHF₂, CMeF₂, —O—(CH₂)₂—OMe, OCF₃, OCHF₂, —C₁— alkylamino, —CN, NH₂, —C₁₋₄ alkylhydroxy, and halogen, such as F, Cl or Br, e.g. F or Cl.

In some embodiments of a compound of formula IV and formulas IVa, IVb or IVc, R¹, R², R³, and R⁴ are independently of each other selected from H, linear or branched —C₁₋₆ alkyl, —C₁₋₄ alkoxy, CF₃, CHF₂, CMeF₂, OCF₃, OCHF₂, —CN, and halogen, such as F, Cl or Br, e.g. F or Cl.

In some embodiments of a compound of formula IV and formulas IVa, IVb or IVc, R¹, R², R³, and R⁴ are independently of each other selected from H, linear or branched —C₁₋₆ alkyl, OCF₃, OCHF₂, and halogen, such as F, Cl or Br, e.g. F or Cl.

In some embodiments of a compound of formula IV and formulas IVa, IVb or IVc, R¹ is H and R², R³, R⁴ are independently of each other selected from H, linear or branched —C₁₋₆ alkyl, linear or branched C₁₋₄ heteroalkyl, —C₁₋₄ alkoxy, CF₃, CHF₂, CMeF₂, —O—(CH₂)₂—OMe, OCF₃, OCHF₂, —C₁₋₆ alkylamino, —CN, NH₂, —C₁₋₄ alkylhydroxy, and halogen, such as F, Cl or Br, e.g. F or Cl.

In some embodiments of a compound of formula IV and formulas IVa, IVb or IVc, R¹ is H and R², R³, R⁴ are independently of each other selected from H, linear or branched —C₁₋₆ alkyl, —C₁₋₄ alkoxy, CF₃, CHF₂, CMeF₂, OCF₃, OCHF₂, —CN, and halogen, such as F, Cl or Br, e.g. F or Cl.

In some embodiments of a compound of formula IV and formulas IVa, IVb or IVc, R¹ is H and R², R³, and R⁴ are independently of each other selected from H, linear or branched —C₁₋₄ alkyl, OCF₃, OCHF₂, and halogen, such as F, Cl or Br, e.g. F or Cl.

In some embodiments of a compound of formula IV and formulas IVa, IVb or IVc, L¹ is —CH₂— and R¹, R², R³, and R⁴ are independently of each other selected from H, linear or branched —C₁₋₆ alkyl, linear or branched C₁₋₆ heteroalkyl, —C₁₋₄ alkoxy, CF₃, CHF₂, CMeF₂, —O—(CH₂)₂—OMe, OCF₃, OCHF₂, —C₁— alkylamino, —CN, NH₂, —C₁₋₄ alkylhydroxy, and halogen, such as F, Cl or Br, e.g. F or Cl.

In some embodiments of a compound of formula IV and formulas IVa, IVb or IVc, L¹ is —CH₂— and R¹, R², R³, and R⁴ are independently of each other selected from H, linear or branched —C₁₋₆ alkyl, —C₁₋₄ alkoxy, CF₃, CHF₂, CMeF₂, OCF₃, OCHF₂, —CN, and halogen, such as F, Cl or Br, e.g. F or Cl.

In some embodiments of a compound of formula IV and formulas IVa, IVb or IVc, L¹ is —CH₂— and R¹, R², R³, and R⁴ are independently of each other selected from H, linear or branched —C₁₋₄ alkyl, OCF₃, OCHF₂, and halogen, such as F, Cl or Br, e.g. F or Cl.

In some embodiments of a compound of formula IV and formulas IVa, IVb or IVc, L¹ is —CH₂—, R¹ is H and R², R³, R⁴ are independently of each other selected from H, linear or branched —C₁₋₆ alkyl, linear or branched C₁₋₆ heteroalkyl, —C₁₋₄ alkoxy, CF₃, CHF₂, CMeF₂, —O—(CH₂)₂—OMe, OCF₃, OCHF₂, —C₁₋₆ alkylamino, —CN, NH₂, —C₁₋₄ alkylhydroxy, and halogen, such as F, Cl or Br, e.g. F or Cl.

In some embodiments of a compound of formula IV and formulas IVa, IVb or IVc, L¹ is —CH₂—, R¹ is H and R², R³, R⁴ are independently of each other selected from H, linear or branched —C₁₋₆ alkyl, —C₁₋₄ alkoxy, CF₃, CHF₂, CMeF₂, OCF₃, OCHF₂, —CN, and halogen, such as F, Cl or Br, e.g. F or Cl.

In some embodiments of a compound of formula IV and formulas IVa, IVb or IVc, L¹ is —CH₂— and R¹ is H and R², R³, and R⁴ are independently of each other selected from H, linear or branched —C₁₋₄ alkyl, OCF₃, OCHF₂, and halogen, such as F, Cl or Br, e.g. F or Cl.

In some embodiments of a compound of formula IV and formulas IVa, IVb or IVc, L² is a covalent bond and R¹, R², R³, and R⁴ are independently of each other selected from H, linear or branched —C₁₋₆ alkyl, linear or branched C₁₋₆ heteroalkyl, —C₁₋₄ alkoxy, CF₃, CHF₂, CMeF₂, —O—(CH₂)₂—OMe, OCF₃, OCHF₂, —C₁— alkylamino, —CN, NH₂, —C₁₋₄ alkylhydroxy, and halogen, such as F, Cl or Br, e.g. F or Cl.

In some embodiments of a compound of formula IV and formulas IVa, IVb or IVc, L² is a covalent bond and R¹, R², R³, and R⁴ are independently of each other selected from H, linear or branched —C₁₋₆ alkyl, —C₁₋₄ alkoxy, CF₃, CHF₂, CMeF₂, OCF₃, OCHF₂, —CN, and halogen, such as F, Cl or Br, e.g. F or Cl.

In some embodiments of a compound of formula IV and formulas IVa, IVb or IVc, L² is a covalent bond and R¹, R², R³, and R⁴ are independently of each other selected from H, linear or branched —C₁₋₄ alkyl, OCF₃, OCHF₂, and halogen, such as F, Cl or Br, e.g. F or Cl. In some embodiments of a compound of formula IV and formulas IVa, IVb or IVc, L² is a covalent bond, R¹ is H and R², R³, R⁴ are independently of each other selected from H, linear or branched —C₁₋₆ alkyl, linear or branched C₁₋₆ heteroalkyl, —C₁₋₄ alkoxy, CF₃, CHF₂, CMeF₂, —O—(CH₂)₂—OMe, OCF₃, OCHF₂, —C₁₋₆ alkylamino, —CN, NH₂, —C₁₋₄ alkylhydroxy, and halogen, such as F, Cl or Br, e.g. F or Cl.

In some embodiments of a compound of formula IV and formulas IVa, IVb or IVc, L² is a covalent bond, R¹ is H and R², R³, R⁴ are independently of each other selected from H, linear or branched —C₁₋₆ alkyl, —C₁₋₄ alkoxy, CF₃, CHF₂, CMeF₂, OCF₃, OCHF₂, —CN, and halogen, such as F, Cl or Br, e.g. F or Cl.

In some embodiments of a compound of formula IV and formulas IVa, IVb or IVc, L² is a covalent bond, and R¹ is H and R², R³, and R⁴ are independently of each other selected from H, linear or branched —C₁₋₄ alkyl, OCF₃, OCHF₂, and halogen, such as F, Cl or Br, e.g. F or Cl.

In some embodiments of a compound of formula IV and formulas IVa, IVb or IVc, one of R¹, R², R³, and R⁴ is a group of formula -L³-X², wherein L³ is a covalent bond and X² is H, C₃₋₆ cycloalkyl, C₆ aryl, 6-membered heteroaryl, 5-6 membered heterocycloalkyl, wherein X² is unsubstituted or substituted with one or more of linear or branched C₁₋₄ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy.

In some embodiments of a compound of formula IV and formulas IVa, IVb or IVc, one of R¹, R², R³, and R⁴ is a group of formula -L³-X², wherein L³ is a covalent bond and X² is H, C₃₋₆ cycloalkyl, C₆ aryl, 6 membered heteroaryl, 4-8 membered heterocycloalkyl, wherein X² is unsubstituted or substituted with one or more of linear or branched C₁₋₄ alkyl, and halogen, e.g. F.

In some embodiments of a compound of formula IV and formulas IVa, IVb or IVc, one of R¹, R², R³, and R⁴ is a group of formula -L³-X², wherein L³ is a covalent bond and X² is H, cyclopropyl, cyclobutyl, C₆ aryl, pyridinyl, pyrrolidinyl, piperdinyl, wherein X² is unsubstituted or substituted with one or more of linear or branched C₁₋₄ alkyl, such as methyl, and halogen, such as F.

In some embodiments of a compound of formula IV and formulas IVa, IVb or IVc, one of R¹, R², R³, and R⁴ is a group of formula -L³-X², wherein L³ is —O— and X² is H, C₃₋₆ cycloalkyl, C₆ aryl, 6-membered heteroaryl, 5-6 membered heterocycloalkyl, wherein X² is unsubstituted or substituted with one or more of linear or branched C₁₋₄ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy.

In some embodiments of a compound of formula IV and formulas IVa, IVb or IVc, one of R¹, R², R³, and R⁴ is a group of formula -L³-X², wherein L³ is —O— and X² is H, C₃₋₆ cycloalkyl, C₆ aryl, 6 membered heteroaryl, 4-8 membered heterocycloalkyl, wherein X² is unsubstituted or substituted with one or more of linear or branched C₁₋₄ alkyl, and halogen, e.g. F.

In some embodiments of a compound of formula IV and formulas IVa, IVb or IVc, one of R¹, R², R³, and R⁴ is a group of formula -L³-X², wherein L³ is —O— and X² is H, cyclopropyl, cyclobutyl, C₆ aryl, pyridinyl, pyrrolidinyl, piperdinyl, wherein X² is unsubstituted or substituted with one or more of linear or branched C₁₋₄ alkyl, such as methyl, and halogen, such as F.

In some embodiments of a compound of formula IV and formulas IVa, IVb or IVc, one of R¹, R², R³, and R⁴ is a group of formula -L³-X², wherein L³ is —O— and X² is cyclopropyl.

In some embodiments the compound of formula IV and formulas IVa, IVb or IVc is a compound or pharmaceutically acceptable salts or stereoisomers thereof of formula IV-1, such as formula IV-1a, IV-1b or IV-1c:

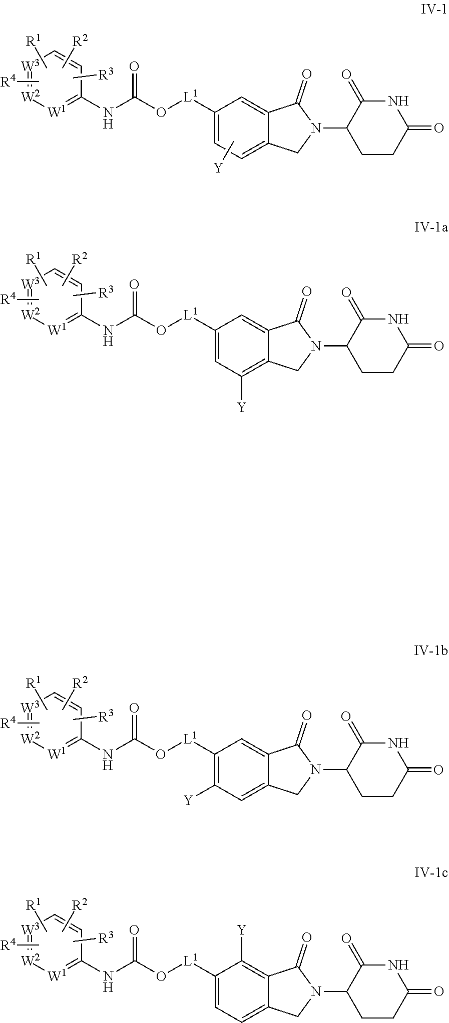

wherein
Y is linear or branched C₁₋₆ alkyl, —C₁₋₄ alkoxy, —CN, halogen, CF₃, CHF₂, CMeF₂, OCF₃, OCHF₂;
w¹, w², w³ are independently of each other selected from C, N, with the proviso that two or three of w¹, w², w³ are C;
R¹, R², R³, R⁴ are independently of each other selected from H, linear or branched —C₁a alkyl, linear or branched C₁₋₆ heteroalkyl, —C₁₋₄ alkoxy, CF₃, CHF₂, CMeF₂, —O—(CH₂)₂—OMe, OCF₃, OCHF₂, —C₁₋₆ alkylamino, —CN, —NH₂, —C₁₋₄ alkylhydroxy, and halogen, such as F, Cl or Br, e.g. F or Cl, or a group of formula -L³-X², wherein L³ is a covalent bond, linear or branched C₁₋₆ alkyl, —O—, and X² is C₃₋₆ cycloalkyl, C₆₋₁₀ aryl, 5-10 membered heteroaryl, 4-8 membered heterocycloalkyl, wherein X² is unsubstituted or substituted with one or more of linear or branched C₁₋₄ alkyl, —C₁₋₄ alkoxy, NH₂, NMe₂, halogen, CF₃, CHF₂, CMeF₂, —O—(CH₂)₂—OMe, OCF₃, OCHF₂, and C₁₋₄ alkylhydroxy.

In some embodiments of formula IV-1 and formulas IV-1a, IV-1b or IV-1c, Y is C₁₋₄ alkyl, such as methyl, —C₁₋₄ alkoxy, such as —OMe, —CN, halogen, such as F, Cl, Br.

In some embodiments of a compound of formula IV-1 and formulas IV-1a, IV-1b or IV-1c, L³ is a covalent bond. In some embodiments of a compound of formula IV and formulas IVa, IVb or IVc, L³ is —O—.

In some embodiments of a compound of formula IV-1 and formulas IV-1a, IV-1b or IV-1c, w¹, w², w³ are C. In some embodiments of a compound of formula IV-1 and formulas IV-1a, IV-1b or IV-1c, either w¹ is N and w², w³ are C. In some embodiments of a compound of formula IV-1 and formulas IV-1a, IV-1b or IV-1c, w² is N and w¹, w³ are C. In some embodiments of a compound of formula IV-1 and formulas IV-1a, IV-1b or IV-1c, w³ is N and w¹, w² are C.

In some embodiments of a compound of formula IV-1 and formulas IV-1a, IV-1b or IV-1c, R¹, R², R³, and R⁴ are independently of each other selected from H, linear or branched —C₁₋₆ alkyl, linear or branched C₁₋₆ heteroalkyl, —C₁₋₄ alkoxy, CF₃, CHF₂, CMeF₂, —O—(CH₂)₂—OMe, OCF₃, OCHF₂, —C₁₋₆ alkylamino, —CN, NH₂, —C₁₋₄ alkylhydroxy, and halogen, such as F, Cl or Br, e.g. F or Cl.

In some embodiments of a compound of formula IV-1 and formulas IV-1a, IV-1b or IV-1c, R¹, R², R³, and R⁴ are independently of each other selected from H, linear or branched —C₁₋₆ alkyl, —C₁₋₄ alkoxy, CF₃, CHF₂, CMeF₂, OCF₃, OCHF₂, —CN, and halogen, such as F, Cl or Br, e.g. F or Cl.

In some embodiments of a compound of formula IV-1 and formulas IV-1a, IV-1b or IV-1c, R¹, R², R³, and R⁴ are independently of each other selected from H, linear or branched —C₁₋₄ alkyl, OCF₃, OCHF₂, and halogen, such as F, Cl or Br, e.g. F or Cl.

In some embodiments of a compound of formula IV-1 and formulas IV-1a, IV-1b or IV-1c, R¹ is H and R², R³, R⁴ are independently of each other selected from H, linear or branched —C₁₋₆ alkyl, linear or branched C₁₋₆ heteroalkyl, —C₁₋₄ alkoxy, CF₃, CHF₂, CMeF₂, —O—(CH₂)₂—OMe, OCF₃, OCHF₂, —C₁— alkylamino, —CN, NH₂, —C₁₋₄ alkylhydroxy, and halogen, such as F, Cl or Br, e.g. F or Cl.

In some embodiments of a compound of formula IV-1 and formulas IV-1a, IV-1b or IV-1c, R¹ is H and R², R³, R⁴ are independently of each other selected from H, linear or branched —C₁₋₆ alkyl, —C₁₋₄ alkoxy, CF₃, CHF₂, CMeF₂, OCF₃, OCHF₂, —CN, and halogen, such as F, Cl or Br, e.g. F or Cl.

In some embodiments of a compound of formula IV-1 and formulas IV-1a, IV-1b or IV-1c, R¹ is H and R², R³, and R⁴ are independently of each other selected from H, linear or branched —C₁₋₄ alkyl, OCF₃, OCHF₂, and halogen, such as F, Cl or Br, e.g. F or Cl.

In some embodiments of a compound of formula IV-1 and formulas IV-1a, IV-1b or IV-1c, one of R¹, R², R³, and R⁴ is a group of formula -L³-X², wherein L³ is a covalent bond and X² is H, C₃₋₆ cycloalkyl, C₆ aryl, 6-membered heteroaryl, 5-6 membered heterocycloalkyl, wherein X² is unsubstituted or substituted with one or more of linear or branched C₁₋₄ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy.

In some embodiments of a compound of formula IV-1 and formulas IV-1a, IV-1b or IV-1c, one of R¹, R², R³, and R⁴ is a group of formula -L³-X², wherein L³ is a covalent bond and X² is H, C₃₋₆ cycloalkyl, C₆ aryl, 6 membered heteroaryl, 4-8 membered heterocycloalkyl, wherein X² is unsubstituted or substituted with one or more of linear or branched C₁₋₄ alkyl, and halogen, e.g. F.

In some embodiments of a compound of formula IV-1 and formulas IV-1a, IV-1b or IV-1c, one of R¹, R², R³, and R⁴ is a group of formula -L³-X², wherein L³ is a covalent bond and X² is H, cyclopropyl, cyclobutyl, C₆ aryl, pyridinyl, pyrrolidinyl, piperdinyl, wherein X² is unsubstituted or substituted with one or more of linear or branched C₁₋₄ alkyl, such as methyl, and halogen, such as F.

In some embodiments of a compound of formula IV-1 and formulas IV-1a, IV-1b or IV-1c, one of R¹, R², R³, and R⁴ is a group of formula -L³-X², wherein L³ is —O— and X² is H, C₃₋₆ cycloalkyl, C₆ aryl, 6-membered heteroaryl, 5-6 membered heterocycloalkyl, wherein X² is unsubstituted or substituted with one or more of linear or branched C₁₋₄ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy.

In some embodiments of a compound of formula IV-1 and formulas IV-1a, IV-1b or IV-1c, one of R¹, R², R³, and R⁴ is a group of formula -L³-X², wherein L³ is —O— and X² is H, C₃₋₆ cycloalkyl, C₆ aryl, 6 membered heteroaryl, 4-8 membered heterocycloalkyl, wherein X² is unsubstituted or substituted with one or more of linear or branched C₁₋₄ alkyl, and halogen, e.g. F.

In some embodiments of a compound of formula IV-1 and formulas IV-1a, IV-1b or IV-1c, one of R¹, R², R³, and R⁴ is a group of formula -L³-X², wherein L³ is —O— and X² is H, cyclopropyl, cyclobutyl, C₆ aryl, pyridinyl, pyrrolidinyl, piperdinyl, wherein X² is unsubstituted or substituted with one or more of linear or branched C₁₋₄ alkyl, such as methyl, and halogen, such as F.

In some embodiments of a compound of formula IV-1 and formulas IV-1a, IV-1b or IV-1c, one of R¹, R², R³, and R⁴ is a group of formula -L³-X², wherein L³ is —O— and X² is cyclopropyl.

In some embodiments, the compound of formula IV is a compound or pharmaceutically acceptable salts or stereoisomers thereof of formula V, such as Va, Vb or Vc, wherein w¹, w², w³ are C:

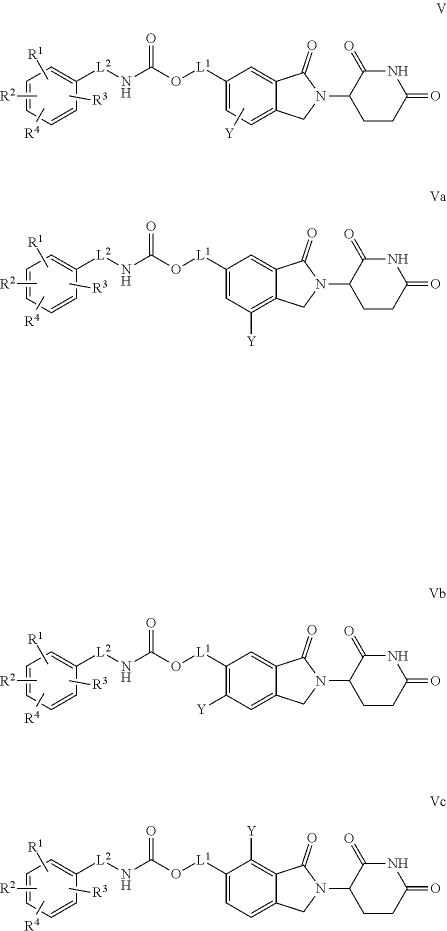

wherein
Y is linear or branched C₁₋₆ alkyl, —C₁₋₄ alkoxy, —CN, halogen, CF₃, CHF₂, CMeF₂, OCF₃, OCHF₂;
R¹, R², R³, R⁴ are independently of each other selected from H, linear or branched —C₁₋₆ alkyl, linear or branched C₁₋₄ heteroalkyl, —C₁₋₄ alkoxy, CF₃, CHF₂, CMeF₂, —O—(CH₂)₂—OMe, OCF₃, OCHF₂, —C₁₋₆ alkylamino, —CN, —NH₂, —C₁₋₄ alkylhydroxy, and halogen, such as F, Cl or Br, e.g. F or Cl, or a group of formula -L³-X², wherein L³ is a covalent bond, linear or branched C₁₋₆ alkyl, —O—, and X² is C₃₋₆ cycloalkyl, C₆₋₁₀ aryl, 5-10 membered heteroaryl, 4-8 membered heterocycloalkyl, wherein X² is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, and C₁₋₄ alkylhydroxy;
L¹ is linear or branched C₁₋₆ alkyl; L² is a covalent bond, linear or branched C₁₋₆ alkyl.

In some embodiments of formula V and formulas Va, Vb or Vc, Y is C₁₋₄ alkyl, such as methyl, —C₁₋₄ alkoxy, such as —OMe, —CN, halogen, such as F, Cl, Br.

In some embodiments of a compound of formula V and formulas Va, Vb or Vc, L¹ is linear or branched C₁₋₄ alkyl. In some embodiments of a compound of formula V and formulas Va, Vb or Vc, L¹ is —CH₂—.

In some embodiments of a compound of formula V and formulas Va, Vb or Vc, L² is a covalent bond. In some embodiments of a compound of formula V and formulas Va, Vb or Vc, L² is —CH₂—.

In some embodiments of a compound of formula V and formulas Va, Vb or Vc, L³ is a covalent bond. In some embodiments of a compound of formula V and formulas Va, Vb or Vc, L³ is —O—.

In some embodiments of a compound of formula V and formulas Va, Vb or Vc, L¹ is —CH₂— and L² is a covalent bond.

In some embodiments of a compound of formula V and formulas Va, Vb or Vc, L¹ is —CH₂—, L² is a covalent bond and L³ is a covalent bond. In some embodiments of a compound of formula V and formulas Va, Vb or Vc, L¹ is —CH₂—, L² is a covalent bond and L³ is —O—.

In some embodiments of a compound of formula V and formulas Va, Vb or Vc, R¹, R², R³, and R⁴ are independently of each other selected from H, linear or branched —C₁₋₆ alkyl, linear or branched C₁₋₆ heteroalkyl, —C₁₋₄ alkoxy, CF₃, CHF₂, CMeF₂, —O—(CH₂)₂—OMe, OCF₃, OCHF₂, —C₁— alkylamino, —CN, NH₂, —C₁₋₄ alkylhydroxy, and halogen, such as F, Cl or Br, e.g. F or Cl.

In some embodiments of a compound of formula V and formulas Va, Vb or Vc, R¹, R², R³, and R⁴ are independently of each other selected from H, linear or branched —C₁₋₆ alkyl, —C₁₋₄ alkoxy, CF₃, CHF₂, CMeF₂, OCF₃, OCHF₂, —CN, and halogen, such as F, Cl or Br, e.g. F or Cl.

In some embodiments of a compound of formula V and formulas Va, Vb or Vc, R¹, R², R³, and R⁴ are independently of each other selected from H, linear or branched —C₁₋₄ alkyl, OCF₃, OCHF₂, and halogen, such as F, Cl or Br, e.g. F or Cl.

In some embodiments of a compound of formula V and formulas Va, Vb or Vc, R¹ is H and R², R³, R⁴ are independently of each other selected from H, linear or branched —C₁₋₆ alkyl, linear or branched C₁₋₆ heteroalkyl, —C₁₋₄ alkoxy, CF₃, CHF₂, CMeF₂, —O—(CH₂)₂—OMe, OCF₃, OCHF₂, —C₁₋₆ alkylamino, —CN, NH₂, —C₁₋₄ alkylhydroxy, and halogen, such as F, Cl or Br, e.g. F or Cl.

In some embodiments of a compound of formula V and formulas Va, Vb or Vc, R¹ is H and R², R³, R⁴ are independently of each other selected from H, linear or branched —C₁₋₆ alkyl, —C₁₋₄ alkoxy, CF₃, CHF₂, CMeF₂, OCF₃, OCHF₂, —CN, and halogen, such as F, Cl or Br, e.g. F or Cl.

In some embodiments of a compound of formula V and formulas Va, Vb or Vc, R¹ is H and R², R³, and R⁴ are independently of each other selected from H, linear or branched —C₁₋₄ alkyl, OCF₃, OCHF₂, and halogen, such as F, Cl or Br, e.g. F or Cl.

In some embodiments of a compound of formula V and formulas Va, Vb or Vc, L¹ is —CH₂— and R¹, R², R³, and R⁴ are independently of each other selected from H, linear or branched —C₁₋₆ alkyl, linear or branched C₁₋₆ heteroalkyl, —C₁₋₄ alkoxy, CF₃, CHF₂, CMeF₂, —O—(CH₂)₂—OMe, OCF₃, OCHF₂, —C₁₋₆ alkylamino, —CN, NH₂, —C₁₋₄ alkylhydroxy, and halogen, such as F, Cl or Br, e.g. F or Cl.

In some embodiments of a compound of formula V and formulas Va, Vb or Vc, L¹ is —CH₂— and R¹, R², R³, and R⁴ are independently of each other selected from H, linear or branched —C₁₋₆ alkyl, —C₁₋₄ alkoxy, CF₃, CHF₂, CMeF₂, OCF₃, OCHF₂, —CN, and halogen, such as F, Cl or Br, e.g. F or Cl.

In some embodiments of a compound of formula V and formulas Va, Vb or Vc, L¹ is —CH₂— and R¹, R², R³, and R⁴ are independently of each other selected from H, linear or branched —C₁₋₄ alkyl, OCF₃, OCHF₂, and halogen, such as F, Cl or Br, e.g. F or Cl.

In some embodiments of a compound of formula V and formulas Va, Vb or Vc, L¹ is —CH₂—, R¹ is H and R², R³, R⁴ are independently of each other selected from H, linear or branched —C₁₋₆ alkyl, linear or branched C₁₋₆ heteroalkyl, —C₁₋₄ alkoxy, CF₃, CHF₂, CMeF₂, —O—(CH₂)₂—OMe, OCF₃, OCHF₂, —C₁₋₆ alkylamino, —CN, NH₂, —C₁₋₄ alkylhydroxy, and halogen, such as F, Cl or Br, e.g. F or Cl.

In some embodiments of a compound of formula V and formulas Va, Vb or Vc, L¹ is —CH₂—, R¹ is H and R², R³, R⁴ are independently of each other selected from H, linear or branched —C₁₋₆ alkyl, —C₁₋₄ alkoxy, CF₃, CHF₂, CMeF₂, OCF₃, OCHF₂, —CN, and halogen, such as F, Cl or Br, e.g. F or Cl.

In some embodiments of a compound of formula V and formulas Va, Vb or Vc, L¹ is —CH₂— and R¹ is H and R², R³, and R⁴ are independently of each other selected from H, linear or branched —C₁₋₄ alkyl, OCF₃, OCHF₂, and halogen, such as F, Cl or Br, e.g. F or Cl.

In some embodiments of a compound of formula V and formulas Va, Vb or Vc, L² is a covalent bond and R¹, R², R³, and R⁴ are independently of each other selected from H, linear or branched —C₁₋₆ alkyl, linear or branched C₁₋₆ heteroalkyl, —C₁₋₄ alkoxy, CF₃, CHF₂, CMeF₂, —O—(CH₂)₂—OMe, OCF₃, OCHF₂, —C₁₋₆ alkylamino, —CN, NH₂, —C₁₋₄ alkylhydroxy, and halogen, such as F, Cl or Br, e.g. F or Cl.

In some embodiments of a compound of formula V and formulas Va, Vb or Vc, L² is a covalent bond and R¹, R², R³, and R⁴ are independently of each other selected from H, linear or branched —C₁₋₆ alkyl, —C₁₋₄ alkoxy, CF₃, CHF₂, CMeF₂, OCF₃, OCHF₂, —CN, and halogen, such as F, Cl or Br, e.g. F or Cl.

In some embodiments of a compound of formula V and formulas Va, Vb or Vc, L² is a covalent bond and R¹, R², R³, and R⁴ are independently of each other selected from H, linear or branched —C₁₋₄ alkyl, OCF₃, OCHF₂, and halogen, such as F, Cl or Br, e.g. F or Cl.

In some embodiments of a compound of formula V and formulas Va, Vb or Vc, L² is a covalent bond, R¹ is H and R², R³, R⁴ are independently of each other selected from H, linear or branched —C₁₋₆ alkyl, linear or branched C₁₋₆ heteroalkyl, —C₁₋₄ alkoxy, CF₃, CHF₂, CMeF₂, —O—(CH₂)₂—OMe, OCF₃, OCHF₂, —C₁₋₆ alkylamino, —CN, NH₂, —C₁₋₄ alkylhydroxy, and halogen, such as F, Cl or Br, e.g. F or Cl.

In some embodiments of a compound of formula V and formulas Va, Vb or Vc, L² is a covalent bond, R¹ is H and R², R³, R⁴ are independently of each other selected from H, linear or branched —C₁₋₆ alkyl, —C₁₋₄ alkoxy, CF₃, CHF₂, CMeF₂, OCF₃, OCHF₂, —CN, and halogen, such as F, Cl or Br, e.g. F or Cl.

In some embodiments of a compound of formula V and formulas Va, Vb or Vc, L² is a covalent bond, and R¹ is H and R², R³, R⁴ are independently of each other selected from H, linear or branched —C₁₋₄ alkyl, OCF₃, OCHF₂, and halogen, such as F, Cl or Br, e.g. F or Cl.

In some embodiments of a compound of formula V and formulas Va, Vb or Vc, one of R¹, R², R³, R⁴ is a group of formula -L³-X², wherein L³ is a covalent bond and X² is H, C₃₋₆ cycloalkyl, C₆ aryl, 6-membered heteroaryl, 5-6 membered heterocycloalkyl, wherein X² is unsubstituted or substituted with one or more of linear or branched C₁₋₄ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy.

In some embodiments of a compound of formula V and formulas Va, Vb or Vc, one of R¹, R², R³, R⁴ is a group of formula -L³-X², wherein L³ is a covalent bond and X² is H, C₃₋₆ cycloalkyl, C₆ aryl, 6 membered heteroaryl, 4-8 membered heterocycloalkyl, wherein X² is unsubstituted or substituted with one or more of linear or branched C₁₋₄ alkyl, and halogen, e.g. F.

In some embodiments of a compound of formula V and formulas Va, Vb or Vc, one of R¹, R², R³, R⁴ is a group of formula -L³-X², wherein L³ is a covalent bond and X² is H, cyclopropyl, cyclobutyl, C₆ aryl, pyridinyl, pyrrolidinyl, piperdinyl, wherein X² is unsubstituted or substituted with one or more of linear or branched C₁₋₄ alkyl, such as methyl, and halogen, such as F.

In some embodiments of a compound of formula V and formulas Va, Vb or Vc, one of R¹, R², R³, R⁴ is a group of formula -L³-X², wherein L³ is —O— and X² is H, C₃₋₆ cycloalkyl, C₆ aryl, 6-membered heteroaryl, 5-6 membered heterocycloalkyl, wherein X² is unsubstituted or substituted with one or more of linear or branched C₁₋₄ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy.

In some embodiments of a compound of formula V and formulas Va, Vb or Vc, one of R¹, R², R³, R⁴ is a group of formula -L³-X², wherein L³ is —O— and X² is H, C₃₋₆ cycloalkyl, C₆ aryl, 6 membered heteroaryl, 4-8 membered heterocycloalkyl, wherein X² is unsubstituted or substituted with one or more of linear or branched C₁₋₄ alkyl, and halogen, e.g. F.

In some embodiments of a compound of formula V and formulas Va, Vb or Vc, one of R¹, R², R³, R⁴ is a group of formula -L³-X², wherein L³ is —O— and X² is H, cyclopropyl, cyclobutyl, C₆ aryl, pyridinyl, pyrrolidinyl, piperdinyl, wherein X² is unsubstituted or substituted with one or more of linear or branched C₁₋₄ alkyl, such as methyl, and halogen, such as F.

In some embodiments of a compound of formula V and formulas Va, Vb or Vc, one of R¹, R², R³, R⁴ is a group of formula -L³-X², wherein L³ is —O— and X² is cyclopropyl.

In some embodiments the compound of formula V and formulas Va, Vb or Vc is a compound or pharmaceutically acceptable salts or stereoisomers thereof of formula V-1, such as formula V-1a, V-1b or V-1c:

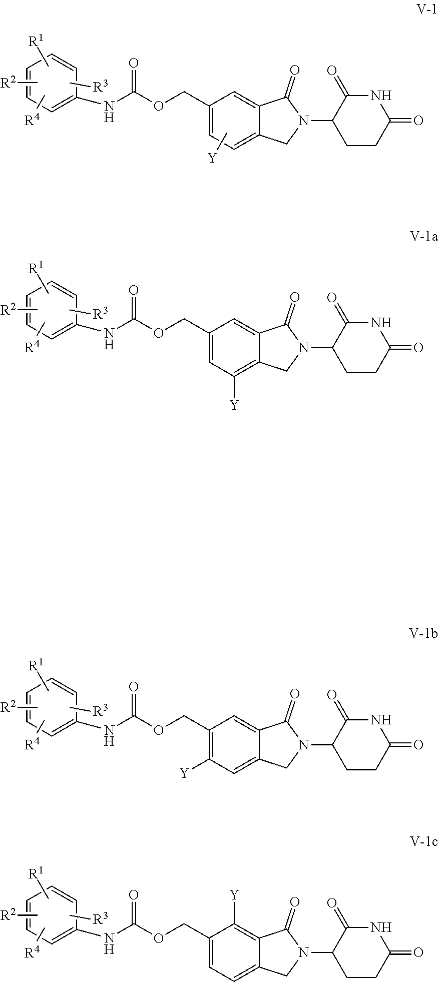

wherein
Y is linear or branched C₁₋₆ alkyl, —C₁₋₄ alkoxy, —CN, halogen, CF₃, CHF₂, CMeF₂, OCF₃, OCHF₂;
R¹, R², R³, R⁴ are independently of each other selected from H, linear or branched —C₁₋₆ alkyl, linear or branched C₁₋₆ heteroalkyl, —C₁₋₄ alkoxy, CF₃, CHF₂, CMeF₂, —O—(CH₂)₂—OMe, OCF₃, OCHF₂, —C₁₋₆ alkylamino, —CN, —NH₂, —C₁₋₄ alkylhydroxy, and halogen, such as F, Cl or Br, e.g. F or Cl, or a group of formula -L³-X², wherein L³ is a covalent bond, linear or branched C₁₋₆ alkyl, —O—, and X² is C₃₋₆ cycloalkyl, C₆₋₁₀ aryl, 5-10 membered heteroaryl, 4-8 membered heterocycloalkyl, wherein X² is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, and C₁₋₄ alkylhydroxy.

In some embodiments of formula V-1 and formulas V-Ia, V-1b or V-Ic, Y is C₁₋₄ alkyl, such as methyl, —C₁₋₄ alkoxy, such as —OMe, —CN, halogen, such as F, Cl, Br.

In some embodiments of a compound of formula V-1 and formulas V-1a, V-1b or V-1c, L³ is a covalent bond. In some embodiments of a compound of formula V and formulas Va, Vb or Vc, L³ is —O—.

In some embodiments of a compound of formula V-1 and formulas V-Ia, V-1b or V-Ic, R¹, R², R³, R⁴ are independently of each other selected from H, linear or branched —C₁₋₆ alkyl, linear or branched C₁₋₆ heteroalkyl, —C₁₋₄ alkoxy, CF₃, CHF₂, CMeF₂, —O—(CH₂)₂—OMe, OCF₃, OCHF₂, —C₁₋₆ alkylamino, —CN, NH₂, —C₁₋₄ alkylhydroxy, and halogen, such as F, Cl or Br, e.g. F or Cl.

In some embodiments of a compound of formula V-1 and formulas V-Ia, V-1b or V-Ic, R¹, R², R³, R⁴ are independently of each other selected from H, linear or branched —C₁₋₆ alkyl, —C₁₋₄ alkoxy, CF₃, CHF₂, CMeF₂, OCF₃, OCHF₂, —CN, and halogen, such as F, Cl or Br, e.g. F or Cl.

In some embodiments of a compound of formula V-1 and formulas V-Ia, V-Ib or V-Ic, R¹, R², R³, R⁴ are independently of each other selected from H, linear or branched —C₁₋₄ alkyl, OCF₃, OCHF₂, and halogen, such as F, Cl or Br, e.g. F or Cl.

In some embodiments of a compound of formula V-1 and formulas V-1a, V-1b or V-1c, R¹ is H and R², R³, R⁴ are independently of each other selected from H, linear or branched —C₁₋₆ alkyl, linear or branched C₁₋₆ heteroalkyl, —C₁₋₄ alkoxy, CF₃, CHF₂, CMeF₂, —O—(CH₂)₂—OMe, OCF₃, OCHF₂, —C₁₋₆ alkylamino, —CN, NH₂, —C₁₋₄ alkylhydroxy, and halogen, such as F, Cl or Br, e.g. F or Cl.

In some embodiments of a compound of formula V-1 and formulas V-1a, V-1b or V-1c, R¹ is H and R², R³, R⁴ are independently of each other selected from H, linear or branched —C₁₋₆ alkyl, —C₁₋₄ alkoxy, CF₃, CHF₂, CMeF₂, OCF₃, OCHF₂, —CN, and halogen, such as F, Cl or Br, e.g. F or Cl.

In some embodiments of a compound of formula V-1 and formulas V-1a, V-1b or V-1c, R¹ is H and R², R³, R⁴ are independently of each other selected from H, linear or branched —C₁₋₄ alkyl, OCF₃, OCHF₂, and halogen, such as F, Cl or Br, e.g. F or Cl.

In some embodiments of a compound of formula V-1 and formulas V-Ia, V-Ib or V-Ic, one of R¹, R², R³, R⁴ is a group of formula -L³-X², wherein L³ is a covalent bond and X² is H, C₃₋₆ cycloalkyl, C₆ aryl, 6-membered heteroaryl, 5-6 membered heterocycloalkyl, wherein X² is unsubstituted or substituted with one or more of linear or branched C₁₋₄ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy.

In some embodiments of a compound of formula V-1 and formulas V-Ia, V-Ib or V-Ic, one of R¹, R², R³, R⁴ is a group of formula -L³-X², wherein L³ is a covalent bond and X² is H, C₃₋₆ cycloalkyl, C₆ aryl, 6 membered heteroaryl, 4-8 membered heterocycloalkyl, wherein X² is unsubstituted or substituted with one or more of linear or branched C₁₋₄ alkyl, and halogen, e.g. F.

In some embodiments of a compound of formula V-1 and formulas V-Ia, V-1b or V-Ic, one of R¹, R², R³, R⁴ is a group of formula -L³-X², wherein L³ is a covalent bond and X² is H, cyclopropyl, cyclobutyl, C₆ aryl, pyridinyl, pyrrolidinyl, piperdinyl, wherein X² is unsubstituted or substituted with one or more of linear or branched C₁₋₄ alkyl, such as methyl, and halogen, such as F.

In some embodiments of a compound of formula V-1 and formulas V-Ia, V-Ib or V-Ic, one of R¹, R², R³, R⁴ is a group of formula -L³-X², wherein L³ is —O— and X² is H, C₃₋₆ cycloalkyl, C₆ aryl, 6-membered heteroaryl, 5-6 membered heterocycloalkyl, wherein X² is unsubstituted or substituted with one or more of linear or branched C₁₋₄ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy.

In some embodiments of a compound of formula V-1 and formulas V-Ia, V-Ib or V-Ic, one of R¹, R², R³, R⁴ is a group of formula -L³-X², wherein L³ is —O— and X² is H, C₃₋₆ cycloalkyl, C₆ aryl, 6 membered heteroaryl, 4-8 membered heterocycloalkyl, wherein X² is unsubstituted or substituted with one or more of linear or branched C₁₋₄ alkyl, and halogen, e.g. F.

In some embodiments of a compound of formula V-1 and formulas V-Ia, V-Ib or V-Ic, one of R¹, R², R³, R⁴ is a group of formula -L³-X², wherein L³ is —O— and X² is H, cyclopropyl, cyclobutyl, C₆ aryl, pyridinyl, pyrrolidinyl, piperdinyl, wherein X² is unsubstituted or substituted with one or more of linear or branched C₁₋₄ alkyl, such as methyl, and halogen, such as F.

In some embodiments of a compound of formula V-1 and formulas V-Ia, V-Ib or V-Ic, one of R¹, R², R³, R⁴ is a group of formula -L³-X², wherein L³ is —O— and X² is cyclopropyl.

In some embodiments the compound of formula V is a compound or pharmaceutically acceptable salts or stereoisomers thereof of formula VI-1, VI-2, VI-3, VI-4, VI-5, VI-6:

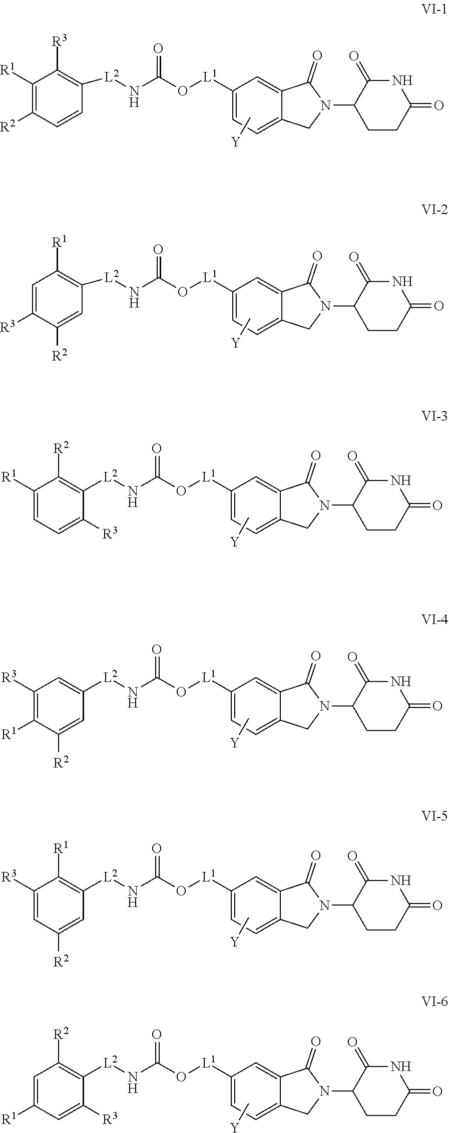

wherein
Y is linear or branched C₁₋₆ alkyl, —C₁₋₄ alkoxy, —CN, halogen, CF₃, CHF₂, CMeF₂, OCF₃, OCHF₂;
R¹, R², R³ are independently of each other selected from H, linear or branched —C₁₋₆ alkyl, linear or branched C₁₋₆ heteroalkyl, —C₁₋₄ alkoxy-C₁₋₄ alkylhydroxy, and halogen, such as F, Cl or Br, e.g. F or Cl, or a group of formula -L³-X², wherein L³ is a covalent bond, linear or branched C₁₋₆ alkyl, —O—, —C₁₋₄ alkoxy and X² is C₃₋₆ cycloalkyl, C₆₋₁₀ aryl, 5-10 membered heteroaryl, 4-8 membered heterocycloalkyl, wherein X² is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy;
L¹ is linear or branched C₁₋₆ alkyl; L² is a covalent bond, linear or branched C₁₋₆ alkyl.

In some embodiments of a compound of formula VI-1, VI-2, VI-3, VI-4, VI-5, VI-6, Y is in the 4-position. In some embodiments of a compound of formula I, Y is in the 5-position. In some embodiments of a compound of formula I, Y is in the 7-position.

In some embodiments of formula VI-1, VI-2, VI-3, VI-4, VI-5, VI-6, Y is C₁₋₆ alkyl, such as methyl, —C1-4 alkoxy, such as —OMe, —CN, halogen, such as F, Cl, Br.

In some embodiments of a compound of formula VI-1, VI-2, VI-3, VI-4, VI-5, VI-6, L¹ is linear or branched C₁₋₄ alkyl. In some embodiments of a compound of formula I, L¹ is —CH₂—.

In some embodiments of a compound of formula VI-1, VI-2, VI-3, VI-4, VI-5, VI-6, L² is a covalent bond. In some embodiments of a compound of formula I, L² is —CH₂—.

In some embodiments of a compound of formula VI-1, VI-2, VI-3, VI-4, VI-5, VI-6, L³ is a covalent bond. In some embodiments of a compound of formula VI-1, VI-2, VI-3, VI-4, VI-5, VI-6, L³ is —O—.

In some embodiments of a compound of formula VI-1, VI-2, VI-3, VI-4, VI-5, VI-6, L¹ is —CH₂— and L² is a covalent bond.

In some embodiments of a compound of formula VI-1, VI-2, VI-3, VI-4, VI-5, VI-6, L¹ is —CH₂—, L² is a covalent bond and L³ is a covalent bond. In some embodiments of a compound of formula VI-1, VI-2, VI-3, VI-4, VI-5, VI-6, L¹ is —CH₂—, L² is a covalent bond and L³ is —O—.

In some embodiments of a compound of formula VI-1, VI-2, VI-3, VI-4, VI-5, VI-6, R¹, R², R³ are independently of each other selected from H, linear or branched —C₁₋₆ alkyl, linear or branched C₁₋₆ heteroalkyl, —C₁₋₄ alkoxy, CF₃, CHF₂, CMeF₂, —O—(CH₂)₂—OMe, OCF₃, OCHF₂, —C₁₋₆ alkylamino, —CN, NH₂, —C₁₋₄ alkylhydroxy, and halogen, such as F, Cl or Br, e.g. F or Cl.

In some embodiments of a compound of formula VI-1, VI-2, VI-3, VI-4, VI-5, VI-6, R¹, R², R³ are independently of each other selected from H, linear or branched —C₁₋₆ alkyl, —C₁₋₄ alkoxy, CF₃, CHF₂, CMeF₂, OCF₃, OCHF₂, —CN, and halogen, such as F, Cl or Br, e.g. F or Cl.

In some embodiments of a compound of formula VI-1, VI-2, VI-3, VI-4, VI-5, VI-6, R¹, R², R³ are independently of each other selected from H, linear or branched —C₁₋₄ alkyl, OCF₃, OCHF₂, and halogen, such as F, Cl or Br, e.g. F or Cl.

In some embodiments of a compound of formula VI-1, VI-2, VI-3, VI-4, VI-5, VI-6, L¹ is —CH₂— and R¹, R², R³ are independently of each other selected from H, linear or branched —C₁₋₆ alkyl, linear or branched C₁₋₆ heteroalkyl, —C₁₋₄ alkoxy, CF₃, CHF₂, CMeF₂, —O—(CH₂)₂—OMe, OCF₃, OCHF₂, —C₁₋₆ alkylamino, —CN, NH₂, —C₁₋₄ alkylhydroxy, and halogen, such as F, Cl or Br, e.g. F or Cl.

In some embodiments of a compound of formula VI-1, VI-2, VI-3, VI-4, VI-5, VI-6, L¹ is —CH₂— and R¹, R², R³ are independently of each other selected from H, linear or branched —C₁₋₆ alkyl, —C₁₋₄ alkoxy, CF₃, CHF₂, CMeF₂, OCF₃, OCHF₂, —CN, and halogen, such as F, Cl or Br, e.g. F or Cl.

In some embodiments of a compound of formula VI-1, VI-2, VI-3, VI-4, VI-5, VI-6, L¹ is —CH₂— and R¹, R², R³ are independently of each other selected from H, linear or branched —C₁₋₄ alkyl, OCF₃, OCHF₂, and halogen, such as F, Cl or Br, e.g. F or Cl.

In some embodiments of a compound of formula VI-1, VI-2, VI-3, VI-4, VI-5, VI-6, L² is a covalent bond and R¹, R², R³ are independently of each other selected from H, linear or branched —C₁₋₆ alkyl, linear or branched C₁₋₆ heteroalkyl, —C₁₋₄ alkoxy, CF₃, CHF₂, CMeF₂, —O—(CH₂)₂—OMe, OCF₃, OCHF₂, —C₁₋₆ alkylamino, —CN, NH₂, —C₁₋₄ alkylhydroxy, and halogen, such as F, Cl or Br, e.g. F or Cl.

In some embodiments of a compound of formula VI-1, VI-2, VI-3, VI-4, VI-5, VI-6, L² is a covalent bond and R¹, R², R³ are independently of each other selected from H, linear or branched —C₁₋₆ alkyl, —C₁₋₄ alkoxy, CF₃, CHF₂, CMeF₂, OCF₃, OCHF₂, —CN, and halogen, such as F, Cl or Br, e.g. F or Cl.

In some embodiments of a compound of formula VI-1, VI-2, VI-3, VI-4, VI-5, VI-6, L² is a covalent bond and R¹, R², R³ are independently of each other selected from H, linear or branched —C₁₋₄ alkyl, OCF₃, OCHF₂, and halogen, such as F, Cl or Br, e.g. F or Cl.

In some embodiments of a compound of formula VI-1, VI-2, VI-3, VI-4, VI-5, VI-6, one of R¹, R², R³ is a group of formula -L³-X², wherein L³ is a covalent bond and X² is H, C₃₋₆ cycloalkyl, C₆ aryl, 6-membered heteroaryl, 5-6 membered heterocycloalkyl, wherein X² is unsubstituted or substituted with one or more of linear or branched C₁₋₄ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy.

In some embodiments of a compound of formula VI-1, VI-2, VI-3, VI-4, VI-5, VI-6, one of R¹, R², R³ is a group of formula -L³-X², wherein L³ is a covalent bond and X² is H, C₃₋₆ cycloalkyl, C₆ aryl, 6 membered heteroaryl, 4-8 membered heterocycloalkyl, wherein X² is unsubstituted or substituted with one or more of linear or branched C₁₋₄ alkyl, and halogen, e.g. F.

In some embodiments of a compound of formula VI-1, VI-2, VI-3, VI-4, VI-5, VI-6, one of R¹, R², R³ is a group of formula -L³-X², wherein L³ is a covalent bond and X² is H, cyclopropyl, cyclobutyl, C₆ aryl, pyridinyl, pyrrolidinyl, piperdinyl, wherein X² is unsubstituted or substituted with one or more of linear or branched C₁₋₄ alkyl, such as methyl, and halogen, such as F.

In some embodiments of a compound of formula VI-1, VI-2, VI-3, VI-4, VI-5, VI-6, one of R¹, R², R³ is a group of formula -L³-X², wherein L³ is —O— and X² is H, C₃₋₆ cycloalkyl, C₆ aryl, 6-membered heteroaryl, 5-6 membered heterocycloalkyl, wherein X² is unsubstituted or substituted with one or more of linear or branched C₁₋₄ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy.

In some embodiments of a compound of formula VI-1, VI-2, VI-3, VI-4, VI-5, VI-6, one of R¹, R², R³ is a group of formula -L³-X², wherein L³ is —O— and X² is H, C₃₋₆ cycloalkyl, C₆ aryl, 6 membered heteroaryl, 4-8 membered heterocycloalkyl, wherein X² is unsubstituted or substituted with one or more of linear or branched C₁₋₄ alkyl, and halogen, e.g. F.

In some embodiments of a compound of formula VI-1, VI-2, VI-3, VI-4, VI-5, VI-6, one of R¹, R², R³ is a group of formula -L³-X², wherein L³ is —O— and X² is H, cyclopropyl, cyclobutyl, C₆ aryl, pyridinyl, pyrrolidinyl, piperdinyl, wherein X² is unsubstituted or substituted with one or more of linear or branched C₁₋₄ alkyl, such as methyl, and halogen, such as F.

In some embodiments of a compound of formula VI-1, VI-2, VI-3, VI-4, VI-5, VI-6, one of R¹, R², R³ is a group of formula -L³-X², wherein L³ is —O— and X² is cyclopropyl.

In some embodiments of a compound of formula VI-1, VI-2, VI-3, VI-4, VI-5, VI-6, in which one of R¹, R², R³ is a group of formula -L³-X², as defined for these formulae in the paragraphs above, the other two of R¹, R², R³ are independently of each other selected from H, linear or branched —C₁₋₆ alkyl, linear or branched C₁₋₆ heteroalkyl, —C₁₋₄ alkoxy, CF₃, CHF₂, CMeF₂, —O—(CH₂)₂—OMe, OCF₃, OCHF₂, —C₁₋₆ alkylamino, —CN, NH₂, —C₁₋₄ alkylhydroxy, and halogen, such as F, Cl or Br, e.g. F or Cl.

In some embodiments of a compound of formula VI-1, VI-2, VI-3, VI-4, VI-5, VI-6, in which one of R¹, R², R³ is a group of formula -L³-X², as defined for these formulae in the paragraphs above, the other two of R¹, R², R³ are independently of each other selected from H, linear or branched —C₁₋₆ alkyl, —C₁₋₄ alkoxy, CF₃, CHF₂, CMeF₂, OCF₃, OCHF₂, —CN, and halogen, such as F, Cl or Br, e.g. F or Cl.

In some embodiments of a compound of formula VI-1, VI-2, VI-3, VI-4, VI-5, VI-6, in which one of R¹, R², R³ is a group of formula -L³-X², as defined for these formulae in the paragraphs above, the other two of R¹, R², R³ are independently of each other selected from H, linear or branched —C₁₋₄ alkyl, OCF₃, OCHF₂, and halogen, such as F, Cl or Br, e.g. F or Cl.

In some embodiments the compound of formula VI-1, VI-2, VI-3, VI-4, VI-5, VI-6 is a compound or pharmaceutically acceptable salts or stereoisomers thereof of formula VI-la, VI-2a, VI-3a, VI-4a, VI-5a, VI-6a

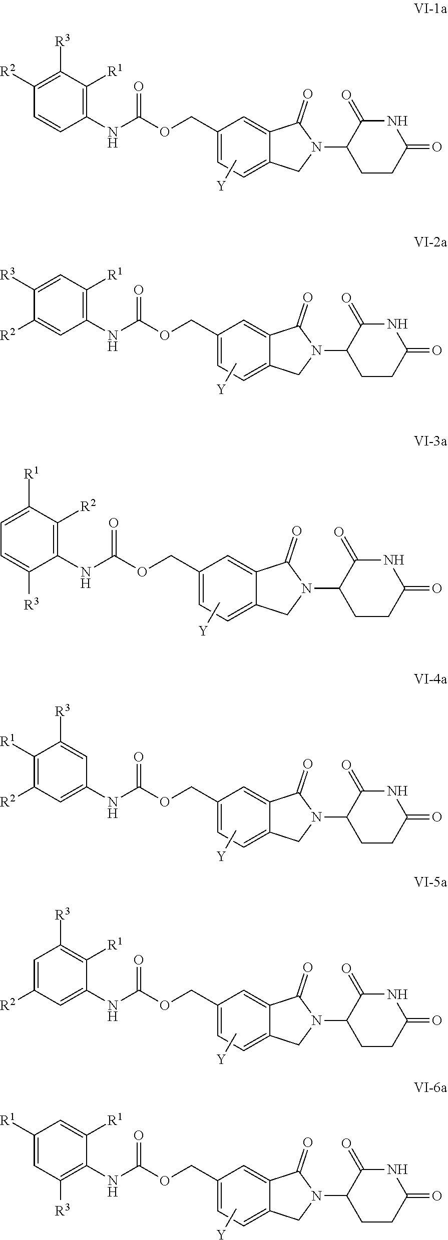

wherein
Y is linear or branched C₁₋₆ alkyl, —C₁₋₄ alkoxy, —CN, halogen, CF₃, CHF₂, CMeF₂, OCF₃, OCHF₂;
R¹, R², R³ are independently of each other selected from H, linear or branched —C₁₋₆ alkyl, linear or branched C₁₋₄ heteroalkyl, —C₁₋₄ alkoxy-C₁₋₄ alkylhydroxy, and halogen, such as F, Cl or Br, e.g. F or Cl, or a group of formula -L³-X², wherein L³ is a covalent bond, linear or branched C₁₋₆ alkyl, —O—, —C₁₋₄ alkoxy and X² is C₃₋₆ cycloalkyl, C₆₋₁₀ aryl, 5-10 membered heteroaryl, 4-8 membered heterocycloalkyl, wherein X² is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy.

In some embodiments of a compound of formula VI-la, VI-2a, VI-3a, VI-4a, VI-5a, VI-6a, Y is in the 4-position. In some embodiments of a compound of formula I, Y is in the 5-position. In some embodiments of a compound of formula I, Y is in the 7-position.

In some embodiments of formula VI-la, VI-2a, VI-3a, VI-4a, VI-5a, VI-6a, Y is C₁₋₄ alkyl, such as methyl, —C₁₋₄ alkoxy, such as —OMe, —CN, halogen, such as F, Cl, Br.

In some embodiments of a compound of formula VI-1, VI-2, VI-3, VI-4, VI-5, VI-6, L³ is a covalent bond. In some embodiments of a compound of formula I, L³ is —O—.

In some embodiments of a compound of formula VI-la, VI-2a, VI-3a, VI-4a, VI-5a, VI-6a, R¹, R², R³ are independently of each other selected from H, linear or branched —C₁₋₆ alkyl, linear or branched C₁₋₆ heteroalkyl, —C₁₋₄ alkoxy, CF₃, CHF₂, CMeF₂, —O—(CH₂)₂—OMe, OCF₃, OCHF₂, —C₁₋₆ alkylamino, —CN, NH₂, —C₁₋₄ alkylhydroxy, and halogen, such as F, Cl or Br, e.g. F or Cl.

In some embodiments of a compound of formula VI-la, VI-2a, VI-3a, VI-4a, VI-5a, VI-6a, R¹, R², R³ are independently of each other selected from H, linear or branched —C₁₋₆ alkyl, —C₁₋₄ alkoxy, CF₃, CHF₂, CMeF₂, OCF₃, OCHF₂, —CN, and halogen, such as F, Cl or Br, e.g. F or Cl.

In some embodiments of a compound of formula VI-la, VI-2a, VI-3a, VI-4a, VI-5a, VI-6a, R¹, R², R³ are independently of each other selected from H, linear or branched —C₁₋₄ alkyl, OCF₃, OCHF₂, and halogen, such as F, Cl or Br, e.g. F or Cl.

In some embodiments of a compound of formula VI-la, VI-2a, VI-3a, VI-4a, VI-5a, VI-6a, one of R¹, R², R³ is a group of formula -L³-X², wherein L³ is a covalent bond and X² is H, C₃₋₆ cycloalkyl, C₆ aryl, 6-membered heteroaryl, 5-6 membered heterocycloalkyl, wherein X² is unsubstituted or substituted with one or more of linear or branched C₁₋₄ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy.

In some embodiments of a compound of formula VI-la, VI-2a, VI-3a, VI-4a, VI-5a, VI-6a, one of R¹, R², R³ is a group of formula -L³-X², wherein L³ is a covalent bond and X² is H, C₃₋₆ cycloalkyl, C₆ aryl, 6 membered heteroaryl, 4-8 membered heterocycloalkyl, wherein X² is unsubstituted or substituted with one or more of linear or branched C₁₋₄ alkyl, and halogen, e.g. F.

In some embodiments of a compound of formula VI-la, VI-2a, VI-3a, VI-4a, VI-5a, VI-6a, one of R¹, R², R³ is a group of formula -L³-X², wherein L³ is a covalent bond and X² is H, cyclopropyl, cyclobutyl, C₆ aryl, pyridinyl, pyrrolidinyl, piperdinyl, wherein X² is unsubstituted or substituted with one or more of linear or branched C₁₋₄ alkyl, such as methyl, and halogen, such as F.

In some embodiments of a compound of formula VI-la, VI-2a, VI-3a, VI-4a, VI-5a, VI-6a, one of R¹, R², R³ is a group of formula -L³-X², wherein L³ is —O— and X² is H, C₃₋₆ cycloalkyl, C₆ aryl, 6-membered heteroaryl, 5-6 membered heterocycloalkyl, wherein X² is unsubstituted or substituted with one or more of linear or branched C₁₋₄ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy.

In some embodiments of a compound of formula VI-la, VI-2a, VI-3a, VI-4a, VI-5a, VI-6a, one of R¹, R², R³ is a group of formula -L³-X², wherein L³ is —O— and X² is H, C₃₋₆ cycloalkyl, C₆ aryl, 6 membered heteroaryl, 4-8 membered heterocycloalkyl, wherein X² is unsubstituted or substituted with one or more of linear or branched C₁₋₄ alkyl, and halogen, e.g. F.

In some embodiments of a compound of formula VI-la, VI-2a, VI-3a, VI-4a, VI-5a, VI-6a, one of R¹, R², R³ is a group of formula -L³-X², wherein L³ is —O— and X² is H, cyclopropyl, cyclobutyl, C₆ aryl, pyridinyl, pyrrolidinyl, piperdinyl, wherein X² is unsubstituted or substituted with one or more of linear or branched C₁₋₄ alkyl, such as methyl, and halogen, such as F.

In some embodiments of a compound of formula VI-la, VI-2a, VI-3a, VI-4a, VI-5a, VI-6a, one of R¹, R², R³ is a group of formula -L³-X², wherein L³ is —O— and X² is cyclopropyl.

In some embodiments, the compound of formula IV is a compound or pharmaceutically acceptable salts or stereoisomers thereof of formula VII-1, VII-2, VII-3, wherein one of w¹, w², w³ is N and the other two of w¹, w², w³ are C:

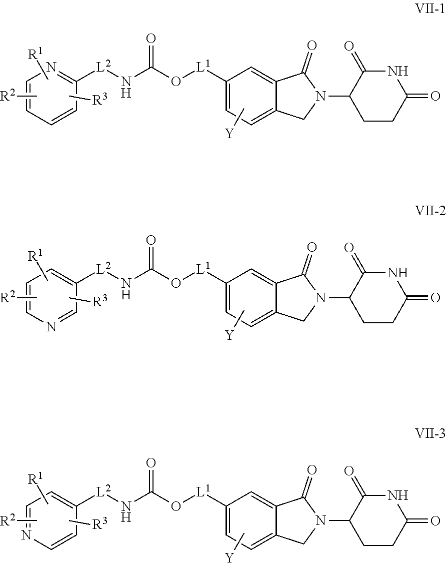

wherein
Y is linear or branched C₁₋₆ alkyl, —C₁₋₄ alkoxy, —CN, halogen, CF₃, CHF₂, CMeF₂, OCF₃, OCHF₂;
R¹, R², R³ are independently of each other selected from H, linear or branched —C₁a alkyl, linear or branched C₁₋₆ heteroalkyl, —C₁₋₄ alkoxy, CF₃, CHF₂, CMeF₂, —O—(CH₂)₂—OMe, OCF₃, OCHF₂, —C₁₋₆ alkylamino, —CN, —NH₂, —C₁₋₄ alkylhydroxy, and halogen, such as F, Cl or Br, e.g. F or Cl, or a group of formula -L³-X², wherein L³ is a covalent bond, linear or branched C₁₋₆ alkyl, —O—, and X² is C₃₋₆ cycloalkyl, C₆₋₁₀ aryl, 5-10 membered heteroaryl, 4-8 membered heterocycloalkyl, wherein X² is unsubstituted or substituted with one or more of linear or branched C₁₋₄ alkyl, —C₁₋₄ alkoxy, NH₂, NMe₂, halogen, CF₃, CHF₂, CMeF₂, —O—(CH₂)₂—OMe, OCF₃, OCHF₂, and C₁₋₄ alkylhydroxy;
L¹ is linear or branched C₁₋₆ alkyl; L² is a covalent bond, linear or branched C₁₋₆ alkyl.

In some embodiments of formula VII-1, VII-2, VII-3, Y is C₁₋₄ alkyl, such as methyl, —C₁₋₄ alkoxy, such as —OMe, —CN, halogen, such as F, Cl, Br.

In some embodiments of a compound of formula VII-1, VII-2, VII-3, L¹ is linear or branched C₁₋₄ alkyl. In some embodiments of a compound of formula VII-1, VII-2, VII-3, L¹ is —CH₂—.

In some embodiments of a compound of formula VII-1, VII-2, VII-3, L² is a covalent bond.

In some embodiments of a compound of formula VII-1, VII-2, VII-3, L² is —CH₂—.

In some embodiments of a compound of formula VII-1, VII-2, VII-3, L³ is a covalent bond.

In some embodiments of a compound of formula VII-1, VII-2, VII-3, L³ is —O—.

In some embodiments of a compound of formula VII-1, VII-2, VII-3, L¹ is —CH₂— and L² is a covalent bond.

In some embodiments of a compound of formula VII-1, VII-2, VII-3, L¹ is —CH₂—, L² is a covalent bond and L³ is a covalent bond. In some embodiments of a compound of formula VII-1, VII-2, VII-3, L¹ is —CH₂—, L² is a covalent bond and L³ is —O—.

In some embodiments of a compound of formula VII-1, VII-2, VII-3, R¹, R², R³ are independently of each other selected from H, linear or branched —C₁₋₆ alkyl, linear or branched C₁₋₆ heteroalkyl, —C₁₋₄ alkoxy, CF₃, CHF₂, CMeF₂, —O—(CH₂)₂—OMe, OCF₃, OCHF₂, —C₁₋₆ alkylamino, —CN, NH₂, —C₁₋₄ alkylhydroxy, and halogen, such as F, Cl or Br, e.g. F or Cl.

In some embodiments of a compound of formula VII-1, VII-2, VII-3, R¹, R², R³ are independently of each other selected from H, linear or branched —C₁₋₆ alkyl, —C₁₋₄ alkoxy, CF₃, CHF₂, CMeF₂, OCF₃, OCHF₂, —CN, and halogen, such as F, Cl or Br, e.g. F or Cl.

In some embodiments of a compound of formula VII-1, VII-2, VII-3, R¹, R², R³ are independently of each other selected from H, linear or branched —C₁₋₄ alkyl, OCF₃, OCHF₂, and halogen, such as F, Cl or Br, e.g. F or Cl.

In some embodiments of a compound of formula VII-1, VII-2, VII-3, R¹ is H and R², R³ are independently of each other selected from H, linear or branched —C₁₋₆ alkyl, linear or branched C₁₋₆ heteroalkyl, —C₁₋₄ alkoxy, CF₃, CHF₂, CMeF₂, —O—(CH₂)₂—OMe, OCF₃, OCHF₂, —C₁₋₆ alkylamino, —CN, NH₂, —C₁₋₄ alkylhydroxy, and halogen, such as F, Cl or Br, e.g. F or Cl.

In some embodiments of a compound of formula VII-1, VII-2, VII-3, R¹ is H and R², R³ are independently of each other selected from H, linear or branched —C₁₋₆ alkyl, —C₁₋₄ alkoxy, CF₃, CHF₂, CMeF₂, OCF₃, OCHF₂, —CN, and halogen, such as F, Cl or Br, e.g. F or Cl. In some embodiments of a compound of formula VII-1, VII-2, VII-3, R¹ is H and R², R³ are independently of each other selected from H, linear or branched —C₁₋₄ alkyl, OCF₃, OCHF₂, and halogen, such as F, Cl or Br, e.g. F or Cl.

In some embodiments of a compound of formula VII-1, VII-2, VII-3, L¹ is —CH₂— and R¹, R², R³ are independently of each other selected from H, linear or branched —C₁₋₆ alkyl, linear or branched C₁₋₆ heteroalkyl, —C₁₋₄ alkoxy, CF₃, CHF₂, CMeF₂, —O—(CH₂)₂—OMe, OCF₃, OCHF₂, —C₁₋₆ alkylamino, —CN, NH₂, —C₁₋₄ alkylhydroxy, and halogen, such as F, Cl or Br, e.g. F or Cl.

In some embodiments of a compound of formula VII-1, VII-2, VII-3, L¹ is —CH₂— and R¹, R², R³ are independently of each other selected from H, linear or branched —C₁₋₆ alkyl, —C₁₋₄ alkoxy, CF₃, CHF₂, CMeF₂, OCF₃, OCHF₂, —CN, and halogen, such as F, Cl or Br, e.g. F or Cl.

In some embodiments of a compound of formula VII-1, VII-2, VII-3, L¹ is —CH₂— and R¹, R², R³ are independently of each other selected from H, linear or branched —C₁₋₄ alkyl, OCF₃, OCHF₂, and halogen, such as F, Cl or Br, e.g. F or Cl.

In some embodiments of a compound of formula VII-1, VII-2, VII-3, L¹ is —CH₂—, R¹ is H and R², R³ are independently of each other selected from H, linear or branched —C₁₋₆ alkyl, linear or branched C₁₋₆ heteroalkyl, —C₁₋₄ alkoxy, CF₃, CHF₂, CMeF₂, —O—(CH₂)₂—OMe, OCF₃, OCHF₂, —C₁₋₆ alkylamino, —CN, NH₂, —C₁₋₄ alkylhydroxy, and halogen, such as F, Cl or Br, e.g. F or Cl.

In some embodiments of a compound of formula VII-1, VII-2, VII-3, L¹ is —CH₂—, R¹ is H and R², R³ are independently of each other selected from H, linear or branched —C₁₋₆ alkyl, —C₁₋₄ alkoxy, CF₃, CHF₂, CMeF₂, OCF₃, OCHF₂, —CN, and halogen, such as F, Cl or Br, e.g. F or Cl.

In some embodiments of a compound of formula VII-1, VII-2, VII-3, L¹ is —CH₂— and R¹ is H and R², R³ are independently of each other selected from H, linear or branched —C₁₋₄ alkyl, OCF₃, OCHF₂, and halogen, such as F, Cl or Br, e.g. F or Cl.

In some embodiments of a compound of formula VII-1, VII-2, VII-3, L² is a covalent bond and R¹, R², R³ are independently of each other selected from H, linear or branched —C₁₋₆ alkyl, linear or branched C₁₋₆ heteroalkyl, —C₁₋₄ alkoxy, CF₃, CHF₂, CMeF₂, —O—(CH₂)₂—OMe, OCF₃, OCHF₂, —C₁— alkylamino, —CN, NH₂, —C₁₋₄ alkylhydroxy, and halogen, such as F, Cl or Br, e.g. F or Cl.

In some embodiments of a compound of formula VII-1, VII-2, VII-3, L² is a covalent bond and R¹, R², R³ are independently of each other selected from H, linear or branched —C₁₋₆ alkyl, —C₁₋₄ alkoxy, CF₃, CHF₂, CMeF₂, OCF₃, OCHF₂, —CN, and halogen, such as F, Cl or Br, e.g. F or Cl.

In some embodiments of a compound of formula VII-1, VII-2, VII-3, L² is a covalent bond and R¹, R², R³ are independently of each other selected from H, linear or branched —C₁₋₄ alkyl, OCF₃, OCHF₂, and halogen, such as F, Cl or Br, e.g. F or Cl.

In some embodiments of a compound of formula VII-1, VII-2, VII-3, L² is a covalent bond, R¹ is H and R², R³ are independently of each other selected from H, linear or branched —C₁₋₆ alkyl, linear or branched C₁₋₆ heteroalkyl, —C₁₋₄ alkoxy, CF₃, CHF₂, CMeF₂, —O—(CH₂)₂—OMe, OCF₃, OCHF₂, —C₁₋₆ alkylamino, —CN, NH₂, —C₁₋₄ alkylhydroxy, and halogen, such as F, Cl or Br, e.g. F or Cl.

In some embodiments of a compound of formula VII-1, VII-2, VII-3, L² is a covalent bond, R¹ is H and R², R³ are independently of each other selected from H, linear or branched —C₁₋₆ alkyl, —C₁₋₄ alkoxy, CF₃, CHF₂, CMeF₂, OCF₃, OCHF₂, —CN, and halogen, such as F, Cl or Br, e.g. F or Cl.

In some embodiments of a compound of formula VII-1, VII-2, VII-3, L² is a covalent bond, and R¹ is H and R², R³ are independently of each other selected from H, linear or branched —C₁₋₄ alkyl, OCF₃, OCHF₂, and halogen, such as F, Cl or Br, e.g. F or Cl.

In some embodiments of a compound of formula VII-1, VII-2, VII-3, one of R¹, R², R³ is a group of formula -L³-X², wherein L³ is a covalent bond and X² is H, C₃₋₆ cycloalkyl, C₆ aryl, 6-membered heteroaryl, 5-6 membered heterocycloalkyl, wherein X² is unsubstituted or substituted with one or more of linear or branched C₁₋₄ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy.

In some embodiments of a compound of formula VII-1, VII-2, VII-3, one of R¹, R², R³ is a group of formula -L³-X², wherein L³ is a covalent bond and X² is H, C₃₋₆ cycloalkyl, C₆ aryl, 6 membered heteroaryl, 4-8 membered heterocycloalkyl, wherein X² is unsubstituted or substituted with one or more of linear or branched C₁₋₄ alkyl, and halogen, e.g. F.

In some embodiments of a compound of formula VII-1, VII-2, VII-3, one of R¹, R², R³ is a group of formula -L³-X², wherein L³ is a covalent bond and X² is H, cyclopropyl, cyclobutyl, C₆ aryl, pyridinyl, pyrrolidinyl, piperdinyl, wherein X² is unsubstituted or substituted with one or more of linear or branched C₁₋₄ alkyl, such as methyl, and halogen, such as F.

In some embodiments of a compound of formula VII-1, VII-2, VII-3, one of R¹, R², R³ is a group of formula -L³-X², wherein L³ is —O— and X² is H, C₃₋₆ cycloalkyl, C₆ aryl, 6-membered heteroaryl, 5-6 membered heterocycloalkyl, wherein X² is unsubstituted or substituted with one or more of linear or branched C₁₋₄ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy.

In some embodiments of a compound of formula VII-1, VII-2, VII-3, one of R¹, R², R³ is a group of formula -L³-X², wherein L³ is —O— and X² is H, C₃₋₆ cycloalkyl, C₆ aryl, 6 membered heteroaryl, 4-8 membered heterocycloalkyl, wherein X² is unsubstituted or substituted with one or more of linear or branched C₁₋₄ alkyl, and halogen, e.g. F.

In some embodiments of a compound of formula VII-1, VII-2, VII-3, one of R¹, R², R³ is a group of formula -L³-X², wherein L³ is —O— and X² is H, cyclopropyl, cyclobutyl, C₆ aryl, pyridinyl, pyrrolidinyl, piperdinyl, wherein X² is unsubstituted or substituted with one or more of linear or branched C₁₋₄ alkyl, such as methyl, and halogen, such as F.

In some embodiments, the compound of formula VII-1, VII-2, VII-3 is a compound or pharmaceutically acceptable salts or stereoisomers thereof of formula VII-1a, VII-2a, VII-3a:

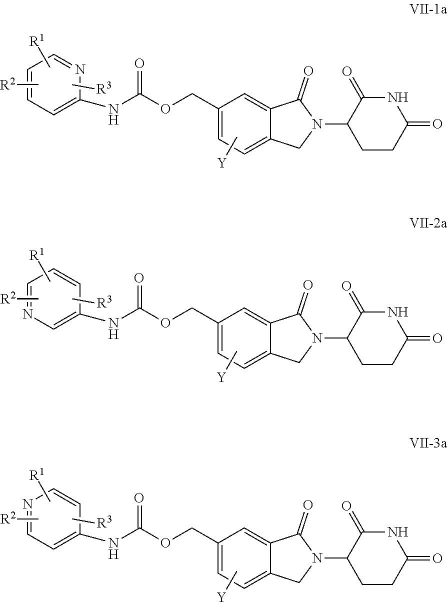

wherein
Y is linear or branched C₁₋₆ alkyl, —C₁₋₄ alkoxy, —CN, halogen, CF₃, CHF₂, CMeF₂, OCF₃, OCHF₂;
R¹, R², R³ are independently of each other selected from H, linear or branched —C₁₋₆ alkyl, linear or branched C₁₋₆ heteroalkyl, —C₁₋₄ alkoxy, CF₃, CHF₂, CMeF₂, —O—(CH₂)₂—OMe, OCF₃, OCHF₂, —C₁₋₆ alkylamino, —CN, —NH₂, —C₁₋₄ alkylhydroxy, and halogen, such as F, Cl or Br, e.g. F or Cl, or a group of formula -L³-X², wherein L³ is a covalent bond, linear or branched C₁₋₆ alkyl, —O—, and X² is C₃₋₆ cycloalkyl, C₆₋₁₀ aryl, 5-10 membered heteroaryl, 4-8 membered heterocycloalkyl, wherein X² is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, and C₁₋₄ alkylhydroxy.

In some embodiments of formula VII-1a, VII-2a, VII-3a, Y is C₁₋₄ alkyl, such as methyl, —C₁₋₄ alkoxy, such as —OMe, —CN, halogen, such as F, Cl, Br.

In some embodiments of a compound of formula VII-1a, VII-2a, VII-3a, L³ is a covalent bond. In some embodiments of a compound of formula VII-1a, VII-2a, VII-3a, L³ is —O—.

In some embodiments of a compound of formula VII-1a, VII-2a, VII-3a, R¹, R², R³ are independently of each other selected from H, linear or branched —C₁₋₆ alkyl, linear or branched C₁₋₆ heteroalkyl, —C₁₋₄ alkoxy, CF₃, CHF₂, CMeF₂, —O—(CH₂)₂—OMe, OCF₃, OCHF₂, —C₁— alkylamino, —CN, NH₂, —C₁₋₄ alkylhydroxy, and halogen, such as F, Cl or Br, e.g. F or Cl.

In some embodiments of a compound of formula VII-1a, VII-2a, VII-3a, R¹, R², R³ are independently of each other selected from H, linear or branched —C₁₋₆ alkyl, —C₁₋₄ alkoxy, CF₃, CHF₂, CMeF₂, OCF₃, OCHF₂, —CN, and halogen, such as F, Cl or Br, e.g. F or Cl.

In some embodiments of a compound of formula VII-1a, VII-2a, VII-3a, R¹, R², R³ are independently of each other selected from H, linear or branched —C₁₋₄ alkyl, OCF₃, OCHF₂, and halogen, such as F, Cl or Br, e.g. F or Cl.

In some embodiments of a compound of formula VII-1a, VII-2a, VII-3a, R¹ is H and R², R³ are independently of each other selected from H, linear or branched —C₁₋₆ alkyl, linear or branched C₁₋₄ heteroalkyl, —C₁₋₄ alkoxy, CF₃, CHF₂, CMeF₂, —O—(CH₂)₂—OMe, OCF₃, OCHF₂, —C₁₋₆ alkylamino, —CN, NH₂, —C₁₋₄ alkylhydroxy, and halogen, such as F, Cl or Br, e.g. F or Cl.

In some embodiments of a compound of formula VII-1a, VII-2a, VII-3a, R¹ is H and R², R³ are independently of each other selected from H, linear or branched —C₁₋₆ alkyl, —C₁₋₄ alkoxy, CF₃, CHF₂, CMeF₂, OCF₃, OCHF₂, —CN, and halogen, such as F, Cl or Br, e.g. F or Cl.

In some embodiments of a compound of formula VII-1a, VII-2a, VII-3a, R¹ is H and R², R³ are independently of each other selected from H, linear or branched —C₁₋₄ alkyl, OCF₃, OCHF₂, and halogen, such as F, Cl or Br, e.g. F or Cl.

In some embodiments of a compound of formula VII-1a, VII-2a, VII-3a, one of R¹, R², R³ is a group of formula -L³-X², wherein L³ is a covalent bond and X² is H, C₃₋₆ cycloalkyl, C₆ aryl, 6-membered heteroaryl, 5-6 membered heterocycloalkyl, wherein X² is unsubstituted or substituted with one or more of linear or branched C₁₋₄ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy.

In some embodiments of a compound of formula VII-1a, VII-2a, VII-3a, one of R¹, R², R³ is a group of formula -L³-X², wherein L³ is a covalent bond and X² is H, C₃₋₆ cycloalkyl, C₆ aryl, 6 membered heteroaryl, 4-8 membered heterocycloalkyl, wherein X² is unsubstituted or substituted with one or more of linear or branched C₁₋₄ alkyl, and halogen, e.g. F.

In some embodiments of a compound of formula VII-1a, VII-2a, VII-3a, one of R¹, R², R³ is a group of formula -L³-X², wherein L³ is a covalent bond and X² is H, cyclopropyl, cyclobutyl, C₆ aryl, pyridinyl, pyrrolidinyl, piperdinyl, wherein X² is unsubstituted or substituted with one or more of linear or branched C₁₋₄ alkyl, such as methyl, and halogen, such as F.

In some embodiments of a compound of formula VII-1a, VII-2a, VII-3a, one of R¹, R², R³ is a group of formula -L³-X², wherein L³ is —O— and X² is H, C₃₋₆ cycloalkyl, C₆ aryl, 6-membered heteroaryl, 5-6 membered heterocycloalkyl, wherein X² is unsubstituted or substituted with one or more of linear or branched C₁₋₄ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy.

In some embodiments of a compound of formula VII-1a, VII-2a, VII-3a, one of R¹, R², R³ is a group of formula -L³-X², wherein L³ is —O— and X² is H, C₃₋₆ cycloalkyl, C₆ aryl, 6 membered heteroaryl, 4-8 membered heterocycloalkyl, wherein X² is unsubstituted or substituted with one or more of linear or branched C₁₋₄ alkyl, and halogen, e.g. F.

In some embodiments of a compound of formula VII-1a, VII-2a, VII-3a, one of R¹, R², R³ is a group of formula -L³-X², wherein L³ is —O— and X² is H, cyclopropyl, cyclobutyl, C₆ aryl, pyridinyl, pyrrolidinyl, piperdinyl, wherein X² is unsubstituted or substituted with one or more of linear or branched C₁₋₄ alkyl, such as methyl, and halogen, such as F.

In some embodiments, the compound of formula VII-1, VII-2, VII-3 is a compound or pharmaceutically acceptable salts or stereoisomers thereof of formula VIIa-1a, VIIa-1b, VIIa-1c, VIIa-2a, VIIa-2b, VIIa-2c, VIIa-3a, VIIa-3b, VIIa-3c

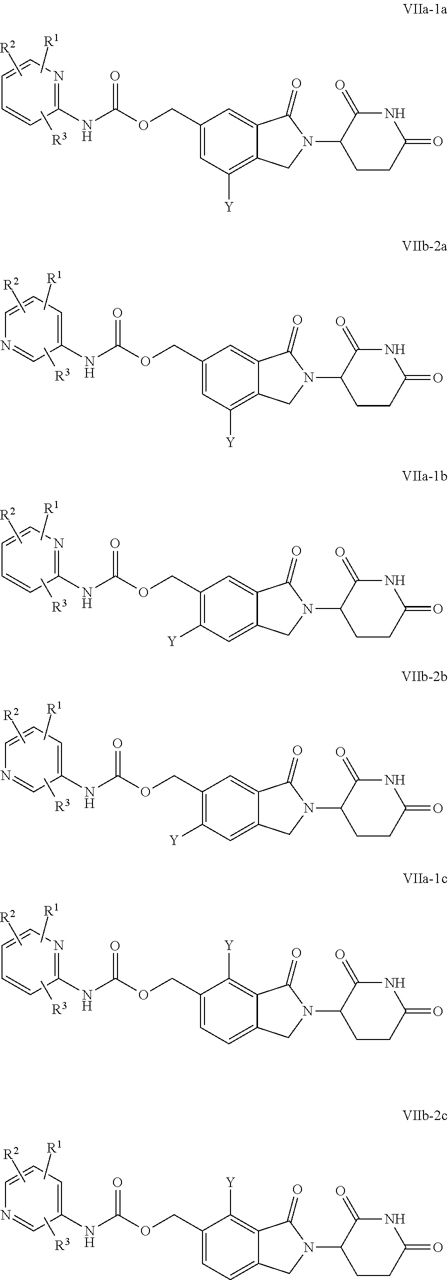

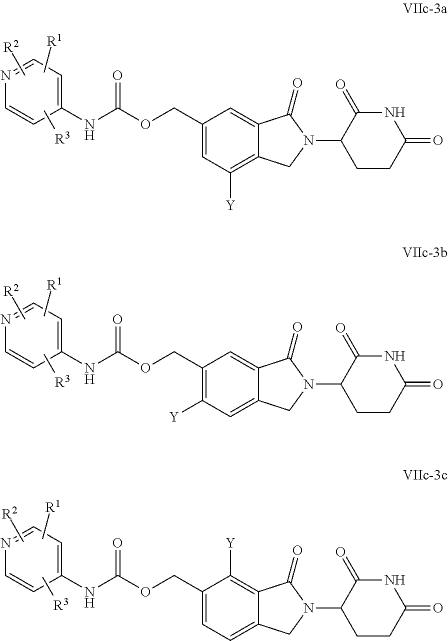

wherein
Y is linear or branched C₁₋₆ alkyl, —C₁₋₄ alkoxy, —CN, halogen, CF₃, CHF₂, CMeF₂, OCF₃, OCHF₂;
R¹, R², R³ are independently of each other selected from H, linear or branched —C₁a alkyl, linear or branched C₁₋₆ heteroalkyl, —C₁₋₄ alkoxy, CF₃, CHF₂, CMeF₂, —O—(CH₂)₂—OMe, OCF₃, OCHF₂, —C₁₋₆ alkylamino, —CN, —NH₂, —C₁₋₄ alkylhydroxy, and halogen, such as F, Cl or Br, e.g. F or Cl, or a group of formula -L³-X², wherein L³ is a covalent bond, linear or branched C₁₋₆ alkyl, —O—, and X² is C₃₋₆ cycloalkyl, C₆₋₁₀ aryl, 5-10 membered heteroaryl, 4-8 membered heterocycloalkyl, wherein X² is unsubstituted or substituted with one or more of linear or branched C₁₋₄ alkyl, —C₁₋₄ alkoxy, NH₂, NMe₂, halogen, CF₃, CHF₂, CMeF₂, —O—(CH₂)₂—OMe, OCF₃, OCHF₂, and C₁₋₄ alkylhydroxy.

In some embodiments of formula VIIa-1a to c, VIIb-2a to c, VIIc-3a to c, Y is C₁₋₄ alkyl, such as methyl, —C₁₋₄ alkoxy, such as —OMe, —CN, halogen, such as F, Cl, Br.

In some embodiments of a compound of formula VIIa-1a to c, VIIb-2a to c, VIIc-3a to c, L³ is a covalent bond. In some embodiments of a compound of formula VIIa-1a to c, VIIb-2a to c, VIIc-3a, L³ is —O—.

In some embodiments of a compound of formula VIIa-1a to c, VIIb-2a to c, VIIc-3a to c, R¹, R², R³ are independently of each other selected from H, linear or branched —C₁₋₆ alkyl, linear or branched C₁₋₄ heteroalkyl, —C₁₋₄ alkoxy, CF₃, CHF₂, CMeF₂, —O—(CH₂)₂—OMe, OCF₃, OCHF₂, —C₁₋₆ alkylamino, —CN, NH₂, —C₁₋₄ alkylhydroxy, and halogen, such as F, Cl or Br, e.g. F or Cl.

In some embodiments of a compound of formula VIIa-1a to c, VIIb-2a to c, VIIc-3a to c, R¹, R², R³ are independently of each other selected from H, linear or branched —C₁₋₆ alkyl, —C₁₋₄ alkoxy, CF₃, CHF₂, CMeF₂, OCF₃, OCHF₂, —CN, and halogen, such as F, Cl or Br, e.g. F or Cl.

In some embodiments of a compound of formula VIIa-1a to c, VIIb-2a to c, VIIc-3a to c, R¹, R², R³ are independently of each other selected from H, linear or branched —C₁₋₄ alkyl, OCF₃, OCHF₂, and halogen, such as F, Cl or Br, e.g. F or Cl.

In some embodiments of a compound of formula VIIa-1a to c, VIIb-2a to c, VIIc-3a to c, R¹ is H and R², R³ are independently of each other selected from H, linear or branched —C₁₋₆ alkyl, linear or branched C₁₋₆ heteroalkyl, —C₁₋₄ alkoxy, CF₃, CHF₂, CMeF₂, —O—(CH₂)₂—OMe, OCF₃, OCHF₂, —C₁— alkylamino, —CN, NH₂, —C₁₋₄ alkylhydroxy, and halogen, such as F, Cl or Br, e.g. F or Cl.

In some embodiments of a compound of formula VIIa-1a to c, VIIb-2a to c, VIIc-3a to c, R¹ is H and R², R³ are independently of each other selected from H, linear or branched —C₁₋₆ alkyl, —C₁₋₄ alkoxy, CF₃, CHF₂, CMeF₂, OCF₃, OCHF₂, —CN, and halogen, such as F, Cl or Br, e.g. F or Cl.

In some embodiments of a compound of formula VIIa-1a to c, VIIb-2a to c, VIIc-3a to c, R¹ is H and R², R³ are independently of each other selected from H, linear or branched —C₁₋₄ alkyl, OCF₃, OCHF₂, and halogen, such as F, Cl or Br, e.g. F or Cl.

In some embodiments of a compound of formula VIIa-1a to c, VIIb-2a to c, VIIc-3a to c, one of R¹, R², R³ is a group of formula -L³-X², wherein L³ is a covalent bond and X² is H, C₃₋₆ cycloalkyl, C₆ aryl, 6-membered heteroaryl, 5-6 membered heterocycloalkyl, wherein X² is unsubstituted or substituted with one or more of linear or branched C₁₋₄ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy.

In some embodiments of a compound of formula VIIa-1a to c, VIIb-2a to c, VIIc-3a to c, one of R¹, R², R³ is a group of formula -L³-X², wherein L³ is a covalent bond and X² is H, C₃₋₆ cycloalkyl, C₆ aryl, 6 membered heteroaryl, 4-8 membered heterocycloalkyl, wherein X² is unsubstituted or substituted with one or more of linear or branched C₁₋₄ alkyl, and halogen, e.g. F.

In some embodiments of a compound of formula VIIa-1a to c, VIIb-2a to c, VIIc-3a to c, one of R¹, R², R³ is a group of formula -L³-X², wherein L³ is a covalent bond and X² is H, cyclopropyl, cyclobutyl, C₆ aryl, pyridinyl, pyrrolidinyl, piperdinyl, wherein X² is unsubstituted or substituted with one or more of linear or branched C₁₋₄ alkyl, such as methyl, and halogen, such as F.

In some embodiments of a compound of formula VIIa-1a to c, VIIb-2a to c, VIIc-3a to c, one of R¹, R², R³ is a group of formula -L³-X², wherein L³ is —O— and X² is H, C₃₋₆ cycloalkyl, C₆ aryl, 6-membered heteroaryl, 5-6 membered heterocycloalkyl, wherein X² is unsubstituted or substituted with one or more of linear or branched C₁₋₄ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy.

In some embodiments of a compound of formula VIIa-1a to c, VIIb-2a to c, VIIc-3a to c, one of R¹, R², R³ is a group of formula -L³-X², wherein L³ is —O— and X² is H, C₃₋₆ cycloalkyl, C₆ aryl, 6 membered heteroaryl, 4-8 membered heterocycloalkyl, wherein X² is unsubstituted or substituted with one or more of linear or branched C₁₋₄ alkyl, and halogen, e.g. F.

In some embodiments of a compound of formula VIIa-1a to c, VIIb-2a to c, VIIc-3a to c, one of R¹, R², R³ is a group of formula -L³-X², wherein L³ is —O— and X² is H, cyclopropyl, cyclobutyl, C₆ aryl, pyridinyl, pyrrolidinyl, piperdinyl, wherein X² is unsubstituted or substituted with one or more of linear or branched C₁₋₄ alkyl, such as methyl, and halogen, such as F.

In some embodiments of a compound of formula I, X¹ is a partially aromatic 6 to 10 membered aryl or heteroaryl, such as a 5-6 fused ring system with the 6 membered ring being a phenyl group. In some embodiments, a compound of formula I is a compound of formula XI, such as formulas XIa, XIb, XIc

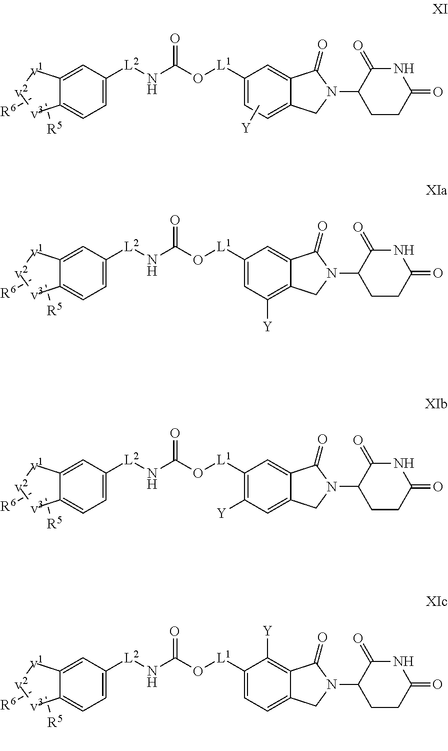

wherein
Y is linear or branched C₁₋₆ alkyl, —C₁₋₄ alkoxy, —CN, halogen, CF₃, CHF₂, CMeF₂, OCF₃, OCHF₂;
v¹, v², v³ are independently of each other selected from C, O, with the proviso that at least one of v¹, v², v³ is C;
R⁵, R⁶ are independently of each other selected from H, linear or branched —C₁₋₄ alkyl and halogen, such as F, Cl, e.g. F;
L¹ is linear or branched C₁₋₆ alkyl; L² is a covalent bond, linear or branched C₁₋₆ alkyl.

In some embodiments of formula XI, such as formulas XIa, XIb, XIc, Y is C₁₋₄ alkyl, such as methyl, —C₁₋₄ alkoxy, such as —OMe, —CN, halogen, such as F, Cl, Br.

In some embodiments of formula XI, such as formulas XIa, XIb, XIc, R⁵, R⁶ are C₁₋₄ alkyl, such as methyl

In some embodiments of formula XI, such as formulas XIa, XIb, XIc, R⁵, R⁶ are attached to the same ring atom. In some embodiments of formula XI, such as formulas XIa, XIb, XIc, R⁵, R⁶ are attached to different ring atoms. In some embodiments of formula XI, such as formulas XIa, XIb, XIc, R⁵, R⁶ are C₁₋₄ alkyl, such as methyl. In some embodiments of formula XI, such as formulas XIa, XIb, XIc, R⁵, R⁶ are C₁₋₄ alkyl, such as methyl, and are attached to the same ring atom.

In some embodiments of a compound of formula XI, such as formulas XIa, XIb, XIc, L¹ is linear or branched C₁₋₄ alkyl. In some embodiments of a compound of formula XI, such as formulas XIa, XIb, XIc, L¹ is —CH₂—.

In some embodiments of a compound of formula XI, such as formulas XIa, XIb, XIc, L² is a covalent bond.

In some embodiments of a compound of formula XI, such as formulas XIa, XIb, XIc, L³ is a covalent bond.

In some embodiments of a compound of formula XI, such as formulas XIa, XIb, XIc, L¹ is —CH₂— and L² is a covalent bond.

In some embodiments of a compound of formula XI, such as formulas XIa, XIb, XIc, L¹ is —CH₂—, L² is a covalent bond and L³ is a covalent bond.

In some embodiments of a compound of formula XI, such as formulas XIa, XIb, XIc, v¹, v², v³ are C. In some embodiments of a compound of formula XI, such as formulas XIa, XIb, XIc, either v¹ is O and v², v³ are C. In some embodiments of a compound of formula XI, such as formulas XIa, XIb, XIc, v² is O and v¹, v³ are C. In some embodiments of a compound of formula XI, such as formulas XIa, XIb, XIc, v³ is O and v¹, v² are C. In some embodiments of a compound of formula XI, such as formulas XIa, XIb, XIc, v¹, v³ are O and v² is C.

In some embodiments, a compound of formula X¹ is a compound of formula XI-1, such as formulas XI-1a, XI-1b, XI-1c

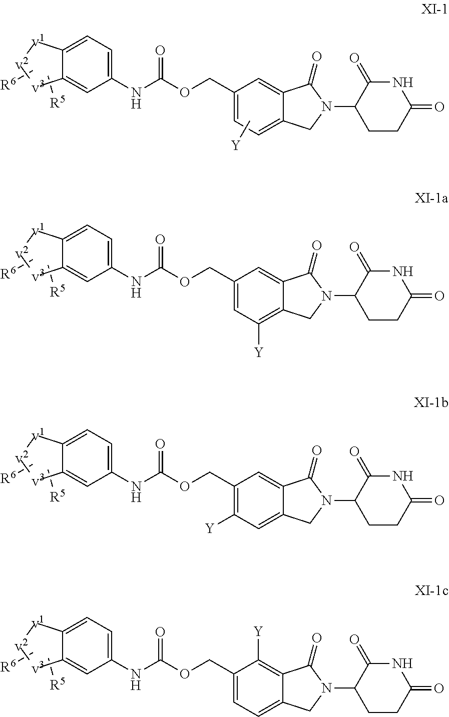

wherein
Y is linear or branched C₁₋₆ alkyl, —C₁₋₄ alkoxy, —CN, halogen, CF₃, CHF₂, CMeF₂, OCF₃, OCHF₂;
v¹, v², v³ are independently of each other selected from C, O, with the proviso that at least one of v¹, v², v³ is C;
R⁵, R⁶ are independently of each other selected from H, linear or branched —C₁₋₄ alkyl and halogen, such as F, Cl, e.g. F.

In some embodiments of a compound of formula XI-1, such as formulas XI-1a, XI-1b, XI-1c, Y is C₁₋₄ alkyl, such as methyl, —C₁₋₆ alkoxy, such as —OMe, —CN, halogen, such as F, Cl, Br.

In some embodiments of formula XI-1, such as formulas XI-1a, XI-1b, XI-1c, R⁵, R⁶ are C₁₋₄ alkyl, such as methyl.

In some embodiments of a compound of formula XI-1, such as formulas XI-1a, XI-1b, XI-1c, R⁵, R⁶ are attached to the same ring atom. In some embodiments of formula XI-1, such as formulas XI-1a, XI-1b, XI-1c, R⁵, R⁶ are attached to different ring atoms. In some embodiments of formula XI-1, such as formulas XI-1a, XI-1b, XI-1c, R⁵, R⁶ are C₁₋₄ alkyl, such as methyl. In some embodiments of formula XI-1, such as formulas XI-1a, XI-1b, XI-1c, R⁵, R⁶ are C₁₋₄ alkyl, such as methyl, and are attached to the same ring atom.

In some embodiments of a compound of formula XI-1, such as formulas XI-1a, XI-1b, XI-1c, L³ is a covalent bond. In some embodiments of a compound of formula XI, such as formulas XIa, XIb, XIc, L³ is —O—.

In some embodiments of a compound of formula XI-1, such as formulas XI-1a, XI-1b, XI-1c, v¹, v², v³ are C. In some embodiments of a compound of formula XI-1, such as formulas XI-1a, XI-1b, XI-1c, either v¹ is O and v², v³ are C. In some embodiments of a compound of formula XI-1, such as formulas XI-1a, XI-1b, XI-1c, v² is O and v¹, v³ are C. In some embodiments of a compound of formula XI-1, such as formulas XI-1a, XI-1b, XI-1c, v³ is O and v¹, v² are C. In some embodiments of a compound of formula XI-1, such as formulas XI-1a, XI-1b, XI-1c, v¹, v³ are O and v² is C.

In some embodiments, a compound of formula X¹ is a compound of formula XII, such as XIIa, XIIb, XIIc, and a compound of formula XII-1, such as XII-1a, XII-1b, XII-1c

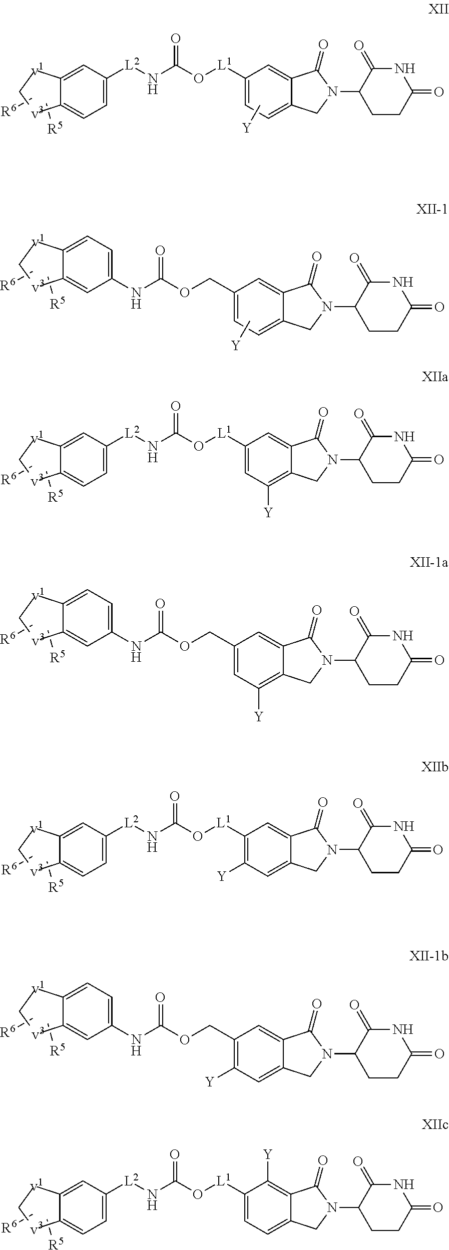

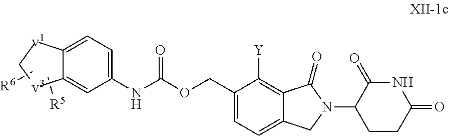

wherein
Y is linear or branched C₁₋₆ alkyl, —C₁₋₄ alkoxy, —CN, halogen, CF₃, CHF₂, CMeF₂, OCF₃, OCHF₂;
v¹, v³ are independently of each other selected from C, O;
R⁵, R⁶ are independently of each other selected from H, linear or branched —C₁₋₄ alkyl and halogen, such as F, Cl, e.g. F;
L¹ is linear or branched C₁₋₆ alkyl; L² is a covalent bond, linear or branched C₁₋₆ alkyl; In some embodiments of a compound of formula XII, such as XIIa, XIIb, XIIc, and a compound of formula XII-1, such as XII-1a, XII-1b, XII-1c, Y is C₁₋₄ alkyl, such as methyl, —C₁₋₄ alkoxy, such as —OMe, —CN, halogen, such as F, Cl, Br.

In some embodiments of a compound of formula XII, such as XIIa, XIIb, XIIc, and a compound of formula XII-1, such as XII-1a, XII-1b, XII-1c, R⁵, R⁶ are C₁₋₄ alkyl, such as methyl.

In some embodiments of XII, such as XIIa, XIIb, XIIc, and a compound of formula XII-1, such as XII-1a, XII-1b, XII-1c, R⁵, R⁶ are attached to the same ring atom. In some embodiments of a compound of formula XII, such as XIIa, XIIb, XIIc, and a compound of formula XII-1, such as XII-1a, XII-1b, XII-1c, R⁵, R⁶ are attached to different ring atoms. In some embodiments of a compound of formula XII, such as XIIa, XIIb, XIIc, and a compound of formula XII-1, such as XII-1a, XII-1b, XII-1c, R⁵, R⁶ are C₁₋₄ alkyl, such as methyl. In some embodiments of a compound of formula XII, such as XIIa, XIIb, XIIc, and a compound of formula XII-1, such as XII-1a, XII-1b, XII-1c, R⁵, R⁶ are C₁₋₄ alkyl, such as methyl, and are attached to the same ring atom.

In some embodiments of a compound of formula XII, such as XIIa, XIIb, XIIc, and a compound of formula XII-1, such as XII-1a, XII-1b, XII-1c, L¹ is linear or branched C₁₋₄ alkyl. In some embodiments of a compound of formula XII, such as XIIa, XIIb, XIIc, and a compound of formula XII-1, such as XII-1a, XII-1b, XII-1c, L¹ is —CH₂—.

In some embodiments of a compound of formula XII, such as XIIa, XIIb, XIIc, and a compound of formula XII-1, such as XII-1a, XII-1b, XII-1c, L² is a covalent bond.

In some embodiments of a compound of formula XII, such as XIIa, XIIb, XIIc, and a compound of formula XII-1, such as XII-1a, XII-1b, XII-1c, L³ is a covalent bond.

In some embodiments of a compound of formula XII, such as XIIa, XIIb, XIIc, and a compound of formula XII-1, such as XII-1a, XII-1b, XII-1c, L¹ is —CH₂— and L² is a covalent bond.

In some embodiments of a compound of formula XII, such as XIIa, XIIb, XIIc, and a compound of formula XII-1, such as XII-1a, XII-1b, XII-1c, L¹ is —CH₂—, L² is a covalent bond and L³ is a covalent bond.

In some embodiments of a compound of formula XII, such as XIIa, XIIb, XIIc, and a compound of formula XII-1, such as XII-1a, XII-1b, XII-1c, v¹, v³ are C. In some embodiments of a compound of formula XII, such as XIIa, XIIb, XIIc, and a compound of formula XII-1, such as XII-1a, XII-1b, XII-1c, v¹ is O and v³ is C. In some embodiments of a compound of formula XII, such as XIIa, XIIb, XIIc, and a compound of formula XII-1, such as XII-1a, XII-1b, XII-1c, v³ is O and v¹ is C. In some embodiments of a compound of formula XI, such as formulas XIa, XIb, XIc, v¹, v³ are O.

In some embodiments, the compound of formula I is a compound or pharmaceutically acceptable salts or stereoisomers thereof of formula XIII, such as XIIIa, XIIIb, XIIIc:

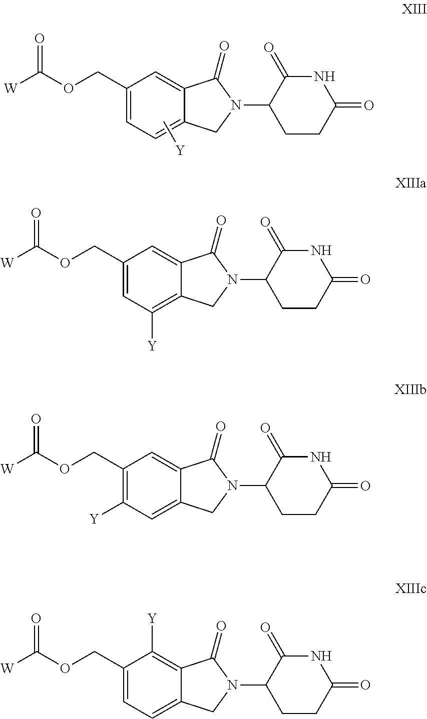

wherein Y is linear or branched C₁₋₆ alkyl, —C₁₋₄ alkoxy, —CN, halogen, CF₃, CHF₂, CMeF₂, OCF₃, OCHF₂, in particular C₁₋₄ alkyl, such as methyl, —C₁₋₄ alkoxy, such as —OMe, —CN, halogen, such as F, Cl, Br; and W is selected from

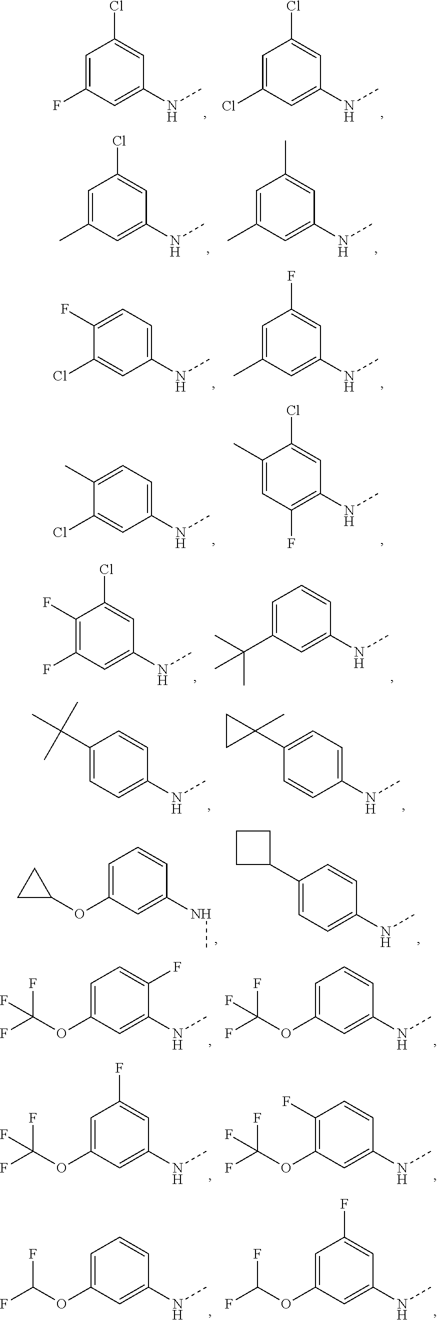

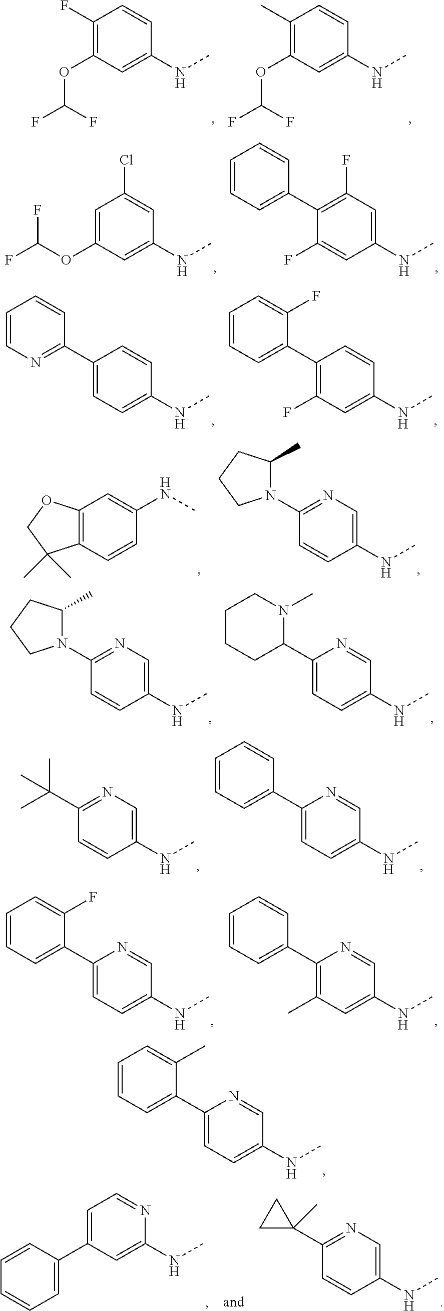

In some embodiments, provided herein is a compound of formula I:

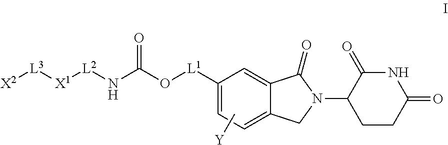

or a pharmaceutically acceptable salt or stereoisomer thereof, wherein
X¹ is selected from the group consisting of linear or branched C₁₋₆ alkyl, C₃₋₄ cycloalkyl, C₆₋₁₀ aryl, 5-10 membered heteroaryl, and 4-8 membered heterocycloalkyl, wherein X is unsubstituted or substituted with one or more substituents independently selected from the group consisting of halogen, linear or branched C₁₋₆ alkyl, linear or branched C₁₋₆ heteroalkyl, CF₃, CHF₂, CMeF₂, —O—CHF₂, —O—(CH₂)₂—OMe, OCF₃, C₁₋₆ alkylamino, —CN, NH₂, C₁₋₄ alkoxy and C₁₋₄ alkylhydroxy;
X² is selected from the group consisting of H, C₃₋₆ cycloalkyl, C₆₋₁₀ aryl, 5-10 membered heteroaryl, and 4-8 membered heterocycloalkyl, wherein X² is unsubstituted or substituted with one or more substituents independently selected from the group consisting of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, and C₁₋₄ alkylhydroxy;
Y is selected from the group consisting of linear or branched C₁₋₆ alkyl, —C₁₋₄ alkoxy, —CN, halogen, CF₃, CHF₂, CMeF₂, OCF₃, and OCHF₂;
L¹ is linear or branched C₁₋₆ alkyl;
L² is selected from a covalent bond, and linear or branched C₁₋₆ alkyl; and
L³ is selected from a covalent bond, linear or branched C₁₋₆ alkyl, —O—, and —C₁₋₄ alkoxy.

In some embodiments, Y is in the 4-position or in the 5-position or in the 7-position of the ring.

In some embodiments, L¹ is —CH₂—, L² is a covalent bond and L³ is a covalent bond or wherein L¹ is —CH₂—, L² is a covalent bond and L³ is —O—.

In some embodiments, X¹ is selected from —C₆₋₁₀ aryl, and 5-10 membered heteroaryl, wherein X¹ is unsubstituted or substituted with one or more substituents independently selected from the group consisting of linear or branched —C₁₋₄ alkyl, halogen, —CF₃, —CHF₂, —CMeF₂, —O—(CH₂)₂—OMe, —OCF₃, —OCHF₂, C₁₋₆ alkylamino, —CN, —NH₂, —C₁₋₄ alkylhydroxy, and —C₁₋₄ alkoxy.

In some embodiments, X² is selected from the group consisting of H, C₃₋₆ cycloalkyl, C₆ aryl, 6-membered heteroaryl, and 5-6 membered heterocycloalkyl, wherein X² is unsubstituted or substituted with one or more substituents independently selected from the group consisting of linear or branched C₁₋₄ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy.

In some embodiments, the compound is of formula II, such as formula IIIa, IIIb or IIIc:

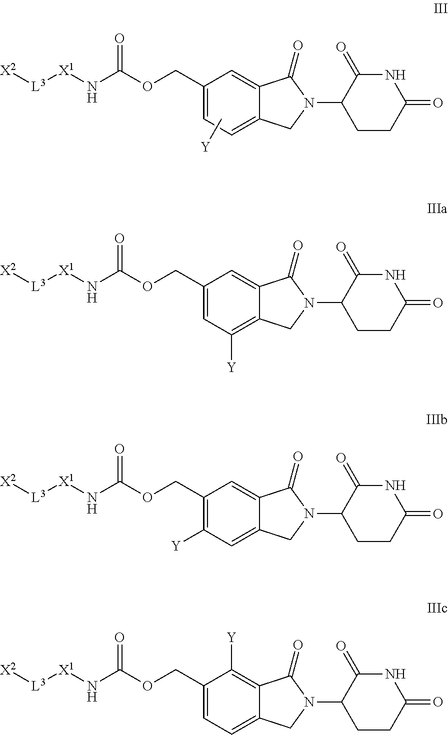

or a pharmaceutically acceptable salt or stereoisomer thereof,
wherein
X¹ is selected from the group consisting of linear or branched C₁₋₆ alkyl, C₃₋₆ cycloalkyl, C₆₋₁₀ aryl, 5-10 membered heteroaryl, and 4-8 membered heterocycloalkyl, wherein X¹ is unsubstituted or substituted with one or more substituents independently selected from the group consisting of halogen, linear or branched C₁₋₆ alkyl, linear or branched C₁₋₆ heteroalkyl, CF₃, CHF₂, CMeF₂, —O—CHF₂, —O—(CH₂)₂—OMe, OCF₃, C₁₋₆ alkylamino, —CN, NH₂, C₁₋₄ alkoxy, and C₁₋₄ alkylhydroxy;
X² is selected from the group consisting of H, C₃₋₆ cycloalkyl, C₆₋₁₀ aryl, 5-10 membered heteroaryl, and 4-8 membered heterocycloalkyl, wherein X² is unsubstituted or substituted with one or more substituents independently selected from the group consisting of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, and C₁₋₄ alkylhydroxy;
Y is selected from the group consisting of linear or branched C₁₋₆ alkyl, —C₁₋₄ alkoxy, —CN, halogen, CF₃, CHF₂, CMeF₂, OCF₃, and OCHF₂; and
L³ is selected from the group consisting of a covalent bond, linear or branched C₁₋₆ alkyl, —O—, and —C₁₋₄ alkoxy.

In some embodiments, X¹ is selected from —C₆₋₁₀ aryl, and 5-10 membered heteroaryl, wherein X¹ is unsubstituted or substituted with one or more substituents independently selected from the group consisting of linear or branched —C₁₋₄ alkyl, halogen, —CF₃, —CHF₂, —CMeF₂, —O—(CH₂)₂—OMe, —OCF₃, —OCHF₂, C₁₋₆ alkylamino, —CN, —NH₂, —C₁₋₄ alkylhydroxy, and —C₁₋₄ alkoxy.

In some embodiments, X² is selected from the group consisting of H, C₃₋₆ cycloalkyl, C₆ aryl, 6-membered heteroaryl, and 5-6 membered heterocycloalkyl, wherein X² is unsubstituted or substituted with one or more substituents independently selected from the group consisting of linear or branched C₁₋₄ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy.

In some embodiments, the compound is of formula IV, such as IVa, IVb or IVc:

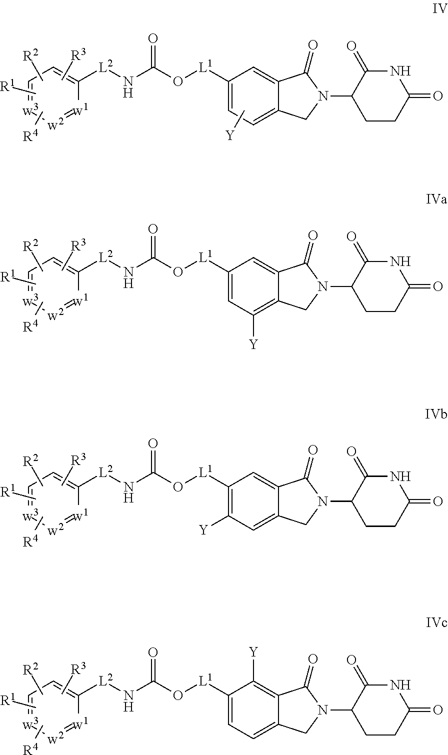

or a pharmaceutically acceptable salt or stereoisomer thereof,
wherein
Y is selected from the group consisting of linear or branched C₁₋₆ alkyl, —C₁₋₄ alkoxy, —CN, halogen, CF₃, CHF₂, CMeF₂, OCF₃, and OCHF₂;
each of w¹, w², and w³ is independently selected from C and N, with the proviso that two or three of w¹, w², w³ are C;
each of R¹, R², R³, and R⁴ is independently selected from the group consisting of H, linear or branched —C₁₋₆ alkyl, linear or branched C₁₋₆ heteroalkyl, —C₁₋₄ alkoxy, CF₃, CHF₂, CMeF₂, —O—(CH₂)₂—OMe, OCF₃, OCHF₂, —C₁₋₆ alkylamino, —CN, —NH₂, —C₁₋₄ alkylhydroxy, and halogen, such as F, Cl or Br, e.g. F or Cl, or a group of formula -L³-X², wherein L³ is selected from the group consisting of a covalent bond, linear or branched C₁₋₆ alkyl, and —O—, and X² is selected from the group consisting of C₃₋₆ cycloalkyl, C₆₋₁₀ aryl, 5-10 membered heteroaryl, and 4-8 membered heterocycloalkyl, wherein X² is unsubstituted or substituted with one or more substituents independently selected from the group consisting of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, and C₁₋₄ alkylhydroxy;
L¹ is linear or branched C₁₋₆ alkyl; and
L² is selected from a covalent bond, and linear or branched C₁₋₆ alkyl.

In some embodiments, L¹ is —CH₂— and L² is a covalent bond.

In some embodiments, w¹, w², and w³ are C, or w¹ is N and w², and w³ are C, or w² is N and w¹, and w³ are C, or w³ is N and w¹, and w² are C.

In some embodiments, the compound is of formula XI, such as XIa, XIb or XIc:

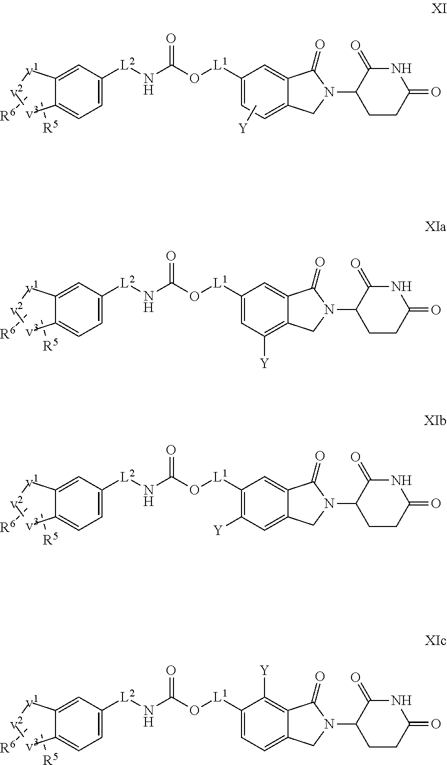

or a pharmaceutically acceptable salt or stereoisomer thereof,
wherein
Y is selected from the group consisting of linear or branched C₁₋₆ alkyl, —C₁₋₄ alkoxy, —CN, halogen, CF₃, CHF₂, CMeF₂, OCF₃, and OCHF₂
each of v¹, v², and v³ is independently selected from C, and O, with the proviso that at least one of v¹, v², and v³ is C;
each of R⁵ and R⁶ is independently selected from the group consisting of H, linear or branched —C₁₋₄ alkyl and halogen, such as F, Cl, e.g. F;
L¹ is linear or branched C₁₋₆ alkyl;
L² is selected from a covalent bond, and linear or branched C₁₋₆ alkyl.

In some embodiments, the compound is of formula XIII, such as XIIIa, XIIIb or XIIIc:

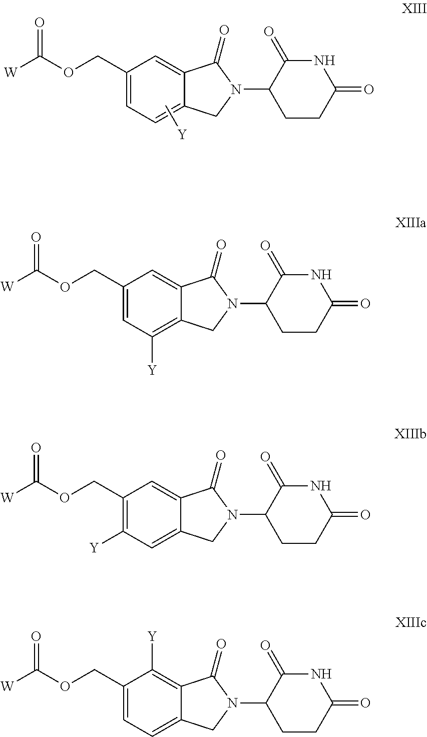

or pharmaceutically acceptable salts or stereoisomers thereof,
wherein
Y is selected from the group consisting of linear or branched C₁₋₆ alkyl, —C₁₋₄ alkoxy, —CN, halogen, CF₃, CHF₂, CMeF₂, OCF₃, and OCHF₂, such as C₁₋₄ alkyl, such as methyl, —C₁₋₄ alkoxy, such as —OMe, —CN, halogen, such as F, Cl, Br; and
W is selected from the group consisting of:

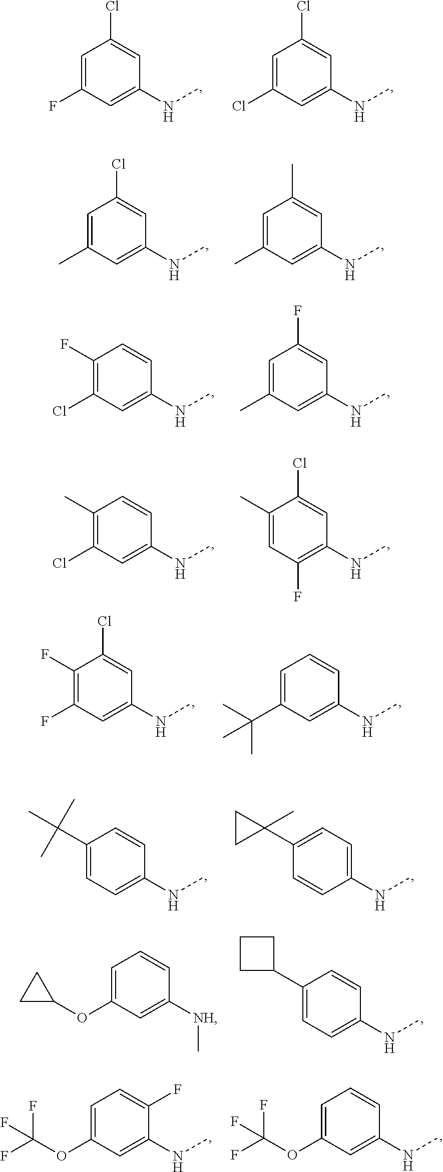

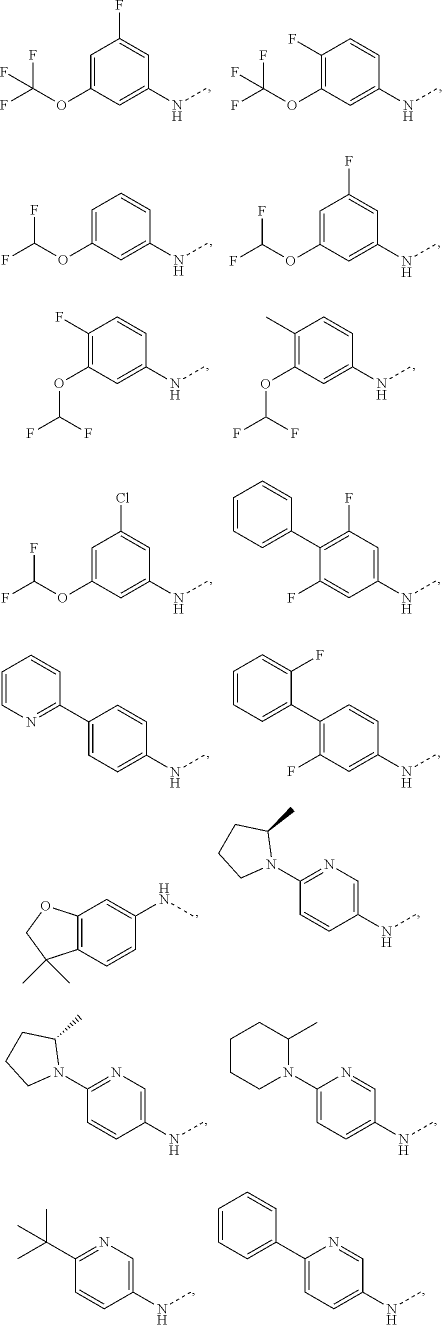

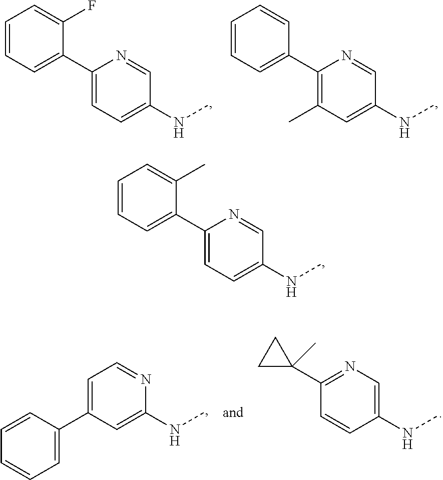

In further specific embodiments, the disclosure is directed to the specific examples disclosed in Table 1.

In some embodiments, the disclosure is directed to the (S) enantiomer of the compounds of any of formula I-XIII. In some embodiments, the disclosure is directed to the (R) enantiomer of the compounds of any of formula I-XIII. In some embodiments, the disclosure is directed to the racemate of the compounds of any of formula I-XIII.

The compounds of the disclosure may contain one or more asymmetric centers in the molecule. A compound without designation of the stereochemistry is to be understood to include all the optical isomers (e.g., diastereomers, enantiomers, etc.) in pure or substantially pure form, as well as mixtures thereof (e.g. a racemic mixture, or an enantiomerically enriched mixture). It is well known in the art how to prepare such optically active forms (e.g. by resolution of the racemic form by recrystallization techniques, by synthesis from optically-active starting materials, by chiral synthesis, by chromatographic separation using a chiral stationary phase, and other methods).

The compounds may be isotopically-labeled compounds, for example, compounds including various isotopes of hydrogen, carbon, nitrogen, oxygen, phosphorous, fluorine, iodine, or chlorine. The disclosed compounds may exist in tautomeric forms and mixtures and separate individual tautomers are contemplated. In addition, some compounds may exhibit polymorphism.

The compounds of the disclosure include the free form as well as the pharmaceutically acceptable salts and stereoisomers thereof. The pharmaceutically acceptable salts include all the typical pharmaceutically acceptable salts. The pharmaceutically acceptable salts of the present compounds can be synthesized from the compounds of this disclosure which contain a basic or acidic moiety by conventional chemical methods, see e.g. Berge et al, “Pharmaceutical Salts,” J. Pharm. ScL, 1977:66:1-19. Furthermore, the compounds of the disclosure also include lyophilized and polymorphs of the free form.

For example, conventional pharmaceutically acceptable salts for a basic compound include those derived from inorganic acids such as hydrochloric, hydrobromic, sulfuric, sulfamic, phosphoric, nitric and the like, as well as salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxy-benzoic, fumaric, toluenesulfonic, methanesulfonic, ethane disulfonic, oxalic, isethionic, trifluoroacetic and the like. Conventional pharmaceutically acceptable salts for an acidic compound include those derived from inorganic bases include aluminum, ammonium, calcium, copper, ferric, ferrous, lithium, magnesium, manganic salts, manganous, potassium, sodium, zinc and the like. Salts derived from pharmaceutically acceptable organic bases include salts of primary, secondary and tertiary amines, substituted amines including naturally occurring substituted amines, cyclic amines and basic ion exchange resins, such as arginine, betaine caffeine, choline, N,N-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine tripropylamine, tromethamine and the like.

The compounds of the disclosure may exist in solid, i.e. crystalline (e.g., polymorphs, i.e., different crystalline structures that have the same chemical composition but differ in packing, geometrical arrangement) or noncrystalline form (optionally as solvates) or liquid form. In the solid state, it may exist in, or as a mixture thereof. In crystalline solvates, solvent molecules are incorporated into the crystalline lattice during crystallization. The formation of solvates may include non-aqueous solvents such as, but not limited to, ethanol, isopropanol, DMSO, acetic acid, ethanolamine, or ethyl acetate, or aqueous solvents such as water (also called “hydrates”). Different polymorphs may be produced, for example, by changing or adjusting the reaction conditions or reagents.

In a further aspect, the disclosure also provides methods of preparation of the compounds of formula I-XIII of the disclosure. In some embodiments, they are prepared according to the general procedure A.

In yet another aspect, the disclosure further provides a pharmaceutical composition comprising a therapeutically-effective amount of one or more of the compounds of the disclosure or pharmaceutically acceptable salt thereof and one or more pharmaceutically acceptable carriers and/or excipients (also referred to as diluents). The excipients are acceptable in the sense of being compatible with the other ingredients of the formulation and not deleterious to the recipient thereof (i.e., the patient). The term “therapeutically-effective amount” as used herein refers to the amount of a compound (as such or in form of a pharmaceutical composition) of the present disclosure which is effective for producing some desired therapeutic effect.

Pharmaceutical compositions may be in unit dose form containing a predetermined amount of a compound of the disclosure per unit dose. Such a unit may contain a therapeutically effective dose of a compound of the disclosure or salt thereof or a fraction of a therapeutically effective dose such that multiple unit dosage forms might be administered at a given time to achieve the desired therapeutically effective dose. Preferred unit dosage formulations are those containing a daily dose or sub-dose, or an appropriate fraction thereof, of a compound of the disclosure or salt thereof.

The compounds of the disclosure may be administered by any acceptable means in solid or liquid form, including (1) oral administration, for example, drenches (aqueous or non-aqueous solutions or suspensions), tablets, e.g., those targeted for buccal, sublingual, and systemic absorption, boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; (8) nasally; (9) pulmonary; or (10) intrathecally.

The phrase “pharmaceutically-acceptable carrier” as used herein means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; and (22) other non-toxic compatible substances employed in pharmaceutical compositions.

Such compositions may contain further components conventional in pharmaceutical preparations, e.g. wetting agents, emulsifiers and lubricants, such as sodium lauryl sulfate and magnesium stearate, as well as coloring agents, release agents, coating agents, sweetening, flavoring and perfuming agents, preservatives and antioxidants, pH modifiers, bulking agents, and further active agents. Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Such compositions may be prepared by any method known in the art, for example, by bringing into association the active ingredient with one or more carriers and/or excipients. Different compositions and examples of carriers and/or excipients are well known to the skilled person and are described in detail in, e.g., Remington: The Science and Practice of Pharmacy. Pharmaceutical Press, 2013; Rowe, Sheskey, Quinn: Handbook of Pharmaceutical Excipients. Pharmaceutical Press, 2009. Excipients that may be used in the preparation of the pharmaceutical compositions may include one or more of buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents, diluents and other known additives to provide a composition suitable for an administration of choice.

As indicated above, the compounds of the present disclosure may be in solid or liquid form and administered by various routes in any convenient administrative form, e.g., tablets, powders, capsules, solutions, dispersions, suspensions, syrups, sprays, suppositories, gels, emulsions, patches, etc.

In solid dosage forms of the disclosure for oral administration (capsules, tablets, pills, dragees, powders, granules, trouches and the like), a compound is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds and surfactants, such as poloxamer and sodium lauryl sulfate; (7) wetting agents, such as, for example, cetyl alcohol, glycerol monostearate, and non-ionic surfactants; (8) absorbents, such as kaolin and bentonite clay; (9) lubricants, such as talc, calcium stearate, magnesium stearate, solid polyethylene glycols, sodium lauryl sulfate, zinc stearate, sodium stearate, stearic acid, and mixtures thereof, (10) coloring agents; and (11) controlled release agents such as crospovidone or ethyl cellulose. In the case of capsules, tablets and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like. A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent. The tablets, and other solid dosage forms of the pharmaceutical compositions of the present disclosure, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the compounds of the disclosure include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof. An oral composition can also include adjuvants such as wetting agents, emulsifying and suspending agents, sweetening, flavoring, coloring, perfuming and preservative agents.

In form of suspensions, a compound may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Dosage forms for rectal or vaginal administration of a compound of the disclosure include a suppository, which may be prepared by mixing one or more compounds of the disclosure with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound. Other suitable forms include pessaries, tampons, creams, gels, pastes, foams or spray formulations containing such carriers as are known in the art to be appropriate.

Dosage forms for the topical or transdermal administration of a compound of the disclosure include powders, sprays, ointments, pastes, creams, lotions, gels, solutions, patches and inhalants. The active compound may be mixed under sterile conditions with a pharmaceutically-acceptable carrier, and with any preservatives, buffers, or propellants which may be required. Such ointments, pastes, creams and gels may contain, in addition to a compound of the disclosure, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Dosage forms such as powders and sprays for administration of a compound of the disclosure, may contain excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Dosage forms such as transdermal patches for administration of a compound of the disclosure may include absorption enhancers or retarders to increase or decrease the flux of the compound across the skin. The rate of such flux can be controlled by either providing a rate controlling membrane or dispersing the compound in a polymer matrix or gel. Other dosage forms contemplated include ophthalmic formulations, eye ointments, powders, solutions and the like. It is understood that all contemplated compositions must be stable under the conditions of manufacture and storage, and preserved against the contaminating action of microorganisms, such as bacteria and fungi.

The dosage levels of a compound of the disclosure in the pharmaceutical compositions of the disclosure may be adjusted in order to obtain an amount of a compound of the disclosure which is effective to achieve the desired therapeutic response for a particular patient, composition, and mode of administration, without being deleterious to the patient. The dosage of choice will depend upon a variety of factors including the nature of the particular compound of the present disclosure used, the route of administration, the time of administration, the rate of excretion or metabolism of the particular compound used, the rate and extent of absorption, the duration of the treatment, other drugs, compounds and/or materials used in combination with the particular compound, the age, sex, weight, condition, general health and prior medical history of the patient being treated, and like factors well known in the medical arts. A medical practitioner having ordinary skill in the art can readily determine and prescribe the effective amount of the pharmaceutical composition required.

Typically, a suitable daily dose of a compound of the disclosure will be that amount of the compound, which is the lowest dose effective to produce a therapeutic effect. Such an effective dose will generally depend upon the factors described above. Generally, oral, intravenous, intracerebroventricular and subcutaneous doses of the compounds of this disclosure for a patient, when used for the indicated analgesic effects, will range from about 0.0001 to about 100 mg, more usual 0.1 to 100 mg/kg per kilogram of body weight of recipient (patient, mammal) per day. Acceptable daily dosages may be from about 1 to about 1000 mg/day, and for example, from about 1 to about 100 mg/day.

A compound of the disclosure, or a pharmaceutically acceptable salt, or stereoisomer, thereof, may be administered once daily (QD) or divided into multiple daily doses such as twice daily (BID), three times daily (TID), and four times daily (QID) or may be administered in regular intervals of more than one day, such as every two days (Q2D). Administration may be continuous (i.e., daily for consecutive days or every day) or intermittent, e.g., in cycles (i.e., including days, weeks, or months of rest without drug). In some embodiments, a compound of the disclosure, or a pharmaceutically acceptable salt, or stereoisomer thereof, is administered every day for at least 21 days. In some embodiments, a compound of the disclosure, or a pharmaceutically acceptable salt or stereoisomer thereof, is administered every two days for at least 21 days.

The term “intermittent” or “intermittently” as used herein is intended to mean stopping and starting at either regular or irregular intervals. For example, intermittent administration of a compound of the disclosure, or a pharmaceutically acceptable salt, or stereoisomer, thereof, is administration for one to six days per week, administration in cycles (e.g., daily administration for two to eight consecutive weeks, then a rest (or holiday) period with no administration for up to one week), or administration on alternate days. The term “cycling” as used herein is intended to mean that a compound of the disclosure, or a pharmaceutically acceptable salt, or stereoisomer, thereof, is administered daily or continuously but with a rest period. In some embodiments, the rest period is the same length as the treatment period. In other embodiments, the rest period has different length from the treatment period. In some embodiments, a compound of the disclosure, or a pharmaceutically acceptable salt, or stereoisomer, thereof, is administered intermittently once per day for 5 days followed by a rest of 3 days (i.e. 5 days on/3 days off). This intermittent administration is repeated for 3 to 4 cycles. In some embodiments, a compound of the disclosure, or a pharmaceutically acceptable salt, or stereoisomer thereof, is administered intermittently once per day for 5 days followed by a rest of 9 days (i.e. 5 days on/9 days off). This intermittent administration is repeated for 2 cycles. It is understood that dosing regimen also depend on factors as indicated above, e.g. on the administration, and can be readily arrived at by one skilled in medicine or the pharmacy art.

The compounds of the disclosure modulate the activity of cereblon. Thus, the compounds and compositions of the disclosure can be useful as a medicament, i.e. as a medicament in therapy, more specifically for the treatment of cancer, as detailed below. Therefore, in a further aspect, the present disclosure provides a method of treatment of a mammal, for example, a human, suffering from cancer, as detailed below. The term “treatment” is intended to encompass prophylaxis, therapy and cure. Such treatment comprises the step of administering a therapeutically effective amount of a compound of Formula I or salt thereof (or of a pharmaceutical composition containing a compound of Formula I or salt thereof) to said mammal, for example, a human.

Thus, the disclosure is directed towards the use of the compounds of the disclosure or pharmaceutically acceptable salts or stereoisomers thereof or a pharmaceutical composition thereof for the treatment of a disease associated or caused with GSPT1, in particular the treatment of cancer, as detailed below, in a mammal, for example a human.

Myc-Driven Cancers

Described herein, in some embodiments, are cancers exhibiting increased expression of one or more of c-Myc, L-Myc, N-Myc, EIF4EBP1, and EIF4EBP2 as well as ones with increase phosphorylation of one or both of EIF4EBP1 and EIF4EBP2.

Myc-driven cancers refer to cancers where there is abnormal activation of Myc oncogene, either due to transcriptional overexpression (e.g., caused by gene amplification, translocation, alterations in upstream signaling pathways) and/or protein stabilization. A myc-driven cancer cell includes a cancer cell that has an increased expression or overexpression (and/or increased activity) of at least one myc transcription factor such as N-myc and/or L-myc and/or C-myc, or a surrogate marker thereof, relative to a control cell such as a normal (e.g., non-cancerous) cell of the same or corresponding cell type. The term “cancerous” when referring to a sample such as a cell or tissue, generally refers to any sample, such as cells or tissues that exhibit, or are predisposed to exhibiting, unregulated growth, including, for example, a neoplastic cell/tissue such as a premalignant cell/tissue or a cancer cell (e.g., carcinoma cell or sarcoma cell).

In some embodiments the Myc-driven cancer or tumor as defined herein refers to a blood borne tumor cancer, such as a hematological cancer, preferably a cancer of hematopoietic and lymphoid tissues and lymphatic system, such as blood cancer, bone marrow cancer, lymph node cancer, acute lymphoblastic leukemia (ALL), chronic lymphocytic lymphoma (CLL), small lymphocytic lymphoma (SLL), acute myelogenous leukemia (AML), chronic myelogenous leukemia (CML), acute monocytic leukemia (AMoL), Hodgkin's lymphoma, non-Hodgkin's lymphomas and multiple myeloma (MM).

In some embodiments, the Myc-driven cancer or tumour is a solid tumor cancer, such as breast cancer, colorectal cancer, lung cancer, e.g. SCLC, NSCLC, neuroendocrine cancer, e.g., neuroendocrine prostate cancer (for example, NEPC (castration-resistant neuroendocrine prostate cancer)) and lung neuroendocrine tumors (Lu-NETs), liver cancer, stomach cancer, pancreatic cancer, gastric cancer, esophageal cancer, bladder cancer, skin cancer, brain cancer, cervical cancer, ovarian cancer, melanoma and head and neck cancer.

In some embodiments the Myc-driven cancer as used herein refers in particular to breast cancer and SCLC. In some embodiments the myc-driven cancer as used herein refers in particular to NSCLC. In some embodiments, the cancer is solid tumor cancer exhibiting amplification of the N-Myc gene and/or the L-Myc gene. In some embodiments the Myc-driven cancer as used herein refers to neuroendocrine cancer, for example, neuroendocrine prostate cancer (for example, NEPC (castration-resistant neuroendocrine prostate cancer)) and lung neuroendocrine tumors (Lu-NETs), acute myelogenous leukemia (AML), lymphoma, and multiple myeloma (MM).

Solid and Liquid Cancers

The term “solid cancer” or “solid tumor” refers to disease of tissues or organs, such as to malignant, neoplastic, or cancerous solid tumors, i.e. sarcomas, carcinomas. The tissue structure of solid tumors includes interdependent tissue compartments and usually does not contain cysts or fluid areas. A solid cancer or solid tumor includes cancers of the bladder, bone, brain, breast, cervix, chest, colon, endrometrium, esophagus, eye, head, kidney, liver, lymph nodes, lung, upper aerodigestive tract (including nasal cavity and paranasal sinuses, nasopharynx or cavum, oral cavity, oropharynx, larynx, hypopharynx and salivary glands), neck, ovaries, pancreas, prostate, rectum, skin, stomach, testis, throat, and uterus. Specific cancers include, but are not limited to, advanced malignancy, amyloidosis, neuroblastoma, meningioma, hemangiopericytoma, multiple brain metastase, glioblastoma multiforms, glioblastoma, brain stem glioma, poor prognosis malignant brain tumor, malignant glioma, recurrent malignant glioma, anaplastic astrocytoma, anaplastic oligodendroglioma, neuroendocrine tumor, e.g., neuroendocrine prostate cancer (for example, NEPC (castration-resistant neuroendocrine prostate cancer)) and lung neuroendocrine tumors (Lu-NETs), rectal adenocarcinoma, colorectal cancer, including stage 3 and stage 4 colorectal cancer, unresectable colorectal carcinoma, metastatic hepatocellular carcinoma, Kaposi's sarcoma, malignant melanoma, cervical cancer, ovarian cancer, malignant mesothelioma, malignant pleural effusion mesothelioma syndrome, peritoneal carcinoma, papillary serous carcinoma, gynecologic sarcoma, soft tissue sarcoma, scleroderma, cutaneous vasculitis, Langerhans cell histiocytosis, leiomyosarcoma, fibrodysplasia ossificans progressive, hormone refractory prostate cancer, resected high-risk soft tissue sarcoma, unrescectable hepatocellular carcinoma, fallopian tube cancer, androgen independent prostate cancer, androgen dependent stage IV non-metastatic prostate cancer, hormone-insensitive prostate cancer, chemotherapy-insensitive prostate cancer, papillary thyroid carcinoma, follicular thyroid carcinoma, medullary thyroid carcinoma, and leiomyoma. In some embodiments, a solid cancer or solid tumor is a cancer of the breast, lung, stomach, colon, bladder, brain, pancreas, liver, head and neck, prostate, ovaries, upper aerodigestive tract and the like.

The term “blood borne cancer” or “blood borne tumor” (also typically referred to as “hematological cancer”) refers to cancer of the body's blood-forming and immune system—the bone marrow and lymphatic tissue. The tissue structure of blood-borne cancers or tumors includes an abnormal mass of cells that is fluid in nature. Such cancers include leukemias (malignant neoplasms of the blood-forming tissues), lymphomas (Non-Hodgkin's Lymphoma), Hodgkin's disease (Hodgkin's Lymphoma) and myeloma. In one embodiment, the myeloma is multiple myeloma (MM). In some embodiments, the leukemia is, for example, acute myelogenous leukemia (AML), acute lymphocytic leukemia (ALL), adult T-cell leukemia, chronic lymphocytic leukemia (CLL), small lymphocytic lymphoma (SLL), hairy cell leukemia, myelodysplasia, myeloproliferative disorders, chronic myelogenous leukemia (CML), acute monocytic leukemia (AMoL), myelodysplastic syndrome (MDS), human lymphotropic virus-type 1 (HTLV-1) leukemia, mastocytosis, or B-cell acute lymphoblastic leukemia. The leukemia can be relapsed, refractory or resistant to conventional therapy. In some embodiments, the lymphoma is, for example, diffuse large B-cell lymphoma (DLBCL), B-cell immunoblastic lymphoma, small non-cleaved cell lymphoma, human lymphotropic virus-type 1 (HTLV-1) leukemia/lymphoma, adult T-cell lymphoma, peripheral T-cell lymphoma (PTCL), cutaneous T-cell lymphoma (CTCL), mantle cell lymphoma (MCL), Hodgkin's lymphoma (HL), non-Hodgkin's lymphoma (NHL), AIDS-related lymphoma, follicular lymphoma, small lymphocytic lymphoma, T-cell/histiocyte rich large B-cell lymphoma, transformed lymphoma, primary mediastinal (thymic)large B-cell lymphoma, splenic marginal zone lymphoma, Richter's transformation, nodal marginal zone lymphoma, or ALK-positive large B-cell lymphoma. In one embodiment, the hematological cancer is indolent lymphoma including, for example, DLBCL, follicular lymphoma, or marginal zone lymphoma. In some embodiments blood-borne cancers or hematological cancers include acute lymphoblastic leukemia (ALL), chronic lymphocytic lymphoma (CLL), small lymphocytic lymphoma (SLL), acute myelogenous leukemia (AML), chronic myelogenous leukemia (CML), acute monocytic leukemia (AMoL), Hodgkin's lymphoma, non-Hodgkin's lymphomas and multiple myeloma (MM).

In particular embodiments, the compounds of the disclosure or pharmaceutically acceptable salts or stereoisomers thereof or a pharmaceutical composition thereof are used for the treatment of cancer associated with GSPT1, such as solid cancers including but not limited to cancers of the bladder, bone, brain, breast, cervix, chest, colon, endrometrium, esophagus, eye, head, kidney, liver, lymph nodes, lung, upper aerodigestive tract (including nasal cavity and paranasal sinuses, nasopharynx or cavum, oral cavity, oropharynx, larynx, hypopharynx and salivary glands), neck, ovaries, pancreas, prostate, rectum, skin, stomach, testis, throat, uterus, amyloidosis, neuroblastoma, meningioma, hemangiopericytoma, multiple brain metastase, glioblastoma multiforms, glioblastoma, brain stem glioma, poor prognosis malignant brain tumor, malignant glioma, recurrent malignant glioma, anaplastic astrocytoma, anaplastic oligodendroglioma, neuroendocrine tumor, e.g., neuroendocrine prostate cancer such as castration-resistant neuroendocrine prostate cancer (NEPC) and lung neuroendocrine tumors (Lu-NETs), rectal adenocarcinoma, colorectal cancer, including stage 3 and stage 4 colorectal cancer, unresectable colorectal carcinoma, metastatic hepatocellular carcinoma, Kaposi's sarcoma, malignant melanoma, malignant mesothelioma, malignant pleural effusion mesothelioma syndrome, peritoneal carcinoma, papillary serous carcinoma, gynecologic sarcoma, soft tissue sarcoma, scleroderma, cutaneous vasculitis, Langerhans cell histiocytosis, leiomyosarcoma, fibrodysplasia ossificans progressive, hormone refractory prostate cancer, resected high-risk soft tissue sarcoma, unrescectable hepatocellular carcinoma, fallopian tube cancer, androgen independent prostate cancer, androgen dependent stage IV non-metastatic prostate cancer, hormone-insensitive prostate cancer, chemotherapy-insensitive prostate cancer, papillary thyroid carcinoma, follicular thyroid carcinoma, medullary thyroid carcinoma, and leiomyoma; and blood bourne (liquid) or hematological cancers, including but not limited to leukemias, lymphomas, and myelomas, such as diffuse large B-cell lymphoma (DLBCL), B-cell immunoblastic lymphoma, small non-cleaved cell lymphoma, human lymphotropic virus-type 1 (HTLV-1) leukemia/lymphoma, adult T-cell lymphoma, peripheral T-cell lymphoma (PTCL), cutaneous T-cell lymphoma (CTCL), mantle cell lymphoma (MCL), Hodgkin's lymphoma (HL), non-Hodgkin's lymphoma (NHL), AIDS-related lymphoma, follicular lymphoma, small lymphocytic lymphoma, T-cell/histiocyte rich large B-cell lymphoma, transformed lymphoma, primary mediastinal (thymic) large B-cell lymphoma, splenic marginal zone lymphoma, Richter's transformation, nodal marginal zone lymphoma, ALK-positive large B-cell lymphoma, indolent lymphoma (for example, DLBCL, follicular lymphoma, or marginal zone lymphoma), acute myelogenous leukemia (AML), acute lymphocytic leukemia (ALL), adult T-cell leukemia, chronic lymphocytic leukemia (CLL), small lymphocytic lymphoma (SLL), hairy cell leukemia, myelodysplasia, myeloproliferative disorders, chronic myelogenous leukemia (CML), acute monocytic leukemia (AMoL), myelodysplastic syndrome (MDS), human lymphotropic virus-type 1 (HTLV-1) leukemia, mastocytosis, B-cell acute lymphoblastic leukemia, Non-Hodgkin's Lymphoma, Hodgkin's Lymphoma, and multiple myeloma (MM).

Such a use (or method of treatment) of a subject comprises administering to a subject in need of such treatment a therapeutically effective amount of a compound of the disclosure or pharmaceutically acceptable salts thereof or a pharmaceutical composition thereof by targeting cereblon.

Disclosed herein, in part, is a method of treating a Myc-driven cancer in a subject in need thereof, comprising administering the subject a therapeutically effective amount of a compound described herein or a composition as described herein.

In some embodiments, the Myc-driven cancer is a Myc-driven lung cancer.

In some embodiments, the Myc-driven cancer is characterized by high driven Myc tumor.

In some embodiments, the Myc-driven cancer is a Myc-driven small cell lung cancer.

In some embodiments, the Myc-driven small cell lung cancer is a high L-Myc small cell lung cancer.

In some embodiments, the Myc-driven cancer is a Myc-driven non-small cell lung cancer.

In some embodiments, the Myc-driven non-small cell lung cancer is a high N-Myc non-small cell lung cancer.

In some embodiments, the method comprises orally administering the compound to the subject.

In another aspect, provided herein is a method of degrading GSPT1 in a subject suffering from cancer, comprising administering to the subject a therapeutically effective amount of a compound described herein or a composition described herein.

In some embodiments, the cancer is a Myc-driven cancer.

In some embodiments, the Myc-driven cancer is a Myc-driven lung cancer.

In some embodiments, the Myc-driven cancer is a Myc-driven small cell lung cancer.

In some embodiments, the Myc-driven small cell lung cancer is a high L-Myc small cell lung cancer.

In some embodiments, the Myc-driven cancer is a Myc-driven non-small cell lung cancer.

In some embodiments, the Myc-driven non-small cell lung cancer is a high N-Myc non-small cell lung cancer.

In some embodiments, the method comprises orally administering the compound to the subject.

In another aspect, the disclosure is directed to a method of reducing the level of GSPT1 in a subject suffering from cancer, comprising administering the subject a therapeutically effective amount of a compound or a composition as described herein.

In some embodiments, the cancer is a Myc-driven cancer.

In some embodiments, the Myc-driven cancer is a Myc-driven lung cancer.

In some embodiments, the Myc-driven cancer is a Myc-driven small cell lung cancer.

In some embodiments, the Myc-driven small cell lung cancer is a high L-Myc small cell lung cancer.

In some embodiments, the Myc-driven cancer is a Myc-driven non-small cell lung cancer.

In some embodiments, the Myc-driven non-small cell lung cancer is a high N-Myc non-small cell lung cancer.

In some embodiments, the method comprises orally administering the compound to the subject.

The present disclosure contemplates administration of a compound of the disclosure alone or in combination with one or more additional therapeutic agents, such as other Tyrosine kinase inhibitors: Erlotinib hydrochloride (e.g. Tarceva® by Genentech/Roche), Linifanib (or ABT 869, by Genentech), sunitinib malate (e.g. Sutent® by Pfizer), bosutinib (or SKI-606, described in U.S. Pat. No. 6,780,996), dasatinib (e.g. Sprycel® by Bristol-Myers Squibb), armala (e.g. pazopanib, e.g. Votrient® by GlaxoSmithKline), imatinib and imatinib mesylate (e.g. Gilvec® and Gleevec® by Novartis); Vascular Endothelial Growth Factor (VEG) receptor inhibitors (Bevacizumab, or Avastin® by Genentech/Roche), axitinib, (or AG013736, described in WO 01/002369), Brivanib Alaninate (or BMS-582664), motesanib (or AMG-706, described in PCT WO 02/066470), pasireotide (e.g. SOM230, described in WO 02/010192), sorafenib (e.g. Nexavar®); HER2 receptor inhibitors: Trastuzumab (e.g. Herceptin® by Genentech/Roche), neratinib (or HKI-272, described WO 05/028443), lapatinib or lapatinib ditosylate (e.g. Tykerb® by GlaxoSmithKline); CD20 antibodies: Rituximab (e.g. Riuxan® and MabThera® by Genentech/Roche), tositumomab (e.g. Bexxar® by GlaxoSmithKline), ofatumumab (e.g. Arzerra® by GlaxoSmithKline); Bcr/Abl kinase inhibitors: nilotinib hydrochloride (e.g. Tasigna® by Novartis); DNA Synthesis inhibitors: Capecitabine (e.g. Xeloda® by Roche), gemcitabine hydrochloride (e.g. Gemzar® by Eli Lilly and Company), nelarabine (or Arranon® and Atriance® by GlaxoSmithKline); Antineoplastic agents: oxaliplatin (e.g. Eloxatin® ay Sanofi-Aventis described in U.S. Pat. No. 4,169,846); Epidermal growth factor receptor (EGFR) inhibitors: Gefitinib (or Iressa®), Afatinib (or Tovok® by Boehringer Ingelheim), cetuximab (e.g. Erbitux® by Bristol-Myers Squibb), panitumumab (e.g. Vectibix® by Amgen); HER dimerization inhibitors: Pertuzumab (e.g. Omnitarg®, by Genentech); Human Granulocyte colony-stimulatingfactor (G-CSF) modulators: Filgrastim (e.g. Neupogen® by Amgen); Immunomodulators: Afutuzumab (by Roche®), pegfilgrastim (e.g. Neulasta® by Amgen), lenalidomide (e.g. CC-5013, e.g. Revlimid®), thalidomide (e.g. Thalomid®); CD40 inhibitors: Dacetuzumab (e.g. SGN-40 or huS2C6, by Seattle Genetics, Inc); Pro-apoptotic receptor agonists (PARAs): Dulanermin (e.g. AMG-951, by Amgen/Genentech); Hedgehog antagonists: Vismodegib (or GDC-0449, described in WO 06/028958); Phospholipase A2 inhibitors: Anagrelide (e.g. Agrylin®); BCL-2 inhibitors: Navitoclax (or ABT-263, described in WO 09/155386); Mitogen-activated protein kinase kinase (MEK) inhibitors: XL-518 (Cas No. 1029872-29-4, by ACC Corp.); Aromatase inhibitors: Exemestane (e.g. Aromasin® by Pfizer), letrozole (e.g. Femara® by Novartis), anastrozole (e.g. Arimidex®); Topoisomerase I inhibitors: Irinotecan (e.g. Camptosar® by Pfizer), topotecan hydrochloride (e.g. Hycamtin® by GlaxoSmithKline); Topoisomerase II inhibitors: etoposide (e.g. VP-16 and Etoposide phosphate, e.g. Toposar®, VePesid® and Etopophos®), teniposide (e.g. VM-26, e.g. Vumon®); mTOR inhibitors: Temsirolimus (e.g. Torisel® by Pfizer), ridaforolimus (formally known as deferolimus, (or AP23573 and MK8669, described in WO 03/064383), everolimus (e.g. Afinitor® by Novartis); Osteoclastic bone resorption inhibitors: zoledronic acid (or Zometa® by Novartis); CD33 Antibody Drug Conjugates: Gemtuzumab ozogamicin (e.g. Mylotarg® by Pfizer/Wyeth); CD22 Antibody Drug Conjugates: Inotuzumab ozogamicin (also referred to as CMC-544 and WAY-207294, by Hangzhou Sage Chemical Co., Ltd.); CD20 Antibody Drug Conjugates: Ibritumomab tiuxetan (e.g. Zevalin®); Somatostain analogs: octreotide (e.g. octreotide acetate, e.g. Sandostatin® and Sandostatin LAR®); Synthetic Interleukin-11 (IL-11): oprelvekin (e.g. Neumega® by Pfizer/Wyeth); Synthetic erythropoietin: Darbepoetin alfa (e.g. Aranesp® by Amgen); Receptor Activator for Nuclear Factor kappa B (RANK) inhibitors: Denosumab (e.g. Prolia® by Amgen); Thrombopoietin mimetic peptibodies: Romiplostim (e.g. Nplate® by Amgen; Cell growth stimulators: Palifermin (e.g. Kepivance® by Amgen); Anti-Insulin-like Growth Factor-1 receptor (IGF-1R) antibodies: Figitumumab (e.g. CP-751,871, by ACC Corp), robatumumab (CAS No. 934235-44-6); Anti-CS1 antibodies: Elotuzumab (HuLuc63, CAS No. 915296-00-3); CD52 antibodies: Alemtuzumab (e.g. Campath®); CTLA-4 inhibitors: Tremelimumab (IgG2 monoclonal antibody by Pfizer, formerly known as ticilimumab, CP-675,206), ipilimumab (CTLA-4 antibody, e.g. MDX-010, CAS No. 477202-00-9); Histone deacetylase inhibitors (HDI): Voninostat (e.g. Zolinza® by Merck); Alkylating agents: Temozolomide (e.g. Temodar® and Temodal® by Schering-Plough/Merck), dactinomycin (e.g. actinomycin-D and e.g. Cosmegen®), melphalan (e.g. L-PAM, L-sarcolysin, and phenylalanine mustard, e.g. Alkeran®), altretamine (e.g. hexamethylmelamine (HMM), e.g. Hexalen®), carmustine (e.g. BiCNU®), bendamustine (e.g. Treanda®), busulfan (e.g. Busulfex® and Myleran®), carboplatin (e.g. Paraplatin®), lomustine (e.g. CCNU, e.g. CeeNU®), cisplatin (e.g. CDDP, e.g. Platinol® and Platinol®-AQ), chlorambucil (e.g. Leukeran®), cyclophosphamide (e.g. Cytoxan® and Neosar®), dacarbazine (e.g. DTIC, DIC and imidazole carboxamide, e.g. DTIC-Dome®), altretamine (e.g. hexamethylmelamine (HMM) e.g. Hexalen®), ifosfamide (e.g. Ifex®), procarbazine (e.g. Matulane®), mechlorethamine (e.g. nitrogen mustard, mustine and mechloroethamine hydrochloride, e.g. Mustargen®), streptozocin (e.g. Zanosar®), thiotepa (e.g. thiophosphoamide, TESPA and TSPA, e.g. Thioplex®; Biologic response modifiers: bacillus calmette-guerin (e.g. theraCys® and TICE® BCG), denileukin diftitox (e.g. Ontak®); Anti-tumor antibiotics: doxorubicin (e.g. Adriamycin® and Rubex®), bleomycin (e.g. Lenoxane®), daunorubicin (e.g. dauorubicin hydrochloride, daunomycin, and rubidomycin hydrochloride, e.g. Cerubidine®), daunorubicin liposomal (daunorubicin citrate liposome, e.g. DaunoXome®), mitoxantrone (e.g. DHAD, e.g. Novantrone®), epirubicin (e.g. Ellence™), idarubicin (e.g. Idamycin®, Idamycin PFS®), mitomycin C (e.g. Mutamycin®); Anti-microtubule agents: Estramustine (e.g. Emcyl®); Cathepsin K inhibitors: Odanacatib (or MK-0822, by Lanzhou Chon Chemicals, ACC Corp., and ChemieTek, described in WO 03/075836); Epothilone B analogs: Ixabepilone (e.g. Lxempra® by Bristol-Myers Squibb); Heat Shock Protein (HSP) inhibitors: Tanespimycin (17-allylamino-17-demethoxygeldanamycin, e.g. KOS-953 and 17-AAG, by SIGMA, described in U.S. Pat. No. 4,261,989); TpoR agonists: Eltrombopag (e.g. Promacta® and Revolade® by GlaxoSmithKline); Anti-mitotic agents: Docetaxel (e.g. Taxotere® by Sanofi-Aventis); Adrenal steroid inhibitors: aminoglutethimide (e.g. Cytadren®); Anti-androgens: Nilutamide (e.g. Nilandron® and Anandron®), bicalutamide (sold under tradename Casodex®), flutamide (e.g. Fulexin™); Androgens: Fluoxymesterone (e.g. Halotestin®); Proteasome inhibitors: Bortezomib (e.g. Velcade®); CDK1 inhibitors: Alvocidib (e.g. flovopirdol or HMR-1275, described in U.S. Pat. No. 5,621,002); Gonadotropin-releasing hormone (GnRH) receptor agonists: Leuprolide or leuprolide acetate (e.g. Viadure® by Bayer AG, Eligard® by Sanofi-Aventis and Lupron® by Abbott Lab); Taxane anti-neoplastic agents: Cabazitaxel, larotaxel; 5HT1a receptor agonists: Xaliproden (or SR57746, described in U.S. Pat. No. 5,266,573); HPC vaccines: Cervarix® sold by GlaxoSmithKline, Gardasil® sold by Merck; Iron Chelating agents: Deferasinox (e.g. Exjade® by Novartis); Anti-metabolites: Claribine (2-chlorodeoxyadenosine, e.g. Leustatin®), 5-fluorouracil (e.g. Adrucil®), 6-thioguanine (e.g. Purinethol®), pemetrexed (e.g. Alimta®), cytarabine (e.g. arabinosylcytosine (Ara-C), e.g. Cytosar-U®), cytarabine liposomal (e.g. Liposomal Ara-C, e.g. DepoCyt™), decitabine (e.g. Dacogen®), hydroxyurea (e.g. Hydrea®, Droxia™ and Mylocel™), fludarabine (e.g. Fludara®), floxuridine (e.g. FUDR®), cladribine (e.g. 2-chlorodeoxyadenosine (2-CdA) e.g. Leustatin™), methotrexate (e.g. amethopterin, methotrexate sodim (MTX), e.g. Rheumatrex® and Trexall™), pentostatin (e.g. Nipent®); Bisphosphonates: Pamidronate (e.g. Aredia®), zoledronic acid (e.g. Zometa®); Demethylating agents: 5-azacitidine (e.g. Vidaza®), decitabine (e.g. Dacogen®); Plant Alkaloids: Paclitaxel protein-bound (e.g. Abraxane®), vinblastine (e.g. vinblastine sulfate, vincaleukoblastine and VLB, e.g. Alkaban-AQ® and Velban®), vincristine (e.g. vincristine sulfate, LCR, and VCR, e.g. Oncovin® and Vincasar Pfs®), vinorelbine (e.g. Navelbine®), paclitaxel (e.g. Taxol and Onxal™); Retinoids: Alitretinoin (e.g. Panretin®), tretinoin (all-trans retinoic acid, e.g. ATRA, e.g. Vesanoid®), Isotretinoin (13-cis-retinoic acid, e.g. Accutane®, Amnesteem®, Claravis®, Clarus®, Decutan®, Isotane®, Izotech®, Oratane®, Isotret®, and Sotret®), bexarotene (e.g. Targretin®); Glucocorticosteroids: Hydrocortisone (e.g. cortisone, hydrocortisone sodium succinate, hydrocortisone sodium phosphate, and e.g. Ala-Cort®, Hydrocortisone Phosphate, Solu-Cortef®, Hydrocort Acetate® and Lanacort®), dexamethasone, prednisolone (e.g. Delta-Cortel®, Orapred®, Pediapred® and Prelone®), prednisone (e.g. Deltasone®, Liquid Red®, Meticorten® and Orasone®), methylprednisolone (e.g. 6-Methylprednisolone, Methylprednisolone Acetate, Methylprednisolone Sodium Succinate, e.g. Duralone®, Medralone®, Medrol®, M-Prednisol® and Solu-Medrol®); Cytokines: interleukin-2 (e.g. aldesleukin and IL-2, e.g. Proleukin®), interleukin-11 (e.g. oprevelkin, e.g. Neumega®), alpha interferon alfa (e.g. IFN-alpha, e.g. Intron® A, and Roferon-A®); Lutinizing hormone releasing hormone (LHRH) agonists: Goserelin (e.g. Zoladex®); Progesterones: megestrol (e.g. megestrol acetate, e.g. Megace®); Miscellaneous cytotoxic agents: Arsenic trioxide (e.g. Trisenox®), asparaginase (e.g. L-asparaginase, Erwinia L-asparaginase, e.g. Elspar® and Kidrolase®); Anti-nausea drugs: NK-1 receptor antagonists: Casopitant (e.g. Rezonic® and Zunrisa® by GlaxoSmithKline); and Cytoprotective agents: Amifostine (e.g. Ethyol®), leucovorin (e.g. calcium leucovorin, citrovorum factor and folinic acid).

### EXAMPLES

### Example 1: Synthesis of Intermediate Compounds

Intermediate II: To a solution of methyl 5-bromo-2-methylbenzoate (10.0 g, 43.6 mmol, 1.00 eq) in methanol (100 mL) and triethylamine (10.9 g, 107 mmol, 15.0 mL, 2.47 eq) was added [1,1-bis(diphenylphosphino)ferrocene] dichloropalladium(II) (2.50 g, 3.42 mmol, 0.0783 eq). The mixture was stirred at 80° C. for 12 h under carbon monoxide (50 Psi). After being cooled to room temperature, the mixture was concentrated under reduced pressure. The residue was purified by column chromatography on silica gel (petroleum ether/ethyl acetate=10/1) to give dimethyl 4-methylisophthalate. ¹H NMR (400 MHz, CDCl₃) δ=8.59 (d, J=1.6 Hz, 1H), 8.07 (dd, J=1.8, 8.0 Hz, 1H), 7.35 (d, J=7.7 Hz, 1H), 3.94 (d, J=3.2 Hz, 6H), 2.68 (s, 3H).

Intermediate III: To a solution of dimethyl 4-methylisophthalate (7.00 g, 33.6 mmol, 1.00 eq) in sulfuric acid (20.0 mL) was added N-bromosuccinimide (7.18 g, 40.3 mmol, 1.20 eq). The mixture was stirred at 50° C. for 2 h. The reaction mixture was added into water (500 ml) at 0° C. White solid was precipitated from the mixture to give 5-bromo-4-methylisophthalic acid.

Intermediate IV: To a solution of 5-bromo-4-methylisophthalic acid (8.00 g, 30.8 mmol, 1.00 eq) in methanol (80.0 mL) was added thionyl chloride (18.3 g, 154 mmol, 11.2 mL, 5.00 eq). The mixture was stirred at 80° C. for 1 h. The reaction mixture was concentrated under reduced pressure to give a residue. The crude product was purified by column chromatography (petroleum ether/ethyl acetate=10/1) to give dimethyl 5-bromo-4-methylisophthalate.

Intermediate V: To a solution of dimethyl 5-bromo-4-methylisophthalate (8.80 g, 30.6 mmol, 1.00 eq) in trichloromethane (80.0 mL) was added N-Bromosuccinimide (6.00 g, 33.7 mmol, 1.10 eq) and benzoyl peroxide (1.48 g, 6.13 mmol, 0.200 eq). The mixture was stirred at 80° C. for 1 h. The reaction mixture was concentrated under reduced pressure to give a residue. The crude product was purified by column chromatography (petroleum ether/ethyl acetate=10/1) to give dimethyl 5-bromo-4-(bromomethyl)isophthalate. ¹H NMR (400 MHz, DMSO-d₆) δ=8.37-8.36 (m, 1H), 8.36-8.34 (m, 1H), 5.07-5.06 (m, 2H), 3.93 (s, 3H), 3.91 (s, 3H).

Intermediate VI: To a solution of dimethyl 5-bromo-4-(bromomethyl)isophthalate (11.0 g, 30.0 mmol, 1.00 eq) in acetonitrile (110 mL) was added N,N-diisopropylethylamine (11.6 g, 90.1 mmol, 15.7 mL, 3.00 eq) and 3-aminopiperidine-2,6-dione hydrochloride (5.94 g, 36.0 mmol, 1.20 eq, HCl). The mixture was stirred at 80° C. for 1 h. The reaction mixture was concentrated under reduced pressure to give a residue. The crude product was triturated with the mixture of hydrochloric acid (1.00 M, 50.0 ml) and ethyl acetate (100 ml). After filtered, the filter cake was washed with water (5.00 ml) and dried to give methyl 7-bromo-2-(2,6-dioxopiperidin-3-yl)-3-oxoisoindoline-5-carboxylate. ¹H NMR (400 MHz, DMSO-d₆) δ=11.04 (s, 1H), 8.36-8.24 (m, 1H), 8.24-8.15 (m, 1H), 5.17 (dd, J=5.1, 13.2 Hz, 1H), 4.58-4.47 (m, 1H), 4.43-4.29 (m, 1H), 3.92 (s, 3H), 3.00-2.85 (m, 1H), 2.61 (br d, J=17.0 Hz, 1H), 2.50-2.43 (m, 1H), 2.07-1.99 (m, 1H).

Intermediate VII: To a solution of methyl 7-bromo-2-(2,6-dioxopiperidin-3-yl)-3-oxoisoindoline-5-carboxylate (1.00 g, 2.62 mmol, 1.00 eq) in tetrahydrofuran (50.0 mL) was added lithium aluminum hydride (100 mg, 2.63 mmol, 1.00 eq) at 0° C. The mixture was stirred at 0° C. for 0.5 h. The mixture was quenched with hydrochloric acid (10.0 M, 2.00 mL) at 0° C. The mixture was concentrated under reduced pressure. The residue was purified by reverse phase HPLC (0.1% formic acid). The desired fraction was concentrated under reduced pressure to give 3-(4-bromo-6-(hydroxymethyl)-1-oxoisoindolin-2-yl)piperidine-2,6-dione. ¹H NMR (400 MHz, DMSO-d₆) δ=11.01 (s, 1H), 7.80 (s, 1H), 7.71 (s, 1H), 5.49 (t, J=5.9 Hz, 1H), 5.14 (dd, J=5.1, 13.3 Hz, 1H), 4.62 (d, J=5.6 Hz, 2H), 4.44-4.36 (m, 1H), 4.29-4.20 (m, 1H), 2.97-2.85 (m, 1H), 2.62 (br s, 1H), 2.48-2.41 (m, 1H), 2.06-1.98 (m, 1H). MS (ESI) m/z 355.0 [M+H]⁺.

Intermediate VIII: To a solution of 3-(4-bromo-6-(hydroxymethyl)-1-oxoisoindolin-2-yl)piperidine-2,6-dione (80.0 mg, 226 umol, 1.00 eq) in dimethyl formamide (2.00 mL) was added zinc cyanide (265 mg, 2.27 mmol, 143 uL, 10.0 eq), 1,1′bis(diphenylphosphino)ferrocene (25.1 mg, 45.3 umol, 0.200 eq) and tris(dibenzylideneacetone) dipalladium(0) (20.7 mg, 22.6 umol, 0.100 eq) under nitrogen. The mixture was stirred at 120° C. for 2 h. The mixture was filtered. The liquor was diluted with water (30.0 mL) and exacted with ethyl acetate (3×30.0 mL). The organic phase was separated, washed with brine (2×10.0 mL), dried over sodium sulfate, filtered and concentrated under reduced pressure to give a residue. The crude product was purified by reverse phase HPLC to give 2-(2,6-dioxopiperidin-3-yl)-6-(hydroxymethyl)-1-oxoisoindoline-4-carbonitrile. MS (ESI) m/z 300.1 [M+H]⁺

Intermediate X: A mixture of 3-fluoro-2-methyl-benzoic acid (10.0 g, 64.9 mmol, 1.00 eq) and N-iodosuccinimide (14.6 g, 64.9 mmol, 1.00 eq) in sulfuric acid (200 mL) was stirred at 25° C. for 12 h. The mixture was diluted with water (1000 mL) and extracted with ethyl acetate (3×500 mL). The combined organic layer was washed with brine (500 mL), dried over sodium sulfate, filtered and concentrated in vacuum. The residue was purified by silica gel chromatography to give 3-fluoro-5-iodo-2-methylbenzoic acid. MS (ESI) m/z 279.1 [M−H]⁻

Intermediate XI: A mixture of 3-fluoro-5-iodo-2-methyl-benzoic acid (3.00 g, 10.7 mmol, 1.00 eq) and thionyl chloride (6.37 g, 53.6 mmol, 3.89 mL, 5.00 eq) in methanol (30.0 mL) was stirred at 80° C. for 12 h. The mixture was concentrated in vacuum. The crude product was purified by reversed-phase column (0.1% formic acid condition). The desired fraction was collected and lyophilized to give methyl 3-fluoro-5-iodo-2-methylbenzoate. ¹H NMR (400 MHz, CDCl₃) δ=7.92 (s, 1H), 7.49-7.42 (m, 1H), 3.83 (s, 3H), 2.36 (d, J=2.3 Hz, 3H).

Intermediate XII: A mixture of methyl 3-fluoro-5-iodo-2-methyl-benzoate (2.80 g, 9.52 mmol, 1.00 eq), N-Bromosuccinimide (1.69 g, 9.52 mmol, 1.00 eq) and benzoyl benzenecarboperoxoate (461 mg, 1.90 mmol, 0.200 eq) in trichloromethane (30.0 mL) was stirred at 80° C. for 12 h. The mixture was diluted with water (100 mL) and extracted with ethyl acetate (3×100 mL). The combined organic layer was washed with brine (50.0 mL), dried over sodium sulfate, filtered and concentrated in vacuum. The residue was purified by silica gel chromatography to give methyl 2-(bromomethyl)-3-fluoro-5-iodobenzoate. ¹H NMR (400 MHz, DMSO-d₆) δ=8.06-7.99 (m, 2H), 4.97 (d, J=1.8 Hz, 1H), 4.89 (d, J=1.7 Hz, 2H), 3.90-3.88 (m, 3H).

Intermediate XIII: A mixture of methyl 2-(bromomethyl)-3-fluoro-5-iodobenzoate (1.40 g, 3.75 mmol, 1.00 eq), 3-aminopiperidine-2,6-dione (617.84 mg, 3.75 mmol, 1.00 eq, HCl) and N,N-diisopropylethylamine (1.94 g, 15.0 mmol, 2.62 mL, 4.00 eq) in acetonitrile (10.0 mL) was stirred at 80° C. for 12 h. The mixture was concentrated in vacuum. The crude product was triturated with ethyl acetate/water=2/1 (10.0 mL) and filtered. The filter cake was dried in vacuum to give 3-(4-fluoro-6-iodo-1-oxoisoindolin-2-yl) piperidine-2,6-dione. ¹H NMR (400 MHz, DMSO-d₆) δ=11.03 (s, 1H), 7.93-7.93 (m, 1H), 7.93-7.92 (m, 1H), 7.98-7.88 (m, 1H), 7.73-7.73 (m, 1H), 7.98-7.47 (m, 1H), 5.17-5.07 (m, 1H), 4.63-4.49 (m, 1H), 4.45-4.32 (m, 1H), 2.98-2.85 (m, 1H), 2.67-2.57 (m, 1H), 2.46-2.36 (m, 1H), 2.06-1.96 (m, 1H).

Intermediate XIV: To a mixture of 3-(4-fluoro-6-iodo-1-oxo-isoindolin-2-yl)piperidine-2,6-dione (960 mg, 2.47 mmol, 1.00 eq), 2,3,4,6,7,8,9,10-octahydropyrimido(1,2-a)azepine (753 mg, 4.95 mmol, 745 uL, 2.00 eq) in dimethyl formamide (5.00 mL) was added 2-(chloromethoxy)ethyl-trimethylsilane (742 mg, 4.45 mmol, 788 uL, 1.8 eq) dropwise at 0° C. The mixture was stirred at 25° C. for 2 h. The mixture was diluted with water (100 mL) and extracted with ethyl acetate (3×100 mL). The combined organic layer was washed with brine (50.0 mL), dried over sodium sulfate, filtered and concentrated in vacuum. The residue was purified by silica gel chromatography to give 3-(4-fluoro-6-iodo-1-oxoisoindolin-2-yl)-1-((2-(trimethylsilyl)ethoxy)methyl)piperidine-2,6-dione. ¹H NMR (400 MHz, DMSO-d₆) δ=7.92 (br d, J=1.1 Hz, 2H), 5.30-5.18 (m, 1H), 5.10-5.00 (m, 2H), 4.56 (d, J=17.5 Hz, 1H), 4.32 (d, J=17.5 Hz, 1H), 3.56-3.44 (m, 2H), 3.09-3.00 (m, 1H), 2.81-2.75 (m, 1H), 2.45-2.35 (m, 1H), 2.11-2.02 (m, 1H), 1.17 (t, J=7.1 Hz, 1H), 0.87-0.78 (m, 2H), −0.03 (s, 9H).

Intermediate XV: To a mixture of 3-(4-fluoro-6-iodo-1-oxoisoindolin-2-yl)-1-((2-(trimethylsilyl)ethoxy)methyl)piperidine-2,6-dione (750 mg, 1.45 mmol, 1.00 eq), triethylsilane (1.35 g, 11.6 mmol, 1.85 mL, 8.00 eq) and N,N-diisopropylethylamine (935 mg, 7.23 mmol, 1.26 mL, 5.00 eq) in dimethyformamide (10.0 mL) was added [1,1-bis(diphenylphosphino)ferrocene]dichloropalladium(II) (106 mg, 145 umol, 0.100 eq) in one portion. The mixture was stirred at 80° C. for 12 h under carbon monoxide (50 Psi). The mixture was diluted with water (40.0 mL) and extracted with ethyl acetate (3×50.0 mL). The combined organic layer was washed with brine (20.0 mL), dried over sodium sulfate, filtered and concentrated in vacuum. The residue was purified by silica gel chromatography to give 2-(2,6-dioxo-1-((2-(trimethylsilyl)ethoxy)methyl)piperidin-3-yl)-7-fluoro-3-oxoisoindoline-5-carbaldehyde. ¹H NMR (400 MHz, DMSO-d₆) δ=10.12 (d, J=1.6 Hz, 1H), 8.18 (s, 1H), 7.98 (d, J=8.8 Hz, 1H), 5.28 (dd, J=5.0, 13.4 Hz, 1H), 5.12-4.98 (m, 2H), 4.73 (d, J=18.3 Hz, 1H), 4.49 (d, J=18.3 Hz, 1H), 3.59-3.50 (m, 2H), 3.14-3.02 (m, 1H), 2.85-2.78 (m, 1H), 2.48-2.40 (m, 1H), 2.13-2.04 (m, 1H), 0.84 (ddd, J=3.1, 6.3, 9.9 Hz, 2H), −0.02 (s, 9H).

Intermediate XVI: To a mixture of 2-(2,6-dioxo-1-((2-(trimethylsilyl)ethoxy)methyl)piperidin-3-yl)-7-fluoro-3-oxoisoindoline-5-carbaldehyde (130 mg, 309 umol, 1.00 eq) and acetic acid (92.8 mg, 1.55 mmol, 88.4 uL, 5.00 eq) in dimethyformamide (3.00 mL) and dichloromethane (3.00 mL) was added sodium triacetoxyhydroborate (328 mg, 1.55 mmol, 5.00 eq) in portions. The mixture was stirred at 50° C. for 1 h. The mixture was diluted with water (50.0 mL) and extracted with ethyl acetate (3×30.0 mL). The combined organic layer was washed with brine (20.0 mL), dried over sodium sulfate, filtered and concentrated in vacuum to give 3-(4-fluoro-6-(hydroxymethyl)-1-oxoisoindolin-2-yl)-1-((2-(trimethylsilyl)ethoxy)methyl)piperidine-2,6-dione.

Intermediate XVII: A solution of 3-(4-fluoro-6-(hydroxymethyl)-1-oxoisoindolin-2-yl)-1-((2-(trimethylsilyl)ethoxy)methyl) piperidine-2,6-dione (130 mg, 308 umol, 1.00 eq) in 4 M hydrochloric acid/dioxane (4.00 mL) was stirred at 50° C. for 1 h. The mixture was concentrated in vacuum to give 3-(4-fluoro-6-(hydroxymethyl)-1-oxoisoindolin-2-yl)-1-(hydroxymethyl)piperidine-2,6-dione.

Intermediate XVIII: A solution of 3-(4-fluoro-6-(hydroxymethyl)-1-oxoisoindolin-2-yl)-1-(hydroxymethyl)piperidine-2,6-dione (100 mg, 310 umol, 1.00 eq) in ammonium hydroxide (0.100 mL, 26% purity) and acetonitrile (5.00 mL) was stirred at 25° C. for 2 h. The mixture was adjusted PH=3-4 with 1 M hydrochloric acid. The mixture was purified by prep-HPLC and lyophilized to give 3-(4-fluoro-6-(hydroxymethyl)-1-oxoisoindolin-2-yl)piperidine-2,6-dione. ¹H NMR (400 MHz, DMSO-d₆) δ=10.99 (br d, J=4.9 Hz, 1H), 7.55 (s, 1H), 7.40 (br d, J=9.8 Hz, 1H), 5.11 (br dd, J=5.0, 13.2 Hz, 1H), 4.61 (s, 2H), 4.54 (br d, J=17.4 Hz, 1H), 4.41-4.33 (m, 1H), 2.96-2.86 (m, 1H), 2.60 (br d, J=17.6 Hz, 1H), 2.45-2.37 (m, 1H), 2.05-1.95 (m, 1H).

Intermediate XX: A solution of 3-chloro-2-methylbenzoic acid (9.70 g, 56.9 mmol, 1.00 eq) in concentrated sulfuric acid (150 mL) was added N-Iodosuccinimide (12.8 g, 56.9 mmol, 1.00 eq) at 0° C. The resulting mixture was stirred at 25° C. for 12 h. The reaction mixture was poured into water (500 mL) and extracted with ethyl acetate (3×200 mL). The combined organic layer was washed with brine (100 mL), dried over sodium sulfate, filtered, and concentrated in vacuum. The residue was purified by silica gel chromatography to give 3-chloro-5-iodo-2-methylbenzoic acid. 1H NMR (400 MHz, CDCl3) δ=8.21 (d, J=1.8 Hz, 1H), 7.92 (d, J=1.8 Hz, 1H), 2.63 (s, 3H).

Intermediate XXI: A mixture of 3-chloro-5-iodo-2-methylbenzoic acid (14.0 g, 47.2 mmol, 1.00 eq) and thionyl chloride (16.9 g, 142 mmol, 10.3 mL, 3.00 eq) in methanol (100 mL) was stirred at 80° C. for 12 h. The mixture was concentrated in vacuum. The residue was diluted with water (150 mL) and extracted with ethyl acetate (3×100 mL). The combined organic layer was washed with brine (60.0 mL), dried over sodium sulfate, filtered, and concentrated in vacuum to give methyl 3-chloro-5-iodo-2-methylbenzoate. 1H NMR (400 MHz, CDCl3) δ=7.93 (d, J=1.8 Hz, 1H), 7.75 (d, J=1.7 Hz, 1H), 3.82 (s, 3H), 2.46 (s, 3H).

Intermediate XXII: To a mixture of methyl 3-chloro-5-iodo-2-methylbenzoate (11.0 g, 35.4 mmol, 1.00 eq) and N-Bromosuccinimide (7.57 g, 42.5 mmol, 1.20 eq) in trichloromethane (100 mL) was added benzoyl peroxide (1.72 g, 7.08 mmol, 0.200 eq) in one portion. The mixture was stirred at 90° C. for 12 h. The mixture was concentrated in vacuum. The residue was diluted with water (150 mL) and extracted with ethyl acetate (3×80.0 mL). The combined organic layer was washed with brine (60 mL), dried over sodium sulfate, filtered, and concentrated in vacuum. The residue was purified by silica gel chromatography to give methyl 2-(bromomethyl)-3-chloro-5-iodobenzoate. 1H NMR (400 MHz, CDCl3) δ=8.18 (d, J=1.7 Hz, 1H), 7.93 (d, J=1.7 Hz, 1H), 5.05 (s, 2H), 3.97 (s, 3H).

Intermediate XXIII: To a mixture of methyl 2-(bromomethyl)-3-chloro-5-iodobenzoate (13.0 g, 33.4 mmol, 1.00 eq) and 3-aminopiperidine-2,6-dione (5.49 g, 33.4 mmol, 1.00 eq, HCl) in acetonitrile (100 mL) was added N,N-diisopropylethylamine (12.9 g, 100 mmol, 17.4 mL, 3.00 eq) dropwise. The mixture was stirred at 90° C. for 3 h. The mixture was concentrated in vacuum. The residue was triturate with ethyl acetate/water=2/1 (30.0 mL) and filtered. The filter cake was dried in vacuum to give 3-(4-chloro-6-iodo-1-oxoisoindolin-2-yl)piperidine-2,6-dione. ¹H NMR (400 MHz, DMSO-d6) δ=11.01 (br s, 1H), 8.10 (d, J=1.3 Hz, 1H), 8.01 (d, J=1.2 Hz, 1H), 5.12 (br dd, J=5.2, 13.3 Hz, 1H), 4.48-4.41 (m, 1H), 4.32-4.25 (m, 1H), 2.96-2.86 (m, 1H), 2.63-2.56 (m, 1H), 2.49-2.39 (m, 1H), 2.05-1.98 (m, 1H).

Intermediate XXIV: To a mixture of 3-(4-chloro-6-iodo-1-oxoisoindolin-2-yl)piperidine-2,6-dione (5.00 g, 12.4 mmol, 1.00 eq) and 1,8-diazabicyclo[5.4.0]undec-7-ene (3.76 g, 24.7 mmol, 3.73 mL, 2.00 eq) in dimethyformamide (30.0 mL) was added 2-(trimethylsilyl)ethoxymethyl chloride (3.71 g, 22.2 mmol, 3.94 mL, 1.80 eq) dropwise at 0° C. The mixture was stirred at 25° C. for 2 h. The mixture was diluted with water (150 mL) and extracted with ethyl acetate (3×80.0 mL). The combined organic layer was washed with brine (40.0 mL), dried over sodium sulfate, filtered, and concentrated in vacuum. The residue was purified by silica gel chromatography to give 3-(4-chloro-6-iodo-1-oxoisoindolin-2-yl)-1-((2-(trimethylsilyl) ethoxy)methyl) piperidine-2,6-dione. ¹H NMR (400 MHz, DMSO-d6) δ=8.13 (d, J=1.1 Hz, 1H), 8.03 (d, J=1.1 Hz, 1H), 5.27 (dd, J=5.1, 13.4 Hz, 1H), 5.05 (q, J=9.8 Hz, 2H), 4.47 (d, J=17.8 Hz, 1H), 4.24 (d, J=17.8 Hz, 1H), 3.56-3.48 (m, 2H), 3.11-3.00 (m, 1H), 2.83-2.77 (m, 1H), 2.44 (br dd, J=4.3, 13.0 Hz, 1H), 2.11-2.03 (m, 1H), 0.90-0.78 (m, 2H), −0.03 (s, 9H).

Intermediate XXV: To a mixture of 3-(4-chloro-6-iodo-1-oxoisoindolin-2-yl)-1-((2-(trimethylsilyl)ethoxy)methyl) piperidine-2,6-dione (3.50 g, 6.54 mmol, 1.00 eq), triethylsilane (6.09 g, 52.3 mmol, 8.36 mL, 8.00 eq) and N,N-diisopropylethylamine (4.23 g, 32.7 mmol, 5.70 mL, 5.00 eq) in dimethyformamide (20.0 mL) was added [1,1-bis(diphenylphosphino)ferrocene]dichloropalladium(II) (479 mg, 654 umol, 0.100 eq) in one portion. The mixture was stirred at 80° C. for 12 h under carbon monoxide (50 Psi). The mixture was diluted with water (100 mL) and extracted with ethyl acetate (3×80.0 mL). The combined organic layer was washed with brine (40.0 mL), dried over sodium sulfate, filtered and concentrated in vacuum. The residue was purified by silica gel chromatography to give 7-chloro-2-(2,6-dioxo-1-((2-(trimethylsilyl) ethoxy)methyl)piperidin-3-yl)-3-oxoisoindoline-5-carbaldehyde. ¹H NMR (400 MHz, DMSO-d6) δ=10.11 (s, 1H), 8.31-8.17 (m, 2H), 5.31 (dd, J=5.1, 13.3 Hz, 1H), 5.06 (q, J=9.8 Hz, 2H), 4.64 (br d, J=18.5 Hz, 1H), 4.41 (d, J=18.6 Hz, 1H), 3.58-3.49 (m, 2H), 3.13-3.04 (m, 1H), 2.85-2.78 (m, 1H), 2.49-2.41 (m, 1H), 2.15-2.03 (m, 1H), 0.84 (ddd, J=3.9, 6.9, 9.2 Hz, 2H), −0.01-−0.03 (m, 9H).

Intermediate XXVI To a mixture of 7-chloro-2-(2,6-dioxo-1-((2-(trimethylsilyl)ethoxy)methyl)piperidin-3-yl)-3-oxoisoindoline-5-carbaldehyde (760 mg, 1.74 mmol, 1.00 eq), acetic acid (522 mg, 8.70 mmol, 497 uL, 5.00 eq) in dimethyformamide (3.00 mL) and dichloromethane (3.00 mL) was added sodium triacetoxyhydroborate (1.84 g, 8.70 mmol, 5.00 eq) in portions. The mixture was stirred at 50° C. for 1 h. The mixture was diluted with water (50.0 mL) and extracted with ethyl acetate (3×30.0 mL). The combined organic layer was washed with brine (20.0 mL), dried over sodium sulfate, filtered, and concentrated in vacuum. The residue was purified by silica gel chromatography to give 3-(4-chloro-6-(hydroxymethyl)-1-oxoisoindolin-2-yl)-1-((2-(trimethylsilyl) ethoxy)methyl)piperidine-2,6-dione. ¹H NMR (400 MHz, DMSO-d6) δ=7.67 (d, J=8.8 Hz, 2H), 5.49 (br dd, J=2.1, 4.9 Hz, 1H), 5.27 (dd, J=5.0, 13.4 Hz, 1H), 5.06 (q, J=9.8 Hz, 2H), 4.62 (s, 2H), 4.49 (d, J=17.4 Hz, 1H), 4.26 (d, J=17.4 Hz, 1H), 3.58-3.48 (m, 2H), 3.13-3.01 (m, 1H), 2.83-2.77 (m, 1H), 2.49-2.38 (m, 1H), 2.07 (ddd, J=2.4, 5.1, 9.9 Hz, 1H), 0.90-0.79 (m, 2H), 0.00-−0.04 (m, 9H).

Intermediate XXVII: A solution of 3-(4-chloro-6-(hydroxymethyl)-1-oxoisoindolin-2-yl)-1-((2-(trimethylsilyl)ethoxy) methyl) piperidine-2,6-dione (470 mg, 1.07 mmol, 1.00 eq) in 4 M hydrochloric acid/dioxane (6.00 mL) was stirred at 50° C. for 1 h. The mixture was concentrated in vacuum to give 3-(4-chloro-6-(hydroxymethyl)-1-oxoisoindolin-2-yl)-1-(hydroxymethyl)piperidine-2,6-dione.

Intermediate XXVIII: A solution of 3-(4-chloro-6-(hydroxymethyl)-1-oxoisoindolin-2-yl)-1-(hydroxymethyl)piperidine-2,6-dione (360 mg, 1.06 mmol, 1.00 eq) in ammonium hydroxide (0.100 mL, 26% purity) and acetonitrile (5.00 mL) was stirred at 25° C. for 2 h. The mixture was adjusted PH=3-4 with 1M hydrochloric acid. The mixture was purified by reverse phase chromatography to give 3-(4-chloro-6-(hydroxymethyl)-1-oxoisoindolin-2-yl)piperidine-2,6-dione. ¹H NMR (400 MHz, DMSO-d6) δ=10.97 (br d, J=1.5 Hz, 1H), 7.66 (d, J=6.6 Hz, 2H), 5.49 (br s, 1H), 5.13 (dd, J=5.1, 13.3 Hz, 1H), 4.61 (br s, 2H), 4.52-4.43 (m, 1H), 4.35-4.26 (m, 1H), 2.98-2.85 (m, 1H), 2.65-2.57 (m, 1H), 2.48-2.41 (m, 1H), 2.07-1.97 (m, 1H).

Intermediate XXX: To a solution of 2-methyl-5-nitro-benzoic acid (25.0 g, 138 mmol, 1.00 eq) in sulfuric acid (125 mL) was added N-iodosuccinimide (37.3 g, 166 mmol, 1.20 eq) and stirred at 60° C. for 2 h. The reaction suspension was cooled to 25° C., poured into ice water (500 mL) and stirred for 10 min. The mixture was filtered, washed with water (500 mL). The filter cake was swapped with toluene to remove residual water to give 3-iodo-2-methyl-5-nitrobenzoic acid. ¹H NMR (400 MHz, CDCl₃) δ=8.88 (d, J=2.4 Hz, 1H), 8.81 (d, J=2.4 Hz, 1H), 2.87 (s, 3H).

Intermediate XXXI: To a reaction mixture of 3-iodo-2-methyl-5-nitrobenzoic acid (41.0 g, 134 mmol, 1.00 eq), cesium carbonate (130 g, 401 mmol, 3.00 eq) and cupric oxide (1.91 g, 13.3 mmol, 1.36 mL, 0.100 eq) in water (400 mL) was added p-toluenesulfonic acid monohydrate (12.7 g, 66.8 mmol, 0.500 eq) slowly and stirred at 120° C. for 12 h under nitrogen atmosphere. The reaction suspension was filtered, the filtrate was acidified to pH=4 and extracted with ethyl acetate (3×200 mL), washed with brine (200 mL), dried over anhydrous sodium sulfate and concentrated in vacuum to give 3-hydroxy-2-methyl-5-nitrobenzoic acid. ¹H NMR (400 MHz, DMSO-d₆) δ=13.49 (br s, 1H), 10.79 (s, 1H), 7.99 (d, J=2.4 Hz, 1H), 7.74 (d, J=2.4 Hz, 1H), 2.40 (s, 3H). MS (ESI) m/z 196.1 [M−H]⁺.

Intermediate XXXII: To a suspension of 3-hydroxy-2-methyl-5-nitrobenzoic acid (19.5 g, 98.9 mmol, 1.00 eq) and potassium carbonate (41.0 g, 297 mmol, 3.00 eq) in dimethyl formamide (200 mL) was added methyl iodide (42.1 g, 297 mmol, 18.5 mL, 3.00 eq) and stirred at 25° C. for 2 h. The reaction suspension was poured into brine (1.00 L), extracted with ethyl acetate (3×300 mL), washed with brine (500 mL), dried over anhydrous sodium sulfate and concentrated in vacuum to give methyl 3-methoxy-2-methyl-5-nitrobenzoate. ¹H NMR (400 MHz, CDCl₃) δ=8.32 (d, J=2.4 Hz, 1H), 7.79 (d, J=2.4 Hz, 1H), 3.96 (d, J=6.0 Hz, 6H), 2.53 (s, 3H).

Intermediate XXXIII: To a solution of methyl 3-methoxy-2-methyl-5-nitro-benzoate (25.0 g, 111 mmol, 1.00 eq) in methanol (250 mL) and water (50.0 mL) was added iron powder (31.0 g, 555 mmol, 5.00 eq) and ammonium chloride (29.7 g, 555 mmol, 5.00 eq). The reaction mixture was stirred at 80° C. for 2 h. The reaction mixture was filtered with celite pad, the filtrate was concentrated in vacuum to afford a residue which was washed with brine (100 mL), extracted with ethyl acetate (3×100 mL), dried over anhydrous sodium sulfate and concentrated in vacuum to give a residue. The residue was purified by silica gel chromatography to give methyl 5-amino-3-methoxy-2-methylbenzoate. ¹H NMR (400 MHz, CDCl₃) δ=6.75 (d, J=2.4 Hz, 1H), 6.37 (d, J=2.4 Hz, 1H), 3.87 (s, 3H), 3.80 (s, 3H), 3.74-3.50 (m, 2H), 2.29 (s, 3H). MS (ESI) m/z 196.2 [M+H]⁺.

Intermediate XXXIV: To a mixture of methyl 5-amino-3-methoxy-2-methylbenzoate (5.00 g, 25.6 mmol, 1.00 eq) in acetonitrile (50.0 mL) was added hydrochloric acid (3 M, 8.54 mL, 1.00 eq) and sodium nitrite (3.53 g, 51.2 mmol, 2.00 eq) at 0° C. After stirred for 30 min, potassium iodide (17.0 g, 102 mmol, 4.00 eq) was added and mixture was stirred at 25° C. for 2 h. The reaction mixture was extracted with ethyl acetate (3×50.0 mL), the combined organic layers were washed with brine (50.0 mL), dried over anhydrous sodium sulfate, filtered and concentrated under reduced pressure to give a residue, which was purified by silica gel chromatography to give methyl 5-iodo-3-methoxy-2-methylbenzoate. ¹H NMR (400 MHz, DMSO-d₆) δ=7.62 (d, J=1.6 Hz, 1H), 7.45 (d, J=1.6 Hz, 1H), 3.83 (d, J=6.4 Hz, 6H), 2.25 (s, 3H). MS (ESI) m/z 307.0 [M+H]⁺.

Intermediate XXXV: To a mixture of methyl 5-iodo-3-methoxy-2-methylbenzoate (6.50 g, 21.2 mmol, 1.00 eq) in trichloromethane (20.0 mL) was added benzoyl peroxide (1.54 g, 6.37 mmol, 0.300 eq) and N-bromosuccinimide (4.16 g, 23.4 mmol, 1.10 eq) at 25° C. The reaction mixture was stirred at 80° C. for 12 h. The reaction mixture was concentrated to give a residue, which was purified by silica gel chromatography to give methyl 2-(bromomethyl)-5-iodo-3-methoxybenzoate. ¹H NMR (400 MHz, DMSO-d₆) δ=7.75 (d, J=1.6 Hz, 1H), 7.63 (d, J=1.6 Hz, 1H), 4.89 (s, 2H), 3.92 (s, 3H), 3.87 (s, 3H).

Intermediate XXXVI: To a mixture of methyl 2-(bromomethyl)-5-iodo-3-methoxybenzoate (5.80 g, 15.1 mmol, 1.00 eq) and 3-aminopiperidine-2,6-dione (2.73 g, 16.6 mmol, 1.10 eq, HCl) in acetonitrile (60.0 mL) was added N,N-diisopropylethylamine (1.95 g, 15.1 mmol, 2.62 mL, 1.00 eq) at 25° C. The reaction mixture was stirred at 80° C. for 12 h. The reaction mixture was concentrated to give a residue, which was triturated with ethyl acetate (50.0 mL) at 25° C. for 10 min to give 3-(6-iodo-4-methoxy-1-oxoisoindolin-2-yl) piperidine-2,6-dione.

Intermediate XXXVII: To a mixture of 3-(6-iodo-4-methoxy-1-oxoisoindolin-2-yl) piperidine-2,6-dione (3.20 g, 8.00 mmol, 1.00 eq) and 1,8-diazabicyclo[5.4.0]undec-7-ene (4.87 g, 32.0 mmol, 4.82 mL, 4.00 eq) in dimethyl formamide (30.0 mL) was added (2-(chloromethoxy)ethyl)trimethylsilane (5.33 g, 32.0 mmol, 5.66 mL, 4.00 eq) at 0° C. The reaction mixture was stirred at 25° C. for 6 h. The reaction mixture was poured into water (100 mL) and extracted with ethyl acetate (3×30.0 mL). The combined organic phase was separated, washed with brine (10.0 mL), dried over anhydrous sodium sulfate, filtered and concentrated to give a residue. The residue was purified by silica gel chromatography to give 3-(6-iodo-4-methoxy-1-oxoisoindolin-2-yl)-1-((2-(trimethylsilyl)ethoxy)methyl) piperidine-2,6-dione. ¹H NMR (400 MHz, DMSO-d₆) δ=7.66 (d, J=1.2 Hz, 1H), 7.58 (d, J=1.2 Hz, 1H), 5.24 (dd, J=5.2, 13.2 Hz, 1H), 5.12-5.01 (m, 2H), 4.39 (d, J=17.6 Hz, 1H), 4.16 (d, J=17.6 Hz, 1H), 3.93 (s, 3H), 3.54 (ddd, J=5.6, 7.6, 8.8 Hz, 2H), 3.17-3.00 (m, 1H), 2.85-2.76 (m, 1H), 2.43 (dq, J=4.4, 13.2 Hz, 1H), 2.06 (tdd, J=2.4, 5.2, 9.8 Hz, 1H), 0.86 (ddd, J=3.6, 6.8, 9.2 Hz, 2H), 0.00 (s, 9H).

Intermediate XXXVIII: To a mixture of 3-(6-iodo-4-methoxy-1-oxoisoindolin-2-yl)-1-((2-(trimethylsilyl) ethoxy)methyl) piperidine-2,6-dione (1.30 g, 2.45 mmol, 1.00 eq), [1,1-bis(diphenylphosphino) ferrocene]dichloropalladium(II) (359 mg, 490 umol, 0.200 eq), triethylsilane (2.85 g, 24.5 mmol, 3.91 mL, 10.0 eq) in dimethyl formamide (15.0 mL) was added N,N-diisopropylethylamine (1.58 g, 12.3 mmol, 2.13 mL, 5.00 eq). The reaction mixture was stirred at 80° C. for 12 h under carbon monoxide (50 psi). The reaction mixture was filtered to give filtrate. The filtrate was poured into water (50.0 mL) and extracted with ethyl acetate (3×20.0 mL). The combined organic layers were washed with brine (50.0 mL), dried over anhydrous sodium sulfate, filtered and the filtrate was concentrated to afford a residue. The residue was purified by silica gel chromatography to give 2-(2,6-dioxo-1-((2-(trimethylsilyl)ethoxy)methyl) piperidin-3-yl)-7-methoxy-3-oxoisoindoline-5-carbaldehyde. ¹H NMR (400 MHz, DMSO-d₆) δ=10.13 (s, 1H), 7.94 (s, 1H), 7.72 (s, 1H), 5.28 (br dd, J=5.2, 13.2 Hz, 1H), 5.18-4.98 (m, 2H), 4.55 (br d, J=18.4 Hz, 1H), 4.32 (br d, J=18.4 Hz, 1H), 4.03-3.94 (m, 3H), 3.63-3.47 (m, 2H), 3.16-3.03 (m, 1H), 2.86-2.77 (m, 1H), 2.50-2.39 (m, 1H), 2.08 (ddd, J=2.0, 5.2, 7.2 Hz, 1H), 0.86 (ddd, J=3.2, 6.4, 9.6 Hz, 2H), 0.14-−0.12 (m, 9H).

Intermediate XXXIX: A mixture of 2-(2,6-dioxo-1-((2-(trimethylsilyl)ethoxy)methyl)piperidin-3-yl)-7-methoxy-3-oxoisoindoline-5-carbaldehyde (800 mg, 1.85 mmol, 1.00 eq) and acetic acid (555 mg, 9.25 mmol, 529 uL, 5.00 eq) in dimethyl formamide (5.00 mL) and dichloromethane (5.00 mL) was added sodium triacetoxyborohydride (1.96 g, 9.25 mmol, 5.00 eq). The reaction mixture was stirred at 50° C. for 2 h. The reaction mixture was poured into water (50.0 mL) and extracted with ethyl acetate (3×20.0 mL). The combined organic layers were washed with brine (50.0 mL), dried over anhydrous sodium sulfate, filtered and the filtrate was concentrated to afford a residue. The residue was purified by silica gel chromatography to give 3-(6-(hydroxymethyl)-4-methoxy-1-oxoisoindolin-2-yl)-1-((2-(trimethylsilyl)ethoxy) methyl) piperidine-2,6-dione. ¹H NMR (400 MHz, DMSO-d₆) δ=7.31 (s, 1H), 7.22 (s, 1H), 5.40 (t, J=6.0 Hz, 1H), 5.24 (dd, J=5.2, 13.2 Hz, 1H), 5.07 (q, J=9.6 Hz, 2H), 4.62 (d, J=5.6 Hz, 2H), 4.39 (d, J=17.2 Hz, 1H), 4.18 (d, J=17.2 Hz, 1H), 3.90 (s, 3H), 3.61-3.48 (m, 2H), 3.14-3.02 (m, 1H), 2.84-2.76 (m, 1H), 2.43 (br dd, J=4.4, 13.2 Hz, 1H), 2.10-2.02 (m, 1H), 0.92-0.80 (m, 2H), 0.04-−0.04 (m, 9H).

Intermediate XL: A mixture of 3-(6-(hydroxymethyl)-4-methoxy-1-oxoisoindolin-2-yl)-1-((2-(trimethylsilyl) ethoxy)methyl) piperidine-2,6-dione (400 mg, 920 umol, 1.00 eq) in hydrochloric acid/dioxane (4 M, 16.0 mL) was stirred at 50° C. for 2 h. The reaction mixture was concentrated to give 1-(hydroxymethyl)-3-(6-(hydroxymethyl)-4-methoxy-1-oxoisoindolin-2-yl)piperidine-2,6-dione. MS (ESI) m/z 335.1 [M+H]⁺.

Intermediate XLI: A mixture of 1-(hydroxymethyl)-3-(6-(hydroxymethyl)-4-methoxy-1-oxoisoindolin-2-yl)piperidine-2,6-dione (300 mg, 897 umol, 1.00 eq) in ammonium hydroxide (0.100 mL) and acetonitrile (5.00 mL) was stirred at 25° C. for 2 h. The reaction mixture was concentrated to give a residue, which was purified by reversed phase to give 3-(6-(hydroxymethyl)-4-methoxy-1-oxoisoindolin-2-yl)piperidine-2,6-dione. ¹H NMR (400 MHz, DMSO-d₆) δ=11.19-10.73 (m, 1H), 7.29 (s, 1H), 7.20 (s, 1H), 5.63-5.18 (m, 1H), 5.09 (dd, J=5.2, 13.2 Hz, 1H), 4.58 (d, J=16.8 Hz, 2H), 4.41-4.31 (m, 1H), 4.26-4.14 (m, 1H), 3.89 (s, 3H), 2.97-2.85 (m, 1H), 2.62-2.56 (m, 1H), 2.46-2.37 (m, 1H), 2.04-1.94 (m, 1H).

### Example 2: Synthesis of Specific Examples

TABLE 1
Specific examples

| Compound | No. |
|---|---|
| 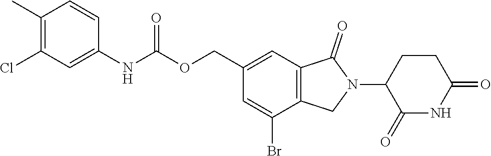 [SMILES: Cc1ccc(NC(=O)OCc2cc(Br)c3c(c2)C(=O)N(C2CCC(=O)NC2=O)C3)cc1Cl] | 1 |
| 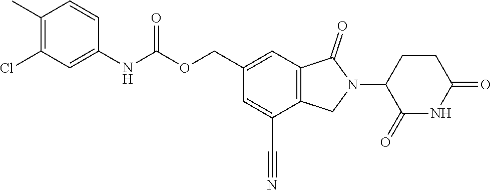 [SMILES: Cc1ccc(NC(=O)OCc2cc(C#N)c3c(c2)C(=O)N(C2CCC(=O)NC2=O)C3)cc1Cl] | 2 |
| 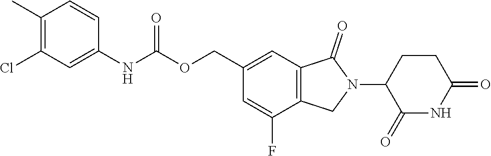 [SMILES: Cc1ccc(NC(=O)OCc2cc(F)c3c(c2)C(=O)N(C2CCC(=O)NC2=O)C3)cc1Cl] | 3 |
| 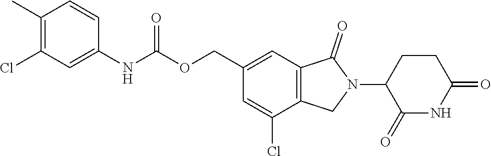 [SMILES: Cc1ccc(NC(=O)OCc2cc(Cl)c3c(c2)C(=O)N(C2CCC(=O)NC2=O)C3)cc1Cl] | 4 |
| 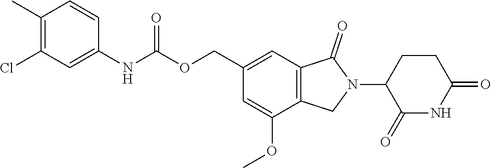 [SMILES: COc1cc(COC(=O)Nc2ccc(C)c(Cl)c2)cc2c1CN(C1CCC(=O)NC1=O)C2=O] | 5 |
| 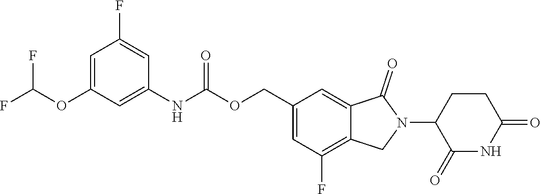 [SMILES: O=C1CCC(N2Cc3c(F)cc(COC(=O)Nc4cc(F)cc(OC(F)F)c4)cc3C2=O)C(=O)N1] | 6 |
| 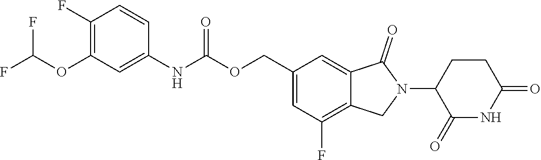 [SMILES: O=C1CCC(N2Cc3c(F)cc(COC(=O)Nc4ccc(F)c(OC(F)F)c4)cc3C2=O)C(=O)N1] | 7 |
| 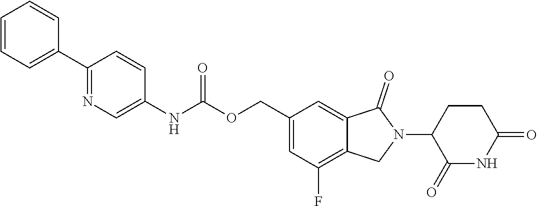 [SMILES: O=C1CCC(N2Cc3c(F)cc(COC(=O)Nc4ccc(-c5ccccc5)nc4)cc3C2=O)C(=O)N1] | 8 |
| 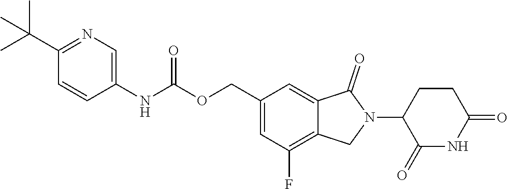 [SMILES: CC(C)(C)c1ccc(NC(=O)OCc2cc(F)c3c(c2)C(=O)N(C2CCC(=O)NC2=O)C3)cn1] | 9 |
| 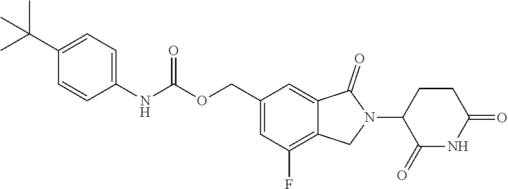 [SMILES: CC(C)(C)c1ccc(NC(=O)OCc2cc(F)c3c(c2)C(=O)N(C2CCC(=O)NC2=O)C3)cc1] | 10 |
| 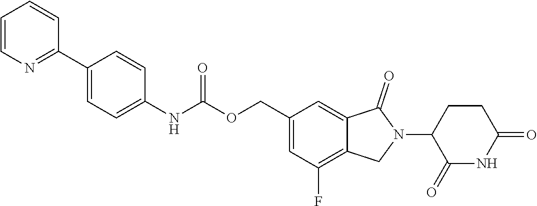 [SMILES: O=C1CCC(N2Cc3c(F)cc(COC(=O)Nc4ccc(-c5ccccn5)cc4)cc3C2=O)C(=O)N1] | 11 |
| 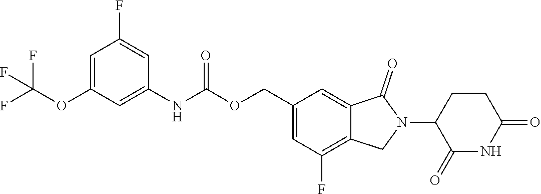 [SMILES: O=C1CCC(N2Cc3c(F)cc(COC(=O)Nc4cc(F)cc(OC(F)(F)F)c4)cc3C2=O)C(=O)N1] | 12 |
| 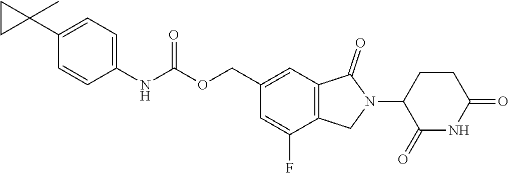 [SMILES: CC1(c2ccc(NC(=O)OCc3cc(F)c4c(c3)C(=O)N(C3CCC(=O)NC3=O)C4)cc2)CC1] | 13 |
| 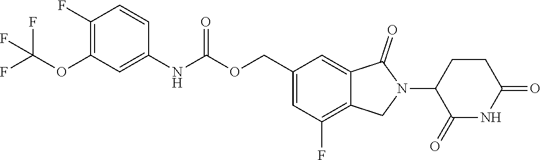 [SMILES: O=C1CCC(N2Cc3c(F)cc(COC(=O)Nc4ccc(F)c(OC(F)(F)F)c4)cc3C2=O)C(=O)N1] | 14 |
| 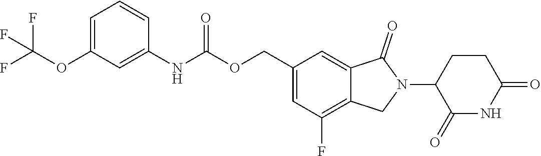 [SMILES: O=C1CCC(N2Cc3c(F)cc(COC(=O)Nc4cccc(OC(F)(F)F)c4)cc3C2=O)C(=O)N1] | 15 |
| 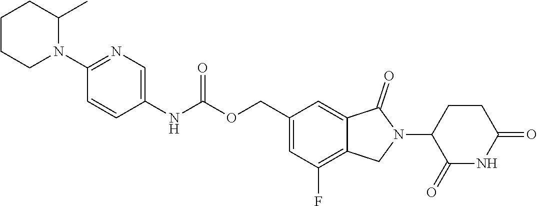 [SMILES: CC1CCCCN1c1ccc(NC(=O)OCc2cc(F)c3c(c2)C(=O)N(C2CCC(=O)NC2=O)C3)cn1] | 16 |
| 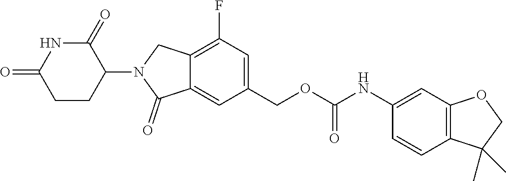 [SMILES: CC1(C)COc2cc(NC(=O)OCc3cc(F)c4c(c3)C(=O)N(C3CCC(=O)NC3=O)C4)ccc21] | 17 |
| 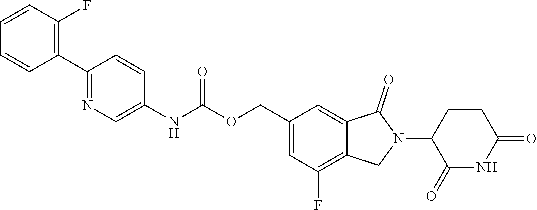 [SMILES: O=C1CCC(N2Cc3c(F)cc(COC(=O)Nc4ccc(-c5ccccc5F)nc4)cc3C2=O)C(=O)N1] | 18 |
| 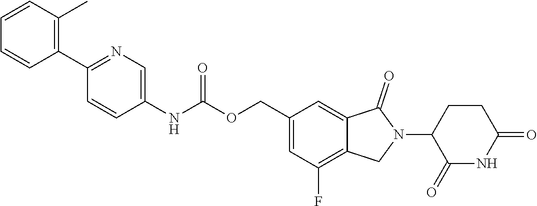 [SMILES: Cc1ccccc1-c1ccc(NC(=O)OCc2cc(F)c3c(c2)C(=O)N(C2CCC(=O)NC2=O)C3)cn1] | 19 |
| 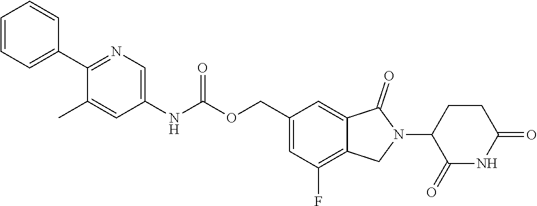 [SMILES: Cc1cc(NC(=O)OCc2cc(F)c3c(c2)C(=O)N(C2CCC(=O)NC2=O)C3)cnc1-c1ccccc1] | 20 |
| 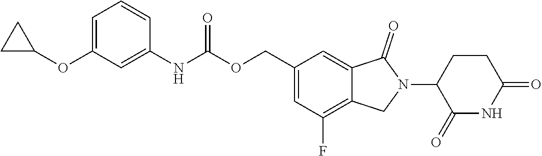 [SMILES: O=C1CCC(N2Cc3c(F)cc(COC(=O)Nc4cccc(OC5CC5)c4)cc3C2=O)C(=O)N1] | 21 |
| 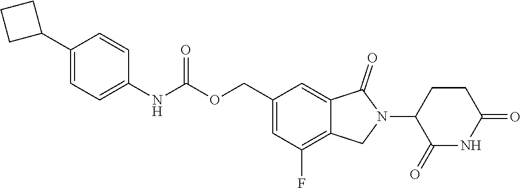 [SMILES: O=C1CCC(N2Cc3c(F)cc(COC(=O)Nc4ccc(C5CCC5)cc4)cc3C2=O)C(=O)N1] | 22 |
| 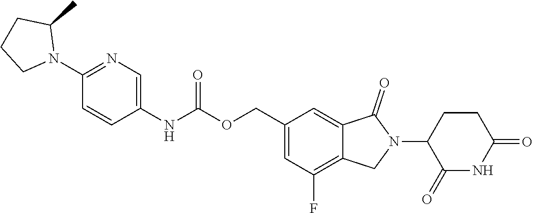 [SMILES: C[C@@H]1CCCN1c1ccc(NC(=O)OCc2cc(F)c3c(c2)C(=O)N(C2CCC(=O)NC2=O)C3)cn1] | 23 |
| 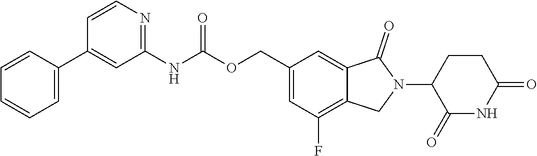 [SMILES: O=C1CCC(N2Cc3c(F)cc(COC(=O)Nc4cc(-c5ccccc5)ccn4)cc3C2=O)C(=O)N1] | 24 |
| 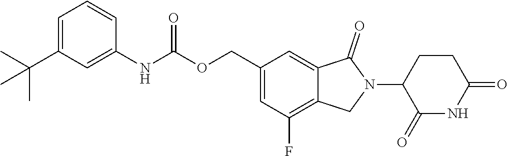 [SMILES: CC(C)(C)c1cccc(NC(=O)OCc2cc(F)c3c(c2)C(=O)N(C2CCC(=O)NC2=O)C3)c1] | 25 |
| 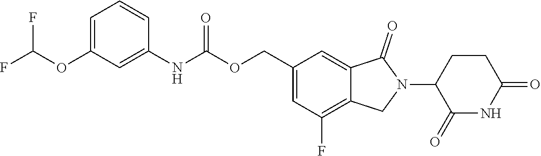 [SMILES: O=C1CCC(N2Cc3c(F)cc(COC(=O)Nc4cccc(OC(F)F)c4)cc3C2=O)C(=O)N1] | 26 |
| 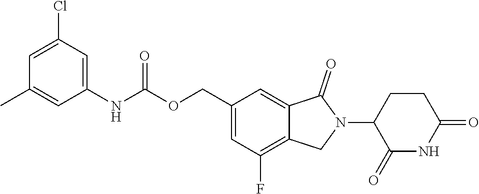 [SMILES: Cc1cc(Cl)cc(NC(=O)OCc2cc(F)c3c(c2)C(=O)N(C2CCC(=O)NC2=O)C3)c1] | 27 |
| 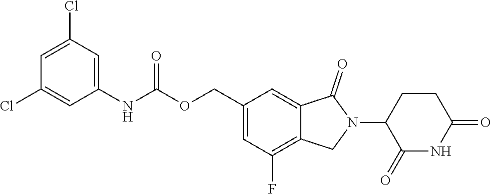 [SMILES: O=C1CCC(N2Cc3c(F)cc(COC(=O)Nc4cc(Cl)cc(Cl)c4)cc3C2=O)C(=O)N1] | 28 |
| 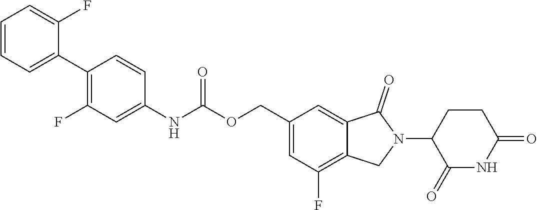 [SMILES: O=C1CCC(N2Cc3c(F)cc(COC(=O)Nc4ccc(-c5ccccc5F)c(F)c4)cc3C2=O)C(=O)N1] | 29 |
| 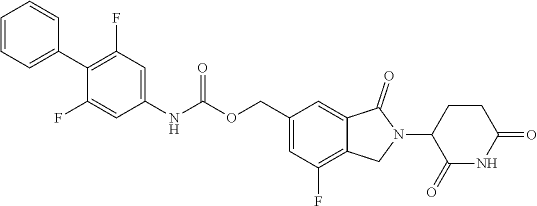 [SMILES: O=C1CCC(N2Cc3c(F)cc(COC(=O)Nc4cc(F)c(-c5ccccc5)c(F)c4)cc3C2=O)C(=O)N1] | 30 |
| 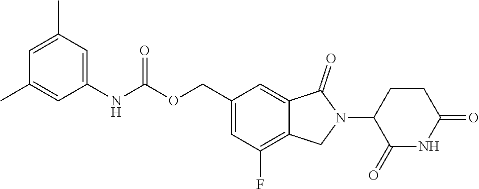 [SMILES: Cc1cc(C)cc(NC(=O)OCc2cc(F)c3c(c2)C(=O)N(C2CCC(=O)NC2=O)C3)c1] | 31 |
| 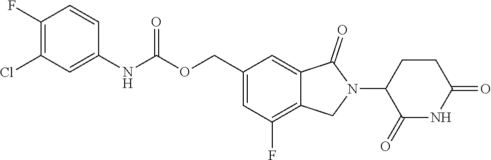 [SMILES: O=C1CCC(N2Cc3c(F)cc(COC(=O)Nc4ccc(F)c(Cl)c4)cc3C2=O)C(=O)N1] | 32 |
| 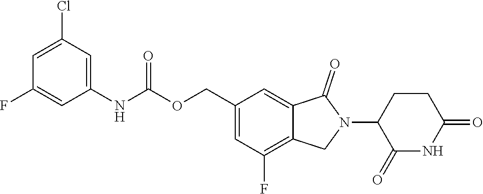 [SMILES: O=C1CCC(N2Cc3c(F)cc(COC(=O)Nc4cc(F)cc(Cl)c4)cc3C2=O)C(=O)N1] | 33 |
| 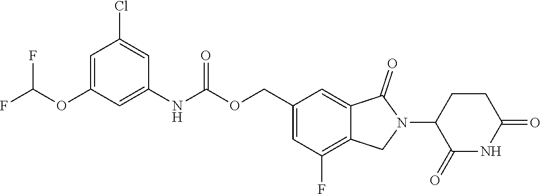 [SMILES: O=C1CCC(N2Cc3c(F)cc(COC(=O)Nc4cc(Cl)cc(OC(F)F)c4)cc3C2=O)C(=O)N1] | 34 |
| 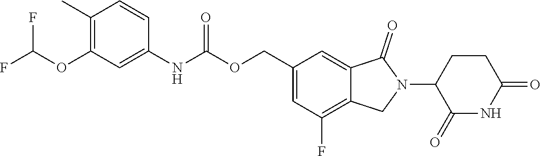 [SMILES: Cc1ccc(NC(=O)OCc2cc(F)c3c(c2)C(=O)N(C2CCC(=O)NC2=O)C3)cc1OC(F)F] | 35 |
| 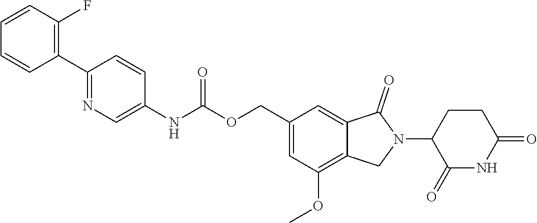 [SMILES: COc1cc(COC(=O)Nc2ccc(-c3ccccc3F)nc2)cc2c1CN(C1CCC(=O)NC1=O)C2=O] | 36 |
| 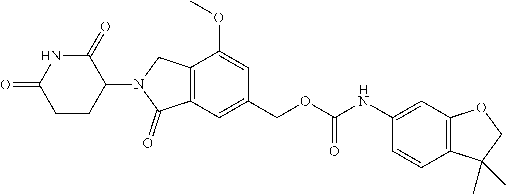 [SMILES: COc1cc(COC(=O)Nc2ccc3c(c2)OCC3(C)C)cc2c1CN(C1CCC(=O)NC1=O)C2=O] | 37 |
| 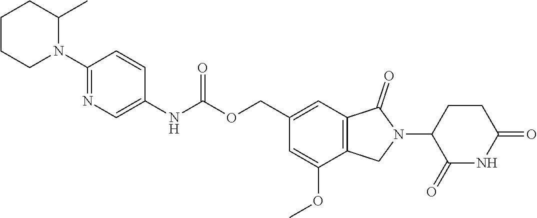 [SMILES: COc1cc(COC(=O)Nc2ccc(N3CCCCC3C)nc2)cc2c1CN(C1CCC(=O)NC1=O)C2=O] | 38 |
| 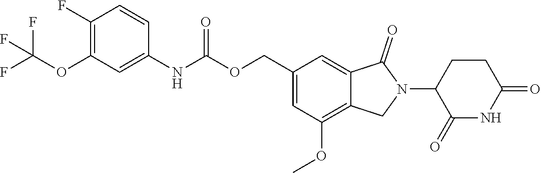 [SMILES: COc1cc(COC(=O)Nc2ccc(F)c(OC(F)(F)F)c2)cc2c1CN(C1CCC(=O)NC1=O)C2=O] | 39 |
| 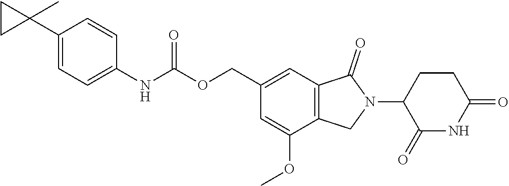 [SMILES: COc1cc(COC(=O)Nc2ccc(C3(C)CC3)cc2)cc2c1CN(C1CCC(=O)NC1=O)C2=O] | 40 |
| 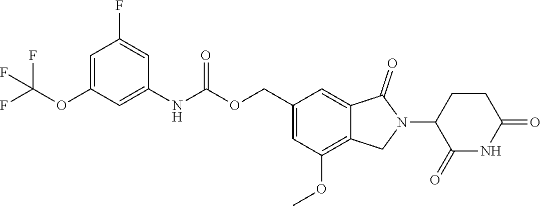 [SMILES: COc1cc(COC(=O)Nc2cc(F)cc(OC(F)(F)F)c2)cc2c1CN(C1CCC(=O)NC1=O)C2=O] | 41 |
| 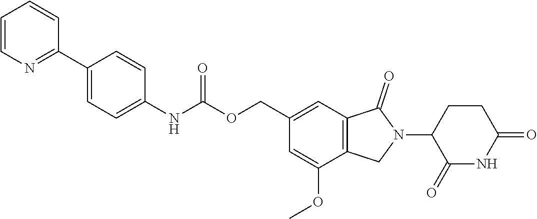 [SMILES: COc1cc(COC(=O)Nc2ccc(-c3ccccn3)cc2)cc2c1CN(C1CCC(=O)NC1=O)C2=O] | 42 |
| 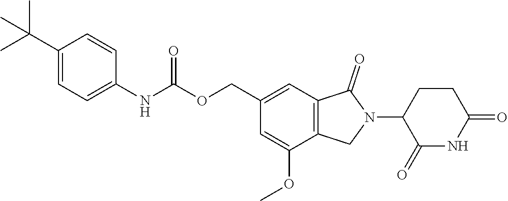 [SMILES: COc1cc(COC(=O)Nc2ccc(C(C)(C)C)cc2)cc2c1CN(C1CCC(=O)NC1=O)C2=O] | 43 |
| 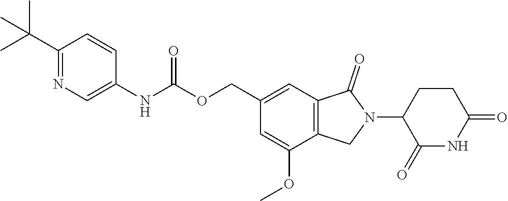 [SMILES: COc1cc(COC(=O)Nc2ccc(C(C)(C)C)nc2)cc2c1CN(C1CCC(=O)NC1=O)C2=O] | 44 |
| 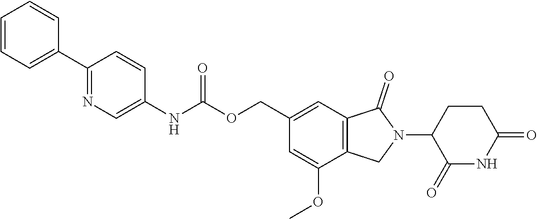 [SMILES: COc1cc(COC(=O)Nc2ccc(-c3ccccc3)nc2)cc2c1CN(C1CCC(=O)NC1=O)C2=O] | 45 |
| 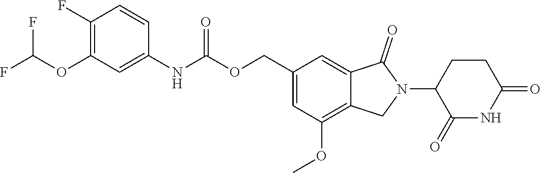 [SMILES: COc1cc(COC(=O)Nc2ccc(F)c(OC(F)F)c2)cc2c1CN(C1CCC(=O)NC1=O)C2=O] | 46 |
| 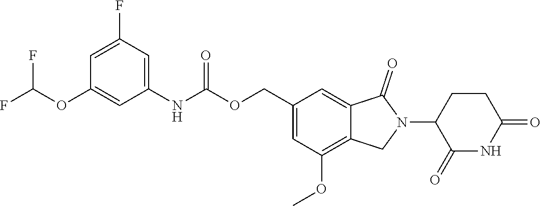 [SMILES: COc1cc(COC(=O)Nc2cc(F)cc(OC(F)F)c2)cc2c1CN(C1CCC(=O)NC1=O)C2=O] | 47 |
| 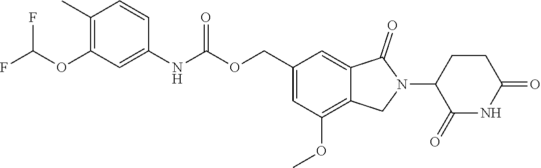 [SMILES: COc1cc(COC(=O)Nc2ccc(C)c(OC(F)F)c2)cc2c1CN(C1CCC(=O)NC1=O)C2=O] | 48 |
| 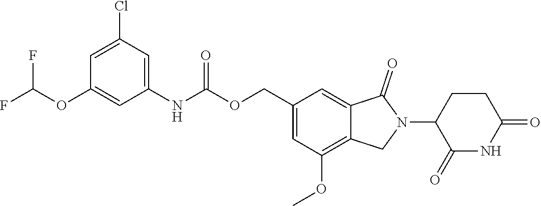 [SMILES: COc1cc(COC(=O)Nc2cc(Cl)cc(OC(F)F)c2)cc2c1CN(C1CCC(=O)NC1=O)C2=O] | 49 |
| 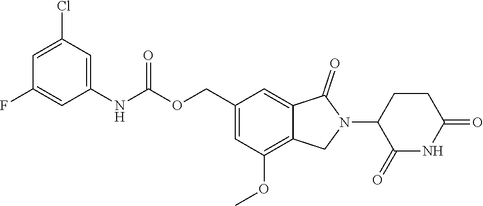 [SMILES: COc1cc(COC(=O)Nc2cc(F)cc(Cl)c2)cc2c1CN(C1CCC(=O)NC1=O)C2=O] | 50 |
| 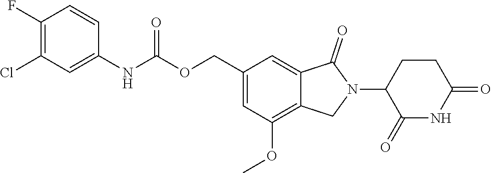 [SMILES: COc1cc(COC(=O)Nc2ccc(F)c(Cl)c2)cc2c1CN(C1CCC(=O)NC1=O)C2=O] | 51 |
| 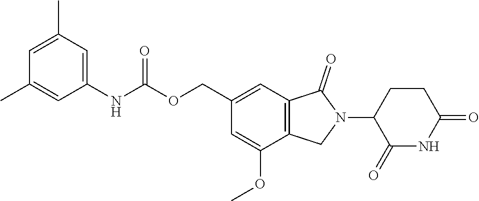 [SMILES: COc1cc(COC(=O)Nc2cc(C)cc(C)c2)cc2c1CN(C1CCC(=O)NC1=O)C2=O] | 52 |
| 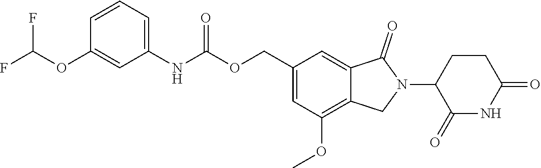 [SMILES: COc1cc(COC(=O)Nc2cccc(OC(F)F)c2)cc2c1CN(C1CCC(=O)NC1=O)C2=O] | 53 |
| 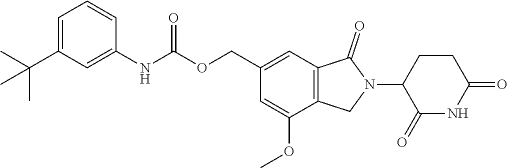 [SMILES: COc1cc(COC(=O)Nc2cccc(C(C)(C)C)c2)cc2c1CN(C1CCC(=O)NC1=O)C2=O] | 54 |
| 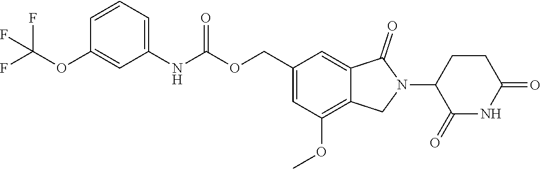 [SMILES: COc1cc(COC(=O)Nc2cccc(OC(F)(F)F)c2)cc2c1CN(C1CCC(=O)NC1=O)C2=O] | 55 |
| 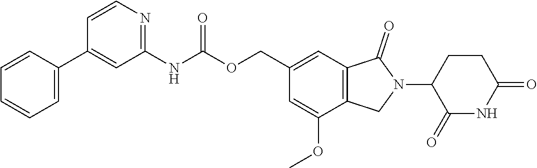 [SMILES: COc1cc(COC(=O)Nc2cc(-c3ccccc3)ccn2)cc2c1CN(C1CCC(=O)NC1=O)C2=O] | 56 |
| 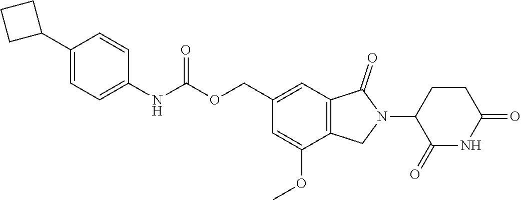 [SMILES: COc1cc(COC(=O)Nc2ccc(C3CCC3)cc2)cc2c1CN(C1CCC(=O)NC1=O)C2=O] | 57 |
| 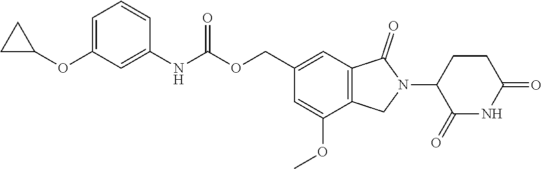 [SMILES: COc1cc(COC(=O)Nc2cccc(OC3CC3)c2)cc2c1CN(C1CCC(=O)NC1=O)C2=O] | 58 |
| 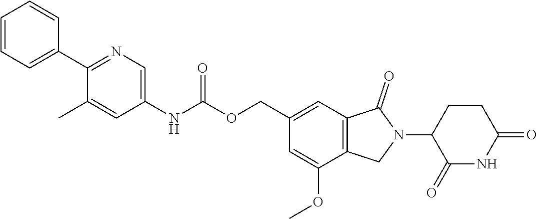 [SMILES: COc1cc(COC(=O)Nc2cnc(-c3ccccc3)c(C)c2)cc2c1CN(C1CCC(=O)NC1=O)C2=O] | 59 |
| 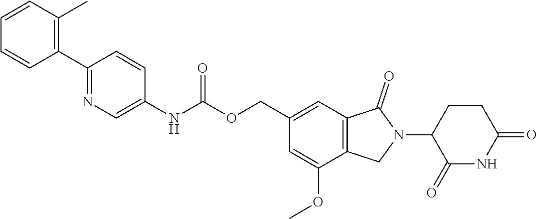 [SMILES: COc1cc(COC(=O)Nc2ccc(-c3ccccc3C)nc2)cc2c1CN(C1CCC(=O)NC1=O)C2=O] | 60 |
| 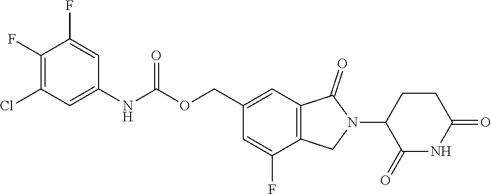 [SMILES: O=C1CCC(N2Cc3c(F)cc(COC(=O)Nc4cc(F)c(F)c(Cl)c4)cc3C2=O)C(=O)N1] | 61 |
| 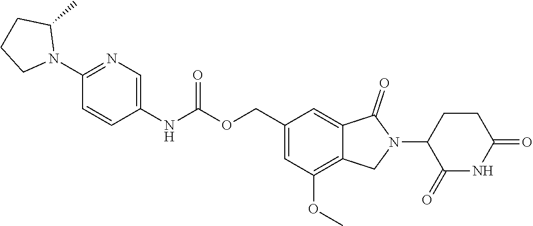 [SMILES: COc1cc(COC(=O)Nc2ccc(N3CCC[C@@H]3C)nc2)cc2c1CN(C1CCC(=O)NC1=O)C2=O] | 62 |
| 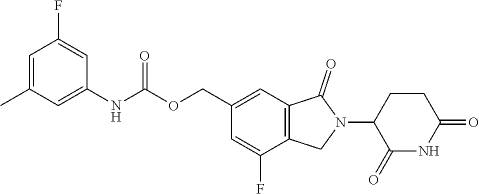 [SMILES: Cc1cc(F)cc(NC(=O)OCc2cc(F)c3c(c2)C(=O)N(C2CCC(=O)NC2=O)C3)c1] | 63 |
| 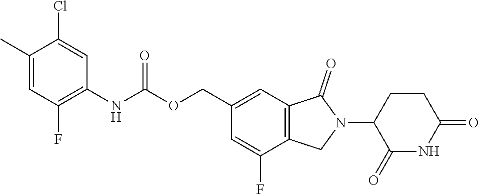 [SMILES: Cc1cc(F)c(NC(=O)OCc2cc(F)c3c(c2)C(=O)N(C2CCC(=O)NC2=O)C3)cc1Cl] | 64 |
| 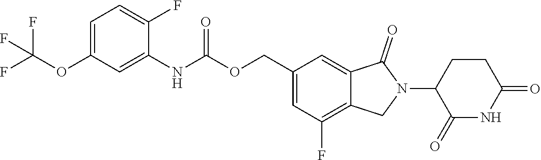 [SMILES: O=C1CCC(N2Cc3c(F)cc(COC(=O)Nc4cc(OC(F)(F)F)ccc4F)cc3C2=O)C(=O)N1] | 65 |
| 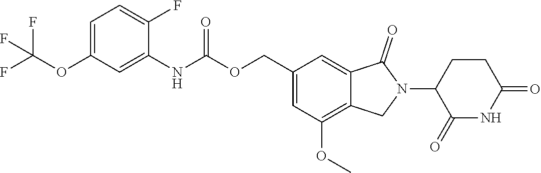 [SMILES: COc1cc(COC(=O)Nc2cc(OC(F)(F)F)ccc2F)cc2c1CN(C1CCC(=O)NC1=O)C2=O] | 66 |
| 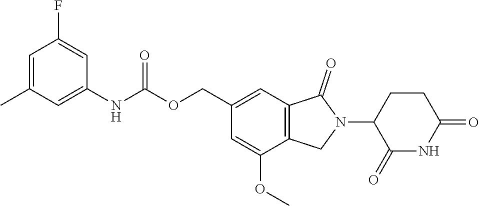 [SMILES: COc1cc(COC(=O)Nc2cc(C)cc(F)c2)cc2c1CN(C1CCC(=O)NC1=O)C2=O] | 67 |
| 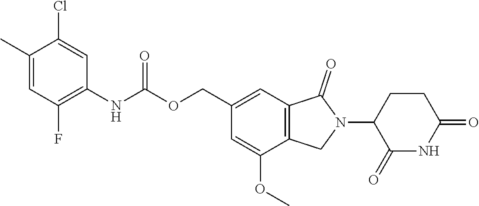 [SMILES: COc1cc(COC(=O)Nc2cc(Cl)c(C)cc2F)cc2c1CN(C1CCC(=O)NC1=O)C2=O] | 68 |
| 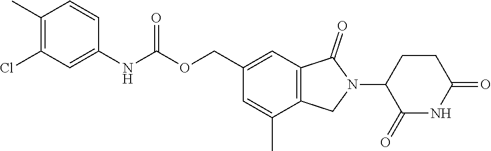 [SMILES: Cc1ccc(NC(=O)OCc2cc(C)c3c(c2)C(=O)N(C2CCC(=O)NC2=O)C3)cc1Cl] | 69 |
| 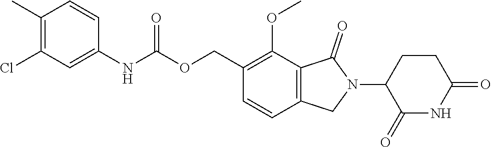 [SMILES: COc1c(COC(=O)Nc2ccc(C)c(Cl)c2)ccc2c1C(=O)N(C1CCC(=O)NC1=O)C2] | 70 |
| 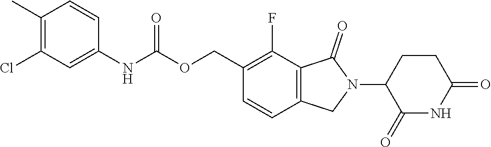 [SMILES: Cc1ccc(NC(=O)OCc2ccc3c(c2F)C(=O)N(C2CCC(=O)NC2=O)C3)cc1Cl] | 71 |
| 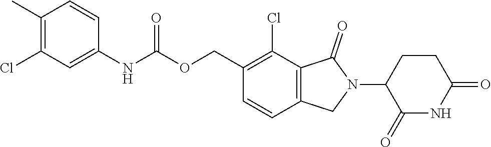 [SMILES: Cc1ccc(NC(=O)OCc2ccc3c(c2Cl)C(=O)N(C2CCC(=O)NC2=O)C3)cc1Cl] | 72 |
| 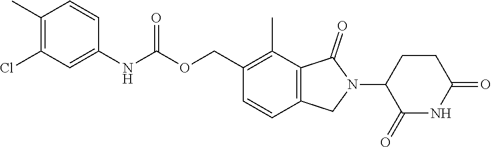 [SMILES: Cc1ccc(NC(=O)OCc2ccc3c(c2C)C(=O)N(C2CCC(=O)NC2=O)C3)cc1Cl] | 73 |
| 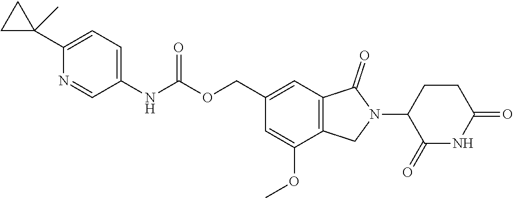 [SMILES: COc1cc(COC(=O)Nc2ccc(C3(C)CC3)nc2)cc2c1CN(C1CCC(=O)NC1=O)C2=O] | 74 |
| 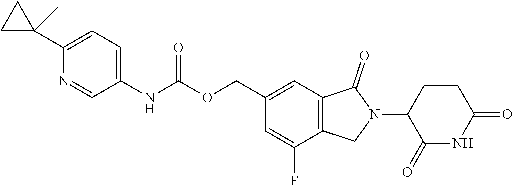 [SMILES: CC1(c2ccc(NC(=O)OCc3cc(F)c4c(c3)C(=O)N(C3CCC(=O)NC3=O)C4)cn2)CC1] | 75 |

Compound 1: Step 1: To a solution of 3-chloro-4-methylaniline (5.00 g, 35.3 mmol, 1.00 eq) in acetonitrile (50.0 mL) was added pyridine (5.59 g, 70.6 mmol, 5.70 mL, 2.00 eq) and phenyl carbonochloridate (6.08 g, 38.8 mmol, 4.87 mL, 1.10 eq) at 0° C. The reaction mixture was stirred at 30° C. for 2 h. The reaction mixture was concentrated in vacuo. The residue was purified by column chromatography to give phenyl (3-chloro-4-methylphenyl)carbamate. ¹H NMR (400 MHz, CDCl₃-d₆) δ=7.64 (s, 1H), 7.55-7.48 (m, 2H), 7.39-7.36 (m, 1H), 7.35-7.28 (m, 4H), 7.05 (br s, 1H), 2.45 (s, 3H).

Step 2: To a solution of 3-(4-bromo-6-(hydroxymethyl)-1-oxoisoindolin-2-yl)piperidine-2,6-dione VU (90.0 mg, 254 umol, 1.00 eq) in dimethyl formamide (2.00 mL) was added phenyl (3-chloro-4-methylphenyl)carbamate (100 mg, 382 umol, 1.50 eq) and sodium hydride (20.3 mg, 509 umol, 60% purity, 2.00 eq) at 0° C. The mixture was stirred at 0° C. for 1 h. The reaction mixture was quenched with hydrochloric acid (1.00M, 1.00 ml) to give a solution. The solution was purified by prep-HPLC and lyophilized to give (7-bromo-2-(2,6-dioxopiperidin-3-yl)-3-oxoisoindolin-5-yl)methyl(3-chloro-4-methylphenyl)carbamate 1. ¹H NMR (400 MHz, DMSO-d₆) δ=11.02 (s, 1H), 9.96 (br s, 1H), 7.94 (s, 1H), 7.83 (s, 1H), 7.60 (s, 1H), 7.33-7.22 (m, 2H), 5.28 (s, 2H), 5.15 (dd, J=5.1, 13.3 Hz, 1H), 4.49-4.38 (m, 1H), 4.33-4.23 (m, 1H), 2.97-2.85 (m, 1H), 2.63-2.58 (m, 1H), 2.48-2.41 (m, 1H), 2.26 (s, 3H), 2.06-1.98 (m, 1H). MS (ESI) m/z 522.0 [M+H]⁺.

Compound 2: To a solution of 2-(2,6-dioxopiperidin-3-yl)-6-(hydroxymethyl)-1-oxoisoindoline-4-carbonitrile VIII (65.0 mg, 217 umol, 1.00 eq) in dimethyl formamide (2.00 mL) was added phenyl(3-chloro-4-methylphenyl) carbamate (see Compound 1) (73.8 mg, 282 umol, 1.30 eq) and sodium hydride (17.3 mg, 434 umol, 60% purity, 2.00 eq) at 0° C. The mixture was stirred at 0° C. for 0.5 h. The reaction mixture was quenched with hydrochloric acid (1M, 1.00 ml) to give solution. The solution was purified by Prep-HPLC and lyophilized to give (7-cyano-2-(2,6-dioxopiperidin-3-yl)-3-oxoisoindolin-5-yl)methyl (3-chloro-4-methylphenyl)carbamate 2. ¹H NMR (400 MHz, DMSO-d₆) δ=11.03 (br s, 1H), 9.98 (br s, 1H), 8.20 (d, J=1.0 Hz, 1H), 8.13 (s, 1H), 7.60 (s, 1H), 7.35-7.23 (m, 2H), 5.32 (s, 2H), 5.17 (dd, J=5.1, 13.3 Hz, 1H), 4.77-4.69 (m, 1H), 4.60-4.51 (m, 1H), 2.98-2.87 (m, 1H), 2.65-2.58 (m, 1H), 2.47 (br d, J=4.3 Hz, 1H), 2.26 (s, 3H), 2.06-1.98 (m, 1H). MS (ESI) m/z 467.1 [M+H]⁺

Compound 3: To a mixture of 3-(4-fluoro-6-(hydroxymethyl)-1-oxoisoindolin-2-yl)piperidine-2,6-dione XVIII (15.0 mg, 51.3 umol, 1.00 eq) and phenyl (3-chloro-4-methylphenyl)carbamate (see Compound 1) (16.1 mg, 61.6 umol, 1.20 eq) in dimethyformamide (0.500 mL) was added sodium hydride (3.08 mg, 76.9 umol, 60% purity, 1.50 eq) in portions at 0° C. The mixture was stirred at 25° C. for 0.5 h. The mixture was quenched with 1 M hydrochloric acid and filtered. The filtrate was purified by prep-HPLC and lyophilized to give (2-(2,6-dioxopiperidin-3-yl)-7-fluoro-3-oxoisoindolin-5-yl)methyl(3-chloro-4-methylphenyl)carbamate 3. ¹H NMR (400 MHz, DMSO-d₆) δ=11.00 (br s, 1H), 9.96 (br s, 1H), 7.68 (s, 1H), 7.63-7.53 (m, 2H), 7.33-7.21 (m, 2H), 5.27 (s, 2H), 5.13 (dd, J=5.0, 13.3 Hz, 1H), 4.63-4.53 (m, 1H), 4.46-4.37 (m, 1H), 2.98-2.86 (m, 1H), 2.60 (br d, J=17.7 Hz, 1H), 2.46-2.38 (m, 1H), 2.25 (s, 3H), 2.06-1.97 (m, 1H). MS (ESI) m/z 460.0 [M+H]⁺

Compound 4: To a mixture of 3-(4-chloro-6-(hydroxymethyl)-1-oxoisoindolin-2-yl)piperidine-2,6-dione XXVIII (120 mg, 389 umol, 1.00 eq) and phenyl (3-chloro-4-methylphenyl)carbamate (see Compound 1) (122 mg, 466 umol, 1.20 eq) in dimethyformamide (1.00 mL) was added sodium hydride (23.3 mg, 583 umol, 60% purity, 1.50 eq) in portions at 0° C. The mixture was stirred at 25° C. for 2 h. The mixture was quenched with 1 M hydrochloric acid, then the mixture was added dimethylsulfoxide (0.500 mL) and acetonitrile (1.00 mL) and filtered. The filter cake was triturated with ethyl acetate (2.00 mL) and filtered. To the filter cake was added water and lyophilized to give (7-chloro-2-(2,6-dioxopiperidin-3-yl)-3-oxoisoindolin-5-yl)methyl (3-chloro-4-methylphenyl)carbamate 4. ¹H NMR (400 MHz, DMSO-d₆) δ=11.01 (br s, 1H), 9.96 (br s, 1H), 7.80 (d, J=2.9 Hz, 2H), 7.59 (s, 1H), 7.32-7.22 (m, 2H), 5.28 (s, 2H), 5.15 (dd, J=5.1, 13.3 Hz, 1H), 4.55-4.47 (m, 1H), 4.39-4.30 (m, 1H), 2.96-2.88 (m, 1H), 2.62 (br d, J=2.2 Hz, 1H), 2.48-2.40 (m, 1H), 2.25 (s, 3H), 2.06-1.98 (m, 1H). MS (ESI) m/z 476.1 [M+H]⁺

Compound 5: To a mixture of 3-(6-(hydroxymethyl)-4-methoxy-1-oxoisoindolin-2-yl)piperidine-2,6-dione XLI (35.0 mg, 115 umol, 1.00 eq) and phenyl (3-chloro-4-methylphenyl)carbamate (see Compound 1) (36.1 mg, 138 umol, 1.20 eq) in dimethyl formamide (1.00 mL) was added sodium hydride (9.20 mg, 230 umol, 60% purity, 2.00 eq) at 0° C. The reaction mixture was stirred at 25° C. for 2 h. The reaction mixture was added hydrochloric acid (1 M, 2.00 mL) and filtered to give a filtrate, which was purified by prep-HPLC to give (2-(2,6-dioxopiperidin-3-yl)-7-methoxy-3-oxoisoindolin-5-yl)methyl(3-chloro-4-methylphenyl)carbamate 5. ¹H NMR (400 MHz, DMSO-d₆) δ=10.98 (s, 1H), 9.93 (br s, 1H), 7.60 (s, 1H), 7.39 (s, 1H), 7.34-7.28 (m, 2H), 7.27-7.24 (m, 1H), 5.26 (s, 2H), 5.10 (dd, J=5.2, 13.2 Hz, 1H), 4.44-4.34 (m, 1H), 4.28-4.18 (m, 1H), 3.91 (s, 3H), 2.96-2.86 (m, 1H), 2.57 (br d, J=1.2 Hz, 1H), 2.46-2.37 (m, 1H), 2.26 (s, 3H), 2.00 (br dd, J=5.2, 10.4 Hz, 1H). MS (ESI) m/z 472.1 [M+H]⁺.

Compound 6: Step 1: A mixture of 3-fluoro-5-nitrophenol (1.50 g, 9.55 mmol, 1.00 eq), sodium 2-chloro-2,2-difluoroacetate (4.37 g, 28.6 mmol, 3.00 eq) and potassium carbonate (2.64 g, 19.1 mmol, 2.00 eq) in dimethylformamide (20.0 mL) and water (4.00 mL) was stirred at 100° C. for 12 h. The mixture was quenched by 1M hydrochloric acid (20.0 mL) and extracted with ethyl acetate (3×15.0 mL). The combined organic phase was washed with brine (10.0 mL), dried over anhydrous sodium sulfate, filtered and concentrated in vacuum to give a residue. The residue was purified by silica gel chromatography to give 1-(difluoromethoxy)-3-fluoro-5-nitrobenzene. ¹H NMR (400 MHz, DMSO-d₆) δ=8.03 (td, J=2.1, 8.5 Hz, 1H), 7.90 (d, J=0.8 Hz, 1H), 7.72 (td, J=2.3, 9.4 Hz, 1H), 7.48 (t, J=72.8 Hz, 1H).

Step 2: A mixture of 1-(difluoromethoxy)-3-fluoro-5-nitrobenzene (1.10 g, 5.31 mmol, 1.00 eq), ferrous powder (890 mg, 15.9 mmol, 3.00 eq) and ammonium chloride (1.42 g, 26.6 mmol, 5.00 eq) in methanol (10.00 mL) and water (5.00 mL) was stirred at 80° C. for 1 h. The reaction mixture was filtered and the filtrate was concentrated in vacuum. The residue was added saturated sodium bicarbonate (50.0 mL). The aqueous phase was extracted with ethyl acetate (3×30.0 mL). The combined organic phase was washed with brine (30.0 mL), dried over anhydrous sodium sulfate, filtered and concentrated in vacuum to give 3-(difluoromethoxy)-5-fluoroaniline. ¹H NMR (400 MHz, DMSO-d₆) δ=7.13 (t, J=74.4 Hz, 1H), 6.26-6.02 (m, 3H), 5.71 (br s, 2H).

Step 3: To a mixture of 3-(difluoromethoxy)-5-fluoroaniline (300 mg, 1.69 mmol, 1.00 eq) and pyridine (402 mg, 5.08 mmol, 410 uL, 3.00 eq) in acetonitrile (5.00 mL) was added phenyl carbonochloridate (345 mg, 2.20 mmol, 276 uL, 1.30 eq) dropwise. The mixture was stirred at 15° C. for 12 h. The mixture was concentrated to give crude product. The crude product was purified by reversed-phase column. The desired fraction was collected and lyophilized to give phenyl (3-(difluoromethoxy)-5-fluorophenyl)carbamate. MS (ESI) m/z 298.0 [M+H]⁺

Step 4: To a solution of 3-(4-fluoro-6-(hydroxymethyl)-1-oxoisoindolin-2-yl)piperidine-2,6-dione XVIII (80.0 mg, 274 umol, 1.00 eq) and phenyl (3-(difluoromethoxy)-5-fluorophenyl)carbamate (97.6 mg, 328 umol, 1.20 eq) in dimethyformamide (2.00 mL) was added sodium hydride (16.4 mg, 411 umol, 60% purity, 1.50 eq) at 0° C. The mixture was stirred at 0° C. for 1 h. The reaction was quenched by acetic acid (1.00 mL) slowly and then filtered and concentrated in vacuum. The residue was purified by prep-HPLC and lyophilized to afford (2-(2,6-dioxopiperidin-3-yl)-7-fluoro-3-oxoisoindolin-5-yl)methyl(3-(difluoromethoxy)-5-fluorophenyl)carbamate 6. ¹H NMR (400 MHz, DMSO-d₆) δ=11.01 (br s, 1H), 10.28 (s, 1H), 7.68 (s, 1H), 7.57 (d, J=9.8 Hz, 1H), 7.24 (t, J=73.6 Hz, 1H), 7.22 (td, J=2.0, 11.1 Hz, 1H), 7.16 (s, 1H), 6.77 (td, J=2.1, 9.8 Hz, 1H), 5.30 (s, 2H), 5.13 (dd, J=5.1, 13.4 Hz, 1H), 4.63-4.52 (m, 1H), 4.47-4.36 (m, 1H), 2.98-2.85 (m, 1H), 2.64-2.57 (m, 1H), 2.46-2.37 (m, 1H), 2.06-1.97 (m, 1H). MS (ESI) m/z 496.1 [M+H]⁺

Compound 7: Step 1: A mixture of 2-fluoro-5-nitrophenol (3.00 g, 19.1 mmol, 1.00 eq), sodium 2-chloro-2,2-difluoroacetate (14.7 g, 95.5 mmol, 5.00 eq), potassium carbonate (5.28 g, 38.2 mmol, 2.00 eq) in dimethyl formamide (30.0 mL) was stirred at 100° C. for 2 h. The reaction mixture was quenched by addition water (100 mL) and extracted with ethyl acetate (3×100 mL). The combined organic layers were washed with brine (3×100 mL), dried over anhydrous sodium sulfate, filtered and concentrated under reduced pressure to give a residue. The residue was purified by column chromatography to give 2-(difluoromethoxy)-1-fluoro-4-nitrobenzene. ¹H NMR (400 MHz, CDCl₃) δ=8.23-8.11 (m, 2H), 7.36 (t, J=9.0 Hz, 1H), 6.65 (t, J=71.6 Hz, 1H).

Step 2: To a solution of 2-(difluoromethoxy)-1-fluoro-4-nitrobenzene (1.70 g, 8.21 mmol, 1.00 eq) in methanol (20.0 mL) and water (4.00 mL) was added iron (2.29 g, 41.0 mmol, 5.00 eq) and saturated ammonium chloride (3.51 g, 65.7 mmol, 8.00 eq). The mixture was stirred at 80° C. for 2 h. The reaction mixture was filtered and concentrated under reduced pressure to give a residue. The residue was quenched by addition saturated sodium bicarbonate (50.0 mL) and extracted with ethyl acetate (3×50.0 mL). The combined organic layers were dried over anhydrous sodium sulfate, filtered and concentrated under reduced pressure to give 3-(difluoromethoxy)-4-fluoroaniline. MS (ESI) m/z. 178.1 [M+H]⁺

Step 3: To a solution of 3-(difluoromethoxy)-4-fluoroaniline (0.580 g, 3.27 mmol, 1.00 eq) and pyridine (777 mg, 9.82 mmol, 793 uL, 3.00 eq) in acetonitrile (10.0 mL) was added phenyl carbonochloridate (564 mg, 3.60 mmol, 452 uL, 1.10 eq) at 0° C. The mixture was stirred at 25° C. for 2 h. The reaction mixture was concentrated under reduced pressure to give a residue. The residue was purified by reversed-phase HPLC to give phenyl (3-(difluoromethoxy)-4-fluorophenyl)carbamate. MS (ESI) m/z. 298.1 [M+H]⁺

Step 4: To a solution of phenyl (3-(difluoromethoxy)-4-fluorophenyl)carbamate (89.5 mg, 301 umol, 1.10 eq) and 3-(4-fluoro-6-(hydroxymethyl)-1-oxoisoindolin-2-yl)piperidine-2,6-dione XVIII (80.0 mg, 273 umol, 1.00 eq) in dimethyl formamide (1.50 mL) was added sodium hydride (21.9 mg, 547 umol, 60% purity, 2.00 eq) at 0° C. The mixture was stirred at 25° C. for 1 h. The pH of the mixture was adjusted to around 6 by with hydrochloric acid (1M). The mixture was extracted with ethyl acetate (3×10.0 mL). The combined organic layers were dried over anhydrous sodium sulfate, filtered and concentrated under reduced pressure to give a residue. The residue was purified by prep-HPLC and lyophilized to afford (2-(2,6-dioxopiperidin-3-yl)-7-fluoro-3-oxoisoindolin-5-yl)methyl(3-(difluoromethoxy)-4-fluorophenyl)carbamate. ¹H NMR (400 MHz, DMSO-d₆) δ=11.01 (s, 1H), 10.07 (br s, 1H), 7.68 (s, 1H), 7.56 (br d, J=9.0 Hz, 2H), 7.40-7.27 (m, 2H), 7.20 (t, J=73.2 Hz, 1H), 5.29 (s, 2H), 5.13 (dd, J=5.1, 13.4 Hz, 1H), 4.62-4.54 (m, 1H), 4.45-4.36 (m, 1H), 2.97-2.86 (m, 1H), 2.63-2.57 (m, 1H), 2.44-2.36 (m, 1H), 2.06-1.95 (m, 1H). MS (ESI) m/z. 496.2[M+H]⁺

Compound 8: Step 1: To a solution of 6-phenylpyridin-3-amine (300 mg, 1.76 mmol, 1.00 eq) in acetonitrile (5.00 mL) was added pyridine (697 mg, 8.81 mmol, 711 uL, 5.00 eq) and phenyl carbonochloridate (331 mg, 2.12 mmol, 265 uL, 1.20 eq) in portions at 0° C. The mixture was stirred at 25° C. for 1 h. The mixture was concentrated under reduced pressure to give a residue which was purified by reverse phase HPLC (0.1% formic acid condition).

The desired fraction was collected to remove acetonitrile. The residual aqueous solution was lyophilized to give a phenyl (6-phenylpyridin-3-yl)carbamate. MS (ESI) m/z 291.1 [M+H]⁺

Step 2: To a solution of phenyl (6-phenylpyridin-3-yl)carbamate (87.4 mg, 301 umol, 1.10 eq) and 3-(4-fluoro-6-(hydroxymethyl)-1-oxoisoindolin-2-yl)piperidine-2,6-dione XVIII (80.0 mg, 274 umol, 1.00 eq) in dimethyl formamide (1.50 mL) was added sodium hydride (21.90 mg, 547 umol, 60% purity, 2.00 eq) at 0° C. The mixture was stirred at 25° C. for 1 hr. The pH of the mixture was adjusted to around 6 by adding hydrochloric acid and extracted with ethyl acetate (3×10.0 mL). The combined organic layers were dried over anhydrous sodium sulfate, filtered and concentrated under reduced pressure to give a residue. The residue was purified by prep-HPLC and lyophilized to give a white solid. The white solid was further purified by prep-HPLC and lyophilized to afford (2-(2,6-dioxopiperidin-3-yl)-7-fluoro-3-oxoisoindolin-5-yl)methyl(6-phenylpyridin-3-yl)carbamate. ¹H NMR (400 MHz, DMSO-d₆) δ=11.01 (br d, J=0.9 Hz, 1H), 10.19 (br s, 1H), 8.73 (d, J=2.3 Hz, 1H), 8.04-7.99 (m, 3H), 7.95-7.91 (m, 1H), 7.71 (s, 1H), 7.59 (d, J=9.5 Hz, 1H), 7.48-7.44 (m, 2H), 7.41-7.36 (m, 1H), 5.33 (s, 2H), 5.13 (dd, J=5.0, 13.2 Hz, 1H), 4.64-4.56 (m, 1H), 4.45-4.39 (m, 1H), 2.96-2.87 (m, 1H), 2.60 (br d, J=13.2 Hz, 1H), 2.46-2.41 (m, 1H), 2.05-1.99 (m, 1H). MS (ESI) m/z. 489.0 [M+H]⁺

Compound 9: Step 1: To a solution of 6-(tert-butyl)pyridin-3-amine (150 mg, 998 umol, 1.00 eq) in acetonitrile (5.00 mL) was added pyridine (394 mg, 4.99 mmol, 402 uL, 5.00 eq) and phenyl carbonochloridate (187 mg, 1.20 mmol, 150 uL, 1.20 eq). The mixture was stirred at 25° C. for 1 h. The reaction mixture was concentrated under reduced pressure to give a residue. The crude product was purified by reverse phase HPLC to give phenyl (6-(tert-butyl)pyridin-3-yl)carbamate. ¹H NMR (400 MHz, DMSO-d₆) δ=10.34 (br s, 1H), 8.61 (d, J=2.3 Hz, 1H), 7.85 (dd, J=2.3, 8.6 Hz, 1H), 7.48-7.37 (m, 3H), 7.32-7.21 (m, 3H), 1.30 (s, 9H). MS (ESI) m/z 271.1 [M+H]⁺.

Step 2: To a mixture of 3-(4-fluoro-6-(hydroxymethyl)-1-oxoisoindolin-2-yl)piperidine-2,6-dione XVIII (80.0 mg, 273 umol, 1.00 eq) and phenyl (6-(tert-butyl)pyridin-3-yl)carbamate (88.8 mg, 328 umol, 1.20 eq) in dimethyl formamide (2.00 mL) was added sodium hydride (21.9 mg, 547 umol, 60% purity, 2.00 eq) at 0° C. The reaction mixture was stirred at 25° C. for 1 h. The reaction mixture was added formic acid (1.00 mL) and filtered to give a filtrate. The filtrate was purified twice by Prep-HPLC and lyophilized to give (2-(2,6-dioxopiperidin-3-yl)-7-fluoro-3-oxoisoindolin-5-yl)methyl(6-(tert-butyl)pyridin-3-yl)carbamate. ¹H NMR (400 MHz, DMSO-d₆) δ=11.02 (br s, 1H), 9.97 (br s, 1H), 8.55 (d, J=2.0 Hz, 1H), 7.83 (br d, J=7.6 Hz, 1H), 7.69 (s, 1H), 7.58 (d, J=9.6 Hz, 1H), 7.37 (d, J=8.8 Hz, 1H), 5.30 (s, 2H), 5.14 (dd, J=5.2, 13.2 Hz, 1H), 4.64-4.53 (m, 1H), 4.48-4.38 (m, 1H), 2.92 (ddd, J=5.6, 13.6, 17.6 Hz, 1H), 2.64-2.58 (m, 1H), 2.47-2.38 (m, 1H), 2.06-1.98 (m, 1H), 1.28 (s, 9H). MS (ESI) m/z 469.1 [M+H]⁺

Compound 10: Step 1: A mixture of phenyl carbonochloridate (787 mg, 5.03 mmol, 630 uL, 1.50 eq), 4-(tert-butyl)aniline (500 mg, 3.35 mmol, 529 uL, 1.00 eq) and pyridine (795 mg, 10.1 mmol, 811 uL, 3.00 eq) in acetonitrile (5.00 mL) was stirred at 25° C. for 12 h. The residue was diluted with ethyl acetate (50.0 mL) and water (50.0 mL). The organic layer was separated and the aqueous phase was extracted with ethyl acetate (3×50.0 mL). The organic layer was dried over sodium sulfate, filtered and concentrated in vacuum. The residue was purified by column chromatography on silica gel to give phenyl (4-(tert-butyl)phenyl)carbamate. MS (ESI) m/z 270.1 [M+H]⁺

Step 2: To a solution of 3-(4-fluoro-6-(hydroxymethyl)-1-oxoisoindolin-2-yl)piperidine-2,6-dione XVIII (70.0 mg, 239 umol, 1.00 eq) in dimethyl formamide (2.00 mL) was added phenyl (4-(tert-butyl)phenyl)carbamate (70.9 mg, 263 umol, 1.10 eq) and sodium hydride (19.1 mg, 479 umol, 60% purity, 2.00 eq) at 0° C. The mixture was stirred at 0° C. for 0.5 h. The reaction mixture was quenched with formic acid (0.500 ml) to give a solution. The solution was purified by prep-HPLC and lyophilized to give (2-(2,6-dioxopiperidin-3-yl)-7-fluoro-3-oxoisoindolin-5-yl)methyl (4-(tert-butyl)phenyl)carbamate. ¹H NMR (400 MHz, DMSO-d₆) δ=11.02 (s, 1H), 9.75 (br s, 1H), 7.68 (s, 1H), 7.56 (d, J=9.8 Hz, 1H), 7.42-7.35 (m, 2H), 7.33-7.28 (m, 2H), 5.27 (s, 2H), 5.14 (dd, J=5.1, 13.3 Hz, 1H), 4.63-4.53 (m, 1H), 4.46-4.37 (m, 1H), 2.97-2.86 (m, 1H), 2.64-2.58 (m, 1H), 2.44 (dd, J=4.5, 13.0 Hz, 1H), 2.06-1.97 (m, 1H), 1.26 (s, 9H). MS (ESI) m/z 468.2 [M+H]⁺

Compound 11: Step 1: To a solution of (4-nitrophenyl)boronic acid (5.00 g, 29.9 mmol, 1.00 eq) and 2-bromopyridine (7.10 g, 44.9 mmol, 4.28 mL, 1.50 eq) in dioxane (40.0 mL) and water (10.0 mL) was added [1,1′-bis(diphenylphosphino)ferrocene]dichloropalladium(II)dichloromethane (2.45 g, 3.00 mmol, 0.100 eq) and potassium phosphate (19.0 g, 89.8 mmol, 3.00 eq) in portions. The mixture was stirred at 80° C. for 12 h under nitrogen. The mixture was diluted with water (100 mL) and extracted with ethyl acetate (3×100 mL). The combined organic layers were washed with saturated brine (40.0 mL), dried with anhydrous sodium sulfate, filtered and concentrated in vacuum. The residue was purified by column chromatography on silica gel to give 2-(4-nitrophenyl)pyridine. ¹H NMR (400 MHz, DMSO-d₆) δ=8.74 (br d, J=4.3 Hz, 1H), 8.34 (q, J=8.9 Hz, 4H), 8.12 (d, J=7.9 Hz, 1H), 7.96 (dt, J=1.5, 7.7 Hz, 1H), 7.46 (dd, J=4.8, 7.3 Hz, 1H).

Step 2: To a mixture of 2-(4-nitrophenyl)pyridine (4.90 g, 24.4 mmol, 1.00 eq) in methanol (100 mL) was added palladium/carbon (500 mg, 10% purity) in one portion. The mixture was stirred at 15° C. for 1 h under hydrogen (15 Psi) atmosphere. The mixture was filtered and the filtrate was concentrated in vacuum to give to give 4-(pyridin-2-yl)aniline.

Step 3: To a mixture of 4-(pyridin-2-yl)aniline (2.00 g, 11.7 mmol, 1.00 eq) and pyridine (2.79 g, 35.2 mmol, 2.85 mL, 3.00 eq) in acetonitrile (20.0 mL) was added phenyl carbonochloridate (2.39 g, 15.2 mmol, 1.91 mL, 1.30 eq) dropwise at 0° C. The mixture was stirred at 15° C. for 2 h. The mixture was concentrated to give crude product. The crude product was triturated with dimethyl formamide (5.00 mL) and ethyl acetate (20.0 mL) to give phenyl (4-(pyridin-2-yl)phenyl)carbamate. ¹H NMR (400 MHz, DMSO-d₆) δ=10.75 (s, 1H), 8.79 (dd, J=0.8, 5.6 Hz, 1H), 8.54-8.40 (m, 1H), 8.33 (d, J=8.2 Hz, 1H), 8.18 (d, J=8.9 Hz, 2H), 7.82 (t, J=6.6 Hz, 1H), 7.77 (d, J=8.8 Hz, 2H), 7.49-7.39 (m, 2H), 7.33-7.21 (m, 3H).

Step 4: To a mixture of phenyl (4-(pyridin-2-yl)phenyl)carbamate (95.3 mg, 328 umol, 1.20 eq) and 3-(4-fluoro-6-(hydroxymethyl)-1-oxoisoindolin-2-yl)piperidine-2,6-dione XVIII (80.0 mg, 273 umol, 1.00 eq) in dimethyl formamide (1.00 mL) was added sodium hydride (16.4 mg, 410 umol, 60% purity, 1.50 eq) in one portion at 0° C. The mixture was stirred at 15° C. for 1 h. The mixture was quenched with 1 M hydrochloric acid (0.500 mL) and filtered. The filtrate was purified by prep-HPLC and lyophilized to give (2-(2,6-dioxopiperidin-3-yl)-7-fluoro-3-oxoisoindolin-5-yl)methyl (4-(pyridin-2-yl) phenyl)carbamate. ¹H NMR (400 MHz, DMSO-d₆) δ=11.02 (s, 1H), 10.28 (s, 1H), 8.74 (br d, J=5.4 Hz, 1H), 8.31 (br t, J=7.7 Hz, 1H), 8.22 (br d, J=8.2 Hz, 1H), 8.10 (br d, J=8.5 Hz, 2H), 7.80-7.67 (m, 4H), 7.59 (d, J=9.8 Hz, 1H), 5.32 (s, 2H), 5.13 (br dd, J=5.0, 13.2 Hz, 1H), 4.65-4.55 (m, 1H), 4.48-4.36 (m, 1H), 3.01-2.86 (m, 1H), 2.60 (br d, J=16.9 Hz, 1H), 2.47-2.33 (m, 1H), 2.08-1.98 (m, 1H). MS (ESI) m/z 489.1 [M+H]⁺

Compound 12: Step 1: To a solution of 3-fluoro-5-(trifluoromethoxy)aniline (150 mg, 769 umol, 1.00 eq) and pyridine (182 mg, 2.31 mmol, 186 uL, 3.00 eq) in acetonitrile (5.00 mL) was added phenyl carbonochloridate (144 mg, 923 umol, 116 uL, 1.20 eq) at 25° C. The reaction mixture was stirred at 25° C. for 2 h. The reaction mixture was concentrated to give a residue, which was purified by reversed-phase HPLC (0.1% formic acid) and lyophilized to give phenyl (3-fluoro-5-(trifluoromethoxy)phenyl)carbamate. MS (ESI) m/z Step 2: To a mixture of XVIII 3-(4-fluoro-6-(hydroxymethyl)-1-oxoisoindolin-2-yl)piperidine-2,6-dione XVIII (80.0 mg, 273 umol, 1.00 eq) and phenyl (3-fluoro-5-(trifluoromethoxy)phenyl)carbamate (103 mg, 328 umol, 1.20 eq) in dimethyl formamide (2.00 mL) was added sodium hydride (21.9 mg, 547 umol, 60% purity, 2.00 eq) at 0° C. The reaction mixture was stirred at 25° C. for 1 h. The reaction mixture was added formic acid (1.00 mL) and filtered to give a filtrate. The filtrate was purified by Prep-HPLC and lyophilized to give (2-(2,6-dioxopiperidin-3-yl)-7-fluoro-3-oxoisoindolin-5-yl)methyl(3-fluoro-5-(trifluoro methoxy)phenyl)carbamate. ¹H NMR (400 MHz, DMSO-d₆) δ=11.02 (br s, 1H), 10.40 (s, 1H), 7.70 (s, 1H), 7.59 (d, J=9.6 Hz, 1H), 7.40-7.33 (m, 2H), 7.01 (br d, J=8.8 Hz, 1H), 5.32 (s, 2H), 5.14 (dd, J=5.2, 13.2 Hz, 1H), 4.63-4.56 (m, 1H), 4.46-4.39 (m, 1H), 2.98-2.87 (m, 1H), 2.61 (td, J=2.0, 15.2 Hz, 1H), 2.47-2.38 (m, 1H), 2.06-1.97 (m, 1H). MS (ESI) m/z 514.0 [M+H]⁺

Compound 13: Step 1: To freshly distilled dichloromethane (50.0 mL) was added diethylzinc (1 M in toluene, 40.6 mL, 4.00 eq). The solution was cooled to −40° C. and then diiodomethane (10.8 g, 40.6 mL, 4.00 eq) in dichloromethane (10.0 mL) was added very slowly into the reaction mixture. The mixture was stirred at −40° C. for 30 min. Then trifluoroacetic acid (231 mg, 2.03 mmol, 150 uL, 0.200 eq) and N,N-dimethylacetamide (910 mg, 10.1 mmol, 1.05 mL, 0.995 eq) in dichloromethane (10.0 mL) was added to and the mixture was stirred at −15° C. for another 0.5 h. Then 1-bromo-4-(prop-1-en-2-yl)benzene (2.00 g, 10.1 mmol, 1.00 eq) in dichloromethane (10.0 mL) was added to at 0° C. The mixture was stirred at 25° C. for another 12 h. The mixture was quenched with ice-water (50.0 mL) at 0° C. The organic layer was separated and concentrated under reduced pressure to give a residue. The residue was purified by column chromatography on silica gel to give 1-bromo-4-(1-methylcyclopropyl)benzene. ¹H NMR (400 MHz, CDCl₃) δ=7.34-7.29 (m, 2H), 7.07-7.01 (m, 2H), 1.30 (s, 3H), 0.77-0.73 (m, 2H), 0.68-0.63 (m, 2H).

Step 2: To a solution of 1-bromo-4-(1-methylcyclopropyl)benzene (2.00 g, 9.47 mmol, 1.00 eq) (crude) in tert-amyl alcohol (100 mL) was added tert-butyl carbamate (2.00 g, 17.1 mmol, 1.80 eq), methanesulfonato(2-di-tbutylphosphino-2,4,6-tri-ipropyl-1,1-biphenyl)(2-amino-1,1-biphenyl-2-yl)palladium(II) (600 mg, 755 umol, 0.0800 eq) and sodium tert-butoxide (2 M in tetrahydrofuran, 14.0 mL, 2.96 eq). The mixture was stirred at 90° C. for 3 h under nitrogen. The mixture was diluted with ethyl acetate (200 mL) and water (200 mL).

The organic layer was separated and concentrated to give a residue. The residue was purified by reverse phase HPLC to give tert-butyl (4-(1-methylcyclopropyl)phenyl) carbamate. ¹H NMR (400 MHz, CDCl₃) δ=7.26 (s, 2H), 7.22-7.17 (m, 2H), 6.43 (br s, 1H), 1.39 (s, 3H), 0.85-0.80 (m, 2H), 0.73-0.67 (m, 2H). MS (ESI) m/z 192.1 [M+H−56]⁺

Step 3: To a solution of tert-butyl (4-(1-methylcyclopropyl)phenyl) carbamate (520 mg, 2.10 mmol, 1.00 eq) in ethyl acetate (10.0 mL) was added hydrogen chloride/ethyl acetate (4 M, 10 mL, 19.0 eq). The mixture was stirred at 25° C. for 1 h. The mixture was concentrated under reduced pressure to give 4-(1-methylcyclopropyl)aniline. ¹H NMR (400 MHz, DMSO-d₆) δ=10.46-9.75 (m, 3H), 7.35-7.29 (m, 2H), 7.29-7.23 (m, 2H), 1.38 (s, 3H), 0.87-0.81 (m, 2H), 0.81-0.75 (m, 2H). MS (ESI) m/z 148.2 [M+H]⁺

Step 4: To a solution of 4-(1-methylcyclopropyl)aniline (200 mg, 1.09 mmol, 1.00 eq, hydrochloric acid) in acetonitrile (50.0 mL) was added pyridine (490 mg, 6.19 mmol, 500 uL, 5.69 eq) and phenyl carbonochloridate (187 mg, 1.20 mmol, 1.10 eq) at 0° C. The mixture was stirred at 25° C. for 1 h. The mixture was concentrated under reduced pressure. The residue was purified by column chromatography on silica gel to give phenyl (4-(1-methylcyclopropyl)phenyl)carbamate. MS (ESI) m/z 268.1 [M+H]⁺

Step 5: To a solution of 3-(4-fluoro-6-(hydroxymethyl)-1-oxoisoindolin-2-yl)piperidine-2,6-dione XVIII (90.0 mg, 308 umol, 1.00 eq) in dimethyl formamide (2.00 mL) was added phenyl (4-(1-methylcyclopropyl)phenyl) carbamate (98.8 mg, 369 umol, 1.20 eq) and sodium hydride (24.6 mg, 616 umol, 60% purity, 2.00 eq) at 0° C. The mixture was stirred at 0° C. for 1 h. The mixture was adjusted pH=6 with hydrochloric acid (1 M). The mixture was diluted with water/ethyl acetate (10.0 mL/10.0 mL). The organic layer was collected and concentrated to give a residue. The residue was purified by prep-HPLC. The desired fraction was collected and lyophilized to give (2-(2,6-dioxopiperidin-3-yl)-7-fluoro-3-oxoisoindolin-5-yl)methyl (4-(1-methylcyclopropyl)phenyl)carbamate. ¹H NMR (400 MHz, DMSO-d₆) δ=11.01 (s, 1H), 9.75 (br s, 1H), 7.67 (s, 1H), 7.55 (d, J=9.7 Hz, 1H), 7.36 (br d, J=8.6 Hz, 2H), 7.13 (d, J=8.7 Hz, 2H), 5.26 (s, 2H), 5.13 (dd, J=5.1, 13.3 Hz, 1H), 4.63-4.53 (m, 1H), 4.46-4.35 (m, 1H), 2.97-2.86 (m, 1H), 2.63-2.57 (m, 1H), 2.47-2.37 (m, 1H), 2.06-1.97 (m, 1H), 1.34 (s, 3H), 0.81-0.64 (m, 4H). MS (ESI) m/z 466.1[M+H]⁺

Compound 14: Step 1: To a solution of 4-fluoro-3-(trifluoromethoxy)aniline (500 mg, 2.56 mmol, 1.00 eq) in acetonitrile (5.00 mL) was added pyridine (607 mg, 7.68 mmol, 620 uL, 3.00 eq) and phenyl carbonochloridate (401 mg, 2.56 mmol, 321 uL, 1.00 eq). The mixture was stirred at 25° C. for 2 h. The reaction mixture was concentrated under reduced pressure to afford a residue. The residue was purified by reverse phase HPLC and lyophilized to afford phenyl (4-fluoro-3-(trifluoromethoxy) phenyl)carbamate. ¹H NMR (400 MHz, DMSO-d₆) δ=10.56 (br s, 1H), 7.55-7.38 (m, 5H), 7.30-7.21 (m, 3H). MS (ESI) m/z 316.0 [M+H]⁺

Step 2: To a solution of phenyl (4-fluoro-3-(trifluoromethoxy)phenyl)carbamate (90.0 mg, 286 umol, 1.00 eq) in dimethyl formamide (500 uL) was added 3-(4-fluoro-6-(hydroxymethyl)-1-oxoisoindolin-2-yl)piperidine-2,6-dione XVIII (83.5 mg, 286 umol, 1.00 eq) and sodium hydride (22.8 mg, 571 umol, 60% purity, 2.00 eq). The mixture was stirred at 25° C. for 1 h. The pH of the mixture was adjusted to 7 with hydrochloric acid (1 M) and diluted with dimethyl formamide (1.00 mL). The solution was purified by Prep-HPLC to afford (2-(2,6-dioxopiperidin-3-yl)-7-fluoro-3-oxoisoindolin-5-yl)methyl (4-fluoro-3-(trifluoromethoxy)phenyl)carbamate 14. ¹H NMR (400 MHz, DMSO-d₆) δ=11.01 (br s, 1H), 10.18 (br s, 1H), 7.73 (br d, J=7.2 Hz, 1H), 7.68 (s, 1H), 7.57 (d, J=9.6 Hz, 1H), 7.50-7.40 (m, 2H), 5.29 (s, 2H), 5.13 (dd, J=5.2, 13.6 Hz, 1H), 4.64-4.54 (m, 1H), 4.46-4.37 (m, 1H), 2.99-2.86 (m, 1H), 2.63-2.58 (m, 1H), 2.48-2.38 (m, 1H), 2.07-1.99 (m, 1H). MS (ESI) m/z 514.0 [M+H]⁺

Compound 15: Step 1: To a mixture of 3-(trifluoromethoxy)aniline (2.00 g, 11.3 mmol, 1.50 mL, 1.00 eq) and pyridine (2.68 g, 33.9 mmol, 2.73 mL, 3.00 eq) in acetonitrile (20.0 mL) was added phenyl carbonochloridate (2.30 g, 14.7 mmol, 1.84 mL, 1.30 eq) dropwise. The mixture was stirred at 15° C. for 2 h. The mixture was concentrated to give crude product. The crude product was purified by reversed-phase column. The desired fraction was collected and lyophilized to give phenyl (3-(trifluoromethoxy)phenyl)carbamate. ¹H NMR (400 MHz, DMSO-d₆) δ=10.55 (br s, 1H), 7.63 (br s, 1H), 7.50-7.42 (m, 4H), 7.30-7.22 (m, 3H), 7.03 (dd, J=1.0, 7.7 Hz, 1H).

Step 2: To a mixture of phenyl (3-(trifluoromethoxy)phenyl)carbamate (97.6 mg, 328 umol, 1.20 eq) and 3-(4-fluoro-6-(hydroxymethyl)-1-oxoisoindolin-2-yl)piperidine-2,6-dione XVIII (80.0 mg, 274 umol, 1.00 eq) in dimethyl formamide (1.00 mL) was added sodium hydride (16.4 mg, 411 umol, 60% purity, 1.50 eq) in one portion at 0° C. The mixture was stirred at 15° C. for 1 h. The mixture was quenched with 1 M hydrochloric acid (0.500 mL) and filtered. The filtrate was purified by prep-HPLC and lyophilized to give (2-(2,6-dioxopiperidin-3-yl)-7-fluoro-3-oxoisoindolin-5-yl)methyl (3-(trifluoromethoxy)phenyl)carbamate. ¹H NMR (400 MHz, DMSO-d₆) δ=11.01 (br s, 1H), 10.18 (s, 1H), 7.69 (s, 1H), 7.61-7.54 (m, 2H), 7.42 (d, J=5.4 Hz, 2H), 7.03-6.94 (m, 1H), 5.30 (s, 2H), 5.13 (dd, J=5.1, 13.3 Hz, 1H), 4.62-4.54 (m, 1H), 4.45-4.38 (m, 1H), 2.97-2.84 (m, 1H), 2.60 (td, J=2.0, 15.4 Hz, 1H), 2.47-2.38 (m, 1H), 2.05-1.98 (m, 1H). MS (ESI) m/z 496.0 [M+H]⁺

Compound 16: Step 1: To a solution of 2-chloro-5-nitropyridine (2.00 g, 12.6 mmol, 1.00 eq) in dimethyl formamide (20.0 mL) was added potassium carbonate (3.49 g, 25.2 mmol, 2.00 eq) and 2-methylpiperidine (1.25 g, 12.6 mmol, 1.49 mL, 1.00 eq). The mixture was stirred at 60° C. for 12 h. The reaction mixture was diluted with water/ethyl acetate (200 ml/200 ml). The organic layer was collected and concentrated to give 2-(2-methylpiperidin-1-yl)-5-nitropyridine. ¹H NMR (400 MHz, DMSO-d₆) δ=8.93 (d, J=2.8 Hz, 1H), 8.16 (dd, J=2.9, 9.7 Hz, 1H), 6.88 (d, J=9.7 Hz, 1H), 4.83 (br s, 1H), 4.39 (br d, J=12.9 Hz, 1H), 3.03 (dt, J=2.9, 13.3 Hz, 1H), 1.77-1.61 (m, 4H), 1.59-1.54 (m, 1H), 1.45-1.36 (m, 1H), 1.17 (d, J=6.8 Hz, 3H).

Step 2: To a solution of 2-(2-methylpiperidin-1-yl)-5-nitropyridine (0.500 g, 2.26 mmol, 1.00 eq) in methanol (21.0 mL) and water (7.00 mL) was added iron power (631 mg, 11.3 mmol, 5.00 eq) and ammonium chloride (967 mg, 18.1 mmol, 8.00 eq). The mixture was stirred at 80° C. for 2 h. The mixture was filtered and the filtrate was concentrated to give 6-(2-methylpiperidin-1-yl)pyridin-3-amine. ¹H NMR (400 MHz, DMSO-d₆) δ=7.58 (d, J=2.5 Hz, 1H), 6.88 (dd, J=2.9, 8.8 Hz, 1H), 6.54 (d, J=8.8 Hz, 1H), 4.45 (s, 2H), 4.33-4.24 (m, 1H), 3.64 (td, J=3.6, 12.7 Hz, 1H), 3.17 (d, J=4.8 Hz, 1H), 2.75-2.69 (m, 1H), 1.68-1.62 (m, 2H), 1.60-1.52 (m, 2H), 1.45-1.38 (m, 1H), 0.93 (d, J=6.6 Hz, 3H).

Step 3: To a solution of 6-(2-methylpiperidin-1-yl)pyridin-3-amine (412 mg, 2.15 mmol, 1.00 eq) in acetonitrile (10.0 mL) was added pyridine (511 mg, 6.46 mmol, 522 uL, 3.00 eq) and phenyl carbonochloridate (405 mg, 2.58 mmol, 324 uL, 1.20 eq). The mixture was stirred at 25° C. for 1 h. The mixture was concentrated to give a residue. The residue was purified by reverse phase HPLC. The desired fraction was collected and concentrated to give phenyl (6-(2-methylpiperidin-1-yl)pyridin-3-yl)carbamate. ¹H NMR (400 MHz, DMSO-d₆) δ=9.92 (s, 1H), 8.20 (br d, J=1.9 Hz, 1H), 7.63 (br dd, J=2.0, 8.9 Hz, 1H), 7.45-7.39 (m, 2H), 7.29-7.18 (m, 3H), 6.77-6.74 (m, 1H), 4.59-4.52 (m, 1H), 4.03-3.99 (m, 1H), 2.81 (dt, J=2.8, 12.9 Hz, 1H), 1.71-1.56 (m, 5H), 1.45-1.36 (m, 1H), 1.04 (d, J=6.8 Hz, 3H).

Step 4: To a solution of 3-(4-fluoro-6-(hydroxymethyl)-1-oxoisoindolin-2-yl)piperidine-2,6-dione XVIII (72.0 mg, 246 umol, 1.00 eq) in dimethyl formamide (2.00 mL) was added phenyl (6-(2-methylpiperidin-1-yl)pyridin-3-yl) carbamate (92.0 mg, 295 umol, 1.20 eq) and sodium hydride (19.7 mg, 493 umol, 60% purity, 2.00 eq) at 0° C. The mixture was stirred at 0° C. for 1 h. The mixture was adjusted pH=6 with hydrochloric acid (1 M, 0.500 ml). The mixture was diluted with water/ethyl acetate (10.0 ml/10.0 ml). The organic layer was collected and concentrated to give a residue. The residue was purified by prep-HPLC. The desired fraction was collected and lyophilized to give a residue. The residue was purified again by prep-HPLC. The desired fraction was collected and concentrated to give a residue. The residue was further purified by prep-HPLC. The desired fraction was collected and lyophilized to give (2-(2,6-dioxopiperidin-3-yl)-7-fluoro-3-oxoisoindolin-5-yl)methyl (6-(2-methylpiperidin-1-yl)pyridin-3-yl)carbamate 16. ¹H NMR (400 MHz, DMSO-d₆) δ=11.01 (br s, 1H), 9.54 (br s, 1H), 8.14 (br s, 1H), 7.73-7.50 (m, 3H), 6.72 (d, J=9.2 Hz, 1H), 5.25 (s, 2H), 5.13 (dd, J=5.1, 13.3 Hz, 1H), 4.61-4.51 (m, 2H), 4.45-4.37 (m, 1H), 3.97 (br d, J=13.3 Hz, 1H), 2.96-2.87 (m, 1H), 2.79 (dt, J=2.8, 12.8 Hz, 1H), 2.63-2.58 (m, 1H), 2.47-2.37 (m, 1H), 2.07-1.97 (m, 1H), 1.71-1.53 (m, 5H), 1.46-1.33 (m, 1H), 1.02 (d, J=6.7 Hz, 3H). MS (ESI) m/z 510.1[M+H]⁺

Compound 17: Step 1: To a mixture of 2-bromo-5-nitrophenol (1.00 g, 4.59 mmol, 1.00 eq) and 3-bromo-2-methylprop-1-ene (805 mg, 5.96 mmol, 601 uL, 1.30 eq) in acetone (5.00 mL) was added potassium carbonate (1.27 g, 9.17 mmol, 2.00 eq) in one portion at 25° C. and stirred for 12 h. The reaction mixture was diluted with water (30.0 mL) and exacted with ethyl acetate (3×30.0 mL). The organic phase was separated, washed with brine (2×10.0 mL), dried over sodium sulfate, filtered and concentrated under reduced pressure to give a residue. The residue was purified by silica gel chromatography to afford 1-bromo-2-((2-methylallyl)oxy)-4-nitrobenzene. ¹H NMR (400 MHz, DMSO-d₆) δ=7.94-7.82 (m, 2H), 7.82-7.68 (m, 1H), 5.18 (s, 1H), 5.04 (br s, 1H), 4.74 (br s, 2H), 1.82 (s, 3H).

Step 2: To a solution of 1-bromo-2-((2-methylallyl)oxy)-4-nitrobenzene (900 mg, 3.31 mmol, 1.00 eq) in dimethylformamide (5.00 mL) was added Sodium acetate (678 mg, 8.27 mmol, 2.50 eq) and palladium acetate (149 mg, 662 umol, 0.200 eq) and tetraethylammonium; iodide (936 mg, 3.64 mmol, 1.10 eq) and sodium formate (225 mg, 3.31 mmol, 179 uL, 1.00 eq) in one portion at 100° C. under nitrogen and stirred for 12 h.

The reaction mixture was diluted with water (30.0 mL) and extracted with ethyl acetate (3×30.0 mL). The organic phase was separated, washed with brine (2×10.0 mL), dried over sodium sulfate, filtered and concentrated under reduced pressure to give a residue. The residue was purified by silica gel chromatography to afford 3,3-dimethyl-6-nitro-2H-benzofuran. ¹H NMR (400 MHz, DMSO-d₆) δ=7.86 (td, J=2.0, 8.0 Hz, 1H), 7.64-7.50 (m, 2H), 4.46-4.40 (m, 2H), 1.42-1.38 (m, 6H).

Step 3: To a solution of 3,3-dimethyl-6-nitro-2,3-dihydrobenzofuran (300 mg, 1.55 mmol, 1.00 eq) in water (3.00 mL) and methanol (6.00 mL) was added ammonium chloride (415 mg, 7.76 mmol, 5.00 eq) and iron powder (434 mg, 7.76 mmol, 5.00 eq) and the mixture was stirred at 80° C. The mixture was stirred at 80° C. for 2 h. The mixture was filtered and filtrate was concentrated under reduced pressure to give a residue. The reaction mixture was diluted with water (30.0 mL) and exacted with ethyl acetate (3×30.0 mL). The organic phase was separated, washed with brine (2×10.0 mL), dried over sodium sulfate, filtered and concentrated under reduced pressure to give 3,3-dimethyl-2,3-dihydrobenzofuran-6-amine. MS (ESI) m/z 164.0 [M+H]⁺.

Step 4: To a mixture of 3,3-dimethyl-2,3-dihydrobenzofuran-6-amine (162 mg, 993 umol, 1.00 eq) and pyridine (236 mg, 2.98 mmol, 240 uL, 3.00 eq) in acetonitrile acetonitrile (5.00 mL) was added phenyl carbonochloridate (171 mg, 1.09 mmol, 137 uL, 1.10 eq) in one portion at 25° C. The mixture was stirred at 25° C. for 12 h. The reaction mixture was diluted with water (30.0 mL) and extracted with ethyl acetate (3×30.0 mL). The organic phase was separated, washed with brine (2×10.0 mL), dried over sodium sulfate, filtered and concentrated under reduced pressure to give a residue. The residue was purified by reversed phase-HPLC to give phenyl (3,3-dimethyl-2,3-dihydrobenzofuran-6-yl)carbamate. MS (ESI) m/z 284.2 [M+H]⁺.

Step 5: To a solution of 3-(4-fluoro-6-(hydroxymethyl)-1-oxoisoindolin-2-yl)piperidine-2,6-dione XVIII (80.0 mg, 274 umol, 1.00 eq) in dimethyl formamide (2.00 mL) was added phenyl (3,3-dimethyl-2,3-dihydrobenzofuran-6-yl)carbamate (93.1 mg, 328 umol, 1.20 eq) and sodium hydride (21.9 mg, 547 umol, 60% purity, 2.00 eq) at 25° C. The mixture was stirred at 25° C. for 1 h. The reaction mixture was quenched with hydrochloric acid (1 M, 1.00 ml) to give a solution. The solution was purified by Prep-HPLC and lyophilized to give (2-(2,6-dioxopiperidin-3-yl)-7-fluoro-3-oxoisoindolin-5-yl)methyl(3,3-dimethyl-2,3-dihydrobenzofuran-6-yl) carbamate 17. ¹H NMR (400 MHz, DMSO-d₆) δ=11.02 (br s, 1H), 9.78 (br s, 1H), 7.68 (s, 1H), 7.56 (d, J=10.0 Hz, 1H), 7.08 (d, J=8.0 Hz, 1H), 6.98 (s, 1H), 6.94-6.90 (m, 1H), 5.26 (s, 2H), 5.14 (dd, J=5.2, 13.2 Hz, 1H), 4.62-4.56 (m, 1H), 4.44-4.38 (m, 1H), 4.18 (s, 2H), 2.98-2.88 (m, 1H), 2.62 (br d, J=13.2 Hz, 1H), 2.44 (br dd, J=4.4, 13.2 Hz, 1H), 2.06-1.98 (m, 1H), 1.26 (s, 6H). MS (ESI) m/z 482.1 [M+H]⁺

Compound 18: Step 1: To a solution of 2-chloro-5-nitropyridine (2.00 g, 12.6 mmol, 1.00 eq) in dimethyl formamide (20.0 mL) was added potassium carbonate (5.23 g, 37.8 mmol, 3.00 eq), (2-fluorophenyl)boronic acid (1.77 g, 12.6 mmol, 1.00 eq) and tetrakis(triphenylphosphine)palladium (728 mg, 630 umol, 0.0500 eq) at nitrogen. The mixture was stirred at 110° C. for 12 h. The reaction mixture was diluted with water (200 mL) and extracted with ethyl acetate (2×100 mL). The organic phase was separated, washed with brine (2×30.0 mL), dried over sodium sulfate, filtered and concentrated under reduced pressure to give a residue. The crude product was purified by column chromatography to give 2-(2-fluorophenyl)-5-nitropyridine. ¹H NMR (400 MHz, DMSO-d₆) δ=9.54-9.46 (m, 1H), 8.71 (dd, J=2.8, 8.7 Hz, 1H), 8.12-8.02 (m, 2H), 7.65-7.59 (m, 1H), 7.46-7.40 (m, 2H).

Step 2: To a solution of 2-(2-fluorophenyl)-5-nitropyridine (2.00 g, 9.17 mmol, 1.00 eq) in the mixture of methanol (15.0 mL) and water (5.00 mL) was added iron powder (2.56 g, 45.8 mmol, 5.00 eq) and ammonium chloride (3.92 g, 73.3 mmol, 8.00 eq). The mixture was stirred at 80° C. for 1 h. The mixture was filtered and the filtrate was concentrated under reduced pressure to give a residue. The crude product was diluted with water (30.0 mL) and exacted with ethyl acetate (3×30.0 mL). The organic phase was separated, washed with brine (2×10.0 mL), dried over sodium sulfate, filtered and concentrated under reduced pressure to give 6-(2-fluorophenyl)pyridin-3-amine.

Step 3: To a solution of 6-(2-fluorophenyl)pyridin-3-amine (1.70 g, 9.03 mmol, 1.00 eq) in acetonitrile (20.0 mL) was added pyridine (3.57 g, 45.1 mmol, 3.65 mL, 5.00 eq) and phenyl carbonochloridate (2.00 g, 12.8 mmol, 1.60 mL, 1.41 eq). The mixture was stirred at 25° C. for 1 h. The reaction mixture was concentrated under reduced pressure to give a residue. The crude product was triturated with water (100 mL) and filtered. The filter cake was washed with water (5 ml) and dried to give phenyl (6-(2-fluorophenyl)pyridin-3-yl)carbamate. ¹H NMR (400 MHz, DMSO-d₆) δ=10.63 (br s, 1H), 8.83 (d, J=2.3 Hz, 1H), 8.05 (dd, J=2.4, 8.7 Hz, 1H), 7.94 (dt, J=1.6, 7.9 Hz, 1H), 7.81 (dd, J=1.7, 8.7 Hz, 1H), 7.52-7.40 (m, 3H), 7.36-7.31 (m, 2H), 7.30-7.21 (m, 3H). MS (ESI) m/z 309.1 [M+H]⁺.

Step 4: To a solution of 3-(4-fluoro-6-(hydroxymethyl)-1-oxoisoindolin-2-yl)piperidine-2,6-dione XVIII (80.0 mg, 273 umol, 1.00 eq) in dimethyl formamide (2.00 mL) was added phenyl (6-(2-fluorophenyl)pyridin-3-yl) carbamate (92.8 mg, 301 umol, 1.10 eq) and sodium hydride (21.9 mg, 547 umol, 60% purity, 2.00 eq) at 0° C. The mixture was stirred at 0° C. for 0.5 h. The reaction mixture was quenched with formic acid (0.500 ml) to give a solution. The solution was purified by prep-HPLC and lyophilized to give (2-(2,6-dioxopiperidin-3-yl)-7-fluoro-3-oxoisoindolin-5-yl)methyl(6-(2-fluorophenyl)pyridin-3-yl)carbamate. ¹H NMR (400 MHz, DMSO-d₆) δ=11.02 (s, 1H), 10.27 (br s, 1H), 8.80 (br d, J=2.4 Hz, 1H), 8.11-8.02 (m, 1H), 7.92 (dt, J=1.5, 7.9 Hz, 1H), 7.83-7.76 (m, 1H), 7.72 (s, 1H), 7.60 (d, J=9.7 Hz, 1H), 7.51-7.42 (m, 1H), 7.39-7.28 (m, 2H), 5.34 (s, 2H), 5.14 (dd, J=5.1, 13.3 Hz, 1H), 4.64-4.55 (m, 1H), 4.47-4.39 (m, 1H), 2.98-2.87 (m, 1H), 2.61 (br d, J=17.9 Hz, 1H), 2.45 (br dd, J=4.5, 13.1 Hz, 1H), 2.06-1.99 (m, 1H). MS (ESI) m/z 507.1 [M+H]⁺

Compound 19: Step 1: To a solution of 2-chloro-5-nitropyridine (3.00 g, 18.9 mmol, 1.00 eq) in dimethyl formamide (30.0 mL) was added O-tolylboronic acid (2.57 g, 18.9 mmol, 1.00 eq), potassium carbonate (7.85 g, 56.7 mmol, 3.00 eq) and tetrakis(triphenylphosphine)palladium (1.09 g, 946 umol, 0.0500 eq) at nitrogen. The mixture was stirred at 110° C. for 12 h. The reaction mixture was diluted with water (300 mL) and exacted with ethyl acetate (3×300 mL). The organic phase was separated, washed with brine (2×100 mL), dried over sodium sulfate, filtered and concentrated under reduced pressure to give a residue. The crude product was purified by column chromatography to give 5-nitro-2-(o-tolyl)pyridine. ¹H NMR (400 MHz, DMSO-d₆) δ=9.49-9.42 (m, 1H), 8.66 (dd, J=2.8, 8.6 Hz, 1H), 7.87 (d, J=8.7 Hz, 1H), 7.50 (d, J=7.7 Hz, 1H), 7.44-7.40 (m, 1H), 7.39-7.32 (m, 2H), 2.37 (s, 3H).

Step 2: To a solution of 5-nitro-2-(o-tolyl)pyridine (3.00 g, 14.0 mmol, 1.00 eq) in methanol (30.0 mL) and water (10.0 mL) was added iron powder (3.91 g, 70.0 mmol, 5.00 eq) and ammonium chloride (5.99 g, 112 mmol, 8.00 eq). The mixture was stirred at 80° C. for 1 h. The mixture was filtered and the filtrate was concentrated under reduced pressure to give a residue. The crude product was diluted with water (50.0 mL) and exacted with ethyl acetate (3×30.0 mL). The organic phase was separated, washed with brine (2×10.0 mL), dried over sodium sulfate, filtered and concentrated under reduced pressure to give 6-(o-tolyl)pyridin-3-amine. ¹H NMR (400 MHz, DMSO-d₆) δ=8.01 (d, J=2.7 Hz, 1H), 7.33-7.27 (m, 1H), 7.26-7.19 (m, 3H), 7.17 (d, J=8.3 Hz, 1H), 7.03-6.95 (m, 1H), 5.38 (s, 2H), 2.31 (s, 3H).

Step 3: To a solution of 6-(o-tolyl)pyridin-3-amine (2.50 g, 13.5 mmol, 1.00 eq) in acetonitrile (25.0 mL) was added pyridine (5.37 g, 67.8 mmol, 5.48 mL, 5.00 eq) and phenyl carbonochloridate (2.76 g, 17.6 mmol, 2.21 mL, 1.30 eq). The mixture was stirred at 25° C. for 1 h. The reaction mixture was concentrated under reduced pressure to give a residue. The crude product was triturated with water (100 mL) and filtered. The filter cake was washed with water (5.00 ml) and dried to give phenyl (6-(o-tolyl)pyridin-3-yl)carbamate. ¹H NMR (400 MHz, DMSO-d₆) δ=10.55 (br s, 1H), 8.78 (d, J=2.3 Hz, 1H), 8.05-7.99 (m, 1H), 7.54-7.51 (m, 1H), 7.48-7.44 (m, 2H), 7.39 (br d, J=6.8 Hz, 1H), 7.32-7.26 (m, 6H), 2.33 (s, 3H).

Step 4: To a solution of 3-(4-fluoro-6-(hydroxymethyl)-1-oxoisoindolin-2-yl)piperidine-2,6-dione XVIII (80.0 mg, 273 umol, 1.00 eq) in dimethyl formamide (2.00 mL) was added phenyl (6-(o-tolyl)pyridin-3-yl)carbamate (100 mg, 328 umol, 1.20 eq) and sodium hydride (21.9 mg, 547 umol, 60% purity, 2.00 eq) at 0° C. The mixture was stirred at 0° C. for 0.5 h. The reaction mixture was quenched with hydrochloric acid (1M, 1.00 ml) to give a solution. The solution was purified by prep-HPLC and lyophilized to give (2-(2,6-dioxopiperidin-3-yl)-7-fluoro-3-oxoisoindolin-5-yl)methyl (6-(o-tolyl)pyridin-3-yl)carbamate. ¹H NMR (400 MHz, DMSO-d₆) δ=11.02 (s, 1H), 10.55 (br s, 1H), 8.85 (s, 1H), 8.21 (br d, J=8.8 Hz, 1H), 7.80-7.70 (m, 2H), 7.61 (d, J=9.3 Hz, 1H), 7.45-7.31 (m, 4H), 5.37 (s, 2H), 5.14 (dd, J=5.1, 13.3 Hz, 1H), 4.60 (d, J=17.5 Hz, 1H), 4.46-4.40 (m, 1H), 2.98-2.87 (m, 1H), 2.61 (br d, J=16.8 Hz, 1H), 2.46-2.42 (m, 1H), 2.34-2.31 (m, 3H), 2.06-1.99 (m, 1H). MS (ESI) m/z 503.1 [M+H]⁺

Compound 20: Step 1: To a mixture of 2-chloro-3-methyl-5-nitropyridine (5.00 g, 29.0 mmol, 1.00 eq) and phenylboronic acid (4.24 g, 34.8 mmol, 1.20 eq) in dioxane (50.0 mL) was added tetrakis[triphenylphosphine]palladium(0) (6.70 g, 5.79 mmol, 0.200 eq) and potassium carbonate (6.01 g, 43.5 mmol, 1.50 eq) in one portion at 25° C. under nitrogen. The mixture was stirred at 100° C. for 12 h. The reaction mixture was diluted with water (30.0 mL) and exacted with ethyl acetate (3×30.0 mL). The organic phase was separated, washed with brine (2×10.0 mL), dried over sodium sulfate, filtered and concentrated under reduced pressure to give a residue. The residue was purified by silica gel chromatography to afford 3-methyl-5-nitro-2-phenyl-pyridine. ¹H NMR (400 MHz, DMSO-d₆) δ=9.28 (br s, 1H), 8.60 (br s, 1H), 7.70-7.58 (m, 2H), 7.58-7.48 (m, 3H), 2.50-2.46 (m, 3H). MS (ESI) m/z 215.2 [M+H]⁺.

Step 2: To a solution of 3-methyl-5-nitro-2-phenylpyridine (1.00 g, 4.67 mmol, 1.00 eq) in methanol (6.00 mL) and water (3.00 mL) was added iron powder (1.30 g, 23.3 mmol, 5.00 eq) and ammonium chloride (1.25 g, 23.3 mmol, 5.00 eq) in one portion at 25° C., and stirred at 80° C. for 2 h. The mixture was filtered to give a filtrate, which was concentrated under reduced pressure to give a residue. The residue was diluted with water (30.0 mL) and exacted with ethyl acetate (3×30.0 mL). The organic phase was separated, washed with brine (2×10.0 mL), dried over sodium sulfate, filtered and concentrated under reduced pressure to give 5-methyl-6-phenylpyridin-3-amine. MS (ESI) m/z 185.2 [M+H]⁺.

Step 3: To a solution of 5-methyl-6-phenylpyridin-3-amine (843 mg, 4.58 mmol, 1.00 eq) and pyridine (1.09 g, 13.7 mmol, 1.11 mL, 3.00 eq) in acetonitrile (2.00 mL) was added phenyl carbonochloridate (788 mg, 5.03 mmol, 630 uL, 1.10 eq) at 25° C. The reaction mixture was stirred at 25° C. for 12 h. The reaction mixture was concentrated to give a residue, which was purified by reversed phase and lyophilized to give phenyl (5-methyl-6-phenylpyridin-3-yl)carbamate. MS (ESI) m/z 305.2 [M+H]⁺.

Step 4: To a mixture of 3-(4-fluoro-6-(hydroxymethyl)-1-oxoisoindolin-2-yl)piperidine-2,6-dione XVIII (100 mg, 342 umol, 1.00 eq) and phenyl (5-methyl-6-phenylpyridin-3-yl)carbamate (125 mg, 411 umol, 1.20 eq) in dimethyl formamide (2.00 mL) was added sodium hydride (27.4 mg, 684 umol, 60% purity, 2.00 eq) at 0° C. The reaction mixture was stirred at 25° C. for 1 h. The reaction mixture was added formic acid (1.00 mL) and filtered to give a filtrate. The filtrate was purified twice by Prep-HPLC and lyophilized to give (2-(2,6-dioxopiperidin-3-yl)-7-fluoro-3-oxoisoindolin-5-yl)methyl(5-methyl-6-phenylpyridin-3-yl)carbamate 20. ¹H NMR (400 MHz, DMSO-d₆) δ=11.02 (br s, 1H), 10.12 (br s, 1H), 8.56 (s, 1H), 7.84 (br s, 1H), 7.72 (s, 1H), 7.60 (d, J=9.6 Hz, 1H), 7.54-7.50 (m, 2H), 7.46 (t, J=7.6 Hz, 2H), 7.42-7.36 (m, 1H), 5.38-5.30 (m, 2H), 5.14 (dd, J=5.2, 13.2 Hz, 1H), 4.64-4.56 (m, 1H), 4.48-4.38 (m, 1H), 2.98-2.86 (m, 1H), 2.64-2.58 (m, 1H), 2.46 (br d, J=8.8 Hz, 1H), 2.32 (s, 3H), 2.08-1.98 (m, 1H). MS (ESI) m/z 503.2 [M+H]⁺

Compound 21: Step 1: To a mixture of 3-nitrophenol (500 mg, 3.59 mmol, 714 uL, 1.00 eq) and bromocyclopropane (1.30 g, 10.8 mmol, 864 uL, 3.00 eq) in 1-methyl-2-pyrrolidinone (5.00 mL) was added cesium carbonate (2.34 g, 7.19 mmol, 2.00 eq) in portions. The mixture was stirred at 180° C. for 2 h under microwave. The mixture was concentrated and purified by silica gel chromatography to give 1-cyclopropoxy-3-nitrobenzene. ¹H NMR (400 MHz, DMSO-d₆)=7.63-7.47 (m, 3H), 7.24 (td, J=2.5, 6.6 Hz, 1H), 4.01-3.94 (m, 1H), 0.87-0.81 (m, 2H), 0.72 (br s, 2H).

Step 2: To a solution of 1-cyclopropoxy-3-nitrobenzene (240 mg, 1.34 mmol, 1.00 eq) in tetrahydrofuran (5.00 mL) was added Pd/C (50.0 mg, 10% purity) under nitrogen. The mixture was stirred at 20° C. for 1 h under hydrogen (15 psi). The mixture was concentrated to give 3-cyclopropoxyaniline. MS (ESI) m/z 150.2 [M+H]⁺

Step 3: To a solution of 3-cyclopropoxyaniline (280 mg, 1.88 mmol, 1.00 eq) and pyridine (742 mg, 9.38 mmol, 757 uL, 5.00 eq) in acetonitrile (3.00 mL) was added phenyl carbonochloridate (441 mg, 2.82 mmol, 353 uL, 1.50 eq) and stirred at 20° C. for 12 h. The mixture was concentrated to give crude product and purified by reversed-phase HPLC to give phenyl (3-cyclopropoxyphenyl)carbamate. ¹H NMR (400 MHz, DMSO-d₆) δ=10.24 (br s, 1H), 7.49 (d, J=1.1 Hz, 1H), 7.48-7.46 (m, 1H), 7.44 (d, J=0.8 Hz, 1H), 7.39-7.37 (m, 1H), 7.33-7.31 (m, 1H), 7.26 (d, J=7.4 Hz, 1H), 7.22-7.20 (m, 1H), 7.09 (dd, J=1.1, 8.1 Hz, 1H), 6.75 (ddd, J=0.8, 2.4, 8.2 Hz, 1H), 3.78 (tt, J=3.0, 6.0 Hz, 1H), 0.80-0.72 (m, 2H), 0.69-0.61 (m, 2H). MS (ESI) m/z 270.2 [M+H]⁺

Step 4: To a mixture of phenyl (3-cyclopropoxyphenyl)carbamate (88.4 mg, 328 umol, 1.20 eq) and 3-(4-fluoro-6-(hydroxymethyl)-1-oxoisoindolin-2-yl)piperidine-2,6-dione XVIII (80.0 mg, 273 umol, 1.00 eq) in dimethyl formamide (1.00 mL) was added sodium hydride (21.9 mg, 547 umol, 60% purity, 2.00 eq) at 0° C. The mixture was stirred at 25° C. for 1 h. The mixture was quenched by 1M hydrochloric acid (1.00 mL) and filtered. The filtrate was purified by prep-HPLC to give (2-(2,6-dioxopiperidin-3-yl)-7-fluoro-3-oxoisoindolin-5-yl)methyl (3-cyclopropoxyphenyl)carbamate 21. ¹H NMR (400 MHz, DMSO-d₆) δ=11.03 (s, 1H), 9.86 (s, 1H), 7.69 (s, 1H), 7.57 (d, J=9.4 Hz, 1H), 7.28 (s, 1H), 7.22-7.15 (m, 1H), 7.05 (br d, J=7.5 Hz, 1H), 6.70 (dd, J=2.1, 7.8 Hz, 1H), 5.28 (s, 2H), 5.18-5.10 (m, 1H), 4.63-4.55 (m, 1H), 4.46-4.38 (m, 1H), 3.77 (qd, J=3.0, 5.9 Hz, 1H), 2.98-2.88 (m, 1H), 2.63 (br s, 1H), 2.46-2.42 (m, 1H), 2.05-1.98 (m, 1H), 0.79-0.72 (m, 2H), 0.68-0.61 (m, 2H). MS (ESI) m/z 468.1 [M+H]⁺

Compound 22: Step 1: To a mixture of 4-bromoaniline (10.0 g, 58.1 mmol, 1.00 eq) and triethylamine (17.4 g, 172 mmol, 24.0 mL, 2.97 eq) in dichloromethane (70.0 mL) was added trifluoroacetic anhydride (18.3 g, 87.2 mmol, 12.1 mL, 1.50 eq) dropwise at 0° C. The mixture was stirred at 25° C. for 12 h. The residue was purified by silica gel chromatography to afford N-(4-bromophenyl)-2,2,2-trifluoro-acetamide.

Step 2: To a mixture of N-(4-bromophenyl)-2,2,2-trifluoro-acetamide (2.00 g, 7.46 mmol, 1.00 eq) in tetrahydrofuran (15.0 mL) was added n-Butyllithium (2.50 M, 6.27 mL, 2.10 eq) dropwise at −78° C. The mixture was stirred at −78° C. for 0.5 h. Then cyclobutanone (627 mg, 8.95 mmol, 669 uL, 1.20 eq) was added to the mixture and stirred at −78° C. for 2.5 h.

The mixture was quenched by saturated ammonium chloride solution and extracted with ethyl acetate (2×20.0 mL). The combined organic phase was washed with brine (20.0 mL), dried over anhydrous sodium sulfate, filtered and concentrated under reduced pressure to give a residue. The residue was purified by silica gel chromatography to afford 2,2,2-trifluoro-N-(4-(1-hydroxycyclobutyl)phenyl)acetamide. ¹H NMR (400 MHz, CDCl₃) δ=8.04 (br s, 1H), 7.52-7.41 (m, 4H), 2.52-2.43 (m, 2H), 2.37-2.25 (m, 2H), 2.02-1.98 (m, 1H), 1.67-1.61 (m, 1H). MS (ESI) m/z 242.0 [M−18+H].

Step 3: To a mixture of 2,2,2-trifluoro-N-(4-(1-hydroxycyclobutyl)phenyl)acetamide (1.60 g, 6.17 mmol, 1.00 eq) in methanol (10.0 mL) was added sodium hydroxide solution (1.00 M, 6.17 mL, 1.00 eq). The mixture was stirred at 25° C. for 12 h. The mixture was concentrated to dryness and then diluted into water (10.0 mL) and extracted with ethyl acetate (2×20.0 mL). The combined organic phase was washed with brine (20.0 mL), dried over anhydrous sodium sulfate, filtered and concentrated under reduced pressure to give a residue. The residue was purified by silica gel chromatography to afford 1-(4-aminophenyl)cyclobutanol. ¹H NMR (400 MHz, CDCl₃) δ=7.31 (d, J=8.6 Hz, 2H), 6.77-6.63 (m, 2H), 3.69 (br s, 2H), 2.62-2.49 (m, 2H), 2.42-2.28 (m, 2H), 2.04-1.97 (m, 1H), 1.72-1.59 (m, 1H).

Step 4: To a solution of 1-(4-aminophenyl)cyclobutanol (0.700 g, 4.29 mmol, 1.00 eq) in tetrahydrofuran (10.0 mL) was added sodium borohydride (923 mg, 24.4 mmol, 5.69 eq) and aluminium trichloride (1.72 g, 12.9 mmol, 704 uL, 3.00 eq) in portions. The mixture was stirred at 70° C. for 3 h. The mixture was poured into water (100 mL) in portions. The aqueous phase was extracted with ethyl acetate (2×20.0 mL). The combined organic phase was washed with brine (30.0 mL), dried over anhydrous sodium sulfate, filtered and concentrated under reduced pressure to give a residue. The residue was purified by silica gel chromatography to afford 4-cyclobutylaniline. ¹H NMR (400 MHz, CDCl₃) δ=7.14-7.01 (m, 2H), 6.76-6.62 (m, 2H), 3.58 (br d, J=1.2 Hz, 2H), 3.49-3.42 (m, 1H), 2.37-2.27 (m, 2H), 2.18-2.05 (m, 2H), 2.04-1.93 (m, 1H), 1.89-1.80 (m, 1H).

Step 5: To a solution of 4-cyclobutylaniline (200 mg, 1.36 mmol, 1.00 eq) and pyridine (537 mg, 6.79 mmol, 548 uL, 5.00 eq) in acetonitrile (3.00 mL) was added phenyl carbonochloridate (255 mg, 1.63 mmol, 204 uL, 1.20 eq), the mixture was stirred at 25° C. for 1 h. The reaction mixture was concentrated to give a residue. The residue was purified by reversed-phase HPLC to afford phenyl N-(4-cyclobutylphenyl)carbamate. MS (ESI) m/z 268.1 [M+H]⁺.

Step 6: To a mixture of 3-(4-fluoro-6-(hydroxymethyl)-1-oxoisoindolin-2-yl)piperidine-2,6-dione XVIII (80.0 mg, 274 umol, 1.00 eq) and phenyl (4-cyclobutylphenyl)carbamate (87.8 mg, 328 umol, 1.20 eq) in dimethyl formamide (1.00 mL) was added sodium hydride (21.9 mg, 547 umol, 60% purity, 2.00 eq) at 0° C. The reaction mixture was stirred at 25° C. for 1 h. The reaction mixture was added formic acid (2.00 mL) and filtered to give a filtrate. The filtrate was purified by Prep-HPLC and lyophilized to give (2-(2,6-dioxopiperidin-3-yl)-7-fluoro-3-oxoisoindolin-5-yl)methyl(4-cyclobutylphenyl) carbamate 22. ¹H NMR (400 MHz, DMSO-d₆) δ=11.08-10.93 (m, 1H), 9.76 (br s, 1H), 7.68 (s, 1H), 7.56 (d, J=9.6 Hz, 1H), 7.39 (br d, J=8.0 Hz, 2H), 7.15 (d, J=8.4 Hz, 2H), 5.27 (s, 2H), 5.14 (dd, J=5.2, 13.2 Hz, 1H), 4.62-4.55 (m, 1H), 4.45-4.38 (m, 1H), 3.45 (quin, J=8.8 Hz, 1H), 2.97-2.87 (m, 1H), 2.64-2.59 (m, 1H), 2.47-2.40 (m, 1H), 2.30-2.25 (m, 1H), 2.25-2.21 (m, 1H), 2.08-1.99 (m, 3H), 1.98-1.90 (m, 1H), 1.84-1.75 (m, 1H). MS (ESI) m/z 466.1 [M+H]⁺

Compound 23: Step 1: To a solution of 2-chloro-5-nitropyridine (652 mg, 4.11 mmol, 1.00 eq) and (R)-2-methylpyrrolidine hydrochloride (500 mg, 4.11 mmol, 1.00 eq, HCl) in dimethyl formamide (3.00 mL) was added potassium carbonate (1.70 g, 12.3 mmol, 3.00 eq) and stirred at 60° C. for 2 h. The mixture was diluted with water (50.0 mL) and extracted with ethyl acetate (3×30.0 mL). The combined organic layer was washed with brine (20.0 mL) and dried over anhydrous sodium sulfate, filtered and concentrated to give (R)-2-(2-methylpyrrolidin-1-yl)-5-nitropyridine. ¹H NMR (400 MHz, DMSO-d₆) δ=8.96 (d, J=2.7 Hz, 1H), 8.18 (dd, J=2.8, 9.5 Hz, 1H), 6.57 (br s, 1H), 4.51-4.13 (m, 1H), 3.61 (br s, 1H), 3.50-3.39 (m, 1H), 2.16-1.94 (m, 3H), 1.73 (br s, 1H), 1.19 (br d, J=6.2 Hz, 3H). MS (ESI) m/z 208.0 [M+H]⁺

Step 2: To a solution of (R)-2-(2-methylpyrrolidin-1-yl)-5-nitropyridine (900 mg, 4.34 mmol, 1.00 eq) in tetrahydrofuran (10.0 mL) was added Pd/C (100 mg, 10% purity) in portions under nitrogen. The mixture was stirred at 20° C. for 1 h under hydrogen (15 Psi). The mixture was filtered and the filtrate was concentrated to give (R)-6-(2-methylpyrrolidin-1-yl)pyridin-3-amine. MS (ESI) m/z 178.2 [M+H]⁺

Step 3: To a solution of (R)-6-(2-methylpyrrolidin-1-yl)pyridin-3-amine (770 mg, 4.34 mmol, 1.00 eq) and pyridine (1.72 g, 21.7 mmol, 1.75 mL, 5.00 eq) in acetonitrile (10.0 mL) was added phenyl carbonochloridate (884 mg, 5.65 mmol, 707 uL, 1.30 eq) dropwise at 0° C. The mixture was stirred at 20° C. for 2 h. The mixture was concentrated to give crude product and purified by reversed-phase HPLC to give (R)-phenyl (6-(2-methylpyrrolidin-1-yl)pyridin-3-yl)carbamate. MS (ESI) m/z 298.1 [M+H]⁺

Step 4: To a solution of 3-(4-fluoro-6-(hydroxymethyl)-1-oxoisoindolin-2-yl)piperidine-2,6-dione XVIII (64.0 mg, 219 umol, 1.00 eq) and (R)-phenyl (6-(2-methylpyrrolidin-1-yl)pyridin-3-yl)carbamate (71.6 mg, 240 umol, 1.10 eq) in dimethyl formamide (1.00 mL) was added sodium hydride (17.5 mg, 437 umol, 60% purity, 2.00 eq) in portions at 0° C. The mixture was stirred at 25° C. for 1 h. The mixture was quenched by 1 M hydrochloric acid (1.00 mL) and filtered. The filtrate was purified by prep-HPLC and lyophilized to give (2-(2,6-dioxopiperidin-3-yl)-7-fluoro-3-oxoisoindolin-5-yl)methyl (6-((R)-2-methylpyrrolidin-1-yl)pyridin-3-yl)carbamate 23. ¹H NMR (400 MHz, DMSO-d₆) δ 11.02 (s, 1H), 10.14 (br s, 1H), 8.17 (br s, 1H), 7.95 (br d, J=9.7 Hz, 1H), 7.68 (s, 1H), 7.58 (d, J=9.5 Hz, 1H), 7.17 (br d, J=9.2 Hz, 1H), 5.31 (s, 2H), 5.13 (dd, J=5.1, 13.3 Hz, 1H), 4.59 (d, J=17.4 Hz, 1H), 4.47-4.34 (m, 1H), 4.32-4.18 (m, 1H), 3.68 (br s, 1H), 3.44-3.36 (m, 1H), 2.99-2.85 (m, 1H), 2.65-2.58 (m, 1H), 2.46-2.40 (m, 1H), 2.13-1.98 (m, 4H), 1.81-1.73 (m, 1H), 1.17 (d, J=6.3 Hz, 3H). MS (ESI) m/z 496.1 [M+H]⁺

Compound 24: Step 1: To a solution of 4-phenylpyridin-2-amine (500 mg, 2.94 mmol, 1.00 eq) in acetonitrile (10.0 mL) was added pyridine (1.16 g, 14.6 mmol, 1.19 mL, 5.00 eq) and phenyl carbonochloridate (597 mg, 3.82 mmol, 478 uL, 1.30 eq). The mixture was stirred at 25° C. for 0.5 h. The reaction mixture was concentrated under reduced pressure to give a residue. The crude product was triturated with water (10.0 ml) and filtered. The filter cake was washed with water (5.00 ml) and dried to give phenyl (4-phenylpyridin-2-yl)carbamate. ¹H NMR (400 MHz, DMSO-d₆) δ=10.86 (s, 1H), 8.40 (d, J=5.3 Hz, 1H), 8.13 (d, J=0.9 Hz, 1H), 7.77-7.70 (m, 2H), 7.53-7.49 (m, 3H), 7.47-7.43 (m, 3H), 7.30-7.22 (m, 3H).

Step 2: To a solution of phenyl (4-phenylpyridin-2-yl)carbamate (100 mg, 344 umol, 1.00 eq) in dimethyl formamide (500 uL) was added 3-(4-fluoro-6-(hydroxymethyl)-1-oxoisoindolin-2-yl)piperidine-2,6-dione XVIII (111 mg, 379 umol, 1.10 eq) and sodium hydride (27.6 mg, 689 umol, 60% purity, 2.00 eq) at 0° C. The mixture was stirred at 25° C. for 1 h. The pH of the mixture was adjusted to 7 with hydrochloric acid (1 M) and diluted with dimethyl formamide (1.00 mL). The mixture was purified by Prep-HPLC and the desired eluent was lyophilized to afford crude product. The crude product was again purified by Prep-HPLC and the desired eluent was lyophilized to afford (2-(2,6-dioxopiperidin-3-yl)-7-fluoro-3-oxoisoindolin-5-yl)methyl(4-phenylpyridin-2-yl)carbamate 24. ¹H NMR (400 MHz, DMSO-d₆) δ=10.97 (m, 1H), 10.51 (s, 1H), 8.35 (d, J=5.2 Hz, 1H), 8.12 (s, 1H), 7.78-7.68 (m, 3H), 7.60 (d, J=10.4 Hz, 1H), 7.56-7.45 (m, 3H), 7.38 (dd, J=1.6, 5.2 Hz, 1H), 5.33 (s, 2H), 5.13 (dd, J=5.6, 13.2 Hz, 1H), 4.63-4.52 (m, 1H), 4.45-4.35 (m, 1H), 3.00-2.81 (m, 1H), 2.62-2.58 (m, 1H), 2.44 (br dd, J=4.4, 13.2 Hz, 1H), 2.06-1.95 (m, 1H). MS (ESI) m/z 489.1 [M+H]⁺

Compound 25: Step 1: A mixture of 3-(tert-butyl)aniline (500 mg, 3.35 mmol, 1.00 eq), phenyl carbonochloridate (787 mg, 5.03 mmol, 629 uL, 1.50 eq) and pyridine (795 mg, 10.1 mmol, 811 uL, 3.00 eq) in acetonitrile (5.00 mL) was stirred at 25° C. for 12 h. The mixture was concentrated to give crude product. The crude product was purified by reversed-phase column. The desired fraction was collected and lyophilized to give phenyl (3-(tert-butyl)phenyl)carbamate. ¹H NMR (400 MHz, DMSO-d₆) δ=10.13 (br s, 1H), 7.59 (br s, 1H), 7.47-7.40 (m, 2H), 7.33 (br d, J=8.1 Hz, 1H), 7.29-7.19 (m, 4H), 7.09 (br d, J=7.6 Hz, 1H), 1.27 (s, 9H).

Step 2: To a mixture of phenyl (3-(tert-butyl)phenyl)carbamate (88.4 mg, 328 umol, 1.20 eq) and 3-(4-fluoro-6-(hydroxymethyl)-1-oxoisoindolin-2-yl)piperidine-2,6-dione XVIII (80.0 mg, 273 umol, 1.00 eq) in dimethyl formamide (1.00 mL) was added sodium hydride (16.4 mg, 410 umol, 60% purity, 1.50 eq) in one portion at 0° C. The mixture was stirred at 15° C. for 1 h. The mixture was quenched with 1 M hydrochloric acid (0.500 mL) and filtered. The filtrate was purified by prep-HPLC and lyophilized to give (2-(2,6-dioxopiperidin-3-yl)-7-fluoro-3-oxoisoindolin-5-yl)methyl (3-(tert-butyl) phenyl)carbamate. ¹H NMR (400 MHz, DMSO-d₆) δ=11.01 (s, 1H), 9.75 (br s, 1H), 7.68 (s, 1H), 7.63-7.47 (m, 2H), 7.31 (br d, J=7.9 Hz, 1H), 7.20 (t, J=7.9 Hz, 1H), 7.04 (d, J=7.8 Hz, 1H), 5.27 (s, 2H), 5.13 (dd, J=5.1, 13.3 Hz, 1H), 4.68-4.54 (m, 1H), 4.45-4.35 (m, 1H), 2.96-2.86 (m, 1H), 2.62 (br d, J=2.0 Hz, 1H), 2.44 (br dd, J=4.4, 13.1 Hz, 1H), 2.06-1.96 (m, 1H), 1.25 (s, 9H). MS (ESI) m/z 468.2 [M+H]⁺

Compound 26: Step 1: To a mixture of 1-(difluoromethoxy)-3-nitrobenzene (2.00 g, 10.6 mmol, 1.00 eq) in methanol (15.0 mL) and water (5.00 mL) was added iron powder (2.95 g, 52.9 mmol, 5.00 eq) and ammonium chloride (4.53 g, 84.6 mmol, 8.00 eq). The mixture was stirred at 80° C. for 2 h. The reaction solution was filtered to remove the iron powder.

The aqueous phase was extracted with ethyl acetate and the combined organic phase was washed with brine, dried with anhydrous sodium sulfate, filtered and concentrated to give 3-(difluoromethoxy)aniline. ¹H NMR (400 MHz, DMSO-d₆) δ=7.08 (t, J=74.4 Hz, 1H), 7.02 (t, J=8.1 Hz, 1H), 6.42 (td, J=1.1, 7.1 Hz, 1H), 6.33 (t, J=2.1 Hz, 1H), 6.26 (dd, J=2.0, 8.0 Hz, 1H).

Step 2: To a mixture of 3-(difluoromethoxy)aniline (1.00 g, 6.28 mmol, 1.00 eq) and pyridine (2.49 g, 31.4 mmol, 2.54 mL, 5.00 eq) in acetonitrile (5.00 mL) was added phenyl carbonochloridate (1.18 g, 7.54 mmol, 944 uL, 1.20 eq). The mixture was stirred at 25° C. for 3 h. The mixture was concentrated under reduced pressure. The crude product was purified by column chromatography on silica gel to give phenyl (3-(difluoromethoxy)phenyl)carbamate.

Step 3: To a solution of 3-(4-fluoro-6-(hydroxymethyl)-1-oxoisoindolin-2-yl)piperidine-2,6-dione XVIII (80.0 mg, 273 umol, 1.00 eq) in dimethyl formamide (2.00 mL) was added phenyl (3-(difluoromethoxy)phenyl)carbamate (91.7 mg, 328 umol, 1.20 eq) and sodium hydride (21.9 mg, 547 umol, 60% purity, 2.00 eq) at 0° C. The mixture was stirred at 0° C. for 0.5 h. The reaction mixture was quenched with formic acid (1.00 ml) to give a solution. The solution was purified by prep-HPLC and lyophilized to give (2-(2,6-dioxopiperidin-3-yl)-7-fluoro-3-oxoisoindolin-5-yl)methyl(3-(difluoromethoxy)phenyl)carbamate. ¹H NMR (400 MHz, DMSO-d₆) δ=11.02 (s, 1H), 10.07 (s, 1H), 7.69 (s, 1H), 7.58 (d, J=9.9 Hz, 1H), 7.40-7.01 (t, 1H), 7.37-7.28 (m, 2H), 7.19 (br d, J=74.4 Hz, 1H), 6.82 (s, 1H), 5.30 (s, 2H), 5.14 (dd, J=5.1, 13.3 Hz, 1H), 4.64-4.55 (m, 1H), 4.46-4.37 (m, 1H), 2.98-2.85 (m, 1H), 2.61 (br d, J=17.2 Hz, 1H), 2.44 (br dd, J=4.5, 12.9 Hz, 1H), 2.06-1.98 (m, 1H). MS (ESI) m/z 478.1 [M+H]⁺

Compound 27: Step 1: To a solution of 3-chloro-5-methylaniline (1.00 g, 7.06 mmol, 1.00 eq) in acetonitrile (5.00 mL) was added pyridine (2.79 g, 35.3 mmol, 2.85 mL, 5.00 eq) and phenyl carbonochloridate (1.66 g, 10.6 mmol, 1.33 mL, 1.50 eq) at 0° C. The mixture was stirred at 0° C. for 2 h. The mixture was filtered to give a filtrate which was concentrated and purified by reversed phase column chromatography. The desired fraction was collected and concentrated to give phenyl (3-chloro-5-methylphenyl)carbamate. ¹H NMR (400 MHz, DMSO-d₆) δ=10.38 (br s, 1H), 7.47-7.41 (m, 3H), 7.31-7.20 (m, 4H), 6.96 (s, 1H), 2.28 (s, 3H). MS (ESI) m/z 262.0 [M+H]⁺

Step 2: To a solution of 3-(4-fluoro-6-(hydroxymethyl)-1-oxoisoindolin-2-yl)piperidine-2,6-dione XVIII (80.0 mg, 274 umol, 1.00 eq) in dimethyl formamide (2.00 mL) was added phenyl (3-chloro-5-methylphenyl)carbamate (86.0 mg, 328 umol, 1.20 eq) and sodium hydride (21.9 mg, 547 umol, 60% purity, 2.00 eq) at 0° C. The mixture was stirred at 25° C. for 1 h. The reaction mixture was quenched with hydrochloric acid (1 M, 1.00 ml) to give a solution. The solution was purified by Prep-HPLC and lyophilized to give (2-(2,6-dioxopiperidin-3-yl)-7-fluoro-3-oxoisoindolin-5-yl)methyl(3-chloro-5-methyl phenyl)carbamate. ¹H NMR (400 MHz, DMSO-d₆) δ=11.02 (br s, 1H), 10.01 (s, 1H), 7.68 (s, 1H), 7.58 (d, J=9.6 Hz, 1H), 7.42 (s, 1H), 7.22 (s, 1H), 6.92 (s, 1H), 5.28 (s, 2H), 5.14 (dd, J=5.2, 13.2 Hz, 1H), 4.64-4.56 (m, 1H), 4.48-4.36 (m, 1H), 3.02-2.86 (m, 1H), 2.64-2.58 (m, 1H), 2.44 (br d, J=9.2 Hz, 1H), 2.28 (s, 3H), 2.06-1.98 (m, 1H). MS (ESI) m/z 460.1 [M+H]⁺

Compound 28: Step 1: To a mixture of 3,5-dichloroaniline (2.00 g, 12.3 mmol, 1.00 eq) and pyridine (2.93 g, 37.0 mmol, 2.99 mL, 3.00 eq) in acetonitrile (20.0 mL) was added phenyl carbonochloridate (2.51 g, 16.0 mmol, 2.01 mL, 1.30 eq) dropwise. The mixture was stirred at 15° C. for 12 h. The mixture was concentrated to give crude product. The crude product was purified by reversed-phase column. The desired fraction was collected and lyophilized to give phenyl (3,5-dichlorophenyl)carbamate. ¹H NMR (400 MHz, DMSO-d₆) δ=10.64 (br s, 1H), 7.56 (d, J=1.8 Hz, 2H), 7.47-7.42 (m, 3H), 7.32-7.29 (m, 1H), 7.27-7.25 (m, 2H).

Step 2: To a mixture of phenyl (3,5-dichlorophenyl)carbamate (92.6 mg, 328 umol, 1.20 eq) and 3-(4-fluoro-6-(hydroxymethyl)-1-oxoisoindolin-2-yl)piperidine-2,6-dione XVIII (80.0 mg, 273 umol, 1.00 eq) in dimethyl formamide (1.00 mL) was added sodium hydride (16.4 mg, 410 umol, 60% purity, 1.50 eq) in one portion at 0° C. The mixture was stirred at 15° C. for 1 h. The mixture was quenched with 1 M hydrochloric acid (0.500 mL) and filtered. The filtrate was purified by prep-HPLC and lyophilized to give (2-(2,6-dioxopiperidin-3-yl)-7-fluoro-3-oxoisoindolin-5-yl)methyl(3,5-dichlorophenyl)carbamate. ¹H NMR (400 MHz, DMSO-d₆) δ=11.01 (s, 1H), 10.27 (s, 1H), 7.69 (s, 1H), 7.57 (d, J=9.7 Hz, 1H), 7.53 (d, J=1.8 Hz, 2H), 7.23 (t, J=1.8 Hz, 1H), 5.30 (s, 2H), 5.13 (dd, J=5.0, 13.3 Hz, 1H), 4.63-4.54 (m, 1H), 4.46-4.36 (m, 1H), 2.99-2.84 (m, 1H), 2.60 (br d, J=17.2 Hz, 1H), 2.46-2.35 (m, 1H), 2.08-1.97 (m, 1H). MS (ESI) m/z 480.0 [M+H]⁺

Compound 29: Step 1: To a solution of 4-bromo-3-fluoroaniline (2.00 g, 10.5 mmol, 1.00 eq) and (2-fluorophenyl)boronic acid (2.21 g, 15.8 mmol, 1.50 eq) in dioxane (20.0 mL) and water (5.00 mL) was added [1,1-bis(diphenylphosphino)ferrocene]dichloropalladium(II) (770 mg, 1.05 mmol, 0.100 eq) and potassium phosphate (6.70 g, 31.6 mmol, 3.00 eq) in portions. The mixture was stirred at 80° C. for 12 h under nitrogen. The mixture was diluted with water (80.0 mL) and extracted with ethyl acetate (3×60.0 mL). The combined organic layer were washed with saturated brine (20.0 mL), dried with anhydrous sodium sulfate, filtered and concentrated in vacuum. The residue was purified by column chromatography on silica gel to give 2,2′-difluoro-[1,1′-biphenyl]-4-amine. ¹H NMR (400 MHz, DMSO-d₆) δ=7.39-7.31 (m, 2H), 7.27-7.19 (m, 2H), 7.05 (t, J=8.5 Hz, 1H), 6.47 (dd, J=2.2, 8.3 Hz, 1H), 6.41 (dd, J=2.1, 13.0 Hz, 1H), 5.59 (s, 2H).

Step 2: To a mixture of 2,2′-difluoro-[1,1′-biphenyl]-4-amine (500 mg, 2.44 mmol, 1.00 eq) and pyridine (578 mg, 7.31 mmol, 590 uL, 3.00 eq) in acetonitrile (5.00 mL) was added phenyl carbonochloridate (496 mg, 3.17 mmol, 397 uL, 1.30 eq) dropwise at 0° C. The mixture was stirred at 15° C. for 2 h. The mixture was concentrated to give crude product. The crude product was purified by reversed-phase column. The desired fraction was collected and lyophilized to give phenyl (2,2′-difluoro-[1,1′-biphenyl]-4-yl)carbamate. ¹H NMR (400 MHz, DMSO-d₆) δ=10.61 (br s, 1H), 7.54 (br d, J=12.3 Hz, 1H), 7.50-7.37 (m, 6H), 7.36-7.21 (m, 5H).

Step 3: To a mixture of phenyl (2,2′-difluoro-[1,1′-biphenyl]-4-yl)carbamate (106 mg, 328 umol, 1.20 eq) and 3-(4-fluoro-6-(hydroxymethyl)-1-oxoisoindolin-2-yl)piperidine-2,6-dione XVIII (80.0 mg, 273 umol, 1.00 eq) in dimethyl formamide (1.00 mL) was added sodium hydride (16.4 mg, 410. umol, 60% purity, 1.50 eq) in one portion at 0° C. The mixture was stirred at 15° C. for 1 h. The mixture was quenched with 1 M hydrochloric acid (0.500 mL) and filtered. The crude product was triturated with dimethyl formamide (1.00 mL) and acetonitrile (1.00 mL), then filtered. The filter cake was added water (20.0 mL) and lyophilized to give (2-(2,6-dioxopiperidin-3-yl)-7-fluoro-3-oxoisoindolin-5-yl)methyl(2,2′-difluoro-[1,1′-biphenyl]-4-yl) carbamate. ¹H NMR (400 MHz, DMSO-d₆) δ=11.02 (br s, 1H), 10.23 (br s, 1H), 7.71 (br s, 1H), 7.59 (br d, J=8.3 Hz, 1H), 7.51 (br d, J=13.4 Hz, 1H), 7.47-7.40 (m, 2H), 7.40-7.25 (m, 4H), 5.32 (br s, 2H), 5.14 (br d, J=10.0 Hz, 1H), 4.66-4.52 (m, 1H), 4.48-4.31 (m, 1H), 2.96-2.84 (m, 1H), 2.61 (br d, J=18.1 Hz, 1H), 2.43 (br s, 1H), 2.02 (br s, 1H). MS (ESI) m/z 524.2 [M+H]⁺

Compound 30: Step 1: To a solution of 4-bromo-3,5-difluoroaniline (2.00 g, 9.62 mmol, 1.00 eq) and phenylboronic acid (1.76 g, 14.4 mmol, 1.50 eq) in dioxane (20.0 mL) and water (5.00 mL) was added [1,1-bis(diphenylphosphino)ferrocene]dichloropalladium(II) (703 mg, 961 umol, 0.100 eq) and potassium phosphate (6.12 g, 28.8 mmol, 3.00 eq) in portions. The mixture was stirred at 80° C. for 12 h under nitrogen. The mixture was diluted with water (80.0 mL) and extracted with ethyl acetate (3×60.0 mL). The combined organic layers were washed with saturated brine (20.0 mL), dried with anhydrous sodium sulfate, filtered and concentrated in vacuum. The residue was purified by column chromatography on silica gel to give 2,6-difluoro-[1,1′-biphenyl]-4-amine. ¹H NMR (400 MHz, DMSO-d₆) δ=7.43-7.39 (m, 2H), 7.34-7.31 (m, 3H), 6.31 (s, 1H), 6.28 (s, 1H), 5.83 (br s, 2H).

Step 2: To a mixture of 2,6-difluoro-[1,1′-biphenyl]-4-amine (1.00 g, 4.87 mmol, 1.00 eq) and pyridine (1.16 g, 14.6 mmol, 1.18 mL, 3.00 eq) in acetonitrile (10.0 mL) was added phenyl carbonochloridate (991 mg, 6.34 mmol, 793 uL, 1.30 eq) dropwise. The mixture was stirred at 15° C. for 2 h. The mixture was concentrated to give crude product. The crude product was purified by reversed-phase column. The desired fraction was collected and lyophilized to give phenyl (2,6-difluoro-[1,1′-biphenyl]-4-yl)carbamate. ¹H NMR (400 MHz, DMSO-d₆) δ=10.75 (br s, 1H), 7.49-7.44 (m, 4H), 7.44-7.41 (m, 4H), 7.35 (s, 1H), 7.33 (s, 1H), 7.29-7.27 (m, 1H), 7.27 (s, 1H).

Step 3: To a mixture of phenyl (2,6-difluoro-[1,1′-biphenyl]-4-yl)carbamate (106 mg, 328 umol, 1.20 eq) and 3-(4-fluoro-6-(hydroxymethyl)-1-oxoisoindolin-2-yl)piperidine-2,6-dione XVIII (80.0 mg, 273 umol, 1.00 eq) in dimethyl formamide (1.00 mL) was added sodium hydride (16.4 mg, 410 umol, 60% purity, 1.50 eq) in one portion at 0° C. The mixture was stirred at 15° C. for 1 h. The mixture was quenched with 1 M hydrochloric acid (0.500 mL) and filtered. The crude product was triturated with dimethyl formamide (1.00 mL) and acetonitrile (1.00 mL) and filtered. The filter cake was added water (20.0 mL) and lyophilized to give (2-(2,6-dioxopiperidin-3-yl)-7-fluoro-3-oxoisoindolin-5-yl)methyl(2,6-difluoro-[1,1′-biphenyl]-4-yl) carbamate. ¹H NMR (400 MHz, DMSO-d₆) δ=11.02 (br s, 1H), 10.37 (br s, 1H), 7.70 (br s, 1H), 7.59 (br d, J=9.0 Hz, 1H), 7.49-7.39 (m, 5H), 7.30 (br d, J=9.4 Hz, 2H), 5.32 (br s, 2H), 5.14 (br d, J=9.2 Hz, 1H), 4.59 (br d, J=17.6 Hz, 1H), 4.49-4.35 (m, 1H), 2.90 (br d, J=13.6 Hz, 1H), 2.61 (br d, J=17.1 Hz, 1H), 2.43 (br s, 1H), 2.03 (br d, J=2.9 Hz, 1H). MS (ESI) m/z 524.2 [M+H]⁺

Compound 31: Step 1: To a solution of 3,5-dimethylaniline (500 mg, 4.13 mmol, 514 uL, 1.00 eq) in acetonitrile (10.00 mL) was added pyridine (1.63 g, 20.6 mmol, 1.67 mL, 5.00 eq) and phenyl carbonochloridate (1.29 g, 8.25 mmol, 1.03 mL, 2.00 eq) at 0° C. in portions. The mixture was stirred at 25° C. for 1 h. The mixture was concentrated under reduced pressure to give a residue which was purified by column chromatography on silica gel to give phenyl (3,5-dimethylphenyl) carbamate. ¹H NMR (400 MHz, DMSO-d₆) δ=10.1 (br s, 1H), 7.45-7.38 (m, 2H), 7.28-7.18 (m, 3H), 7.13 (s, 2H), 6.69 (s, 1H), 2.23 (s, 6H).

Step 2: To a solution of phenyl (3,5-dimethylphenyl)carbamate (72.6 mg, 301.1 umol, 1.10 eq) and 3-(4-fluoro-6-(hydroxymethyl)-1-oxoisoindolin-2-yl)piperidine-2,6-dione (80.0 mg, 273 umol, 1.00 eq) in dimethyl formamide (1.50 mL) was added sodium hydride (21.9 mg, 547 umol, 60% purity, 2.00 eq) at 0° C. The mixture was stirred at 25° C. for 1 h. The pH of the mixture was adjusted to around 6 by adding hydrochloric acid (1 M). The mixture was extracted with ethyl acetate (3×10.0 mL). The combined organic layers were dried over anhydrous sodium sulfate, filtered and concentrated under reduced pressure to give a residue. The residue was purified by prep-HPLC and lyophilized to afford (2-(2,6-dioxopiperidin-3-yl)-7-fluoro-3-oxoisoindolin-5-yl)methyl (3,5-dimethylphenyl)carbamate. ¹H NMR (400 MHz, DMSO-d₆) δ=11.01 (br s, 1H), 9.69 (br s, 1H), 7.67 (s, 1H), 7.55 (d, J=9.4 Hz, 1H), 7.09 (s, 2H), 6.65 (s, 1H), 5.26 (s, 2H), 5.13 (dd, J=5.1, 13.2 Hz, 1H), 4.61-4.55 (m, 1H), 4.44-4.37 (m, 1H), 2.96-2.86 (m, 1H), 2.62 (br d, J=2.2 Hz, 1H), 2.42-2.35 (m, 1H), 2.21 (s, 6H), 2.04-1.98 (m, 1H). MS (ESI) m/z. 440.0 [M+H]⁺

Compound 32: Step 1: To a solution of 3-chloro-4-fluoroaniline (1.00 g, 6.87 mmol, 1.00 eq) in acetonitrile (10.0 mL) was added pyridine (2.72 g, 34.4 mmol, 2.77 mL, 5.00 eq) and phenyl carbonochloridate (2.15 g, 13.7 mmol, 1.72 mL, 2.00 eq) in portions at 0° C. The mixture was stirred at 25° C. for 0.5 h. The mixture was concentrated under reduced pressure to give a residue which was purified by column chromatography on silica gel to give phenyl (3-chloro-4-fluorophenyl)carbamate. ¹H NMR (400 MHz, DMSO-d₆) δ=10.46 (br s, 1H), 7.46-7.38 (m, 4H), 7.29-7.21 (m, 3H). MS (ESI) m/z 266.1 [M+H]⁺

Step 2: To a solution of phenyl (3-chloro-4-fluorophenyl)carbamate (80.0 mg, 301 umol, 1.10 eq) and 3-(4-fluoro-6-(hydroxymethyl)-1-oxoisoindolin-2-yl)piperidine-2,6-dione XVIII (80.0 mg, 273 umol, 1.00 eq) in dimethyl formamide (1.50 mL) was added sodium hydride (21.9 mg, 547 umol, 60% purity, 2.00 eq) at 0° C. The mixture was stirred at 25° C. for 1 h. The pH of the mixture was adjusted to around 6 by adding hydrochloric acid (1M). The mixture was extracted with ethyl acetate (3×10.0 mL). The combined organic layers were dried over anhydrous sodium sulfate, filtered and concentrated under reduced pressure to give a residue. The residue was purified by prep-HPLC and lyophilized to afford (2-(2,6-dioxopiperidin-3-yl)-7-fluoro-3-oxoisoindolin-5-yl)methyl (3-chloro-4-fluorophenyl)carbamate. ¹H NMR (400 MHz, DMSO-d₆) δ=11.02 (s, 1H), 10.09 (br s, 1H), 7.74-7.65 (m, 2H), 7.57 (d, J=9.8 Hz, 1H), 7.43-7.32 (m, 2H), 5.29 (s, 2H), 5.13 (dd, J=5.1, 13.3 Hz, 1H), 4.62-4.55 (m, 1H), 4.44-4.37 (m, 1H), 2.97-2.86 (m, 1H), 2.60 (br d, J=17.5 Hz, 1H), 2.44 (br dd, J=4.4, 13.1 Hz, 1H), 2.05-1.98 (m, 1H). MS (ESI) m/z. 463.9 [M+H]⁺

Compound 33: Step 1: To a mixture of 3-chloro-5-fluoroaniline (500 mg, 3.43 mmol, 1.00 eq) in acetonitrile (5.00 mL) were added pyridine (272 mg, 3.43 mmol, 277 uL, 1.00 eq) and phenyl carbonochloridate (645 mg, 4.12 mmol, 516 uL, 1.20 eq) at 0° C. The mixture was stirred at 20° C. for 2 h. The reaction mixture was concentrated under reduced pressure to give a residue. The residue was purified by column chromatography to afford phenyl (3-chloro-5-fluorophenyl)carbamate. ¹H NMR (400 MHz, CDCl₃) δ=7.49-7.35 (m, 2H), 7.31-7.21 (m, 3H), 7.21-7.16 (m, 2H), 7.13 (br s, 1H), 6.84 (td, J=2.0, 8.2 Hz, 1H). MS (ESI) m/z 265.9 [M+H]⁺

Step 2: To a solution of phenyl N-(3-chloro-5-fluoro-phenyl)carbamate (100 mg, 376 umol, 1.10 eq) and 3-(4-fluoro-6-(hydroxymethyl)-1-oxoisoindolin-2-yl)piperidine-2,6-dione XVIII (0.10 g, 342 umol, 1.00 eq) in dimethylformamide (1.50 mL) was added sodium hydride (16.4 mg, 684 umol, 2.00 eq, 60%) at 0° C. Then the mixture was stirred at 25° C. for 1 h. The reaction mixture was quenched by addition hydrochloric acid (1M, 2.00 mL) and purified by prep-HPLC to afford [2-(2,6-dioxo-3-piperidyl)-7-fluoro-3-oxo-isoindolin-5-yl]methyl N-(3-chloro-5-fluoro-phenyl)carbamate. ¹H NMR (400 MHz, DMSO-d₆) δ=11.02 (br s, 1H), 10.34 (s, 1H), 7.69 (s, 1H), 7.58 (d, J=9.6 Hz, 1H), 7.39 (s, 1H), 7.34 (td, J=2.0, 11.2 Hz, 1H), 7.06 (td, J=2.2, 8.6 Hz, 1H), 5.31 (s, 2H), 5.14 (dd, J=5.2, 13.4 Hz, 1H), 4.66-4.54 (m, 1H), 4.47-4.36 (m, 1H), 2.98-2.87 (m, 1H), 2.65-2.57 (m, 1H), 2.44 (br dd, J=4.6, 13.2 Hz, 1H), 2.06-1.98 (m, 1H). MS (ESI) m/z 464.1 [M+H]⁺

Compound 34: Step 1: To a solution of 3-chloro-5-nitro-phenol (2.00 g, 11.5 mmol, 1.00 eq) in dimethyl formamide (20.0 mL) and water (3.00 mL) was added sodium 2-chloro-2,2-difluoroacetate (8.78 g, 57.6 mmol, 5.00 eq) and potassium carbonate (3.19 g, 23.0 mmol, 2.00 eq). The mixture was stirred at 100° C. for 12 h. The mixture was diluted with water/ethyl acetate (200 mL/200 mL). The organic layer was collected and concentrated to give a residue. The residue was purified by column chromatography on silica gel. The desired fraction was collected and concentrated to give 1-chloro-3-(difluoromethoxy)-5-nitrobenzene. ¹H NMR (400 MHz, DMSO-d₆) δ=8.17 (td, J=1.2, 2.1 Hz, 1H), 7.99 (t, J=2.0 Hz, 1H), 7.86 (s, 1H), 7.48 (t, J=72 Hz, 1H).

Step 2: To a solution of 1-chloro-3-(difluoromethoxy)-5-nitrobenzene (1.49 g, 6.66 mmol, 1.00 eq) in methanol (21.0 mL) and water (7.00 mL) was added iron power (1.86 g, 33.3 mmol, 5.00 eq) and ammonium chloride (2.85 g, 53.3 mmol, 8.00 eq). The mixture was stirred at 80° C. for 2 h. The reaction mixture was filtered to give filtrate that was concentrated to give a residue. The residue was diluted with water/ethyl acetate (100 ml/100 ml). The organic layer was collected and concentrated to give 3-chloro-5-(difluoromethoxy) aniline. ¹H NMR (400 MHz, DMSO-d₆) δ=7.15 (t, J=72 Hz, 1H), 6.45 (t, J=1.6 Hz, 1H), 6.33-6.24 (m, 2H), 5.71 (s, 2H).

Step 3: To a solution of 3-chloro-5-(difluoromethoxy)aniline (0.300 g, 1.55 mmol, 1.00 eq) in acetonitrile (5.00 mL) was added pyridine (368 mg, 4.65 mmol, 375 uL, 3.00 eq) and phenyl carbonochloridate (300 mg, 1.92 mmol, 240 uL, 1.24 eq). The mixture was stirred at 25° C. for 1 h. The mixture was concentrated and purified by reversed phase HPLC. The desired fraction was collected and concentrated to give phenyl (3-chloro-5-(difluoromethoxy)phenyl)carbamate. MS (ESI) m/z 313.9 [M+H]⁺

Step 4: To a solution of 3-(4-fluoro-6-(hydroxymethyl)-1-oxoisoindolin-2-yl)piperidine-2,6-dione XVIII (80.0 mg, 274 umol, 1.00 eq) in dimethyl formamide (2.00 mL) was added phenyl (3-chloro-5-(difluoromethoxy)phenyl) carbamate (103 mg, 328 umol, 1.20 eq) and sodium hydride (21.9 mg, 547 umol, 60% purity, 2.00 eq) at 0° C. The mixture was stirred at 0° C. for 1 h. The mixture was adjusted pH=5 with hydrochloric acid (1 M, 1.00 mL). The mixture was diluted with water/ethyl acetate (10.0 ml/10.0 ml). The organic layer was collected and concentrated to give a residue. The residue was purified by prep-HPLC. The desired fraction was collected and lyophilized to give (2-(2,6-dioxopiperidin-3-yl)-7-fluoro-3-oxoisoindolin-5-yl)methyl(3-chloro-5-(difluoromethoxy)phenyl)carbamate. ¹H NMR (400 MHz, DMSO-d₆) δ=11.19-10.85 (m, 1H), 10.26 (s, 1H), 7.68 (s, 1H), 7.57 (d, J=9.3 Hz, 1H), 7.46-7.40 (m, 1H), 7.30 (s, 1H), 7.26 (t, J=73.6 Hz 1H), 7.02-6.91 (m, 1H), 5.30 (s, 2H), 5.13 (dd, J=5.1, 13.4 Hz, 1H), 4.63-4.55 (m, 1H), 4.46-4.37 (m, 1H), 2.98-2.85 (m, 1H), 2.62 (br s, 1H), 2.43-2.38 (m, 1H), 2.05-1.97 (m, 1H). MS (ESI) m/z 512.1 [M+H]⁺

Compound 35: Step 1: To a solution of 2-methyl-5-nitrophenol (5.00 g, 32.7 mmol, 1.00 eq) and sodium 2-chloro-2,2-difluoroacetate (12.4 g, 81.6 mmol, 2.50 eq) in dimethyl formamide (50.0 mL) was added cesium carbonate (21.3 g, 65.3 mmol, 2.00 eq) in portions. The mixture was stirred at 100° C. for 2 h. The mixture was diluted with water (800 mL) and extracted with ethyl acetate (3×100 mL). The combined organic layer was washed with brine (80.0 mL) and dried over anhydrous sodium sulfate, filtered and concentrated to give crude product. The crude product was purified by silica gel chromatography to give 2-(difluoromethoxy)-1-methyl-4-nitrobenzene. ¹H NMR (400 MHz, DMSO-d₆) δ=8.05 (dd, J=2.3, 8.4 Hz, 1H), 7.99 (d, J=2.1 Hz, 1H), 7.62 (d, J=8.4 Hz, 1H), 7.23 (t, J=73.2 Hz, 1H), 2.35 (s, 3H).

Step 2: To a solution of 2-(difluoromethoxy)-1-methyl-4-nitrobenzene (4.85 g, 23.8 mmol, 1.00 eq) and ammonium chloride (6.39 g, 119 mmol, 5.00 eq) in methanol (40.0 mL) and water (40.0 mL) was added iron powder (4.00 g, 71.6 mmol, 3.00 eq) in portions then stirred at 80° C. for 2 h. The mixture was filtered and the filtrate was concentrated to give crude product. The crude product was diluted with water (100 mL) and extracted with ethyl acetate (3×50.0 mL). The combined organic layer was washed with brine (20 mL) and dried over anhydrous sodium sulfate, filtered and concentrated to give 3-(difluoromethoxy)-4-methylaniline. ¹H NMR (400 MHz, DMSO-d₆) δ=7.00 (t, J=74.8 Hz, 1H), 6.90 (d, J=8.8 Hz, 1H), 6.36 (s, 1H), 5.12 (s, 2H), 2.05 (s, 3H). MS (ESI) m/z 174.0 [M+H]⁺

Step 3: To a solution of 3-(difluoromethoxy)-4-methylaniline (1.00 g, 5.78 mmol, 1.00 eq) and pyridine (1.37 g, 17.3 mmol, 1.40 mL, 3.00 eq) in acetonitrile (10.0 mL) was added phenyl carbonochloridate (1.36 g, 8.66 mmol, 1.09 mL, 1.50 eq) dropwise then stirred at 25° C. for 12 h. The mixture was diluted with water (150 mL) and extracted with ethyl acetate (3×50.0 mL). The combined organic layer was washed with brine (20.0 mL) and dried over anhydrous sodium sulfate, filtered and concentrated to give crude product. The crude product was purified by silica gel chromatography to give phenyl (3-(difluoromethoxy)-4-methylphenyl)carbamate. ¹H NMR (400 MHz, CDCl₃) δ=7.48-7.34 (m, 3H), 7.30-7.25 (m, 1H), 7.25-7.15 (m, 3H), 7.14-7.01 (m, 2H), 6.52 (t, J=74.0 Hz, 1H), 2.27 (s, 3H). MS (ESI) m/z 294.0 [M+H]⁺

Step 4: To a solution of phenyl (3-(difluoromethoxy)-4-methylphenyl)carbamate (90.0 mg, 307 umol, 1.00 eq) in dimethyl formamide (500 uL) was added 3-(4-fluoro-6-(hydroxymethyl)-1-oxoisoindolin-2-yl)piperidine-2,6-dione XVIII (94.0 mg, 322 umol, 1.05 eq) and sodium hydride (24.6 mg, 614 umol, 60% purity, 2.00 eq). The mixture was stirred at 25° C. for 1 h. The pH of the mixture was adjusted to 7 with hydrochloric acid (1 M), then the mixture was diluted with dimethyl formamide (1.00 mL). The mixture was purified by Prep-HPLC to afford (2-(2,6-dioxopiperidin-3-yl)-7-fluoro-3-oxoisoindolin-5-yl) methyl (3-(difluoromethoxy)-4-methylphenyl)carbamate. ¹H NMR (400 MHz, DMSO-d) δ=11.00 (br s, 1H), 9.96 (br s, 1H), 7.67 (s, 1H), 7.56 (d, J=9.6 Hz, 1H), 7.40 (s, 1H), 7.20 (s, 2H), 7.09 (t, J=72 Hz, 1H), 5.28 (s, 2H), 5.13 (dd, J=5.2, 13.2 Hz, 1H), 4.63-4.53 (m, 1H), 4.46-4.36 (m, 1H), 2.91 (ddd, J=5.6, 13.6, 17.6 Hz, 1H), 2.60 (td, J=2.0, 15.2 Hz, 1H), 2.47-2.37 (m, 1H), 2.15 (s, 3H), 2.07-1.94 (m, 1H). MS (ESI) m/z 492.2 [M+H]⁺

Compound 36: Step 1: To a solution of 3-(6-(hydroxymethyl)-4-methoxy-1-oxoisoindolin-2-yl)piperidine-2,6-dione XLI (80.0 mg, 262 umol, 1.00 eq) in dimethyl formamide (2.00 mL) was added phenyl (6-(2-fluorophenyl)pyridin-3-yl) carbamate (see Compound 18) (97.2 mg, 315 umol, 1.20 eq) and sodium hydride (21.0 mg, 525 umol, 60% purity, 2.00 eq) at 0° C. The mixture was stirred at 0° C. for 0.5 h. The reaction mixture was quenched with hydrochloric acid (1M, 1.00 ml) to give a solution. The solution was purified by prep-HPLC and lyophilized. The crude product was dissolved in dimethyl formamide (2.00 mL), purified again by prep-HPLC and lyophilized to give (2-(2,6-dioxopiperidin-3-yl)-7-methoxy-3-oxoisoindolin-5-yl)methyl (6-(2-fluorophenyl)pyridin-3-yl)carbamate. ¹H NMR (400 MHz, DMSO-d₆) δ=10.98 (br s, 1H), 10.22 (br s, 1H), 8.79 (d, J=2.4 Hz, 1H), 8.04 (dd, J=2.1, 8.7 Hz, 1H), 7.92 (dt, J=1.5, 7.9 Hz, 1H), 7.77 (dd, J=1.7, 8.6 Hz, 1H), 7.49-7.42 (m, 2H), 7.38-7.28 (m, 3H), 5.32 (s, 2H), 5.11 (dd, J=5.1, 13.3 Hz, 1H), 4.46-4.35 (m, 1H), 4.29-4.20 (m, 1H), 3.92 (s, 3H), 2.97-2.86 (m, 1H), 2.63-2.57 (m, 1H), 2.44 (br dd, J=4.3, 13.0 Hz, 1H), 2.03-1.96 (m, 1H). MS (ESI) m/z 519.1 [M+H]⁺

Compound 37: To a solution of 3-(6-(hydroxymethyl)-4-methoxy-1-oxoisoindolin-2-yl)piperidine-2,6-dione XLI (100 mg, 329 umol, 1.00 eq) in dimethyl formamide (2.00 mL) was added phenyl (3,3-dimethyl-2,3-dihydrobenzofuran-6-yl)carbamate (see Compound 17) (140 mg, 493 umol, 1.50 eq) and sodium hydride (26.3 mg, 657 umol, 60% purity, 2.00 eq) at 0° C. The mixture was stirred at 25° C. for 1 h. The reaction mixture was quenched with hydrochloric acid (1 M, 1.00 ml) to give a solution. The solution was purified by Prep-HPLC and lyophilized to give (2-(2,6-dioxopiperidin-3-yl)-7-methoxy-3-oxoisoindolin-5-yl) methyl(3,3-dimethyl-2,3-dihydrobenzofuran-6-yl)carbamate. ¹H NMR (400 MHz, DMSO-d₆) δ=10.98 (s, 1H), 9.74 (br s, 1H), 7.38 (s, 1H), 7.32 (s, 1H), 7.08 (d, J=8.0 Hz, 1H), 6.98 (s, 1H), 6.94 (br d, J=8.0 Hz, 1H), 5.26 (s, 2H), 5.12 (dd, J=5.2, 13.2 Hz, 1H), 4.44-4.36 (m, 1H), 4.24 (d, J=17.6 Hz, 1H), 4.18 (s, 2H), 3.92 (s, 3H), 3.02-2.86 (m, 1H), 2.62-2.58 (m, 1H), 2.44-2.38 (m, 1H), 2.04-1.96 (m, 1H), 1.26 (s, 6H). MS (ESI) m/z 494.1 [M+H]⁺

Compound 38: To a solution of 3-(6-(hydroxymethyl)-4-methoxy-1-oxoisoindolin-2-yl)piperidine-2,6-dione XLI (80.0 mg, 263 umol, 1.00 eq) in dimethyl formamide (2.00 mL) was added phenyl (6-(2-methylpiperidin-1-yl)pyridin-3-yl) carbamate (see Compound 16) (98.2 mg, 316 umol, 1.20 eq) and sodium hydride (21.0 mg, 526 umol, 60/purity, 2.00 eq) at 0° C. The mixture was stirred at 0° C. for 1 h. The mixture was adjusted pH=5 with hydrochloric acid (1 M, 1 mL). The mixture was purified by reversed phase HPLC (0.1% formic acid). The desired fraction was collected and concentrated to give a residue. The residue was purified by prep-HPLC. The desired fraction was collected and concentrated to give a residue. The residue was further purified by prep-HPLC. The desired fraction was collected and lyophilized to give (2-(2,6-dioxopiperidin-3-yl)-7-methoxy-3-oxoisoindolin-5-yl)methyl(6-(2-methylpiperidin-1-yl)pyridin-3-yl)carbamate. ¹H NMR (400 MHz, DMSO-d₆) δ=10.97 (br s, 1H), 9.51 (br s, 1H), 8.15 (br s, 1H), 7.60 (br d, J=7.3 Hz, 1H), 7.38 (s, 1H), 7.30 (s, 1H), 6.72 (d, J=9.2 Hz, 1H), 5.23 (s, 2H), 5.10 (dd, J=5.0, 13.3 Hz, 1H), 4.54 (br d, J=4.6 Hz, 1H), 4.42-4.34 (m, 1H), 4.26-4.18 (m, 1H), 3.97 (br d, J=12.6 Hz, 1H), 3.90 (s, 3H), 2.97-2.85 (m, 1H), 2.79 (dt, J=2.8, 12.8 Hz, 1H), 2.62-2.55 (m, 1H), 2.46-2.38 (m, 1H), 2.04-1.94 (m, 1H), 1.71-1.53 (m, 5H), 1.45-1.33 (m, 1H), 1.02 (d, J=6.7 Hz, 3H). MS (ESI) m/z 522.1 [M+H]⁺

Compound 39: Step 1: To a solution of phenyl (4-fluoro-3-(trifluoromethoxy)phenyl)carbamate (see Compound 14) (80.0 mg, 254 umol, 1.00 eq) in dimethyl formamide (500 uL) was added 3-(6-(hydroxymethyl)-4-methoxy-1-oxoisoindolin-2-yl) piperidine-2,6-dione (80.0 mg, 263 umol, 1.04 eq) and sodium hydride (20.3 mg, 508 umol, 60% purity, 2.00 eq). The mixture was stirred at 25° C. for 1 h. The pH of the mixture was adjusted to 7 with hydrochloric acid (1 M). The mixture was diluted with dimethyl formamide (1.00 mL) and purified by Prep-HPLC and the desired fraction was lyophilized to afford (2-(2,6-dioxopiperidin-3-yl)-7-methoxy-3-oxoisoindolin-5-yl)methyl(4-fluoro-3-(trifluoromethoxy)phenyl) carbamate. ¹H NMR (400 MHz, DMSO-d₆) δ=10.97 (br s, 1H), 10.16 (br s, 1H), 7.74 (br d, J=6.4 Hz, 1H), 7.49-7.41 (m, 2H), 7.39 (s, 1H), 7.32 (s, 1H), 5.27 (s, 2H), 5.10 (dd, J=5.2, 13.2 Hz, 1H), 4.44-4.33 (m, 1H), 4.28-4.16 (m, 1H), 3.91 (s, 3H), 2.98-2.83 (m, 1H), 2.63-2.54 (m, 1H), 2.46-2.37 (m, 1H), 2.04-1.92 (m, 1H). MS (ESI) m/z 526.0 [M+H]⁺

Compound 40: Step 1: To a solution of 3-(6-(hydroxymethyl)-4-methoxy-1-oxoisoindolin-2-yl)piperidine-2,6-dione XLI (80.0 mg, 263 umol, 1.00 eq) in dimethyl formamide (1.00 mL) was added phenyl (4-(1-methylcyclopropyl)phenyl) carbamate (see Compound 13) (70.3 mg, 263 umol, 1.00 eq) and sodium hydride (21.0 mg, 526 umol, 60% purity, 2.00 eq) at 0° C. The mixture was stirred at 25° C. for 1 h. The reaction was quenched with hydrochloric acid (1M, 2.00 mL). The mixture was extracted with water/ethyl acetate (20.0 mL/20.0 mL). The organic layer was collected and concentrated to give a residue. The residue was purified by prep-HPLC. The desired fraction was collected and lyophilized to give (2-(2,6-dioxopiperidin-3-yl)-7-methoxy-3-oxoisoindolin-5-yl)methyl (4-(1-methylcyclopropyl)phenyl)carbamate. ¹H NMR (400 MHz, DMSO-d₆) δ=11.09-10.83 (m, 1H), 9.71 (br s, 1H), 7.41-7.33 (m, 3H), 7.31 (s, 1H), 7.16-7.11 (m, 2H), 5.24 (s, 2H), 5.10 (dd, J=5.0, 13.3 Hz, 1H), 4.42-4.34 (m, 1H), 4.26-4.18 (m, 1H), 3.90 (s, 3H), 2.94-2.85 (m, 1H), 2.60 (br d, J=2.4 Hz, 1H), 2.46-2.41 (m, 1H), 2.02-1.93 (m, 1H), 1.34 (s, 3H), 0.79-0.74 (m, 2H), 0.71-0.67 (m, 2H). MS (ESI) m/z 478.2 [M+H]⁺

Compound 41: To a mixture of 3-(6-(hydroxymethyl)-4-methoxy-1-oxoisoindolin-2-yl)piperidine-2,6-dione XLI (80.0 mg, 263 umol, 1.00 eq) and phenyl (3-fluoro-5-(trifluoromethoxy)phenyl)carbamate (see Compound 12) (99.5 mg, 315 umol, 1.20 eq) in dimethyl formamide (2.00 mL) was added sodium hydride (21.0 mg, 526 umol, 60% purity, 2.00 eq) at 0° C. The reaction mixture was stirred at 25° C. for 1 h. The reaction mixture was added formic acid (1.00 mL) and filtered to give a filtrate. The filtrate was purified by Prep-HPLC and lyophilized to give (2-(2,6-dioxopiperidin-3-yl)-7-methoxy-3-oxoisoindolin-5-yl)methyl (3-fluoro-5-(trifluoromethoxy) phenyl) carbamate. ¹H NMR (400 MHz, DMSO-d₆) δ=10.97 (br s, 1H), 10.37 (s, 1H), 7.40 (s, 1H), 7.40-7.34 (m, 2H), 7.33 (s, 1H), 7.00 (br d, J=9.2 Hz, 1H), 5.30 (s, 2H), 5.11 (dd, J=5.2, 13.2 Hz, 1H), 4.43-4.36 (m, 1H), 4.27-4.20 (m, 1H), 3.91 (s, 3H), 2.97-2.86 (m, 1H), 2.59 (br dd, J=2.0, 15.2 Hz, 1H), 2.46-2.38 (m, 1H), 2.03-1.96 (m, 1H). MS (ESI) m/z 526.1 [M+H]⁺

Compound 42: Step 1: To a mixture of phenyl (4-(pyridin-2-yl)phenyl)carbamates (see Compound 11) (91.5 mg, 315 umol, 1.20 eq) and 3-(6-(hydroxymethyl)-4-methoxy-1-oxoisoindolin-2-yl)piperidine-2,6-dione XLI (80.0 mg, 262 umol, 1.00 eq) in dimethyl formamide (1.00 mL) was added sodium hydride (15.7 mg, 394 umol, 60% purity, 1.50 eq) in one portion at 0° C. The mixture was stirred at 15° C. for 1 h. The mixture was quenched with 1 M hydrochloric acid (0.500 mL) and filtered. The filtrate was purified by prep-HPLC and lyophilized to give (2-(2,6-dioxopiperidin-3-yl)-7-methoxy-3-oxoisoindolin-5-yl)methyl (4-(pyridin-2-yl) phenyl)carbamate. ¹H NMR (400 MHz, DMSO-d₆) δ=10.98 (s, 1H), 10.33 (s, 1H), 8.78 (d, J=5.3 Hz, 1H), 8.46 (br t, J=7.8 Hz, 1H), 8.32 (d, J=8.2 Hz, 1H), 8.12 (d, J=8.8 Hz, 2H), 7.82 (br t, J=6.5 Hz, 1H), 7.74 (d, J=8.8 Hz, 2H), 7.42 (s, 1H), 7.35 (s, 1H), 5.31 (s, 2H), 5.10 (dd, J=5.1, 13.3 Hz, 1H), 4.44-4.35 (m, 1H), 4.28-4.18 (m, 1H), 3.92 (s, 3H), 2.97-2.83 (m, 1H), 2.59 (br d, J=17.6 Hz, 1H), 2.46-2.37 (m, 1H), 2.06-1.94 (m, 1H). MS (ESI) m/z 501.1 [M+H]⁺

Compound 43: To a solution of phenyl (4-(tert-butyl)phenyl)carbamate (see Compound 10) (97.4 mg, 361 umol, 1.10 eq) and 3-(6-(hydroxymethyl)-4-methoxy-1-oxoisoindolin-2-yl)piperidine-2,6-dione XLI (100 mg, 328 umol, 1.00 eq) in dimethyl formamide (1.50 mL) was added sodium hydride (26.3 mg, 657 umol, 60% purity, 2.00 eq) at 0° C. The mixture was stirred at 0° C. for 1 h. The pH of the mixture was adjusted to around 6 with hydrochloric acid (1M). The mixture was extracted with ethyl acetate (3×10.0 mL). The combined organic layers were dried over anhydrous sodium sulfate, filtered and concentrated under reduced pressure to give a residue. The residue was purified by prep-HPLC and lyophilized to afford (2-(2,6-dioxopiperidin-3-yl)-7-methoxy-3-oxoisoindolin-5-yl)methyl (4-(tert-butyl)phenyl)carbamate. ¹H NMR (400 MHz, DMSO-d₆) δ=10.97 (s, 1H), 9.71 (br s, 1H), 7.38 (br d, J=5.7 Hz, 3H), 7.31-7.27 (m, 3H), 5.25 (s, 2H), 5.10 (dd, J=5.1, 13.3 Hz, 1H), 4.43-4.33 (m, 1H), 4.25-4.20 (m, 1H), 3.90 (s, 3H), 2.96-2.85 (m, 1H), 2.61 (br s, 1H), 2.44-2.37 (m, 1H), 2.02-1.95 (m, 1H), 1.25 (s, 9H). MS (ESI) m/z. 480.1 [M+H]⁺

Compound 44: To a solution of 3-(6-(hydroxymethyl)-4-methoxy-1-oxoisoindolin-2-yl)piperidine-2,6-dione XLI (80.0 mg, 262 umol, 1.00 eq) in dimethyl formamide (2.00 mL) was added phenyl (6-(tert-butyl)pyridin-3-yl)carbamate (see Compound 9) (85.2 mg, 315 umol, 1.20 eq) and sodium hydride (21.0 mg, 525 umol, 60% purity, 2.00 eq) at 0° C. The mixture was stirred at 0° C. for 0.5 h. The reaction mixture was quenched with hydrochloric acid (1M, 1.00 ml) to give a solution. The solution was purified by prep-HPLC and lyophilized. The crude product was purified by prep-HPLC and lyophilized. The crude product was purified again two times by prep-HPLC and lyophilized to give (2-(2,6-dioxopiperidin-3-yl)-7-methoxy-3-oxoisoindolin-5-yl)methyl (6-(tert-butyl) pyridin-3-yl)carbamate. ¹H NMR (400 MHz, DMSO-d₆)=10.98 (br s, 1H), 9.94 (br s, 1H), 8.56 (d, J=2.3 Hz, 1H), 7.83 (br d, J=7.8 Hz, 1H), 7.43-7.29 (m, 3H), 5.28 (s, 2H), 5.11 (dd, J=5.1, 13.3 Hz, 1H), 4.45-4.35 (m, 1H), 4.31-4.19 (m, 1H), 3.92 (s, 3H), 2.97-2.86 (m, 1H), 2.62-2.57 (m, 1H), 2.44 (br dd, J=4.5, 13.1 Hz, 1H), 2.04-1.96 (m, 1H), 1.28 (s, 9H). MS (ESI) m/z 481.1 [M+H]⁺

Compound 45: To a solution of phenyl (6-phenylpyridin-3-yl)carbamate (see Compound 8) (104.9 mg, 361 umol, 1.10 eq) and 3-(6-(hydroxymethyl)-4-methoxy-1-oxoisoindolin-2-yl)piperidine-2,6-dione (100 mg, 328 umol, 1.00 eq) in dimethyl formamide (1.50 mL) was added sodium hydride (26.3 mg, 657 umol, 60% purity, 2.00 eq) at 0° C. The mixture was stirred at 0° C. for 1 h. The pH of the mixture was adjusted to around 6 with hydrochloric acid (1M). The mixture was extracted with ethyl acetate (3×10.0 mL). The combined organic layers were dried over anhydrous sodium sulfate, filtered and concentrated under reduced pressure to give a residue. The residue was purified by prep-HPLC and lyophilized to afford (2-(2,6-dioxopiperidin-3-yl)-7-methoxy-3-oxoisoindolin-5-yl)methyl(6-phenylpyridin-3-yl)carbamate. ¹H NMR (400 MHz, DMSO-d₆) δ=10.98 (s, 1H), 10.53-10.20 (m, 1H), 8.79 (br s, 1H), 8.16-8.01 (m, 4H), 7.53-7.40 (m, 4H), 7.35 (s, 1H), 5.32 (s, 2H), 5.10 (dd, J=5.1, 13.2 Hz, 1H), 4.42 (s, 1H), 4.25-4.21 (m, 1H), 3.92 (s, 3H), 2.97-2.83 (m, 1H), 2.61 (br s, 1H), 2.43 (br dd, J=4.4, 13.1 Hz, 1H), 2.03-1.95 (m, 1H). MS (ESI) m/z. 501.1 [M+H]⁺

Compound 46: To a solution of phenyl (3-(difluoromethoxy)-4-fluorophenyl)carbamate (see Compound 7) (86.0 mg, 289 umol, 1.10 eq) and 3-(6-(hydroxymethyl)-4-methoxy-1-oxoisoindolin-2-yl)piperidine-2,6-dione XLI (80.0 mg, 263 umol, 1.00 eq) in dimethyl formamide (1.50 mL) was added sodium hydride (21.0 mg, 526 umol, 60% purity, 2.00 eq) at 0° C. The mixture was stirred at 25° C. for 1 h. The pH of the mixture was adjusted to around 6 with hydrochloric acid (1M). The mixture was diluted with ethyl acetate/water (30.0 mL/20 mL). The organic layer was dried over anhydrous sodium sulfate, filtered and concentrated under reduced pressure to give a residue. The residue was purified by prep-HPLC and lyophilized to afford (2-(2,6-dioxopiperidin-3-yl)-7-methoxy-3-oxoisoindolin-5-yl)methyl(3-(difluoromethoxy)-4-fluorophenyl)carbamate. ¹H NMR (400 MHz, DMSO-d₆) δ=11.12-10.83 (m, 1H), 10.05 (br s, 1H), 7.58 (br d, J=6.0 Hz, 1H), 7.39 (s, 1H), 7.35-7.27 (m, 3H), 7.20 (t, J=73.2 Hz, 1H), 5.27 (s, 2H), 5.10 (dd, J=5.1, 13.3 Hz, 1H), 4.42-4.35 (m, 1H), 4.25-4.19 (m, 1H), 3.91 (s, 3H), 2.97-2.83 (m, 1H), 2.62-2.56 (m, 1H), 2.43 (br dd, J=4.5, 13.1 Hz, 1H), 2.03-1.94 (m, 1H). MS (ESI) m/z. 508.2 [M+H]⁺

Compound 47: To a solution of 3-(6-(hydroxymethyl)-4-methoxy-1-oxoisoindolin-2-yl)piperidine-2,6-dione XLI (80.0 mg, 263 umol, 1.00 eq) and phenyl (3-(difluoromethoxy)-5-fluorophenyl)carbamate (see Compound 6) (93.8 mg, 315 umol, 1.20 eq) in dimethyl formamide (1.00 mL) was added sodium hydride (15.8 mg, 394 umol, 60% purity, 1.50 eq) at 0° C. The mixture was stirred at 20° C. for 1 h. The mixture was quenched with acetic acid (1.00 mL) at 0° C. to give a solution. The solution was purified by prep-HPLC and lyophilized to afford (2-(2,6-dioxopiperidin-3-yl)-7-methoxy-3-oxoisoindolin-5-yl)methyl(3-(difluoromethoxy)-5-fluorophenyl) carbamate. ¹H NMR (400 MHz, DMSO-d₆) δ=10.98 (s, 1H), 10.26 (br s, 1H), 7.39 (s, 1H), 7.32 (s, 1H), 7.26-7.20 (s, 1H), 7.21 (t, J=60 Hz, 1H), 7.17 (br s, 1H), 6.76 (br dd, J=1.8, 9.7 Hz, 1H), 5.28 (s, 2H), 5.10 (br dd, J=2.9, 13.0 Hz, 1H), 4.42-4.34 (m, 1H), 4.26-4.19 (m, 1H), 3.90 (d, J=1.8 Hz, 3H), 2.95-2.87 (m, 1H), 2.61 (br s, 1H), 2.44-2.37 (m, 1H), 2.03-1.96 (m, 1H). MS (ESI) m/z 508.0 [M+H]⁺

Compound 48: To a solution of phenyl (3-(difluoromethoxy)-4-methylphenyl)carbamate (see Compound 35) (90.1 mg, 307 umol, 1.17 eq) in dimethyl formamide (1.00 mL) was added 3-(6-(hydroxymethyl)-4-methoxy-1-oxoisoindolin-2-yl)piperidine-2,6-dione (80.0 mg, 263 umol, 1.00 eq) and sodium hydride (21.0 mg, 526 umol, 60% purity, 2.00 eq) at 0° C. The mixture was stirred at 25° C. for 1 h. The pH of the mixture was adjusted to 7 with hydrochloric acid (1 M), then the mixture was diluted with dimethyl formamide (1.00 mL).

The residue was purified by Prep-HPLC and the desired fraction was lyophilized to afford (2-(2,6-dioxopiperidin-3-yl)-7-methoxy-3-oxoisoindolin-5-yl)methyl (3-(difluoromethoxy)-4-methylphenyl) carbamate. ¹H NMR (400 MHz, DMSO-d₆) δ=10.97 (br s, 1H), 9.93 (br s, 1H), 7.45-7.35 (m, 2H), 7.31 (s, 1H), 7.20 (s, 2H), 7.09 (t, J=74.0 Hz, 1H), 5.26 (s, 2H), 5.10 (dd, J=4.8, 13.2 Hz, 1H), 4.45-4.33 (m, 1H), 4.27-4.17 (m, 1H), 3.90 (s, 3H), 2.97-2.84 (m, 1H), 2.60 (br d, J=2.4 Hz, 1H), 2.43 (br dd, J=4.4, 12.8 Hz, 1H), 2.15 (s, 3H), 2.05-1.92 (m, 1H). MS (ESI) m/z 504.1 [M+H]⁺

Compound 49: To a solution of 3-(6-(hydroxymethyl)-4-methoxy-1-oxoisoindolin-2-yl)piperidine-2,6-dione XLI (90.0 mg, 296 umol, 1.00 eq) in dimethyl formamide (2.00 mL) was added phenyl (3-chloro-5-(difluoromethoxy) phenyl)carbamate (see Compound 34) (102 mg, 325 umol, 1.10 eq) and sodium hydride (23.7 mg, 591 umol, 60% purity, 2.00 eq) at 0° C. The mixture was stirred at 25° C. for 1 h. The reaction mixture was quenched by hydrochloric acid (1 M, 1 mL). The mixture was diluted with ethyl acetate/water (10.0 ml/10.0 ml). The organic layer was collected and concentrated to give a residue. The residue was purified by prep-HPLC. The desired fraction was collected and lyophilized to give (2-(2,6-dioxopiperidin-3-yl)-7-methoxy-3-oxoisoindolin-5-yl)methyl (3-chloro-5-(difluoromethoxy)phenyl)carbamate. ¹H NMR (400 MHz, DMSO-d₆) δ=10.96 (brs, 1H), 10.24 (s, 1H), 7.43 (d, J=2.1 Hz, 1H), 7.39 (s, 1H), 7.32 (s, 2H), 7.25 (t, J=73.6 Hz, 1H), 6.95 (s, 1H), 5.28 (s, 2H), 5.10 (dd, J=5.0, 13.3 Hz, 1H), 4.42-4.35 (m, 1H), 4.27-4.19 (m, 1H), 3.91 (s, 3H), 2.95-2.85 (m, 1H), 2.61 (br s, 1H), 2.43 (br dd, J=4.6, 13.2 Hz, 1H), 2.03-1.94 (m, 1H). MS (ESI) m/z 524.1 [M+H]⁺

Compound 50: To a solution of 3-[6-(hydroxymethyl)-4-methoxy-1-oxo-isoindolin-2-yl]piperidine-2,6-dione (80 mg, 263 umol, 1.00 eq) and phenyl (3-chloro-5-fluorophenyl)carbamate (see Compound 33) (83.8 mg, 315 umol, 1.20 eq) in dimethyl formamide (1.00 mL) was added sodium hydride (15.8 mg, 394 umol, 60% purity, 1.50 eq) at 0° C. The mixture was stirred at 20° C. for 1 h. The mixture was quenched by acetic acid (1.00 mL) at 0° C. to give a solution. The solution was purified by prep-HPLC and lyophilized to afford (2-(2,6-dioxopiperidin-3-yl)-7-methoxy-3-oxoisoindolin-5-yl)methyl(3-chloro-5-fluorophenyl)carbamate. ¹H NMR (400 MHz, DMSO-d₆) δ=10.98 (br s, 1H), 10.28 (s, 1H), 7.39 (d, J=3.3 Hz, 2H), 7.36-7.31 (m, 2H), 7.05 (td, J=2.0, 8.6 Hz, 1H), 5.28 (s, 2H), 5.10 (dd, J=5.0, 13.2 Hz, 1H), 4.43-4.35 (m, 1H), 4.26-4.19 (m, 1H), 3.91 (s, 3H), 2.96-2.86 (m, 1H), 2.60 (br d, J=2.4 Hz, 1H), 2.43 (br dd, J=4.6, 13.1 Hz, 1H), 2.02-1.95 (m, 1H). MS (ESI) m/z 476.0 [M+H]⁺

Compound 51: A mixture of phenyl (3-chloro-4-fluorophenyl)carbamate (see Compound 32) (76.8 mg, 289 umol, 1.10 eq), 3-(6-(hydroxymethyl)-4-methoxy-1-oxoisoindolin-2-yl)piperidine-2,6-dione XLI (80.0 mg, 263 umol, 1.00 eq) in dimethyl formamide (1.50 mL) was added sodium hydride (21.1 mg, 526 umol, 60% purity, 2.00 eq) at 0° C. And then the mixture was stirred at 25° C. for 1 h. The pH of mixture was adjusted to around 6 by adding hydrochloric acid (1 M). The mixture was purified by prep-HPLC and lyophilized to give (2-(2,6-dioxopiperidin-3-yl)-7-methoxy-3-oxoisoindolin-5-yl)methyl(3-chloro-4-fluorophenyl)carbamate. ¹H NMR (400 MHz, DMSO-d₆) δ=10.97 (s, 1H), 10.05 (br s, 1H), 7.71 (dd, J=2.1, 6.6 Hz, 1H), 7.41-7.29 (m, 4H), 5.26 (s, 2H), 5.09 (dd, J=5.1, 13.4 Hz, 1H), 4.42-4.33 (m, 1H), 4.27-4.18 (m, 1H), 3.90 (s, 3H), 2.96-2.84 (m, 1H), 2.58 (br d, J=18.1 Hz, 1H), 2.43 (br dd, J=4.4, 13.2 Hz, 1H), 2.01-1.95 (m, 1H). MS (ESI) m/z. 476.0 [M+H]⁺

Compound 52: A mixture of phenyl (3,5-dimethylphenyl)carbamate (see Compound 31) (69.8 mg, 289 umol, 1.10 eq), 3-(6-(hydroxymethyl)-4-methoxy-1-oxoisoindolin-2-yl)piperidine-2,6-dione (80.0 mg, 262.9 umol, 1.00 eq) in dimethyl formamide (1.50 mL) was added sodium hydride (21.0 mg, 526 umol, 60% purity, 2.00 eq) at 0° C. And then the mixture was stirred at 25° C. for 1 h. The pH of the mixture was adjusted to around 6 by adding hydrochloric acid. The mixture was purified by prep-HPLC and lyophilized to afford (2-(2,6-dioxopiperidin-3-yl)-7-methoxy-3-oxoisoindolin-5-yl)methyl (3,5-dimethylphenyl)carbamate. ¹H NMR (400 MHz, DMSO-d₆) δ=10.97 (s, 1H), 9.65 (s, 1H), 7.38 (s, 1H), 7.30 (s, 1H), 7.09 (s, 2H), 6.64 (s, 1H), 5.23 (s, 2H), 5.09 (dd, J=5.0, 13.3 Hz, 1H), 4.42-4.33 (m, 1H), 4.27-4.17 (m, 1H), 3.90 (s, 3H), 2.95-2.84 (m, 1H), 2.60 (br s, 1H), 2.44-2.39 (m, 1H), 2.21 (s, 6H), 2.01-1.96 (m, 1H). MS (ESI) m/z. 452.1 [M+H]⁺

Compound 53: To a solution of 3-(6-(hydroxymethyl)-4-methoxy-1-oxoisoindolin-2-yl)piperidine-2,6-dione XLI (80.0 mg, 262 umol, 1.00 eq) in dimethyl formamide (2.00 mL) was added phenyl (3-(difluoromethoxy)phenyl)carbamate (see Compound 26)(88.0 mg, 315 umol, 1.20 eq) and sodium hydride (21.0 mg, 525 umol, 60% purity, 2.00 eq) at 0° C. The mixture was stirred at 0° C. for 0.5 h. The reaction mixture was quenched with formic acid (0.500 ml) to give a solution. The solution was purified by prep-HPLC and lyophilized to give (2-(2,6-dioxopiperidin-3-yl)-7-methoxy-3-oxoisoindolin-5-yl)methyl (3-(difluoromethoxy)phenyl)carbamate. ¹H NMR (400 MHz, DMSO-d₆) δ=10.98 (s, 1H), 10.05 (s, 1H), 7.40 (s, 2H), 7.36-7.28 (m, 3H), 7.19 (t, J=74 Hz, 1H), 6.84-6.80 (m, 1H), 5.28 (s, 2H), 5.11 (dd, J=5.0, 13.2 Hz, 1H), 4.44-4.35 (m, 1H), 4.28-4.19 (m, 1H), 3.92 (s, 3H), 2.97-2.86 (m, 1H), 2.62-2.57 (m, 1H), 2.47-2.41 (m, 1H), 2.04-1.96 (m, 1H). MS (ESI) m/z 490.1 [M+H]⁺

Compound 54: To a mixture of phenyl (3-(tert-butyl)phenyl)carbamate (see Compound 25) (85.0 mg, 315 umol, 1.20 eq) and 3-(6-(hydroxymethyl)-4-methoxy-1-oxoisoindolin-2-yl)piperidine-2,6-dione XLI (80.0 mg, 263 umol, 1.00 eq) in dimethyl formamide (1.00 mL) was added sodium hydride (15.8 mg, 394 umol, 60% purity, 1.50 eq) in one portion at 0° C. The mixture was stirred at 15° C. for 1 h. The mixture was quenched with 1 M hydrochloric acid (0.500 mL) and filtered. The filtrate was purified by prep-HPLC and lyophilized to give (2-(2,6-dioxopiperidin-3-yl)-7-methoxy-3-oxoisoindolin-5-yl)methyl (3-(tert-butyl)phenyl)carbamate. ¹H NMR (400 MHz, DMSO-d₆) δ=10.97 (s, 1H), 9.72 (br s, 1H), 7.52 (br s, 1H), 7.39 (s, 1H), 7.34-7.29 (m, 2H), 7.20 (t, J=7.9 Hz, 1H), 7.04 (br d, J=7.6 Hz, 1H), 5.25 (s, 2H), 5.10 (dd, J=5.1, 13.3 Hz, 1H), 4.43-4.35 (m, 1H), 4.27-4.18 (m, 1H), 3.91 (s, 3H), 2.95-2.86 (m, 1H), 2.60 (br s, 1H), 2.45-2.38 (m, 1H), 2.04-1.95 (m, 1H), 1.25 (s, 9H). MS (ESI) m/z 480.1 [M+H]⁺

Compound 55: To a solution of 3-(6-(hydroxymethyl)-4-methoxy-1-oxoisoindolin-2-yl)piperidine-2,6-dione XLI (90.0 mg, 296 umol, 1.00 eq) in dimethyl formamide (2.00 mL) was added phenyl (3-(trifluoromethoxy)phenyl)carbamate (see Compound 15) (96.7 mg, 325 umol, 1.10 eq) and sodium hydride (23.7 mg, 591 umol, 60% purity, 2.00 eq) at 0° C. The mixture was stirred at 0° C. for 1 h. The reaction mixture was quenched by hydrochloric acid (1 M, 1 mL). The mixture was extracted with ethyl acetate/water (10.0 mL/10.0 mL). The organic layer was collected and concentrated to give a residue. The residue was purified by prep-HPLC. The desired fraction was collected and lyophilized to give (2-(2,6-dioxopiperidin-3-yl)-7-methoxy-3-oxoisoindolin-5-yl) methyl (3-(trifluoromethoxy)phenyl)carbamate. ¹H NMR (400 MHz, DMSO-d₆) δ=10.95 (br dd, J=2.4, 4.9 Hz, 1H), 10.15 (s, 1H), 7.59 (br s, 1H), 7.47-7.37 (m, 3H), 7.32 (s, 1H), 6.99 (br d, J=6.1 Hz, 1H), 5.28 (s, 2H), 5.10 (dd, J=5.0, 13.3 Hz, 1H), 4.45-4.32 (m, 1H), 4.27-4.17 (m, 1H), 3.91 (s, 3H), 2.96-2.85 (m, 1H), 2.61-2.56 (m, 1H), 2.47-2.36 (m, 1H), 2.03-1.94 (m, 1H). MS (ESI) m/z 508.1 [M+H]⁺

Compound 56: To a solution of 3-(6-(hydroxymethyl)-4-methoxy-1-oxoisoindolin-2-yl)piperidine-2,6-dione XLI (80.0 mg, 262 umol, 1.00 eq) in dimethyl formamide (2.00 mL) was added phenyl (4-phenylpyridin-2-yl)carbamate (see Compound 24)(91.5 mg, 315 umol, 1.20 eq) and sodium hydride (21.0 mg, 525 umol, 60% purity, 2.00 eq) at 0° C. The mixture was stirred at 0° C. for 0.5 h. The reaction mixture was quenched with hydrochloric acid (1M, 1.00 ml) to give a solution. The solution was purified by prep-HPLC and lyophilized. The crude product was dissolved in dimethyl formamide (2.00 mL) and purified again by prep-HPLC and lyophilized to give (2-(2,6-dioxopiperidin-3-yl)-7-methoxy-3-oxoisoindolin-5-yl)methyl (4-phenylpyridin-2-yl)carbamate. ¹H NMR (400 MHz, DMSO-d₆) δ=10.98 (s, 1H), 10.92-10.53 (m, 1H), 8.36 (dd, J=2.8, 5.1 Hz, 1H), 8.14-8.08 (m, 1H), 7.78-7.71 (m, 2H), 7.54 (dt, J=6.1, 7.9 Hz, 3H), 7.44 (s, 2H), 7.36 (s, 1H), 5.34 (br s, 2H), 5.11 (dd, J=5.0, 13.3 Hz, 1H), 4.43-4.37 (m, 1H), 4.26-4.21 (m, 1H), 3.92 (s, 3H), 2.97-2.86 (m, 1H), 2.59 (br d, J=16.9 Hz, 1H), 2.44 (br dd, J=4.6, 13.1 Hz, 1H), 2.04-1.96 (m, 1H). MS (ESI) m/z 501.1 [M+H]⁺

Compound 57: To a mixture of 3-(6-(hydroxymethyl)-4-methoxy-1-oxoisoindolin-2-yl)piperidine-2,6-dione XLI (80.0 mg, 263 umol, 1.00 eq) and phenyl (4-cyclobutylphenyl)carbamate (see Compound 22) (84.3 mg, 525 umol, 1.20 eq) in dimethyl formamide (1.00 mL) was added sodium hydride (21.0 mg, 875 umol, 60% purity, 2.00 eq) at 0° C. The reaction mixture was stirred at 25° C. for 1 h. The reaction mixture was added hydrochloric acid (1 M, 2.00 mL) and filtered to give a filtrate. The filtrate was purified by Prep-HPLC and lyophilized to give (2-(2,6-dioxopiperidin-3-yl)-7-methoxy-3-oxoisoindolin-5-yl)methyl(4-cyclobutylphenyl) carbamate. ¹H NMR (400 MHz, DMSO-d₆) δ=11.05-10.89 (m, 1H), 9.73 (br s, 1H), 7.45-7.35 (m, 3H), 7.32 (s, 1H), 7.16 (s, 1H), 7.14 (s, 1H), 5.25 (s, 2H), 5.10 (dd, J=5.2, 13.2 Hz, 1H), 4.43-4.35 (m, 1H), 4.27-4.20 (m, 1H), 3.91 (s, 3H), 3.45 (quin, J=8.8 Hz, 1H), 2.97-2.86 (m, 1H), 2.63-2.57 (m, 1H), 2.44 (br dd, J=4.4, 13.2 Hz, 1H), 2.30-2.25 (m, 1H), 2.25-2.21 (m, 1H), 2.10-2.02 (m, 2H), 2.01-1.90 (m, 2H), 1.84-1.75 (m, 1H). MS (ESI) m/z 478.2 [M+H]⁺.

Compound 58: To a solution of 3-(6-(hydroxymethyl)-4-methoxy-1-oxoisoindolin-2-yl)piperidine-2,6-dione XLI (80.0 mg, 262 umol, 1.00 eq) and phenyl (3-cyclopropoxyphenyl)carbamate (70.8 mg, 262 umol, 1.00 eq) in dimethyl formamide (1.00 mL) was added sodium hydride (21.0 mg, 525 umol, 60% purity, 2.00 eq) in portions at 0° C. The mixture was stirred at 25° C. for 1 h. The mixture was quenched by 1 M hydrochloric acid (1.00 mL) and filtered. The filtrate was purified by prep-HPLC to give (2-(2,6-dioxopiperidin-3-yl)-7-methoxy-3-oxoisoindolin-5-yl)methyl (3-cyclopropoxyphenyl)carbamate. ¹H NMR (400 MHz, DMSO-d₆) δ=10.99 (s, 1H), 9.83 (br s, 1H), 7.40 (s, 1H), 7.32 (s, 1H), 7.29 (br s, 1H), 7.23-7.15 (m, 1H), 7.06 (br d, J=8.4 Hz, 1H), 6.70 (dd, J=1.9, 8.2 Hz, 1H), 5.26 (s, 2H), 5.11 (dd, J=5.1, 13.4 Hz, 1H), 4.46-4.32 (m, 1H), 4.30-4.16 (m, 1H), 3.91 (s, 3H), 3.77 (td, J=3.0, 5.7 Hz, 1H), 2.96-2.87 (m, 1H), 2.60 (br d, J=5.9 Hz, 1H), 2.42 (br d, J=4.4 Hz, 1H), 2.02-1.97 (m, 1H), 0.78-0.73 (m, 2H), 0.68-0.59 (m, 2H). MS (ESI) m/z 480.2 [M+H]⁺

Compound 59: To a mixture of 3-(6-(hydroxymethyl)-4-methoxy-1-oxoisoindolin-2-yl)piperidine-2,6-dione XLI (100 mg, 329 umol, 1.00 eq) and phenyl (5-methyl-6-phenylpyridin-3-yl)carbamate (see Compound 20) (150 mg, 493 umol, 1.50 eq) in dimethyl formamide (1.00 mL) was added sodium hydride (26.3 mg, 657 umol, 60% purity, 2.00 eq) at 0° C. The reaction mixture was stirred at 25° C. for 1 h. The reaction mixture was added hydrochloric acid (1 M, 2.00 mL) and filtered to give a filtrate. The filtrate was purified twice by Prep-HPLC and lyophilized to give (2-(2,6-dioxopiperidin-3-yl)-7-methoxy-3-oxoisoindolin-5-yl)methyl(5-methyl-6-phenylpyridin-3-yl)carbamate. ¹H NMR (400 MHz, DMSO-d₆) δ=10.98 (s, 1H), 10.69 (br s, 1H), 8.76 (s, 1H), 8.22 (br s, 1H), 7.66-7.62 (m, 2H), 7.60-7.54 (m, 3H), 7.44 (s, 1H), 7.36 (s, 1H), 5.36 (s, 2H), 5.12 (br dd, J=5.2, 13.2 Hz, 1H), 4.44-4.38 (m, 1H), 4.28-4.22 (m, 1H), 3.94 (s, 3H), 3.02-2.86 (m, 1H), 2.62-2.58 (m, 1H), 2.48-2.42 (m, 1H), 2.42-2.36 (m, 3H), 2.06-1.98 (m, 1H). MS (ESI) m/z 515.1 [M+H]⁺

Compound 60: To a solution of 3-(6-(hydroxymethyl)-4-methoxy-1-oxoisoindolin-2-yl)piperidine-2,6-dione XLI (80.0 mg, 262 umol, 1.00 eq) in dimethyl formamide (2.00 mL) was added phenyl (6-(o-tolyl)pyridin-3-yl)carbamate (see Compound 19) (96.0 mg, 315 umol, 1.20 eq) and sodium hydride (21.0 mg, 525 umol, 60% purity, 2.00 eq) at 0° C. The mixture was stirred at 0° C. for 0.5 h. The reaction mixture was quenched with hydrochloric acid (1M, 1.00 ml) to give a solution. The solution was purified by prep-HPLC and lyophilized. The solid was further purified by prep-HPLC and lyophilized to give (2-(2,6-dioxopiperidin-3-yl)-7-methoxy-3-oxoisoindolin-5-yl)methyl (6-(o-tolyl)pyridin-3-yl)carbamate. ¹H NMR (400 MHz, DMSO-d₆)=10.99 (s, 1H), 10.15 (br s, 1H), 8.73 (d, J=2.3 Hz, 1H), 8.00 (dd, J=2.3, 8.6 Hz, 1H), 7.49 (d, J=8.6 Hz, 1H), 7.43 (s, 1H), 7.40-7.32 (m, 2H), 7.31-7.23 (m, 3H), 5.32 (s, 2H), 5.11 (dd, J=5.0, 13.3 Hz, 1H), 4.45-4.36 (m, 1H), 4.28-4.19 (m, 1H), 3.93 (s, 3H), 2.97-2.85 (m, 1H), 2.63-2.57 (m, 1H), 2.44 (br dd, J=4.5, 13.1 Hz, 1H), 2.32 (s, 3H), 2.04-1.96 (m, 1H). MS (ESI) m/z 515.2 [M+H]⁺

Compound 61: Step 1: To a mixture of 1-chloro-2,3-difluoro-5-nitrobenzene (1.00 g, 5.17 mmol, 1.00 eq) and iron powder (1.44 g, 25.8 mmol, 5.00 eq) and ammonium chloride (1.38 g, 25.8 mmol, 5.00 eq) in methanol (16.0 mL) was added water (16.0 mL). The reaction mixture was stirred at 80° C. for 2 h. Then the reaction mixture was washed with methanol (80.0 mL) and then filtered. The filtrate was concentrated under reduced pressure to afford a residue. The residue was diluted with water (80.0 mL), and extracted with ethyl acetate (3×25.0 mL). Then the organic phase was washed with brine (20.0 mL), dried over anhydrous sodium sulfate and concentrated under reduced pressure to afford 3-chloro-4,5-difluoroaniline. ¹H NMR (400 MHz, DMSO-d₆) δ=6.56-6.40 (m, 2H), 5.53 (s, 2H). MS (ESI) m/z 164.0 [M+H]⁺

Step 2: To a mixture of 3-chloro-4,5-difluoroaniline (1.00 g, 6.11 mmol, 1.00 eq) and pyridine (1.45 g, 18.3 mmol, 1.48 mL, 3.00 eq) in acetonitrile (12.0 mL) was added phenyl carbonochloridate (1.34 g, 8.56 mmol, 1.07 mL, 1.40 eq) at 25° C. The reaction mixture was stirred at 25° C. for 12 h. The reaction mixture was concentrated under reduced pressure to afford a residue. The residue was diluted with water (80.0 mL) and extracted with ethyl acetate (3×20.0 mL). Then the organic phase was combined and concentrated under reduced pressure to afford a residue. The residue was purified by reverse phase HPLC. The desired eluent was lyophilized to afford phenyl (3-chloro-4,5-difluorophenyl)carbamate. ¹H NMR (400 MHz, DMSO-d₆) δ=9.32 (br s, 1H), 7.68-7.20 (m, 5H), 6.79-6.72 (m, 2H). MS (ESI) m/z 284.1 [M+H]⁺

Step 3: To a solution of O-benzoyl-N-(3-chloro-4,5-difluorophenyl)hydroxylamine (90.0 mg, 317 umol, 1.00 eq) in dimethylformamide (500 uL) was added 3-(4-fluoro-6-(hydroxymethyl)-1-oxoisoindolin-2-yl)piperidine-2,6-dione XVIII (102 mg, 349 umol, 1.10 eq) and sodium hydride (25.4 mg, 635 umol, 60% purity, 2.00 eq). The mixture was stirred at 25° C. for 1 h. The pH of the mixture was adjusted to 7 with formic acid, then the mixture was diluted with dimethyl formamide (1.00 mL). The mixture was filtered and the filter cake was purified twice by Prep-HPLC. Then the desired eluent was lyophilized to afford (2-(2,6-dioxopiperidin-3-yl)-7-fluoro-3-oxoisoindolin-5-yl)methyl(3-chloro-4,5-difluorophenyl)carbamate. ¹H NMR (400 MHz, DMSO-d₆) δ=11.24-10.66 (m, 1H), 10.49 10.10 (m, 1H), 7.68 (s, 1H), 7.57 (d, J=9.6 Hz, 1H), 7.54-7.43 (m, 2H), 5.30 (s, 2H), 5.13 (dd, J=4.9, 13.2 Hz, 1H), 4.62-4.54 (m, 1H), 4.45-4.36 (m, 1H), 2.98-2.84 (m, 1H), 2.60 (td, J=2.0, 15.2 Hz, 1H), 2.44 (br dd, J=4.6, 12.8 Hz, 1H), 2.06-1.97 (m, 1H). MS (ESI) m/z 482.0 [M+H]⁺

Compound 62: To a solution of 3-(6-(hydroxymethyl)-4-methoxy-1-oxoisoindolin-2-yl)piperidine-2,6-dione XLI (80.0 mg, 262 umol, 1.00 eq) and (R)-phenyl (6-(2-methylpyrrolidin-1-yl)pyridin-3-yl)carbamate (see Compound 23) (93.8 mg, 315 umol, 1.20 eq) in dimethyl formamide (1.00 mL) was added sodium hydride (21.0 mg, 525 umol, 60% purity, 2.00 eq) in portions at 0° C. The mixture was stirred at 25° C. for 1 h. The mixture was quenched by 1 M hydrochloric acid (1.00 mL) and filtered. The filtrate was purified three times by prep-HPLC to give (2-(2,6-dioxopiperidin-3-yl)-7-methoxy-3-oxoisoindolin-5-yl)methyl (6-((R)-2-methylpyrrolidin-1-yl) pyridin-3-yl)carbamate. ¹H NMR (400 MHz, DMSO-d₆) δ=10.98 (s, 1H), 10.13 (br s, 1H), 8.18 (br s, 1H), 7.96 (dd, J=2.3, 9.7 Hz, 1H), 7.39 (s, 1H), 7.33 (s, 1H), 7.19 (br d, J=9.8 Hz, 1H), 5.29 (s, 2H), 5.10 (dd, J=5.1, 13.2 Hz, 1H), 4.46-4.35 (m, 1H), 4.28 (br d, J=6.0 Hz, 1H), 4.26-4.18 (m, 1H), 3.92 (s, 3H), 3.66 (br d, J=2.5 Hz, 1H), 3.43-3.37 (m, 1H), 2.98-2.85 (m, 1H), 2.60 (br d, J=17.4 Hz, 1H), 2.47-2.38 (m, 1H), 2.15-2.04 (m, 3H), 2.03-1.96 (m, 1H), 1.78 (br d, J=11.4 Hz, 1H), 1.17 (d, J=6.3 Hz, 3H). MS (ESI) m/z 508.1 [M+H]⁺

Compound 63: To a solution of 3-(4-fluoro-6-(hydroxymethyl)-1-oxoisoindolin-2-yl)piperidine-2,6-dione XVIII (130 mg, 445 umol, 1.00 eq) and phenyl (3-fluoro-5-methylphenyl)carbamate (see Compound 67) (130 mg, 530 umol, 1.19 eq) in dimethyl formamide (3.00 mL) was added sodium hydride (60%, dispersion in paraffin liquid) (35.0 mg, 875 umol, 60% purity, 1.97 eq) in portions at 0° C. The reaction mixture was stirred at 10° C. for 1 h. The reaction mixture was adjusted to pH=9 with formic acid. The mixture was filtered to give the filtrate. The filtrate was purified by prep-HPLC. The desired fraction was collected and concentrated under pressure to give a solution. The solution was lyophilized to give (2-(2,6-dioxopiperidin-3-yl)-7-fluoro-3-oxoisoindolin-5-yl)methyl (3-fluoro-5-methylphenyl)carbamate. ¹H NMR (400 MHz, DMSO-d₆) δ=11.01 (s, 1H), 10.01 (s, 1H), 7.68 (s, 1H), 7.56 (d, J=9.7 Hz, 1H), 7.18 (br d, J=11.5 Hz, 1H), 7.06 (s, 1H), 6.67 (br d, J=9.7 Hz, 1H), 5.28 (s, 2H), 5.13 (dd, J=5.1, 13.3 Hz, 1H), 4.62-4.54 (m, 1H), 4.45-4.36 (m, 1H), 2.98-2.85 (m, 1H), 2.63-2.57 (m, 1H), 2.44 (br dd, J=4.4, 13.2 Hz, 1H), 2.25 (s, 3H), 2.05-1.98 (m, 1H). MS (ESI) m/z 444.1 [M+H]⁺

Compound 64: To a solution of 3-(4-fluoro-6-(hydroxymethyl)-1-oxoisoindolin-2-yl)piperidine-2,6-dione XVIII (100 mg, 342 umol, 1.00 eq) and phenyl N-(5-chloro-2-fluoro-4-methyl-phenyl)carbamate (see Compound 68) (115 mg, 411 umol, 1.20 eq) in dimethyformamide (3.00 mL) was added sodium hydride (20.5 mg, 513 umol, 60% purity, 1.50 eq) at 0° C. The mixture was stirred at 0° C. for 0.5 h. The reaction was quenched by acetic acid (0.500 mL) slowly and then filtered. The filtrate was purified by prep-HPLC and lyophilized to afford (2-(2,6-dioxopiperidin-3-yl)-7-fluoro-3-oxoisoindolin-5-yl)methyl(5-chloro-2-fluoro-4-methylphenyl)carbamate. ¹H NMR (400 MHz, DMSO-d₆) δ=11.02 (br s, 1H), 9.73 (br s, 1H), 7.78 (br d, J=7.2 Hz, 1H), 7.71 (s, 1H), 7.58 (d, J=9.7 Hz, 1H), 7.31 (d, J=11.4 Hz, 1H), 5.29 (s, 2H), 5.14 (dd, J=5.1, 13.3 Hz, 1H), 4.63-4.55 (m, 1H), 4.48-4.37 (m, 1H), 2.99-2.87 (m, 1H), 2.61 (td, J=2.1, 15.3 Hz, 1H), 2.44 (br dd, J=4.5, 13.1 Hz, 1H), 2.29 (s, 3H), 2.07-1.98 (m, 1H). MS (ESI) m/z 478.1 [M+H]⁺

Compound 65: To a solution of 3-(4-fluoro-6-(hydroxymethyl)-1-oxoisoindolin-2-yl)piperidine-2,6-dione XVIII (130 mg, 445 umol, 1.00 eq) and phenyl (2-fluoro-5-(trifluoromethoxy)phenyl)carbamate (see Compound 66) (167 mg, 530 umol, 1.19 eq) in dimethyl formamide (3.00 mL) was added sodium hydride (60%, dispersion in paraffin liquid) (35.0 mg, 875 umol, 60% purity, 1.97 eq) in portions at 0° C. The reaction mixture was stirred at 10° C. for 1 h. The reaction mixture was adjusted to pH=9 with formic acid. The mixture was filtered to give the filtrate. The filtrate was purified by prep-HPLC. The desired fraction was collected and concentrated under pressure to give a solution. The residual solution was lyophilized to give (2-(2,6-dioxopiperidin-3-yl)-7-fluoro-3-oxoisoindolin-5-yl)methyl (2-fluoro-5-(trifluoromethoxy)phenyl) carbamate. ¹H NMR (400 MHz, DMSO-d₆) δ=11.01 (br s, 1H), 9.96 (br s, 1H), 7.85 (br d, J=3.5 Hz, 1H), 7.72 (s, 1H), 7.59 (d, J=9.8 Hz, 1H), 7.39 (dd, J=9.2, 10.3 Hz, 1H), 7.19-7.08 (m, 1H), 5.31 (s, 2H), 5.13 (dd, J=5.1, 13.2 Hz, 1H), 4.63-4.54 (m, 1H), 4.46-4.36 (m, 1H), 2.97-2.86 (m, 1H), 2.65-2.57 (m, 1H), 2.47-2.38 (m, 1H), 2.06-1.97 (m, 1H). MS (ESI) m/z 514.0 [M+H]⁺

Compound 66: Step 1: A mixture of 2-fluoro-5-(trifluoromethoxy)aniline (500 mg, 2.56 mmol, 305 uL, 1.00 eq), phenyl carbonochloridate (441 mg, 2.82 mmol, 353 uL, 1.10 eq) and pyridine (608 mg, 7.69 mmol, 621 uL, 3.00 eq) in acetonitrile (3.00 mL) was stirred at 25° C. for 2 h. The mixture was concentrated to give crude product. The crude product was purified by reversed phase column chromatography. The desired fraction was collected and lyophilized to give phenyl (2-fluoro-5-(trifluoromethoxy)phenyl)carbamate. MS (ESI) m/z 316.1 [M+H]⁺

Step 2: To a solution of 3-(6-(hydroxymethyl)-4-methoxy-1-oxoisoindolin-2-yl)piperidine-2,6-dione XLI (130 mg, 427 umol, 1.00 eq) and phenyl (2-fluoro-5-(trifluoromethoxy)phenyl)carbamate (161 mg, 512 umol, 1.20 eq) in dimethylformamide (1.00 mL) was added sodium hydride (25.6 mg, 640 umol, 60% purity, 1.50 eq), the reaction was stirred at 20° C. for 0.5 h. The reaction was quenched by acetic acid (1.00 ml). The mixture was purified by Prep-HPLC to afford (2-(2,6-dioxopiperidin-3-yl)-7-methoxy-3-oxoisoindolin-5-yl)methyl (2-fluoro-5-(trifluoromethoxy)phenyl)carbamate. ¹H NMR (400 MHz, DMSO-d₆) δ=10.98 (br s, 1H), 9.93 (br s, 1H), 7.84 (br d, J=3.2 Hz, 1H), 7.44-7.35 (m, 2H), 7.32 (s, 1H), 7.18-7.11 (m, 1H), 5.29 (s, 2H), 5.10 (dd, J=5.2, 13.6 Hz, 1H), 4.44-4.35 (m, 1H), 4.27-4.18 (m, 1H), 3.91 (s, 3H), 2.97-2.85 (m, 1H), 2.60 (br s, 1H), 2.43 (br dd, J=4.0, 13.2 Hz, 1H), 2.04-1.96 (m, 1H). MS (ESI) m/z 526.2 [M+H]⁺

Compound 67: Step 1: To a solution of 3-fluoro-5-methylaniline (1.00 g, 7.99 mmol, 1.00 eq) in acetonitrile (5.00 mL) was added pyridine (3.16 g, 39.9 mmol, 3.22 mL, 5.00 eq) and phenyl carbonochloridate (1.88 g, 12.0 mmol, 1.50 mL, 1.50 eq). The mixture was stirred at 25° C. for 1 h. The mixture was filtered to give a filtrate which was purified by reversed phase column chromatography. The desired fraction was collected and concentrated to give phenyl (3-fluoro-5-methylphenyl)carbamate. ¹H NMR (400 MHz, DMSO-d₆) δ=10.39 (br s, 1H), 7.50-7.38 (m, 3H), 7.29-7.21 (m, 3H), 7.12 (s, 1H), 6.72 (br d, J=9.3 Hz, 1H), 2.29 (s, 3H). MS (ESI) m/z 246.0 [M+H]⁺

Step 2: To a solution of 3-(6-(hydroxymethyl)-4-methoxy-1-oxoisoindolin-2-yl)piperidine-2,6-dione XLI (150 mg, 492 umol, 1.00 eq) and phenyl (3-fluoro-5-methylphenyl)carbamate (132 mg, 542 umol, 1.10 eq) in dimethylformamide (1.00 mL) was added sodium hydride (29.5 mg, 739 umol, 60% purity, 1.50 eq), the reaction was stirred at 20° C. for 1 h. The reaction was quenched by acetic acid (1.00 ml). The mixture was purified by Prep-HPLC to afford (2-(2,6-dioxopiperidin-3-yl)-7-methoxy-3-oxoisoindolin-5-yl)methyl(3-fluoro-5-methylphenyl)carbamate. ¹H NMR (400 MHz, DMSO-d₆) δ=10.97 (br s, 1H), 9.98 (s, 1H), 7.39 (s, 1H), 7.31 (s, 1H), 7.19 (br d, J=11.2 Hz, 1H), 7.07 (s, 1H), 6.66 (br d, J=9.6 Hz, 1H), 5.26 (s, 2H), 5.10 (dd, J=5.2, 13.2 Hz, 1H), 4.44-4.34 (m, 1H), 4.27-4.18 (m, 1H), 3.91 (s, 3H), 2.97-2.86 (m, 1H), 2.62-2.55 (m, 1H), 2.43-2.37 (m, 1H), 2.26 (s, 3H), 2.06-1.95 (m, 1H). MS (ESI) m/z 456.2[M+H]⁺

Compound 68: Step 1: A mixture of 1-chloro-4-fluoro-2-methyl-5-nitrobenzene (500 mg, 2.64 mmol, 1.00 eq), ammonium chloride (988 mg, 18.5 mmol, 7.00 eq) and iron powder (1.03 g, 18.5 mmol, 7.00 eq) in methanol (10.0 mL) and water (2.00 mL) was stirred at 80° C. for 2 h. The resulting mixture was filtered over celite, and the filtrate was concentrated under reduced pressure to give 5-chloro-2-fluoro-4-methylaniline.

Step 2: A mixture of phenyl carbonochloridate (236 mg, 1.50 mmol, 188 uL, 1.20 eq), 5-chloro-2-fluoro-4-methylaniline (200 mg, 1.25 mmol, 1.00 eq) and pyridine (298 mg, 3.76 mmol, 304 uL, 3.00 eq) in acetonitrile (10.0 mL) was stirred at 25° C. for 12 h. The mixture was concentrated in vacuum. The crude product was purified by reversed-phase HPLC. The desired fraction was collected and lyophilized to give phenyl (5-chloro-2-fluoro-4-methylphenyl)carbamate. MS (ESI) m/z 280.1 [M+H]⁺

Step 3: A mixture of 3-(6-(hydroxymethyl)-4-methoxy-1-oxoisoindolin-2-yl)piperidine-2,6-dione XLI (120 mg, 394 umol, 1.00 eq) and phenyl (5-chloro-2-fluoro-4-methylphenyl)carbamate (132 mg, 473 umol, 1.20 eq) in dimethylformamide (2.00 mL) was added sodium hydride (31.5 mg, 788 umol, 60% purity, 2.00 eq) at 0° C. The reaction mixture was stirred at 20° C. for 2 h. The reaction mixture was quenched by addition formic acid (2.00 mL) and water (10.0 mL), then filtered to give a filter cake. The filter cake was triturated with ethyl acetate (10.0 mL) at 20° C. for 10 min to give (2-(2,6-dioxopiperidin-3-yl)-7-methoxy-3-oxoisoindolin-5-yl)methyl (5-chloro-2-fluoro-4-methylphenyl)carbamate. ¹H NMR (400 MHz, DMSO-d₆) δ=10.99 (s, 1H), 9.69 (br s, 1H), 7.77 (br d, J=7.2 Hz, 1H), 7.41 (s, 1H), 7.35-7.27 (m, 2H), 5.27 (s, 2H), 5.11 (dd, J=5.2, 13.2 Hz, 1H), 4.44-4.35 (m, 1H), 4.28-4.19 (m, 1H), 3.91 (s, 3H), 2.97-2.86 (m, 1H), 2.59 (br d, J=17.2 Hz, 1H), 2.46-2.38 (m, 1H), 2.29 (s, 3H), 2.04-1.96 (m, 1H). MS (ESI) m/z 490.0 [M+H]⁺.

Compound 69: Step 1: A mixture of methyl 7-bromo-2-(2,6-dioxopiperidin-3-yl)-3-oxoisoindoline-5-carboxylate VI (2.00 g, 5.25 mmol, 1.00 eq), potassium methyltrifluoroborate (4.00 g, 32.80 mmol, 6.25 eq), potassium carbonate (3.26 g, 23.6 mmol, 4.50 eq) in dioxane (50.0 mL) and water (10.0 mL) was degassed and purged with nitrogen for 3 times, then [1,1′-bis(diphenylphosphino)ferrocene]dichloropalladium(II) (575 mg, 787 umol, 0.150 eq) was added into the mixture. The mixture was stirred at 110° C. for 8 h under nitrogen atmosphere. The reaction mixture was concentrated under reduced pressure to give a residue. The residue was purified by column chromatography to give a yellow solid. The yellow solid was purified by reverse phase HPLC and lyophilized to give methyl 2-(2,6-dioxopiperidin-3-yl)-7-methyl-3-oxoisoindoline-5-carboxylate. MS (ESI) m/z 317.1 [M+H]+

Step 2: To a mixture of methyl 2-(2,6-dioxopiperidin-3-yl)-7-methyl-3-oxoisoindoline-5-carboxylate (270 mg, 854 umol, 1.00 eq) in tetrahydrofuran (20.0 mL) was added lithium aluminum hydride (58.3 mg, 1.54 mmol, 1.80 eq), the mixture was stirred at 0° C. for 1 h. After the reaction was completed, the reaction mixture was added very slowly into the hydrochloric acid (6.0 M, 50.0 mL), and the mixture was concentrated to give a residue. The residue was purified by reverse phase HPLC to give a brown solid. The brown solid was purified by Prep-HPLC and lyophilized to give 3-(6-(hydroxymethyl)-4-methyl-1-oxoisoindolin-2-yl)piperidine-2,6-dione. MS (ESI) m/z 289.0 [M+H]+

Step 3: A mixture of 3-(6-(hydroxymethyl)-4-methyl-1-oxoisoindolin-2-yl)piperidine-2,6-dione (15.0 mg, 52.0 umol, 1.00 eq) and phenyl (3-chloro-4-methylphenyl)carbamate (see Compound 1) (20.4 mg, 78.0 umol, 1.50 eq) in dimethyl formamide (1.00 mL) was stirred at 0° C. for 5 min. Then sodium hydride (4.16 mg, 104 umol, 60% purity, 2.00 eq) was added into the mixture. The mixture was stirred at 0° C. for 55 min. The mixture was adjusted to pH=6 with hydrochloric acid (1M) to give a solution. The solution was purified by Prep-HPLC and lyophilized to afford (2-(2,6-dioxopiperidin-3-yl)-7-methyl-3-oxoisoindolin-5-yl)methyl (3-chloro-4-methylphenyl)carbamate 69. 1H NMR (400 MHz, DMSO-d6) δ=11.01 (s, 1H), 9.92 (br s, 1H), 7.60 (br d, J=5.3 Hz, 2H), 7.48 (s, 1H), 7.30-7.22 (m, 2H), 5.23 (s, 2H), 5.19-5.09 (m, 1H), 4.47-4.37 (m, 1H), 4.31-4.23 (m, 1H), 2.99-2.87 (m, 1H), 2.60 (br d, J=18.4 Hz, 1H), 2.41-2.37 (m, 1H), 2.34 (s, 3H), 2.24 (s, 3H), 2.03-1.96 (m, 1H). MS (ESI) m/z 456.2 [M+H]+

### Example 70: Step 1

To a solution of 2-bromo-5-methyl-phenol (5.00 g, 26.7 mmol, 1.00 eq) in acetonitrile (100 mL) was added triethylamine (10.8 g, 106 mmol, 14.9 mL, 4.00 eq) and magnesium chloride (3.82 g, 40.1 mmol, 1.65 mL, 1.50 eq), the mixture was stirred at 25° C. for 15 min, then paraformaldehyde (5.62 g, 187 mmol, 7.00 eq) was added, the mixture was stirred at 80° C. for another 2 h. The reaction mixture was poured into hydrochloric acid (200 mL, 3 M) and stirred for 10 min. The aqueous phase was extracted with ethyl acetate (3×100 mL). The combined organic phase was washed with brine (80.0 mL), dried over anhydrous sodium sulfate, filtered and concentrated in vacuum to give a residue. The residue was purified by silica gel chromatography to afford 3-bromo-2-hydroxy-6-methylbenzaldehyde. 1H NMR (400 MHz, CDCl3) δ=12.54 (s, 1H), 10.28 (s, 1H), 7.64 (d, J=8.0 Hz, 1H), 6.66 (s, 1H), 2.61 (s, 3H)

Step 2: To a solution of 3-bromo-2-hydroxy-6-methylbenzaldehyde (2.90 g, 13.5 mmol, 1.00 eq) and potassium carbonate (3.73 g, 26.9 mmol, 2.00 eq) in dimethylformamide (30.0 mL) was added iodomethane (1.93 g, 13.6 mmol, 848 uL, 1.01 eq) at 25° C., the mixture was stirred at 25° C. for 1 h. The reaction mixture was poured into water (100 mL), extracted with ethyl acetate (3×60.0 mL). The combined organic phase was washed with brine (100 mL), dried over anhydrous sodium sulfate, filtered and concentrated in vacuum to give a residue. The residue was purified by silica gel chromatography to afford 3-bromo-2-methoxy-6-methylbenzaldehyde. 1H NMR (400 MHz, CDCl3) δ=10.44 (s, 1H), 7.55 (d, J=8.4 Hz, 1H), 6.84 (d, J=8.4 Hz, 1H), 3.86 (s, 3H), 2.46 (s, 3H).

Step 3: To a solution of 3-bromo-2-methoxy-6-methyl-benzaldehyde (2.50 g, 10.9 mmol, 1.00 eq) in tetrahydrofuran (20.0 mL), water (10.0 mL) and tertiary butanol (5.00 mL) was added sodium; chlorite (3.95 g, 43.6 mmol, 4.00 eq) and sodium dihydrogen phosphate (5.24 g, 43.6 mmol, 4.00 eq), the mixture was stirred at 15° C. for 15 min, then 2-methylbut-2-ene (5.36 g, 76.4 mmol, 8.09 mL, 7.00 eq) was added, the mixture was stirred at 15° C. for another 45 min. The reaction mixture was poured into water (80.0 mL) and adjust pH=9-10 with 1 M sodium hydroxide. The aqueous phase was extracted with ethyl acetate (3×50.0 mL). The combined organic phase was removed, the aqueous phase was adjusted to pH=5-6 with 1M hydrochloric acid. The aqueous phase was extracted with ethyl acetate (3×50.0 mL). The combined organic phase was washed with brine (100 mL), dried over anhydrous sodium sulfate, filtered and concentrated in vacuum to give 3-bromo-2-methoxy-6-methylbenzoic acid. 1H NMR (400 MHz, DMSO-d6) δ=13.38 (br s, 1H), 7.57 (d, J=8.0 Hz, 1H), 7.00 (d, J=8.8 Hz, 1H), 3.79 (s, 3H), 2.23 (s, 3H).

Step 4: To a solution of 3-bromo-2-methoxy-6-methyl-benzoic acid (2.00 g, 8.16 mmol, 1.00 eq) and potassium carbonate (2.26 g, 16.3 mmol, 2.00 eq) in dimethylformamide (20.0 mL) was added iodomethane (1.16 g, 8.16 mmol, 508 uL, 1.00 eq) at 15° C., the mixture was stirred at 15° C. for 1 h. The reaction mixture was poured into water (120 mL) and stirred for 10 min. The aqueous phase was extracted with ethyl acetate (3×60.0 mL). The combined organic phase was washed with brine (100 mL), dried over anhydrous sodium sulfate, filtered and concentrated in vacuum to give a residue. The residue was purified by silica gel chromatography to afford methyl 3-bromo-2-methoxy-6-methylbenzoate. 1H NMR (400 MHz, DMSO-d6) δ=7.63 (d, J=8.4 Hz, 1H), 7.03 (dd, J=0.8, 8.4 Hz, 1H), 3.88 (s, 3H), 3.78 (s, 3H), 2.20 (s, 3H).

Step 5: To a solution of methyl 3-bromo-2-methoxy-6-methyl-benzoate (2.00 g, 7.72 mmol, 1.00 eq) in trichloromethane (20.0 mL) was added N-bromosuccinimide (1.51 g, 8.49 mmol, 1.10 eq) and benzoyl peroxide (374 mg, 1.54 mmol, 0.200 eq), the mixture was stirred at 90° C. for 12 h. The reaction mixture was concentrated to give a residue. The residue was purified by silica gel chromatography to afford methyl 3-bromo-6-(bromomethyl)-2-methoxy-benzoate. 1H NMR (400 MHz, CDCl3) δ=7.50 (d, J=8.4 Hz, 1H), 6.99 (d, J=8.4 Hz, 1H), 4.40 (s, 2H), 3.91 (s, 3H), 3.84 (s, 3H).

Step 6: To a solution of methyl 3-bromo-6-(bromomethyl)-2-methoxy-benzoate (2.00 g, 5.92 mmol, 1.00 eq) and 3-aminopiperidine-2,6-dione (1.07 g, 6.51 mmol, 1.10 eq, hydrochloric acid) in acetonitrile (20.0 mL) was added N,N-diisopropylethylamine (2.29 g, 17.7 mmol, 3.09 mL, 3.00 eq), the mixture was stirred at 80° C. for 12 h. The reaction mixture was concentrated to give a residue. The residue was triturated with ethyl acetate (20.0 mL) and 1 M hydrochloric acid (10.0 mL) at 25° C. for 10 min to afford 3-(6-bromo-7-methoxy-1-oxo-isoindolin-2-yl)piperidine-2,6-dione. 1H NMR (400 MHz, DMSO-d6) δ=11.01 (s, 1H), 7.87 (d, J=8.0 Hz, 1H), 7.30 (d, J=8.0 Hz, 1H), 5.09 (dd, J=5.2, 13.2 Hz, 1H), 4.51-4.37 (m, 1H), 4.35-4.23 (m, 1H), 3.98 (s, 3H), 2.98-2.83 (m, 1H), 2.69-2.58 (m, 1H), 2.41-2.32 (m, 1H), 2.06-1.99 (m, 1H).

Step 7: To a solution of 3-(6-bromo-7-methoxy-1-oxo-isoindolin-2-yl)piperidine-2,6-dione (800 mg, 2.27 mmol, 1.00 eq) and 1,8-diazabicyclo[5.4.0]undec-7-ene (1.38 g, 9.06 mmol, 1.37 mL, 4.00 eq) in dimethylformamide (10.0 mL) was added (2-(chloromethoxy)ethyl)trimethylsilane (1.36 g, 8.15 mmol, 1.44 mL, 3.60 eq) at 0° C., the mixture was stirred at 25° C. for 2 h. The reaction was poured into water (200 mL) and stirred for 10 min. The aqueous phase was extracted with ethyl acetate (3×60.0 mL). The combined organic phase was washed with brine (100 mL), dried over anhydrous anhydrous sodium sulfate, filtered and concentrated in vacuum to give a residue. The residue was purified by silica gel chromatography to afford 3-(6-bromo-7-methoxy-1-oxoisoindolin-2-yl)-1-((2-(trimethylsilyl)ethoxy)methyl)piperidine-2,6-dione. 1H NMR (400 MHz, DMSO-d6) δ=7.89 (d, J=8.0 Hz, 1H), 7.32 (d, J=8.0 Hz, 1H), 5.28-5.17 (m, 1H), 5.07 (q, J=9.6 Hz, 2H), 4.52-4.41 (m, 1H), 4.34-4.20 (m, 1H), 4.00 (s, 3H), 3.62-3.48 (m, 2H), 3.14-3.00 (m, 1H), 2.87-2.79 (m, 1H), 2.39 (dt, J=9.2, 13.2 Hz, 1H), 2.13-2.05 (m, 1H), 0.90-0.83 (m, 2H), 0.06-0.05 (m, 9H).

Step 8: To a solution of 3-(6-bromo-7-methoxy-1-oxoisoindolin-2-yl)-1-((2-(trimethylsilyl)ethoxy)methyl) piperidine-2,6-dione (600 mg, 1.24 mmol, 1.00 eq), triethylsilane (1.44 g, 12.4 mmol, 1.98 mL, 10.0 eq) and N,N-diisopropylethylamine (802 mg, 6.21 mmol, 1.08 mL, 5.00 eq) in dimethylformamide (10.0 mL) was added [1,1-bis(diphenylphosphino)ferrocene]dichloropalladium(II) (90.8 mg, 124 umol, 0.100 eq), the mixture was stirred at 80° C. for 12 h under carbonic oxide atmosphere. The reaction mixture was poured into water (60.0 mL) and stirred for 10 min. The aqueous phase was extracted with ethyl acetate (3×30.0 mL). The combined organic phase was washed with brine (100 mL), dried with anhydrous sodium sulfate, filtered and concentrated in vacuum to give a residue. The residue was purified by reversed-phase HPLC to give 2-(2,6-dioxo-1-((2-(trimethylsilyl)ethoxy)methyl)piperidin-3-yl)-4-methoxy-3-oxoisoindoline-5-carbaldehyde. 1H NMR (400 MHz, DMSO-d6) δ=10.41 (s, 1H), 7.98 (d, J=7.6 Hz, 1H), 7.48 (d, J=7.6 Hz, 1H), 5.29-5.19 (m, 1H), 5.12-4.99 (m, 2H), 4.62-4.53 (m, 1H), 4.45-4.34 (m, 1H), 4.20 (s, 3H), 3.55 (dq, J=2.4, 8.0 Hz, 2H), 3.12-3.03 (m, 1H), 2.83 (br d, J=15.6 Hz, 1H), 2.41 (br dd, J=4.4, 13.2 Hz, 1H), 2.14-2.04 (m, 1H), 0.89-0.82 (m, 2H), 0.07-0.05 (m, 9H).

Step 9: To a solution of 2-(2,6-dioxo-1-((2-(trimethylsilyl)ethoxy)methyl)piperidin-3-yl)-4-methoxy-3-oxoisoindoline-5-carbaldehyde (250 mg, 578 umol, 1.00 eq) and acetic acid (173 mg, 2.89 mmol, 165 uL, 5.00 eq) in dimethylformamide (2.00 mL) and dichloromethane (2.00 mL) was added sodium triacetoxyhydroborate (612 mg, 2.89 mmol, 5.00 eq), the mixture was stirred at 50° C. for 2 h. The reaction mixture was poured into water (80.0 mL) and stirred for 10 min. The aqueous phase was extracted with ethyl acetate (3×40.0 mL). The combined organic phase was washed with brine (100 mL), dried over anhydrous sodium sulfate, filtered and concentrated in vacuum to give a residue. The residue was purified by reversed-phase HPLC to afford 3-(6-(hydroxymethyl)-7-methoxy-1-oxoisoindolin-2-yl)-1-((2-(trimethylsilyl)ethoxy)methyl)piperidine-2,6-dione. 1H NMR (400 MHz, DMSO-d6) δ=7.66 (d, J=7.6 Hz, 1H), 7.30 (d, J=7.6 Hz, 1H), 5.23-5.15 (m, 2H), 5.13-5.02 (m, 2H), 4.59 (d, J=5.6 Hz, 2H), 4.43 (br d, J=17.2 Hz, 1H), 4.30-4.20 (m, 1H), 3.96 (s, 3H), 3.53 (dq, J=2.4, 8.0 Hz, 2H), 3.09-3.01 (m, 1H), 2.82-2.77 (m, 1H), 2.38 (br dd, J=4.4, 13.2 Hz, 1H), 2.10-2.03 (m, 1H), 0.90-0.80 (m, 2H), 0.02 (s, 9H).

Step 10: A mixture of 3-(6-(hydroxymethyl)-7-methoxy-1-oxoisoindolin-2-yl)-1-((2-(trimethylsilyl)ethoxy) methyl) piperidine-2,6-dione (50.0 mg, 115 umol, 1.00 eq) in hydrochloric acid/dioxane (4 M, 3.00 mL, 104 eq) was stirred at 50° C. for 1 h. The reaction mixture was concentrated to give 1-(hydroxymethyl)-3-(6-(hydroxymethyl)-7-methoxy-1-oxoisoindolin-2-yl)piperidine-2,6-dione.

Step 11: To a solution of 1-(hydroxymethyl)-3-(6-(hydroxymethyl)-7-methoxy-1-oxoisoindolin-2-yl)piperidine-2,6-dione (40.0 mg, 119 umol, 1.00 eq) in acetonitrile (4.00 mL) was added ammonium hydroxide (145 mg, 1.50 mmol, 160 uL, 36% purity, 12.5 eq), the mixture was stirred at 25° C. for 1 h. The reaction mixture was added formic acid (100 uL) and concentrated in vacuum to give a residue. The residue was purified by Prep-HPLC to afford 3-(6-(hydroxymethyl)-7-methoxy-1-oxoisoindolin-2-yl) piperidine-2,6-dione. 1H NMR (400 MHz, DMSO-d6) δ=10.99 (br s, 1H), 7.65 (d, J=7.6 Hz, 1H), 7.29 (d, J=7.6 Hz, 1H), 5.24-5.14 (m, 1H), 5.07 (dd, J=5.2, 13.2 Hz, 1H), 4.58 (br s, 2H), 4.45-4.36 (m, 1H), 4.31-4.20 (m, 1H), 3.96 (s, 3H), 2.96-2.85 (m, 1H), 2.59-2.56 (m, 1H), 2.39 (dd, J=4.4, 13.2 Hz, 1H), 2.05-1.96 (m, 1H).

Step 12: To a solution of 3-(6-(hydroxymethyl)-7-methoxy-1-oxoisoindolin-2-yl)piperidine-2,6-dione (8.00 mg, 26.3 umol, 1.00 eq) and phenyl (3-chloro-4-methylphenyl)carbamate (see Compound 1) (10.3 mg, 39.4 umol, 1.50 eq) in dimethylformamide (1.00 mL) was added sodium hydride (2.10 mg, 52.6 umol, 60% purity, 2.00 eq) at 25° C., the mixture was stirred at 25° C. for 1 h. The reaction mixture was quenched with formic acid (100 uL) and filtered to give a filtrate. The filtrate was purified by Prep-HPLC to afford (2-(2,6-dioxopiperidin-3-yl)-4-methoxy-3-oxoisoindolin-5-yl)methyl(3-chloro-4-methylphenyl) carbamate 70. 1H NMR (400 MHz, DMSO-d6) δ=10.99 (br s, 1H), 9.85 (s, 1H), 7.68 (d, J=7.6 Hz, 1H), 7.60 (s, 1H), 7.34 (d, J=7.6 Hz, 1H), 7.31-7.22 (m, 2H), 5.22 (s, 2H), 5.09 (dd, J=5.2, 13.2 Hz, 1H), 4.48-4.39 (m, 1H), 4.37-4.26 (m, 1H), 4.05 (s, 3H), 2.96-2.85 (m, 1H), 2.61-2.56 (m, 1H), 2.42-2.35 (m, 1H), 2.25 (s, 3H), 2.06-1.97 (m, 1H). MS (ESI) m/z 472.1 [M+H]+

Compound 71: Step 1: A mixture of 3-bromo-2-fluoro-benzoic acid (6.00 g, 27.4 mmol, 1.00 eq), palladium acetate (615 mg, 2.74 mmol, 0.100 eq) and dipotassium phosphate (14.3 g, 82.2 mmol, 3.00 eq) in dibromomethane (50.0 mL) was stirred at 140° C. for 36 h under nitrogen atmosphere. The reaction mixture was poured into dichloromethane (300 mL) and stirred for 30 min, then filtered to give a filtrate. The filtrate was concentrated to give a residue. The residue was purified by silica gel chromatography to afford 6-bromo-7-fluoroisobenzofuran-1(3H)-one. 1H NMR (400 MHz, CDCl3) δ=7.80 (dd, J=6.0, 7.9 Hz, 1H), 7.11 (d, J=8.0 Hz, 1H), 5.21 (s, 2H).

Step 2: To a solution of 6-bromo-7-fluoroisobenzofuran-1(3H)-one (950 mg, 4.11 mmol, 1.00 eq) in ethanol (20.0 mL) was added thionyl chloride (4.89 g, 41.1 mmol, 2.98 mL, 10.0 eq). The mixture was stirred at 100° C. for 12 h. The reaction mixture was concentrated to give a residue. The residue was purified by silica gel chromatography to afford ethyl 3-bromo-6-(chloromethyl)-2-fluorobenzoate. 1H NMR (400 MHz, CDCl3) δ=7.55 (dd, J=6.8, 8.4 Hz, 1H), 7.07 (dd, J=0.6, 8.3 Hz, 1H), 4.62 (s, 2H), 4.39 (q, J=7.2 Hz, 2H), 1.34 (t, J=7.2 Hz, 3H).

Step 3: To a solution of ethyl 3-bromo-6-(chloromethyl)-2-fluorobenzoate (600 mg, 2.03 mmol, 1.00 eq) and 3-aminopiperidine-2,6-dione (400 mg, 2.44 mmol, 1.20 eq, hydrochloric acid) in dimethylformamide (6.00 mL) was added N,N-diisopropylethylamine (1.31 g, 10.1 mmol, 1.77 mL, 5.00 eq), the mixture was stirred at 100° C. for 12 h. The reaction mixture was filtered to give a filter cake. The filter cake was concentrated to give a residue. The residue was triturated with ethyl acetate at 25° C. for 10 min to afford 3-(6-bromo-7-fluoro-1-oxoisoindolin-2-yl)piperidine-2,6-dione. 1H NMR (400 MHz, DMSO-d6) δ=11.02 (br s, 1H), 7.95 (dd, J=6.2, 8.1 Hz, 1H), 7.43 (d, J=8.1 Hz, 1H), 5.10 (dd, J=5.1, 13.3 Hz, 1H), 4.53-4.45 (m, 1H), 4.41-4.31 (m, 1H), 2.98-2.84 (m, 1H), 2.65-2.57 (m, 1H), 2.45-2.32 (m, 1H), 2.03 (tdd, J=2.6, 5.2, 12.5 Hz, 1H).

Step 4: To a solution of 3-(6-bromo-7-fluoro-1-oxoisoindolin-2-yl)piperidine-2,6-dione (170 mg, 498 umol, 1.00 eq) in dioxane (2.00 mL) was added tributylstannylmethanol (200 mg, 622 umol, 1.25 eq) and tetrakis[triphenylphosphine]palladium(0) (172 mg, 149 umol, 0.300 eq), the mixture was stirred at 90° C. for 12 h under nitrogen atmosphere. The reaction mixture was quenched with saturated potassium fluoride (1.00 mL) and stirred for 1 h, then filtered to give a filtrate, the filtrate was concentrated to give a residue. The residue was purified by Prep-HPLC to afford 3-(7-fluoro-6-hydroxy-1-oxoisoindolin-2-yl)piperidine-2,6-dione. MS (ESI) m/z 293.1 [M+H]+

Step 5: To a solution of 3-(7-fluoro-6-hydroxy-1-oxoisoindolin-2-yl) piperidine-2,6-dione (15.0 mg, 51.3 umol, 1.00 eq) and phenyl N-(3-chloro-4-methyl-phenyl)carbamate (see Compound 1) (16.1 mg, 61.5 umol, 1.20 eq) in dimethylformamide (1.00 mL) was added sodium hydride (3.08 mg, 77.0 umol, 60% purity, 1.50 eq), the mixture was stirred at 25° C. for 1 h. The reaction mixture was quenched with water (0.200 mL) and filtered to give a filtrate. The filtrate was purified by Prep-HPLC to afford (2-(2,6-dioxopiperidin-3-yl)-4-fluoro-3-oxoisoindolin-5-yl)methyl(3-chloro-4-methylphenyl)carbamate 71. 1H NMR (400 MHz, DMSO-d6) δ=11.10-10.94 (m, 1H), 9.92 (s, 1H), 7.78 (t, J=7.0 Hz, 1H), 7.58 (s, 1H), 7.47 (d, J=7.7 Hz, 1H), 7.32-7.23 (m, 2H), 5.28 (s, 2H), 5.09 (dd, J=5.1, 13.3 Hz, 1H), 4.55-4.44 (m, 1H), 4.41-4.30 (m, 1H), 2.97-2.86 (m, 1H), 2.64-2.58 (m, 1H), 2.38 (br d, J=4.2 Hz, 1H), 2.25 (s, 3H), 2.07-1.98 (m, 1H). MS (ESI) m/z 459.9 [M+H]+

Compound 72: Step 1:

A mixture of 3-bromo-2-chlorobenzoic acid (7.00 g, 29.7 mmol, 1.00 eq), potassium phosphate dibasic (15.5 g, 89.2 mmol, 3.00 eq) and palladium(II) acetate (667 mg, 2.97 mmol, 0.100 eq) in dibromomethane (50.0 mL) was stirred at 140° C. under nitrogen for 36 h. The reaction mixture was concentrated under reduced pressure to remove dibromomethane. The residue was diluted with water (60.0 mL) and extracted with ethyl acetate (3×80.0 mL). The combined organic layers were washed with brine (60.0 mL), dried over magnesium sulfate, filtered and concentrated under reduced pressure to give a residue. The residue was purified by column chromatography to afford 6-bromo-7-chloroisobenzofuran-1(3H)-one. 1H NMR (400 MHz, CDCl3) δ=7.85 (d, J=8.2 Hz, 1H), 7.21 (d, J=8.2 Hz, 1H), 5.15 (s, 2H).

Step 2: A mixture of 6-bromo-7-chloroisobenzofuran-1(3H)-one (3.00 g, 12.1 mmol, 1.00 eq) and thionyl chloride (14.4 g, 121 mmol, 8.79 mL, 10.0 eq) in ethyl alcohol (30.0 mL) was stirred at 90° C. for 36 h. The reaction mixture was concentrated under reduced pressure to give a residue. The residue was purified by column chromatography to afford ethyl 3-bromo-2-chloro-6-(chloromethyl)benzoate. 1H NMR (400 MHz, CDCl3) δ=7.60 (d, J=8.4 Hz, 1H), 7.17 (d, J=8.4 Hz, 1H), 4.47 (s, 2H), 4.40 (q, J=7.2 Hz, 2H), 1.36 (t, J=7.2 Hz, 3H).

Step 3: A mixture of ethyl 3-bromo-2-chloro-6-(chloromethyl)benzoate (0.500 g, 1.60 mmol, 1.00 eq), 3-aminopiperidine-2,6-dione (290 mg, 1.76 mmol, 1.10 eq, hydrochloric acid) and N,N-diisopropylethylamine (414 mg, 3.21 mmol, 558 uL, 2.00 eq) in acetonitrile (5.00 mL) was stirred at 100° C. for 10 h. The reaction mixture was concentrated under reduced pressure to give a residue. The residue was purified was by reversed phase chromatography and lyophilized to afford 3-(6-bromo-7-chloro-1-oxoisoindolin-2-yl)piperidine-2,6-dione. 1H NMR (400 MHz, DMSO-d6) δ=11.04 (br s, 1H), 8.02 (d, J=8.2 Hz, 1H), 7.55 (br d, J=8.2 Hz, 1H), 5.11 (br dd, J=4.8, 13.4 Hz, 1H), 4.47-4.39 (m, 1H), 4.34-4.27 (m, 1H), 2.98-2.87 (m, 2H), 2.41-2.32 (m, 1H), 2.07-1.97 (m, 1H).

Step 4: A mixture of 3-(6-bromo-7-chloro-1-oxoisoindolin-2-yl)piperidine-2,6-dione (0.100 g, 280 umol, 1.00 eq), (tributylstannyl)methanol (135 mg, 419 umol, 1.50 eq) and (1,1′-bis(diphenylphosphino)ferrocene) palladium(II) dichloride (20.5 mg, 28.0 umol, 0.10 eq) in dioxane (1.00 mL) was stirred at 100° C. for 10 h. The reaction mixture was quenched by addition saturated potassium fluoride (1.00 mL), and then filtered and concentrated under reduced pressure to give a residue. The residue was purified by Prep-HPLC to afford 3-(7-chloro-6-(hydroxymethyl)-1-oxoisoindolin-2-yl)piperidine-2,6-dione. 1H NMR (400 MHz, DMSO-d6) δ=11.13-10.91 (m, 1H), 8.58-8.43 (m, 1H), 7.79 (d, J=7.8 Hz, 1H), 7.58 (d, J=7.6 Hz, 1H), 5.68-5.40 (m, 1H), 5.09 (dd, J=4.8, 13.6 Hz, 1H), 4.66 (br s, 2H), 4.51-4.20 (m, 2H), 2.99-2.87 (m, 2H), 2.44-2.37 (m, 1H), 2.06-1.97 (m, 1H).

Step 5: To a solution of 3-(7-chloro-6-(hydroxymethyl)-1-oxoisoindolin-2-yl)piperidine-2,6-dione (15.0 mg, 48.6 umol, 1.00 eq) and phenyl (3-chloro-4-methylphenyl)carbamate (see Compound 1) (12.7 mg, 48.6 umol, 1.00 eq) in N,N-dimethylformamide (1.00 mL) was added sodium hydride (1.94 mg, 48.6 umol, 60% purity, 1.00 eq). Then the mixture was stirred at 25° C. for 1 h. The reaction mixture was quenched by addition acetic acid (0.50 mL), and then filtered. The filtrate was purified by Prep-HPLC to afford (4-chloro-2-(2,6-dioxopiperidin-3-yl)-3-oxoisoindolin-5-yl)methyl(3-chloro-4-methylphenyl) carbamate 72. 1H NMR (400 MHz, DMSO-d6) δ=11.01 (br s, 1H), 9.97 (br s, 1H), 7.80 (d, J=7.8 Hz, 1H), 7.68-7.56 (m, 2H), 7.38-7.18 (m, 2H), 5.34 (s, 2H), 5.11 (dd, J=5.2, 13.2 Hz, 1H), 4.53-4.42 (m, 1H), 4.38-4.28 (m, 1H), 2.97-2.88 (m, 1H), 2.62 (br s, 1H), 2.40 (br dd, J=4.6, 13.2 Hz, 1H), 2.26 (s, 3H), 2.07-1.99 (m, 1H). MS (ESI) m/z 476.0 [M+H]+

Compound 73: Step 1: A mixture of 3-bromo-2-methylbenzoic acid (6.00 g, 27.9 mmol, 1.00 eq), palladium(II) acetate (626 mg, 2.79 mmol, 0.10 eq) and potassium phosphate dibasic (14.6 g, 83.7 mmol, 3.00 eq) in dibromomethane (50.0 mL) was stirred at 140° C. under nitrogen atmosphere for 10 h. The reaction mixture was concentrated under reduced pressure to remove dibromomethane. The residue was diluted with water (60.0 mL) and extracted with ethyl acetate (3×80.0 mL). The combined organic layers were washed with brine (60.0 mL), dried over magnesium sulfate, filtered and concentrated under reduced pressure to give a residue. The residue was purified by column chromatography to afford 6-bromo-7-methylisobenzofuran-1(3H)-one. 1H NMR (400 MHz, CDCl3) δ=7.83 (d, J=8.2 Hz, 1H), 7.19 (d, J=8.2 Hz, 1H), 5.22 (s, 2H), 2.79 (s, 3H).

Step 2: A mixture of 6-bromo-7-methylisobenzofuran-1(3H)-one (3.30 g, 14.5 mmol, 1.00 eq) and thionyl chloride (17.3 g, 145 mmol, 10.5 mL, 10.0 eq) in ethyl alcohol (30.0 mL) was stirred at 70° C. for 10 h. The reaction mixture was concentrated under reduced pressure to give a residue. The residue was purified by column chromatography to afford ethyl 3-bromo-6-(chloromethyl)-2-methylbenzoate. 1H NMR (400 MHz, CDCl3) δ=7.52 (d, J=8.2 Hz, 1H), 7.07 (d, J=8.2 Hz, 1H), 4.49 (s, 2H), 4.38 (q, J=7.2 Hz, 2H), 2.33 (s, 3H), 1.35 (t, J=7.2 Hz, 3H).

Step 3: A mixture of ethyl 3-bromo-6-(chloromethyl)-2-methylbenzoate (0.140 g, 480 umol, 1.00 eq), 3-aminopiperidine-2,6-dione hydrochloride (94.8 mg, 576 umol, 1.20 eq) and N,N-diisopropylethylamine (310 mg, 2.40 mmol, 418 uL, 5.00 eq) in acetonitrile (2.00 mL) was stirred at 80° C. for 10 h. The reaction mixture was concentrated under reduced pressure to give a residue. The residue was purified was by reversed phase chromatography and lyophilized to afford 3-(6-bromo-7-methyl-1-oxoisoindolin-2-yl)piperidine-2,6-dione. 1H NMR (400 MHz, DMSO-d6) δ=11.00 (s, 1H), 7.82 (d, J=7.8 Hz, 1H), 7.39 (d, J=8.0 Hz, 1H), 5.10 (dd, J=5.2, 13.2 Hz, 1H), 4.42-4.20 (m, 2H), 2.97-2.85 (m, 1H), 2.72 (s, 3H), 2.63 (br s, 1H), 2.39 (br dd, J=4.4, 13.2 Hz, 1H), 2.05-1.96 (m, 1H).

Step 4: A mixture of 3-(6-bromo-7-methyl-1-oxoisoindolin-2-yl)piperidine-2,6-dione (0.100 g, 297 umol, 1.00 eq), (1,1′-bis(diphenylphosphino)ferrocene)palladium(II) dichloride (43.4 mg, 59.3 umol, 0.20 eq), N,N-diisopropylethylamine (192 mg, 1.48 mmol, 258 uL, 5.00 eq) and triethylsilane (345 mg, 2.97 mmol, 474 uL, 10.0 eq) in N,N-dimethylformamide (2.00 mL) was stirred at 80° C. under carbon monoxide (50 psi) atmosphere for 12 h. The reaction mixture was diluted with water (15.0 mL) and extracted with ethyl acetate (3×25.0 mL). The combined organic layers were washed with brine (10.0 mL), dried over sodium sulfate, filtered and concentrated under reduced pressure to give a residue. The residue was purified was by reverse phase chromatography and lyophilized to afford 2-(2,6-dioxopiperidin-3-yl)-4-methyl-3-oxoisoindoline-5-carbaldehyde. 1H NMR (400 MHz, DMSO-d6) δ=11.01 (br s, 1H), 10.39 (s, 1H), 8.04 (d, J=7.8 Hz, 1H), 7.62 (d, J=7.8 Hz, 1H), 5.12 (dd, J=5.2, 13.2 Hz, 1H), 4.54-4.45 (m, 1H), 4.41-4.32 (m, 1H), 3.04 (s, 3H), 2.95-2.87 (m, 2H), 2.63 (br s, 1H), 2.06-1.98 (m, 1H).

Step 5: To a solution of 2-(2,6-dioxopiperidin-3-yl)-4-methyl-3-oxoisoindoline-5-carbaldehyde (20.0 mg, 69.9 umol, 1.00 eq) and acetic acid (8.39 mg, 140 umol, 7.99 uL, 2.00 eq) in N,N-dimethylformamide (1.00 mL) and dichloromethane (1.00 mL) was added sodium triacetoxy borohydride (74.0 mg, 349 umol, 5.00 eq). Then the mixture was stirred at 50° C. for 2 h. The reaction mixture was quenched by addition water (5.00 mL), and then filtered and concentrated under reduced pressure to give a residue. The residue was purified was by reversed phase chromatography and lyophilized to afford 3-(6-(hydroxymethyl)-7-methyl-1-oxoisoindolin-2-yl)piperidine-2,6-dione. MS (ESI) m/z 289.1 [M+H]+

Step 6: To a solution of 3-(6-(hydroxymethyl)-7-methyl-1-oxoisoindolin-2-yl)piperidine-2,6-dione (20.0 mg, 69.4 umol, 1.00 eq) and phenyl (3-chloro-4-methylphenyl)carbamate (see Compound 1) (18.2 mg, 69.4 umol, 1.00 eq) in N,N-dimethylformamide (1.00 mL) was added sodium hydride (5.55 mg, 139 umol, 60% purity, 2.00 eq). Then the mixture was stirred at 25° C. for 1 h. The reaction mixture was quenched by addition acetic acid (1 mL) and filtered. The filtrate was purified by prep-HPLC to afford (2-(2,6-dioxopiperidin-3-yl)-4-methyl-3-oxoisoindolin-5-yl)methyl(3-chloro-4-methylphenyl)carbamate. 1H NMR (400 MHz, DMSO-d6) δ=10.99 (br s, 1H), 9.88 (s, 1H), 8.42 (br s, 1H), 7.67-7.56 (m, 2H), 7.43 (d, J=7.8 Hz, 1H), 7.33-7.20 (m, 2H), 5.27 (s, 2H), 5.09 (dd, J=5.2, 13.4 Hz, 1H), 4.45-4.34 (m, 1H), 4.32-4.21 (m, 1H), 2.98-2.86 (m, 1H), 2.73 (s, 3H), 2.65-2.57 (m, 1H), 2.46-2.37 (m, 1H), 2.25 (s, 3H), 2.05-1.94 (m, 1H). MS (ESI) m/z 456.1 [M+H]+

Compound 74: Step 1: To a solution of bromobenzene (1.00 g, 6.37 mmol, 671 uL, 1.00 eq), tert-butyl azetidin-3-ylcarbamate (2.66 g, 12.7 mmol, 2.00 eq, hydrochloric acid) and cesium carbonate (6.23 g, 19.1 mmol, 3.00 eq) in dioxane (20.0 mL) was added 4,5-bis(diphenylphosphino)-9,9-dimethylxanthene (553 mg, 955 umol, 0.15 eq) and tris(dibenzylideneacetone)dipalladium(0) (292 mg, 318 umol, 0.05 eq). The mixture was stirred at 90° C. for 12 h under nitrogen atmosphere. The reaction mixture was quenched by addition water (50.0 mL), and extracted with ethyl acetate (2×100 mL). The combined organic layers were dried over anhydrous sodium sulfate, filtered and concentrated under reduced pressure to give a residue. The residue was purified by column chromatography (SiO₂, petroleum ether/ethyl acetate=1/0 to 10/1) to give tert-butyl (1-phenylazetidin-3-yl)carbamate (1.20 g, 4.83 mmol, 76% yield) as a yellow solid. ¹H NMR (400 MHz, DMSO-d₆) δ=7.50 (br d, J=7.2 Hz, 1H), 7.15 (t, J=7.8 Hz, 2H), 6.67 (t, J=7.3 Hz, 1H), 6.41 (d, J=7.9 Hz, 2H), 4.47-4.33 (m, 1H), 4.04 (t, J=7.3 Hz, 2H), 3.53 (t, J=6.8 Hz, 2H), 1.39 (s, 9H).

Step 2: To a solution of tert-butyl (1-phenylazetidin-3-yl)carbamate (1.20 g, 4.83 mmol, 1.00 eq) in dichloromethane (20.0 mL) was added trifluoroacetic acid (6.16 g, 54.0 mmol, 4.00 mL, 11.2 eq). The mixture was stirred at 20° C. for 1 h. The reaction mixture was concentrated under reduced pressure to give 1-phenylazetidin-3-amine (700 mg, crude) as yellow oil. ¹H NMR (400 MHz, DMSO-d₆) δ=8.33 (br s, 2H), 7.19 (dd, J=7.5, 8.4 Hz, 2H), 6.73 (t, J=7.3 Hz, 1H), 6.50 (d, J=7.6 Hz, 2H), 4.14-4.04 (m, 3H), 3.81-3.73 (m, 2H).

Step 3: To a solution of 1-phenylazetidin-3-amine (700 mg, 4.72 mmol, 1.00 eq) in acetonitrile (20.0 mL) was added pyridine (2.94 g, 37.2 mmol, 3.00 mL, 7.87 eq). Then phenyl carbonochloridate (813 mg, 5.20 mmol, 650 uL, 1.10 eq) was added into the mixture at 0° C. The mixture was stirred at 20° C. for 1 h. The reaction mixture was concentrated under reduced pressure to give a residue. The residue was purified by reversed-phase HPLC (column: spherical C18, 20-45 um, 100 Å, SW 120, mobile phase: [water (0.1% Formic Acid)-ACN]) to give phenyl (1-phenylazetidin-3-yl)carbamate (1.01 g, 2.94 mmol, 62% yield, 78% purity) as a white solid. MS (ESI) m/z 269.0 [M+H]⁺

Step 4: To a solution of phenyl (1-phenylazetidin-3-yl)carbamate (86.1 mg, 321 umol, 1.10 eq) and 3-(6-(hydroxymethyl)-1-oxoisoindolin-2-yl)piperidine-2,6-dione I (80.0 mg, 292 umol, 1.00 eq) in dimethyl formamide (1.50 mL) was added sodium hydride (23.3 mg, 583 umol, 60% purity, 2.00 eq) at 0° C. The mixture was stirred at 0° C. for 1 h. The pH of the mixture was adjusted to around 6 by adding hydrochloric acid. The mixture was extracted with ethyl acetate (3×10.0 mL). The combined organic layers were dried over anhydrous sodium sulfate, filtered and concentrated under reduced pressure to give a residue. The residue was purified by prep-HPLC (column: Phenomenex Synergi C18 150*25 mm*10 um; mobile phase: [water (0.225% FA)-ACN]; B %: 24%-54%, 10 min), and lyophilized to afford (2-(2,6-dioxopiperidin-3-yl)-3-oxoisoindolin-5-yl)methyl(1-phenylazetidin-3-yl)carbamate #110 (62.7 mg, 138 umol, 47% yield, 99% purity, formate) as a white solid. ¹H NMR (400 MHz, DMSO-d6) δ=10.99 (br s, 1H), 8.01 (br d, J=7.6 Hz, 1H), 7.72 (s, 1H), 7.61 (s, 2H), 7.15 (t, J=7.9 Hz, 2H), 6.68 (t, J=7.3 Hz, 1H), 6.43 (br d, J=7.8 Hz, 2H), 5.18-5.09 (m, 3H), 4.50-4.42 (m, 2H), 4.35-4.28 (m, 1H), 4.08 (t, J=7.3 Hz, 2H), 3.57 (br t, J=6.7 Hz, 2H), 2.96-2.86 (m, 1H), 2.63-2.57 (m, 1H), 2.40 (br d, J=8.6 Hz, 1H), 2.04-1.97 (m, 1H). MS (ESI) m/z 449.3 [M+H]⁺

Compound 75: Step 1: To a solution of 2-chloro-5-nitropyridine (40.0 g, 252 mmol, 1.00 eq), potassium trifluoro(prop-1-en-2-yl) borate (48.5 g, 327 mmol, 1.30 eq) and [1,1′-bis(diphenylphosphino)ferrocene] dichloropalladium(II) (9.23 g, 12.6 mmol, 0.0500 eq) in dioxane (400 mL) and water (80.0 mL) was added potassium carbonate (104 g, 756 mmol, 3.00 eq), then evacuated with vacuum and back filled with nitrogen 3 times. The mixture was stirred at 80° C. for 2 h. The reaction mixture was poured into water (200 mL) and extracted with ethyl acetate (3×80.0 mL). The combined organic phase was separated, washed with brine (60.0 mL), dried over anhydrous sodium sulfate, filtered and concentrated to give a residue which was purified by silica gel chromatography (petroleum ether/ethyl acetate=20/1 to 10/1) to afford 5-nitro-2-(prop-1-en-2-yl)pyridine (36.0 g, 219 mmol, 86% yield) as a pink solid. ¹H NMR (400 MHz, DMSO-d₆) δ=9.34 (d, J=2.4 Hz, 1H), 8.57 (dd, J=2.8, 8.8 Hz, 1H), 7.93 (d, J=8.8 Hz, 1H), 6.17 (s, 1H), 5.60 (t, J=1.6 Hz, 1H), 2.21 (s, 3H). (The H NMR comes from pilot run). MS (ESI) m/z 165.0 [M+H]⁺

Step 2: To a mixture of trimethyl sulfoxonium iodide (10.0 g, 45.6 mmol, 1.50 eq) in dimethylsulfoxide (150 mL) was added potassium tert-butoxide (5.13 g, 45.6 mmol, 1.50 eq). The reaction mixture was stirred at 20° C. for 1 h. Then 5-nitro-2-(prop-1-en-2-yl)pyridine (5.00 g, 30.4 mmol, 1.00 eq) in tetrahydrofuran (50.0 mL) was added and the reaction mixture was stirred at 20° C. for another 3 h. The reaction mixture was poured into water (500 mL) and extracted with ethyl acetate (3×200 mL). The combined organic phase was separated, washed with brine (100 mL), dried over anhydrous sodium sulfate, filtered and concentrated to give a residue, which was purified by silica gel chromatography (petroleum ether/ethyl acetate=10/1 to 0/1) followed by Prep-HPLC (column: Phenomenex luna C18 150*40 mm*15 um; mobile phase: [water (0.1% trifluoroacetic acid)-acetonitrile]; B %: 32%-62%, 11 min) and lyophilized to afford 2-(1-methylcyclopropyl)-5-nitropyridine #101 (1.20 g, 6.73 mmol, 22% yield) as an off-white solid. ¹H NMR (400 MHz, DMSO-d₆) δ=9.23 (d, J=2.4 Hz, 1H), 8.48 (dd, J=2.8, 8.8 Hz, 1H), 7.62 (d, J=8.8 Hz, 1H), 1.54 (s, 3H), 1.34-1.30 (m, 2H), 1.04-1.00 (m, 2H). MS (ESI) m/z 179.2 [M+H]⁺ To a mixture of 2-(1-methylcyclopropyl)-5-nitropyridine (800 mg, 4.49 mmol, 1.00 eq) in tetrahydrofuran (20.0 mL) was added palladium on carbon (80.0 mg, 10% purity). The reaction mixture was stirred at 20° C. for 2 h under hydrogen atmosphere. The reaction mixture was filtered and concentrated to give 6-(1-methylcyclopropyl)pyridin-3-amine (600 mg, crude) as yellow oil.

Step 3: To a solution of 6-(1-methylcyclopropyl)pyridin-3-amine (600 mg, 4.05 mmol, 1.00 eq) and pyridine (960 mg, 12.1 mmol, 980 uL, 3.00 eq) in acetonitrile (6.00 mL) was added phenyl carbonochloridate (760 mg, 4.86 mmol, 608 uL, 1.20 eq). The reaction mixture was stirred at 20° C. for 1 h. The reaction mixture was concentrated to give a residue. The residue was purified by silica gel chromatography (petroleum ether/ethyl acetate=1/0 to 10/1) to give phenyl (6-(1-methylcyclopropyl) pyridin-3-yl)carbamate (600 mg, 2.24 mmol, 55% yield) as a white solid. ¹H NMR (400 MHz, DMSO-d₆) δ=10.32 (br s, 1H), 8.54 (br d, J=2.0 Hz, 1H), 7.92-7.77 (m, 1H), 7.55-7.39 (m, 2H), 7.37-7.19 (m, 4H), 1.45 (s, 3H), 1.11 (br d, J=2.0 Hz, 2H), 0.76 (q, J=3.2 Hz, 2H). MS (ESI) m/z 269.0 [M+H]⁺

Step 4: A mixture of 3-(4-fluoro-6-(hydroxymethyl)-1-oxoisoindolin-2-yl)piperidine-2,6-dione (10.0 mg, 34.2 umol, 1.00 eq) in dimethyl formamide (1.00 mL) was added sodium hydride (2.74 mg, 68.4 umol, 60% purity, 2.00 eq) and phenyl (6-(1-methylcyclopropyl) pyridin-3-yl)carbamate (11.0 mg, 41.0 umol, 1.20 eq) at 0° C. The reaction mixture was stirred at 20° C. for 1 h. The reaction mixture was added formic acid (2.00 mL) and filtered to give a filtrate, which was purified by Prep-HPLC (column: Phenomenex Luna C18 150*25 mm*10 um; mobile phase: [water (0.225% formic acid)-acetonitrile]; B %: 10%-40%, 10 min) and lyophilized to give (2-(2,6-dioxopiperidin-3-yl)-7-fluoro-3-oxoisoindolin-5-yl)methyl(6-(1-methylcyclopropyl)pyridin-3-yl)carbamate (11.16 mg, 21.5 umol, 63% yield, 99% purity, formic acid) as a white solid. ¹H NMR (400 MHz, DMSO-d₆) δ=11.02 (br s, 1H), 9.95 (br s, 1H), 8.48 (d, J=2.4 Hz, 1H), 7.81 (br d, J=7.6 Hz, 1H), 7.69 (s, 1H), 7.57 (d, J=9.6 Hz, 1H), 7.29 (d, J=8.8 Hz, 1H), 5.29 (s, 2H), 5.14 (dd, J=5.2, 13.2 Hz, 1H), 4.64-4.53 (m, 1H), 4.47-4.37 (m, 1H), 2.99-2.87 (m, 1H), 2.64-2.58 (m, 1H), 2.43 (br d, J=4.8 Hz, 1H), 2.07-1.96 (m, 1H), 1.44 (s, 3H), 1.13-1.03 (m, 2H), 0.80-0.68 (m, 2H). MS (ESI) m/z 467.0 [M+H]⁺

### Example 3: Compound Activity by Fluorescent Polarization Assay

Compound activity was monitored in a fluorescence polarization (FP) homogeneous assay using 1-[5-({2-[2-(2-{[2-(2,6-dioxopiperidin-3-yl)-1,3-dioxo-2,3-dihydro-1H-isoindol-4-yl]oxy}acetamido)ethoxy]ethyl}carbamoyl)pentyl]-3,3-dimethyl-2-[(1E,3E)-5-[(2E)-1,3,3-trimethyl-5-sulfo-2,3-dihydro-1H-indol-2-ylidene]penta-1,3-dien-1-yl]-3H-indol-1-ium-5-sulfonate as a fluorescent probe. Unless otherwise stated, all reagents were purchased from Sigma Aldrich. Enzymatic reactions were conducted in Perkin-Elmer Black 384 well ProxiPlate Plus (catalogue no. 6008269) in 10 sL total volume. Full length wild-type cereblon CRBN (80.0 nM, 10 sL) was incubated in assay buffer containing 20 mM HEPES (pH 8.0), 150 NaCl, 0.5 mM TCEP and 0.05% Tween 20 in the presence or absence of compound (300 nL). Inhibitors were stored as 10 mM DMSO stocks in an inert environment (low humidity, dark, low oxygen, room temperature) using the Storage Pod System.

Compounds and DMSO were dispensed using the Echo E5XX (Labcyte Inc. USA) to give concentrations from 300 to 0.937 or 3000 to 9.3 nM in a 12 data point curve. Mutant YWAA CRBN (80.0 nM, 10 μL) which does not interact with the fluorescent probe was used as a negative control for the assay. Following incubation at room temperature for 30 min, the assay was initiated by dispensing the probe to a final concentration of 5 nM (2.5 nL of a 20 μM stock) using the Echo E5XX. FP was measured after a period of 12 hours using a Pherastar plate reader (BMG Labtech, Germany) exciting at 590 nm and measuring the amount of parallel and perpendicular light at 675 nm. The FP signal was subsequently normalized to the no-compound control (i.e., DMSO). Analysis and IC50 values were derived using Dotmatics (Dotmatics UK) software.

TABLE 2
IC50 values determined in the fluorescence polarization
assay indicating the cereblon binding

| | rFP | | rFP | | rFP |
|---|---|---|---|---|---|
| | IC50 | | IC50 | | IC50 |
| Compound | [nM] | Compound | [nM] | Compound | [nM] |
| 1 | D | 2 | D | 3 | D |
| 4 | D | 5 | B | 6 | D |
| 7 | C | 8 | D | 9 | C |
| 10 | D | 11 | A | 12 | D |
| 13 | D | 14 | D | 15 | D |
| 16 | B | 17 | A | 18 | C |
| 19 | A | 20 | B | 21 | C |
| 22 | D | 23 | B | 24 | D |
| 25 | D | 26 | C | 27 | C |
| 28 | D | 29 | D | 30 | D |
| 31 | D | 32 | D | 33 | D |
| 34 | D | 35 | D | 36 | C |
| 37 | A | 38 | A | 39 | C |
| 40 | A | 41 | C | 42 | B |
| 43 | B | 44 | A | 45 | B |
| 46 | B | 47 | B | 48 | C |
| 49 | B | 50 | B | 51 | B |
| 52 | A | 53 | A | 54 | A |
| 55 | B | 56 | A | 57 | A |
| 58 | A | 59 | A | 60 | A |
| 61 | D | 62 | A | 63 | |
| 64 | | 65 | | 66 | |
| 67 | | 68 | | 69 | — |
| 70 | D | 71 | D | 72 | D |
| 73 | A | | | | |

Table 2 assigns each compound a code indicating the ability for cereblon binding by mean of their IC50 values: A, B, C or D. According to the code, A represents an IC50 value of ≤600 nM, B represents an IC50 value >600 nM and <1200 nM, C represents an IC50 value of >1200 nM and ≤1900 nM and D represents an IC50 value of >1900 nM.

In some embodiments, the disclosure is directed to compounds with an IC50 value of less than 1900 nM, i.e. directed to compounds 11, 17, 19, 37, 38, 40, 44, 52, 53, 54, 56, 57, 58, 59, 60, and 62.

In some embodiments, the disclosure is directed to compounds with an IC50 value of less than 1200 nM, i.e. directed to compounds 5, 11, 16, 17, 19, 20, 23, 37, 38, 40, 42, 43, 44, 45, 46, 47, 49, 50, 51, 52, 53, 54, 56, 57, 58, 59, 60, and 62.

In some embodiments, the disclosure is directed to compounds with an IC50 value of less than 600 nM, i.e. directed to compounds 5, 7, 9, 11, 16, 17, 18, 19, 20, 21, 23, 26, 27, 36, 37, 38, 39, 40, 41, 42, 43, 44, 45, 46, 47, 48, 49, 50, 51, 52, 53, 54, 56, 57, 58, 59, 60, and 62.

### Example 4: Compound Binding by Immunofluorescence Assay

The compounds were tested in an immunofluorescence assay for their activity to bind to degrade GSPT1. CAL-Si cells were purchased from DSMZ (cat. Number ACC302), sub-cultured in 90% Dulbecco's MEM (4.5 g/L glucose, Gibco 11965)+10% heat inactivated FBS (BioConcept, 2-01F136I) and incubated at 37° C., 5% CO₂. For the assay, imaging microtiterplate Cell Carrier 96 Ultra (Perkin Elmer 6055302) were pre-coated with Fibronectin (Sigma F085, 30 μl at 0.2 μg/ml) in PBS (100 μl, Gibco 14190) for 45 min at room temperature, rinsed with PBS and CAL-Si cells (30K cells/well) were plated and let to adhere overnight. Cells were treated with compounds typically using a serial dilution ranging from 30 μM to 0.1 nM for 6 hours. Compounds were stored at 10 mM DMSO stocks. Vehicle (DMSO), positive (CC-885, 10 μM) and rescue controls (positive control plus 0.2 μM bortezomib) were also included at this stage. Cells were subsequently rinsed with PBS and fixed in 10% Formalin solution (50 μl, Sigma HT5011)) for 20 mins at room temperature. Following three consecutive PBS washes (100 μl), cells were permeabilized in 0.1% Triton X-100 in PBS (Sigma 93443, 50 μl) for 15 mins at room temperature. Following three further PBS washes, 50 μl blocking buffer (1% BSA, Sigma A4503, in PBS) was added for 45 min for signal-to-noise reduction.

Primary antibody (human GSPT1, Sigma HPA052488) was diluted in blocking buffer (dil.1/300, 35 μl/well) and incubated with the cells overnight at 4° C. After three PBS washes, Alexa-fluor 488 coupled secondary antibodies (Invitrogen, A32731, dil.1/1000), Alexa-fluor 647-Phalloïdin (Invitrogen, A22287, dil.1/200) and DAPI (Thermo, #62248, dil.1/1000) were diluted in blocking buffer and incubated with the samples for 2 hours at room temperature. After three final PBS washes, samples were conserved in 100 μl PBS in the dark, until measurement. Image acquisition was performed on the Operetta High-Content Imager (Perkin-Elmer). Fluorescence intensity of Alexa-Fluor 488 (GSPT1), Alexa-Fluor 647 (Actin) and DAPI (Nucleus) were measured. For the determination of GSPT1 DC₅₀ values, a custom algorithm implemented in the PerkinElmer image analysis software Harmony-Acapella® was developed. After user-defined setting of adjustment parameters, the analysis was run identically without human intervention for all image fields. DAPI staining of the nuclei was used to determine the location of cells using standard nuclei detection modules. Segmentation artifacts were removed by threshold-based filters for area, roundness and intensity. The outline of the cells was determined analogously from the sum of the normalized, smoothed DAPI and Actin channel, starting from each nucleus. The Alexa-Fluor 488 (GSPT1) signal intensity in each cell was finally measured, in order to obtain a Mean intensity per cell. GSPT1 degradation (DC₅₀) was calculated after normalization to controls and data import in CDD vault Database, using non-linear regression.

TABLE 3
Activity for GSPT1 degradation

| Compound | Code | Compound | Code | Compound | Code |
|---|---|---|---|---|---|
| 1 | B | 2 | B | 3 | B |
| 4 | B | 5 | A | 6 | B |
| 7 | B | 8 | A | 9 | A |
| 10 | B | 11 | A | 12 | B |
| 13 | B | 14 | B | 15 | B |
| 16 | A | 17 | A | 18 | A |
| 19 | A | 20 | A | 21 | A |
| 22 | A | 23 | A | 24 | B |
| 25 | A | 26 | B | 27 | B |
| 28 | B | 29 | A | 30 | B |
| 31 | B | 32 | B | 33 | B |
| 34 | B | 35 | A | 36 | A |
| 37 | A | 38 | A | 39 | A |
| 40 | A | 41 | B | 42 | A |
| 43 | A | 44 | A | 45 | A |
| 46 | B | 47 | A | 48 | B |
| 49 | A | 50 | A | 51 | B |
| 52 | B | 53 | A | 54 | A |
| 55 | A | 56 | A | 57 | A |
| 58 | C | 59 | A | 60 | A |
| 61 | A | 62 | B | 63 | B |
| 64 | B | 65 | B | 66 | C |
| 67 | A | 68 | C | 70 | D |
| 71 | A | 72 | D | 73 | D |

Table 3 assigns each compound a code indicating the ability for GSPT1 degradation: A, B or C. According to the code, A represents a DC50 value of ≤30 nM, B represents a DC50 value >30 nM and ≤300 nM and C represents a DC50 value of >300 nM.

In some embodiments, the compounds of any of formula I to XIII exhibits a DC50 value of nM or less, i.e. compounds with code A. In some embodiments the compound is selected from the group consisting of 5, 8, 9, 11, 16, 17, 18, 19, 20, 21, 22, 23, 25, 29, 35, 36, 37, 38, 39, 40, 42, 43, 44, 45, 47, 49, 50, 53, 54, 55, 56, 57, 59, 60, 61, 67 and 71.

TABLE 4
HTRF Binding of compounds to CRBN
and Activity for GSPT1 degradation.

| Compound | HTRF Class | IF Class |
|---|---|---|
| 71 | B | A |
| 72 | C | C |
| 74 | A | A |
| 75 | B | A |

In Table 4 each compound is assigned a class (HTRF class) indicating the ability for Cereblon binding by means of their HTRF IC50 values according to Example 3: A, B, C or D. According to the code, A represents an IC50 value of ≤100 nM, B represents an IC50 value >100 nM and ≤1000 nM, C represents an IC50 value >1000 nM. IF Class assigns each compound a code indicating the ability for GSPT1 degradation:

### Example 5: In Vivo Efficacy Study —Tumor Growth Inhibition in MDA-MB-213 Model (FIG. 1)

MDA-MB-231 cells were maintained in vitro in DMEM medium supplemented with 20% heat inactivated FBS at 37° C. in an atmosphere of 5% CO₂ in air. Cells (10 millions) resuspended in 0.2 mL of PBS with Matrigel (50:50) were inoculated into female BALB/c nude mice and allowed to grow to 150 mm³ in size. Mice were dosed daily i.p. with vehicle or a representative compound of the disclosure (0.3, 1, 3 and 10 mg per kilogram p.o.). Compound formulations were prepared fresh daily in 5% DMSO/95% (30% HP-β-CD in purified water). Mice were dosed for 21 days and tumor volumes measured every 3 days.

1. A compound of formula I: 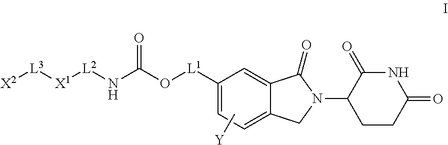
or a pharmaceutically acceptable salt or stereoisomer thereof,
wherein
X¹ is selected from the group consisting of linear or branched C₁₋₆ alkyl, C₃₋₆ cycloalkyl, C₆₋₁₀ aryl, 5-10 membered heteroaryl, and 4-8 membered heterocycloalkyl, wherein X¹ is unsubstituted or substituted with one or more substituents independently selected from the group consisting of halogen, linear or branched C₁₋₆ alkyl, linear or branched C₁₋₆ heteroalkyl, CF₃, CHF₂, CMeF₂, —O—CHF₂, —O—(CH₂)₂—OMe, OCF₃, C₁₋₆ alkylamino, —CN, NH₂, C₁₋₄ alkoxy and C₁₋₄ alkylhydroxy;
X² is selected from the group consisting of H, C₃₋₆ cycloalkyl, C₆₋₁₀ aryl, 5-10 membered heteroaryl, and 4-8 membered heterocycloalkyl, wherein X² is unsubstituted or substituted with one or more substituents independently selected from the group consisting of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, and C₁₋₄ alkylhydroxy;
Y is selected from the group consisting of linear or branched C₁₋₆ alkyl, —C₁₋₄ alkoxy, —CN, halogen, CF₃, CHF₂, CMeF₂, OCF₃, and OCHF₂;
L¹ is linear or branched C₁₋₆ alkyl;
L² is selected from a covalent bond, and linear or branched C₁₋₆ alkyl; and
L³ is selected from the group consisting of a covalent bond, linear or branched C₁₋₆ alkyl, —O—, and —C₁₋₄ alkoxy.

2. The compound of claim 1, or a pharmaceutically acceptable salt or stereoisomer thereof, wherein Y is in the 4-position or in the 5-position or in the 7-position of the ring.

3. The compound of claims 1 or 2 or a pharmaceutically acceptable salt or stereoisomer thereof, wherein L¹ is —CH₂—, L² is a covalent bond and L³ is a covalent bond or wherein L¹ is —CH₂—, L² is a covalent bond and L³ is —O—.

4. The compound of any one of the preceding claims, or a pharmaceutically acceptable salt or stereoisomer thereof, wherein X¹ is selected from —C₆₋₁₀ aryl, and 5-10 membered heteroaryl, wherein X¹ is unsubstituted or substituted with one or more substituents independently selected from the group consisting of linear or branched —C₁₋₄ alkyl, halogen, —CF₃, —CHF₂, —CMeF₂, —O—(CH₂)₂—OMe, —OCF₃, —OCHF₂, C₁₋₆ alkylamino, —CN, —NH₂, —C₁₋₄ alkylhydroxy, and —C₁₋₄ alkoxy.

5. The compound of any one of the preceding claims, wherein X² is selected from the group consisting of H, C₃₋₆ cycloalkyl, C₆ aryl, 6-membered heteroaryl, and 5-6 membered heterocycloalkyl, wherein X² is unsubstituted or substituted with one or more substituents independently selected from the group consisting of linear or branched C₁₋₄ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy.

6. The compound of claim 1, wherein the compound is of formula III, such as formula IIIa, IIIb or IIIc: 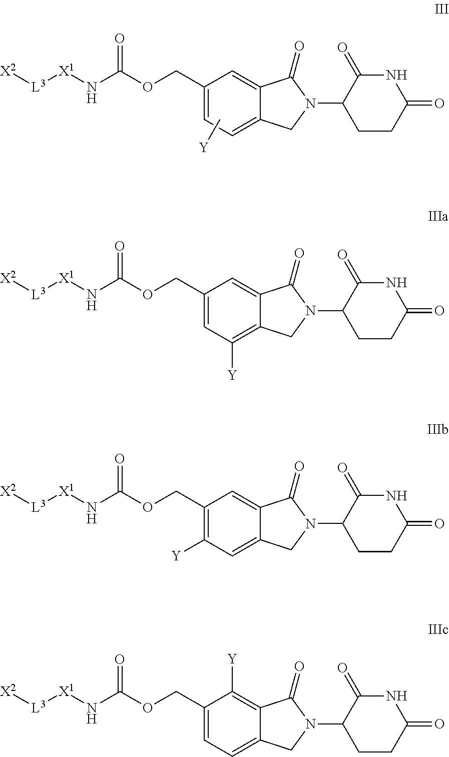
or a pharmaceutically acceptable salt or stereoisomer thereof,
wherein
X¹ is selected from the group consisting of linear or branched C₁₋₆ alkyl, C₃₋₄ cycloalkyl, C₆₋₁₀ aryl, 5-10 membered heteroaryl, and 4-8 membered heterocycloalkyl, wherein X¹ is unsubstituted or substituted with one or more substituents independently selected from the group consisting of halogen, linear or branched C₁₋₆ alkyl, linear or branched C₁₋₆ heteroalkyl, CF₃, CHF₂, CMeF₂, —O—CHF₂, —O—(CH₂)₂—OMe, OCF₃, C₁₋₆ alkylamino, —CN, NH₂, C₁₋₄ alkoxy, and C₁₋₄ alkylhydroxy;
X² is selected from the group consisting of H, C₃₋₆ cycloalkyl, C₆₋₁₀ aryl, 5-10 membered heteroaryl, and 4-8 membered heterocycloalkyl, wherein X² is unsubstituted or substituted with one or more substituents independently selected from the group consisting of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, and C₁₋₄ alkylhydroxy;
Y is selected from the group consisting of linear or branched C₁₋₆ alkyl, —C₁₋₄ alkoxy, —CN, halogen, CF₃, CHF₂, CMeF₂, OCF₃, and OCHF₂; and
L³ is selected from the group consisting of a covalent bond, linear or branched C₁₋₆ alkyl, —O—, and —C₁₋₄ alkoxy.

7. The compound of claim 6 or a pharmaceutically acceptable salt or stereoisomer thereof, wherein X¹ is selected from —C₆₋₁₀ aryl, and 5-10 membered heteroaryl, wherein X¹ is unsubstituted or substituted with one or more substituents independently selected from the group consisting of linear or branched —C₁₋₄ alkyl, halogen, —CF₃, —CHF₂, —CMeF₂, —O—(CH₂)₂—OMe, —OCF₃, —OCHF₂, C₁₋₆ alkylamino, —CN, —NH₂, —C₁₋₄ alkylhydroxy, and —C₁₋₄ alkoxy.

8. The compound of claim 6 or 7 or a pharmaceutically acceptable salt or stereoisomer thereof, wherein X² is selected from the group consisting of H, C₃₋₆ cycloalkyl, C₆ aryl, 6-membered heteroaryl, and 5-6 membered heterocycloalkyl, wherein X² is unsubstituted or substituted with one or more substituents independently selected from the group consisting of linear or branched C₁₋₄ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy.

9. The compound of claim 1, wherein the compound is of formula IV, such as IVa, IVb or IVc: 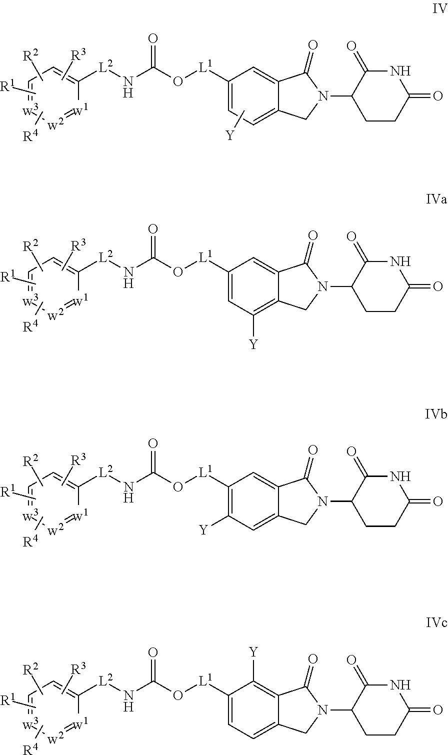
or a pharmaceutically acceptable salt or stereoisomer thereof,
wherein
Y is selected from the group consisting of linear or branched C₁₋₆ alkyl, —C₁₋₄ alkoxy, —CN, halogen, CF₃, CHF₂, CMeF₂, OCF₃, and OCHF₂;
each of w¹, w², and w³ is independently selected from C and N, with the proviso that two or three of w¹, w², w³ are C;
each of R¹, R², R³, and R⁴ is independently selected from the group consisting of H, linear or branched —C₁₋₆ alkyl, linear or branched C₁₋₆ heteroalkyl, —C₁₋₄ alkoxy, CF₃, CHF₂, CMeF₂, —O—(CH₂)₂—OMe, OCF₃, OCHF₂, —C₁₋₆ alkylamino, —CN, —NH₂, —C₁₋₄ alkylhydroxy, and halogen, such as F, Cl or Br, e.g. F or Cl, or a group of formula -L³-X², wherein L³ is selected from the group consisting of a covalent bond, linear or branched C₁₋₆ alkyl, and —O—, and X² is selected from the group consisting of C₃₋₆ cycloalkyl, C₆₋₁₀ aryl, 5-10 membered heteroaryl, and 4-8 membered heterocycloalkyl, wherein X² is unsubstituted or substituted with one or more substituents independently selected from the group consisting of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, and C₁₋₄ alkylhydroxy;
L¹a is linear or branched C₁₋₆ alkyl; and
L² is selected from a covalent bond, and linear or branched C₁₋₆ alkyl.

10. The compound of claim 9 or a pharmaceutically acceptable salt or stereoisomer thereof, wherein L¹ is —CH₂— and wherein L² is a covalent bond.

11. The compound of claims 9 or 10 or pharmaceutically acceptable salts or stereoisomers thereof, wherein w¹, w², and w³ are C, or wherein w¹ is N and w², and w³ are C, or wherein w² is N and w¹, and w³ are C, or wherein w³ is N and w¹, and w² are C.

12. The compound of claim 1, wherein the compound is of formula XI, such as XIa, XIb or XIc: 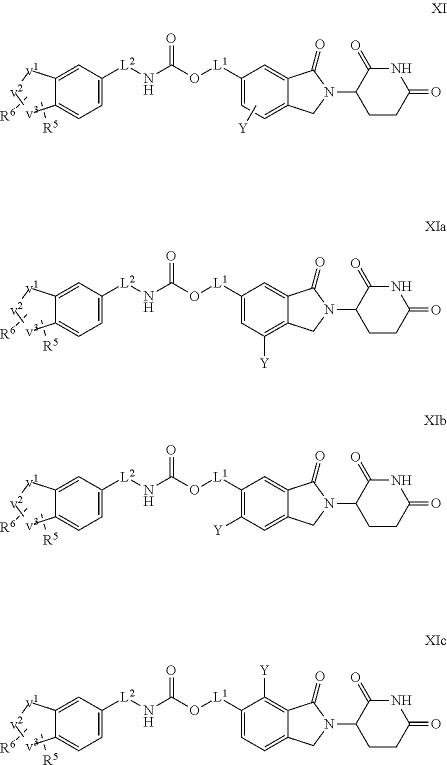
or a pharmaceutically acceptable salt or stereoisomer thereof,
wherein
Y is selected from the group consisting of linear or branched C₁₋₆ alkyl, —C₁₋₄ alkoxy, —CN, halogen, CF₃, CHF₂, CMeF₂, OCF₃, and OCHF₂
each of v¹, v², and v³ is independently selected from C, and O, with the proviso that at least one of v¹, v², and v³ is C;
each of R⁵ and R⁶ is independently selected from the group consisting of H, linear or branched —C₁₋₄ alkyl and halogen, such as F, Cl, e.g. F;
L¹ is linear or branched C₁₋₆ alkyl;
L² is selected from a covalent bond, and linear or branched C₁₋₆ alkyl.

13. The compound of claim 1, wherein the compound is of formula XIII, such as XIIIa, XIIIb or XIIIc: 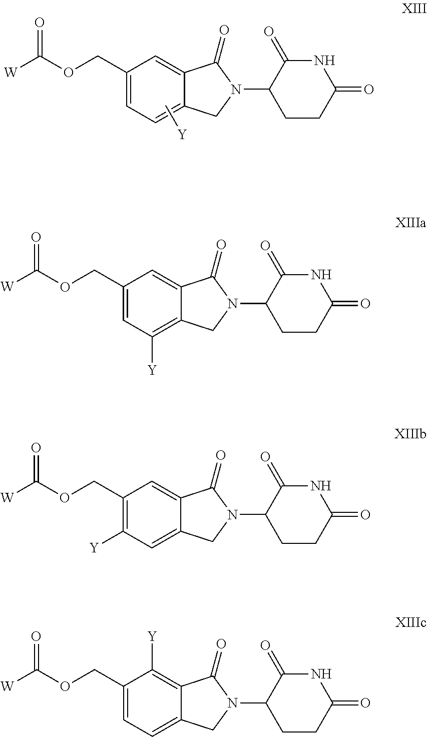
or pharmaceutically acceptable salts or stereoisomers thereof,
wherein
Y is selected from the group consisting of linear or branched C₁₋₆ alkyl, —C₁₋₄ alkoxy, —CN, halogen, CF₃, CHF₂, CMeF₂, OCF₃, and OCHF₂, such as C₁₋₄ alkyl, such as methyl, —C₁₋₄ alkoxy, such as —OMe, —CN, halogen, such as F, Cl, Br; and
W is selected from the group consisting of:

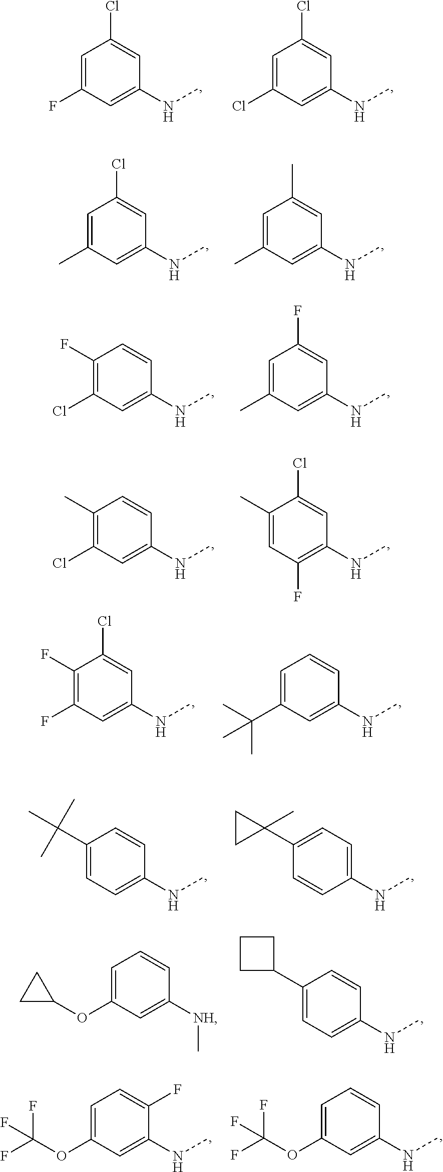

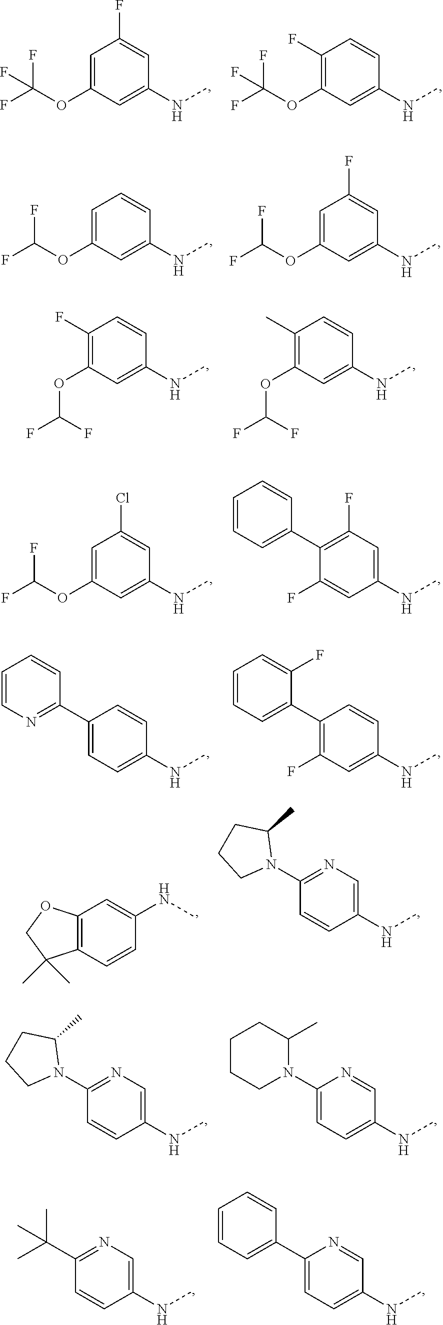

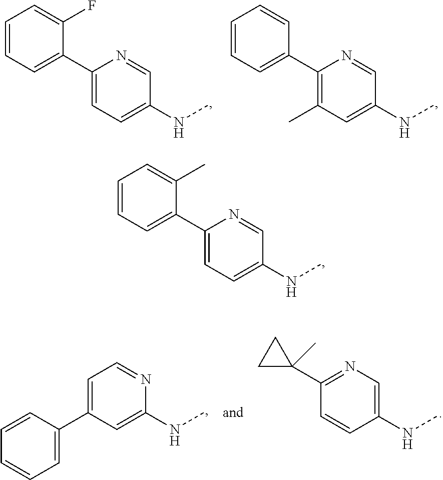

14. A compound selected from the group consisting of 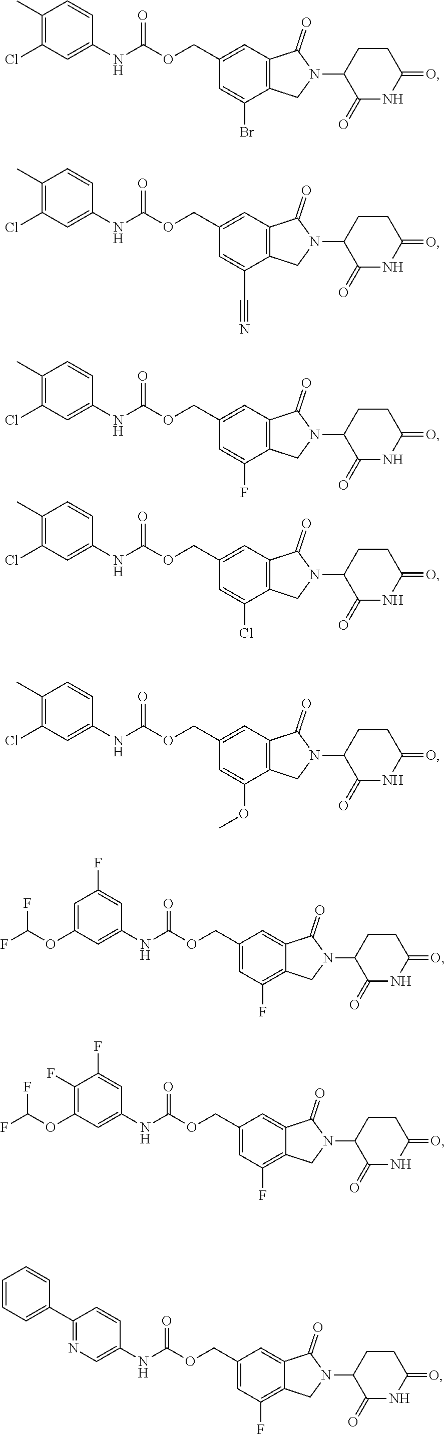 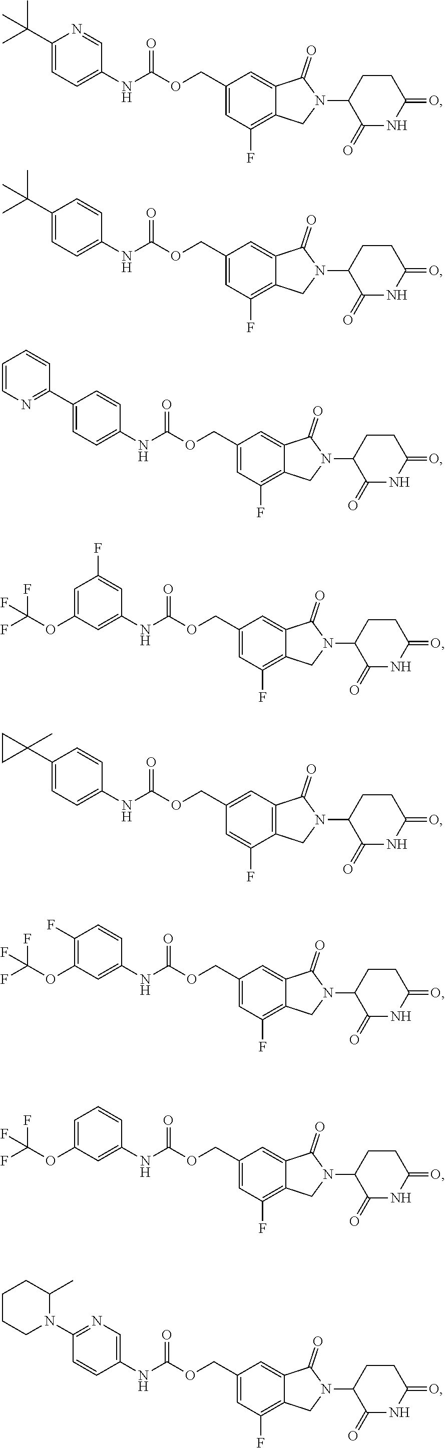 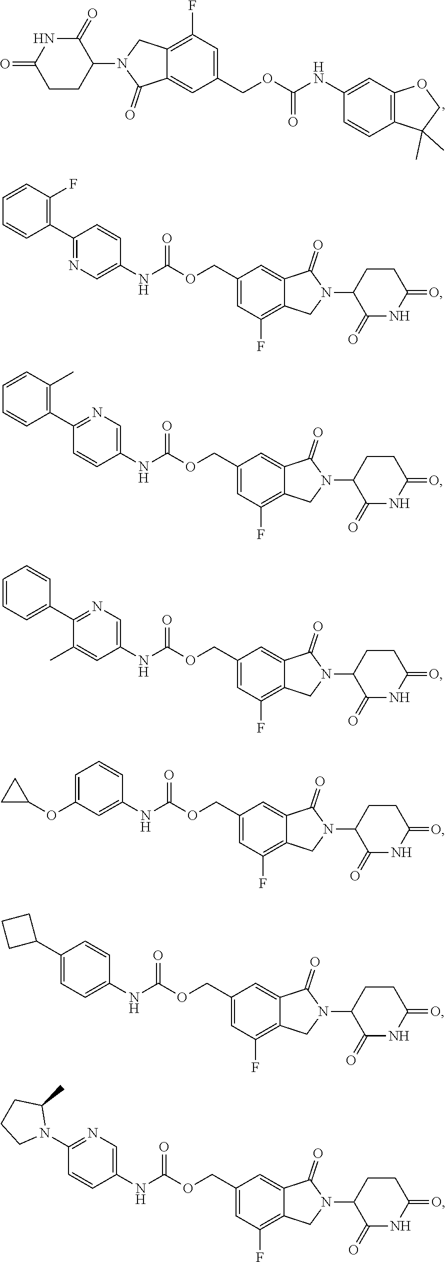 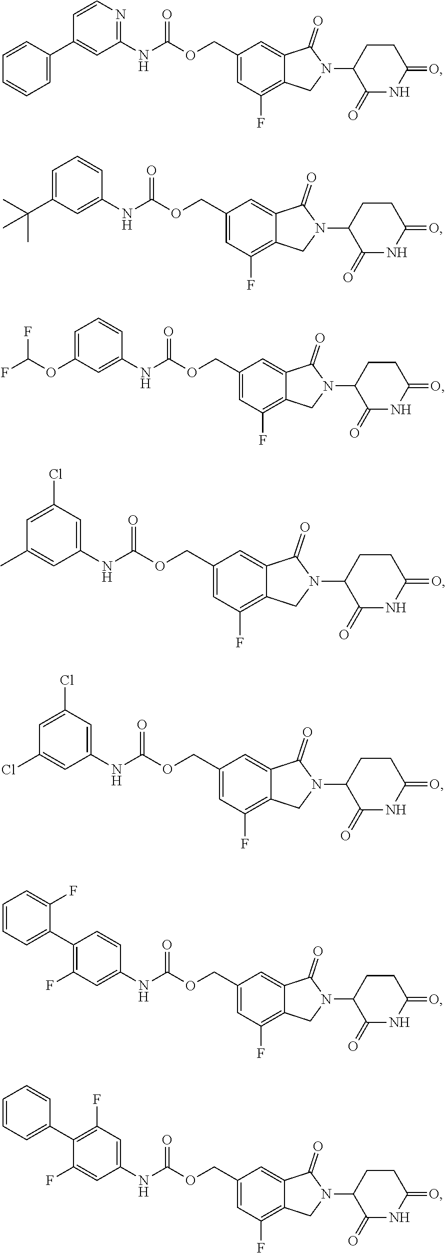 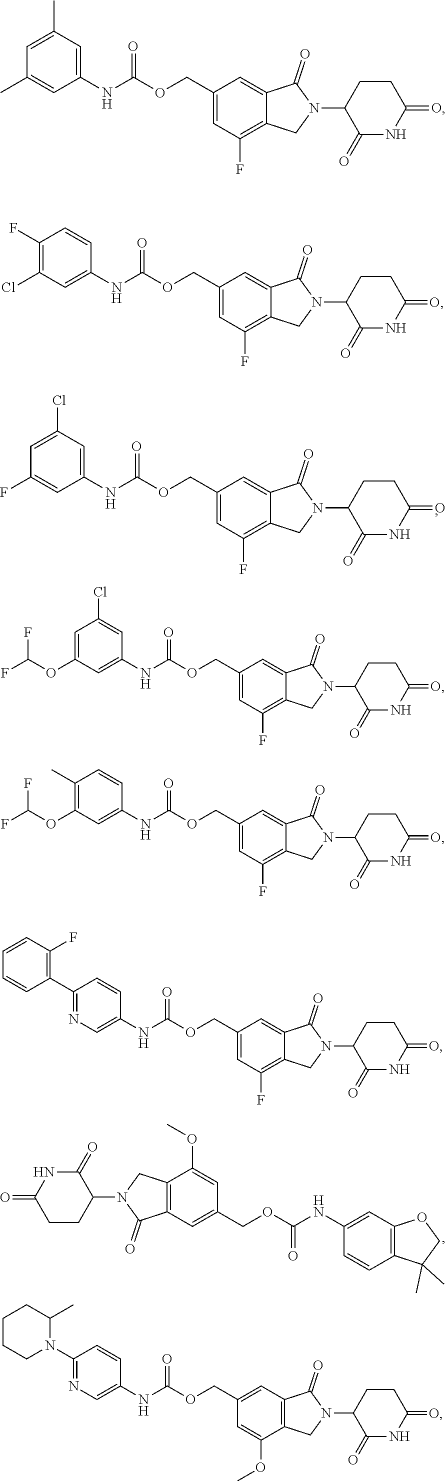 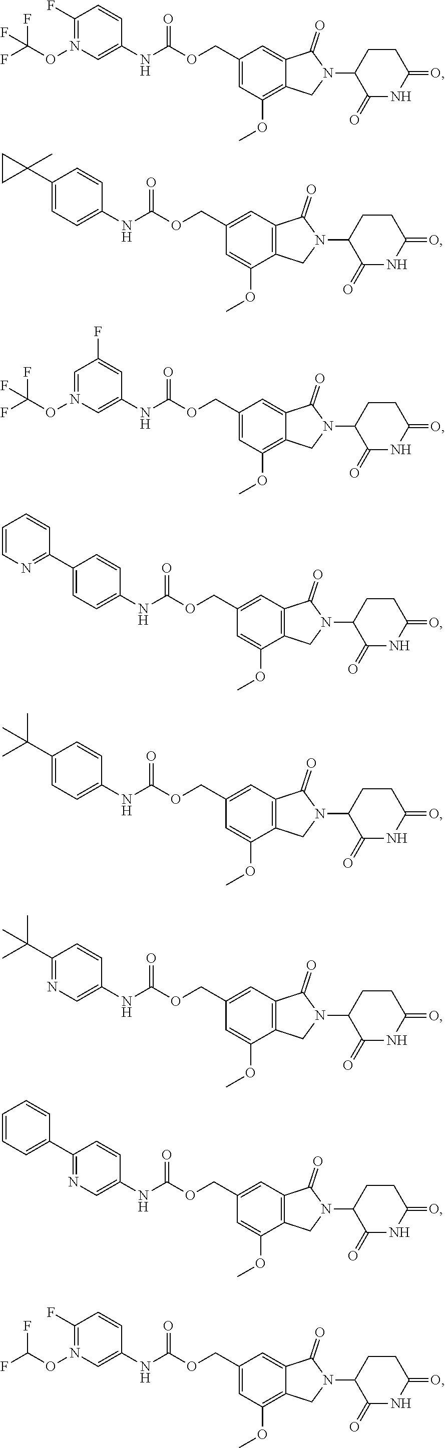 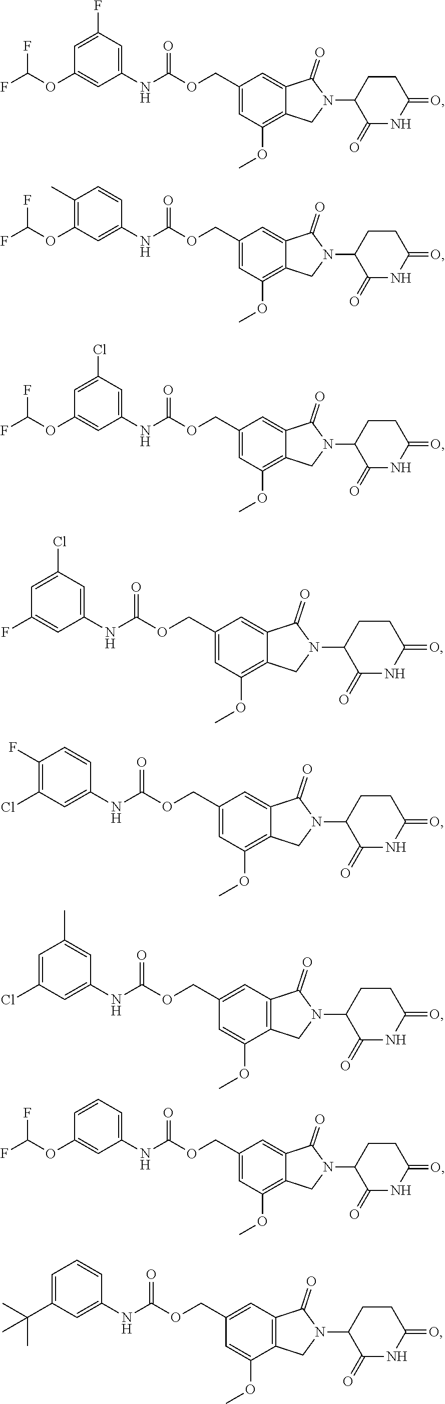 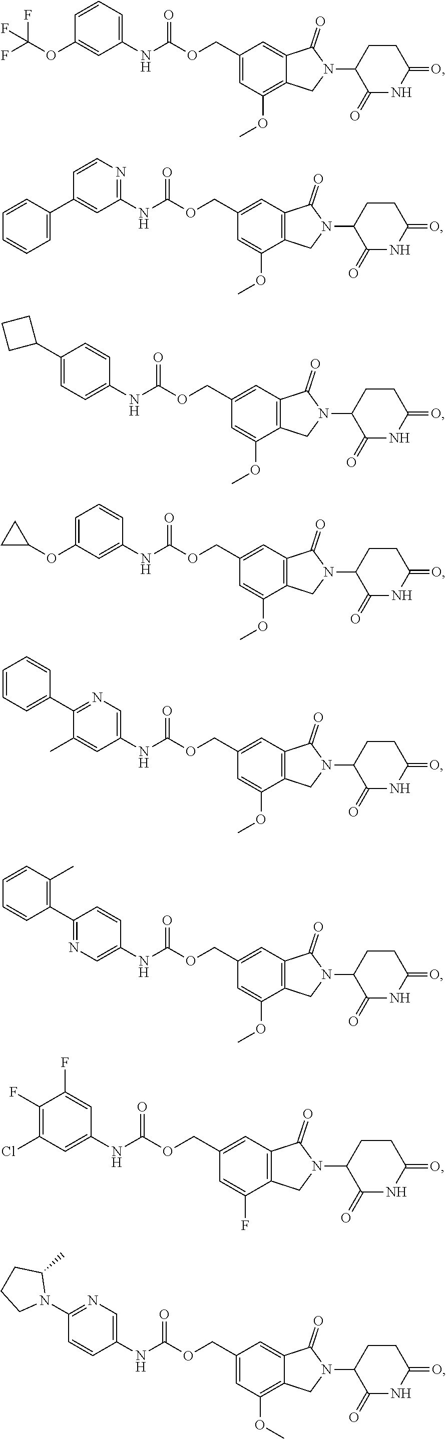 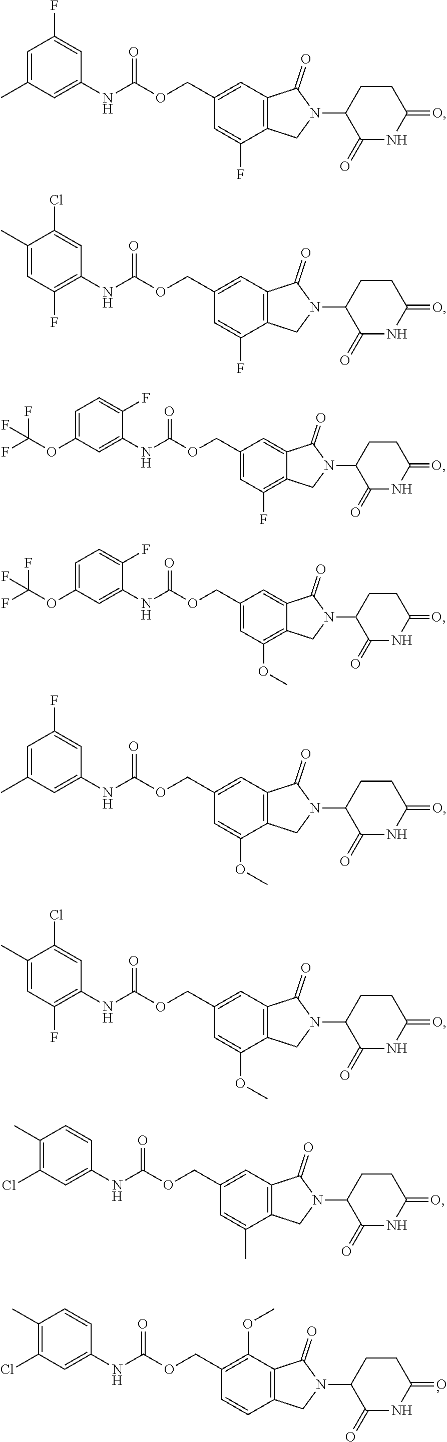 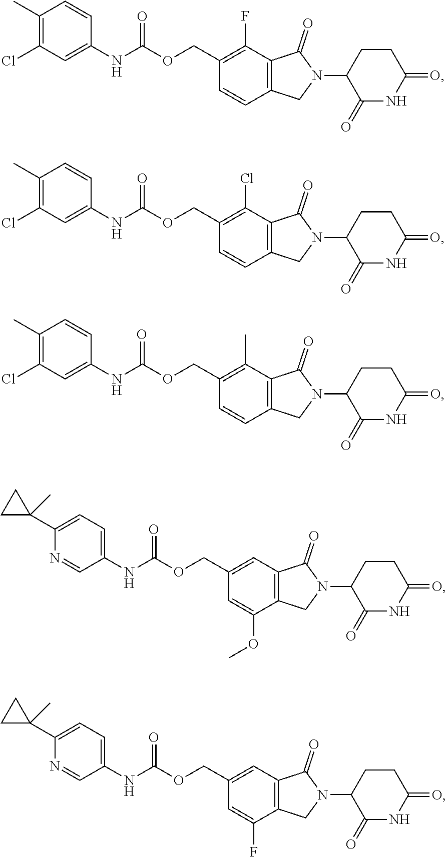 [SMILES: Cc1ccc(NC(=O)OCc2ccc3c(c2F)C(=O)N(C2CCC(=O)NC2=O)C3)cc1Cl ; Cc1ccc(NC(=O)OCc2ccc3c(c2Cl)C(=O)N(C2CCC(=O)NC2=O)C3)cc1Cl ; Cc1ccc(NC(=O)OCc2ccc3c(c2C)C(=O)N(C2CCC(=O)NC2=O)C3)cc1Cl ; COc1cc(COC(=O)Nc2ccc(C3(C)CC3)nc2)cc2c1CN(C1CCC(=O)NC1=O)C2=O ; CC1(c2ccc(NC(=O)OCc3cc(F)c4c(c3)C(=O)N(C3CCC(=O)NC3=O)C4)cn2)CC1]
and pharmaceutically acceptable salts and stereoisomers thereof.

15. A composition comprising a compound according to any one of claims 1 to 14 or a pharmaceutically acceptable salt or stereoisomer thereof.

16. The composition of claim 15, further comprising a pharmaceutically acceptable carrier and/or a second therapeutically active agent.

17. A compound of any one of claims 1 to 16 or a composition of claim 15 or 16 for use in therapy, in particular for use in the treatment of a disease associated with GSPT1, such cancer, e.g. lung cancer, breast cancer and neuroendocrine cancer.

18. A use of a compound of any one of claims 1 to 14 or a composition of claims 15 or 16 in the treatment of a disease associated with GSPT1, such as cancer, in particular lung cancer, breast cancer and neuroendocrine cancer, comprising administering to a subject a therapeutically-effective amount of the composition.

19. A method of treating cancer, in particular lung cancer, breast cancer and neuroendocrine cancer in a subject, comprising administering to a subject a therapeutically effective amount of the compound of any one of claims 1 to 14 or a composition of claims 15 or 16.

20. A method of treating a Myc-driven cancer in a subject in need thereof, comprising administering to the subject a therapeutically effective amount of the compound of claim 1.

21. The method of claim 20, wherein the Myc-driven cancer is a Myc-driven lung cancer.

22. The method of claim 20, wherein the Myc-driven cancer is a Myc-driven small cell lung cancer.

23. The method of claim 22, wherein the Myc-driven small cell lung cancer is a high L-Myc small cell lung cancer.

24. The method of claim 20, wherein the Myc-driven cancer is a Myc-driven non-small cell lung cancer.

25. The method of claim 24, wherein the Myc-driven non-small cell lung cancer is a high N-Myc non-small cell lung cancer.

26. The method of claim 20, comprising orally administering the compound to the subject.

27. A method of degrading GSPT1 in a subject suffering from cancer, comprising administering to the subject a therapeutically effective amount of the compound of claim 1.

28. The method of claim 27, wherein the cancer is a Myc-driven cancer.

29. The method of claim 28, wherein the Myc-driven cancer is a Myc-driven lung cancer.

30. The method of claim 28, wherein the Myc-driven cancer is a Myc-driven small cell lung cancer.

31. The method of claim 30, wherein the Myc-driven small cell lung cancer is a high L-Myc small cell lung cancer.

32. The method of claim 27, wherein the Myc-driven cancer is a Myc-driven non-small cell lung cancer.

33. The method of claim 32, wherein the Myc-driven non-small cell lung cancer is a high N-Myc non-small cell lung cancer.

34. The method of claim 27, comprising orally administering the compound to the subject.

35. A method of reducing the level of GSPT1 in a subject suffering from cancer, comprising administering to the subject a therapeutically effective amount of the compound of claim 1.

36. The method of claim 35, wherein the cancer is a Myc-driven cancer.

37. The method of claim 36, wherein the Myc-driven cancer is a Myc-driven lung cancer.

38. The method of claim 36, wherein the Myc-driven cancer is a Myc-driven small cell lung cancer.

39. The method of claim 38, wherein the Myc-driven small cell lung cancer is a high L-Myc small cell lung cancer.

40. The method of claim 36, wherein the Myc-driven cancer is a Myc-driven non-small cell lung cancer.

41. The method of claim 40, wherein the Myc-driven non-small cell lung cancer is a high N-Myc non-small cell lung cancer.

42. The method of claim 35, comprising orally administering the compound to the subject.
